(19) United States
(12) Patent Application Publication
(10) Pub. No.: US 2024/0246923 A1
(43) Pub. Date: Jul. 25, 2024

(54) HETEROCYCLIC COMPOUND AND ORGANIC LIGHT-EMITTING DEVICE INCLUDING SAME

(71) Applicant: LT MATERIALS CO., LTD., Yongin-si, Gyeonggi-do (KR)

(72) Inventors: Su-Yeon KIM, Yongin-si (KR); GWANG-IL DONG, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/288,681

(22) PCT Filed: Nov. 19, 2021
(86) PCT No.: PCT/KR2021/017093

§ 371 Date: Oct. 27, 2023

(30) Foreign Application Priority Data
May 24, 2021 (KR) 10-2021-0066212

(51) Int. Cl. C07D 311/88; C07D 407/12; C07D 409/12; C07D 409/14; H10K 50/14; H10K 50/18; H10K 85/60

(52) U.S. Cl. CPC C07D 311/88; C07D 407/12; C07D 409/12; C07D 409/14; H10K 50/14; H10K 50/181; H10K 85/6574; H10K 85/6576

(57) ABSTRACT

The present specification relates to a heterocyclic compound of Chemical Formula 1, and an organic light emitting device including the same.

### TECHNICAL FIELD

The present specification relates to a heterocyclic compound, and an organic light emitting device including the same.

This application claims priority to and the benefits of Korean Patent Application No. 10-2021-0066212, filed with the Korean Intellectual Property Office on May 24, 2021, the entire contents of which are incorporated herein by reference.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### DISCLOSURE

### Technical Problem

The present specification is directed to providing a heterocyclic compound, and an organic light emitting device including the same.

### Technical Solution

One embodiment of the present specification provides a heterocyclic compound of the following Chemical Formula 1.

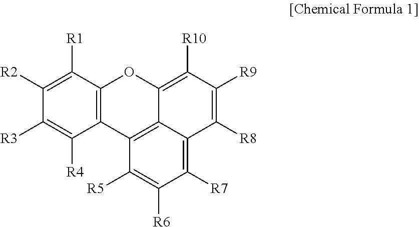

In Chemical Formula 1,
R1 to R10 are the same as or different from each other, and each independently hydrogen; deuterium; a halogen group; a cyano group; —Si(R11) (R12) (R13); —N(R14) (R15); a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R11 to R15 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
at least one of R1 to R10 is -(L)l-N(R24) (R25),
at least one of the rest of R1 to R10 is a substituted or unsubstituted C6 to C60 aryl group,
L is a direct bond; a substituted or unsubstituted C6 to C60 arylene group; or a substituted or unsubstituted C2 to C60 heteroarylene group,
l is an integer of 1 to 3, and when 1 is 2 or greater, Ls are the same as or different from each other, and
R24 and R25 are the same as or different from each other, and each independently a substituted or unsubstituted C6 to C60 aryl group; or a substituted or unsubstituted C2 to C60 heteroaryl group.

Another embodiment of the present specification provides an organic light emitting device including a first electrode; a second electrode; and one or more organic material layers provided between the first electrode and the second electrode, wherein one or more layers of the organic material layers include the heterocyclic compound of Chemical Formula 1.

### Advantageous Effects

A heterocyclic compound described in the present specification can be used as a material of an organic material layer of an organic light emitting device. The heterocyclic compound is capable of performing a role of a hole injection material, a hole transfer material, a light emitting material, an electron transfer material, an electron injection material, a hole blocking material, an electron blocking material, a charge generation material or the like in an organic light emitting device. Particularly, the compound can be used as a hole transfer material or an electron blocking material of an organic light emitting device.

When the heterocyclic compound of Chemical Formula 1 is included and used in a hole transfer layer or an electron blocking layer of an organic light emitting device, an organic light emitting device having excellent driving voltage and lifetime can be provided.

Specifically, in the heterocyclic compound of Chemical Formula 1, benzo[kl]xanthene is substituted with an amine group and an aryl group, which delocalizes a HOMO (highest occupied molecular orbital) energy level and thereby increases a hole transfer ability, and stabilizes the HOMO energy.

Accordingly, when using the heterocyclic compound of Chemical Formula 1 as a material of a hole transfer layer or an electron blocking layer in an organic light emitting device, proper energy level and band gap are formed increasing excitons in a light emitting region, and effects of lowering a driving voltage of the device, enhancing light efficiency, and increasing a lifetime of the device by thermal stability of the compound are obtained.

### DESCRIPTION OF DRAWINGS

FIG. 1 to FIG. 4 are diagrams each illustrating a lamination structure of an organic light emitting device according to one embodiment of the present specification.

### REFERENCE NUMERAL

100: Substrate
200: Anode
300: Organic Material Layer
301: Hole Injection Layer
302: Hole Transfer Layer
303: Electron Blocking Layer
304: Light Emitting Layer
305: Hole Blocking Layer
306: Electron Transfer Layer
307: Electron Injection Layer
400: Cathode

### MODE FOR DISCLOSURE

Hereinafter, the present specification will be described in more detail.

In the present specification, a description of a certain part “including” certain constituents means capable of further including other constituents, and does not exclude other constituents unless particularly stated on the contrary.

A term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or being substituted with a substituent linking two or more substituents selected from among the substituents illustrated above, or being unsubstituted.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or ²H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

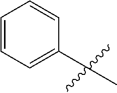 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

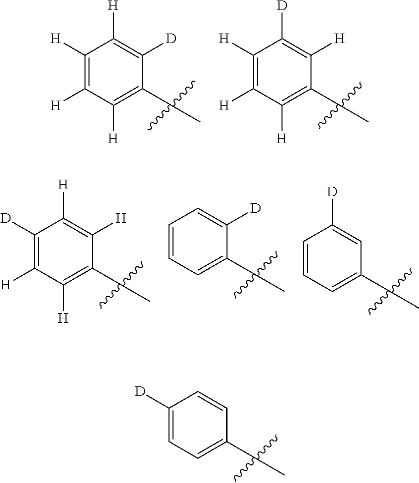 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not include a deuterium atom, that is, a phenyl group that has 5 hydrogen atoms.

In the present specification, the alkyl group includes linear or branched, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched, and may be further substituted with other substituents. The number of carbon atoms of the alkynyl group may be from 2 to 60, specifically from 2 to 40 and more specifically from 2 to 20.

In the present specification, the cycloalkyl group includes monocyclic or polycyclic, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. When the aryl group is dicyclic or higher, the number of carbon atoms may be from 8 to 60, from 8 to 40 or from 8 to 30. Specific examples of the aryl group may include a phenyl group, a biphenyl group, a ter-phenyl group, a naphthyl group, an anthryl group, a chrysene group, a phenanthrene group, a perylene group, a fluoranthene group, a triphenylene group, a phenalene group, a pyrene group, an anthracene group, a tetracene group, a pentacene group, a fluorene group, an indene group, an acenaphthylene group, a benzofluorene group, a spirobifluorene group, a 2,3-dihydro-1H-indene group, a fused ring group thereof, and the like, but are not limited thereto.

In the present specification, the fluorene group may be substituted, and adjacent substituents may bond to each other to form a ring.

When the fluorene group is substituted, the following structures may be included, however, the structure is not limited thereto.

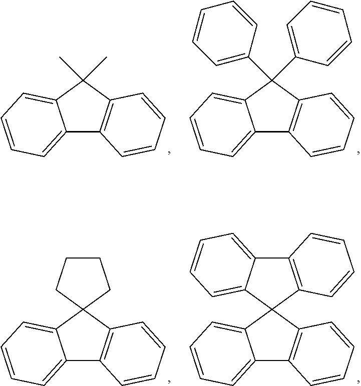

In the present specification, the ter-phenyl group includes linear or branched, and may be represented by the following structures.

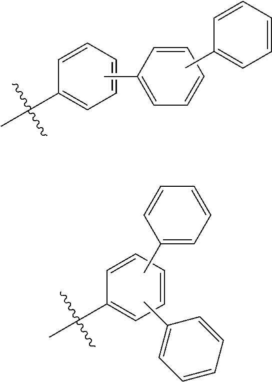

In the present specification, the heteroaryl group includes O, S, SO₂, Se, N or Si as a heteroatom, includes monocyclic or polycyclic, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. When the heteroaryl group is dicyclic or higher, the number of carbon atoms may be from 4 to 60, 4 to 40 or 4 to 25. Specific examples of the heteroaryl group may include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazole group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinoxaline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, an indolo[2,3-a]carbazole group, an indolo[2,3-b]carbazolyl group, an indoline group, a 10,11-dihydro-dibenzo[b,f]azepine group, a 9,10-dihydroacridine group, a phenanthrazine group, a phenothiazine group, a phthalazine group, a naphthylidine group, a phenanthroline group, a benzo[c][1,2,5]thiadiazole group, 5,10-dihydrodibenzo[b,e][1,4]azasilinyl, a pyrazolo[1,5-c]quinazoline group, a pyrido[1,2-b]indazole group, a pyrido[1,2-a]imidazo[1,2-e]indoline group, a benzofuro[2,3-d]pyrimidine group; a benzothieno[2,3-d]pyrimidine group; a benzofuro[2,3-a]carbazole group, a benzothieno[2,3-a]carbazole group, a 1,3-dihydroindolo[2,3-a]carbazole group, a benzofuro[3,2-a]carbazole group, a benzothieno[3,2-a]carbazole group, a 1,3-dihydroindolo[3,2-a]carbazole group, a benzofuro[2,3-b]carbazole group, a benzothieno[2,3-b]carbazole group, a 1,3-dihydroindolo[2,3-b]carbazole group, a benzofuro[3,2-b]carbazole group, a benzothieno[3,2-b]carbazole group, a 1,3-dihydroindolo[3,2-b]carbazole group, a benzofuro[2,3-c]carbazolyl group, a benzothieno[2,3-c]carbazole group, a 1,3-dihydroindolo[2,3-c]carbazole group, a benzofuro[3,2-c]carbazole group, a benzothieno[3,2-c]carbazole group, a 1,3-dihydroindolo[3,2-c]carbazole group, a 1,3-dihydroindeno[2,1-b]carbazole group, a 5,11-dihydroindeno[1,2-b]carbazole group, a 5,12-dihydroindeno[1,2-c]carbazole group, a 5,8-dihydroindeno[2,1-c]carbazole group, a 7,12-dihydroindeno[1,2-a]carbazole group, a 11,12-dihydroindeno[2,1-a]carbazole group and the like, but are not limited thereto.

In the present specification, the silyl group is a substituent including Si and having the Si atom directly linked as a radical, and is represented by —Si(R101) (R102) (R103). R101 to R103 are the same as or different from each other, and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group may include the following structures, but are not limited thereto.

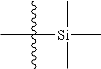 [SMILES: *[Si](C)(C)C]

(trimethylsilyl group),

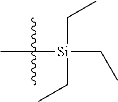 [SMILES: *[Si](CC)(CC)CC]

(triethylsilyl group),

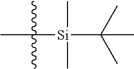 [SMILES: *[Si](C)(C)C(C)(C)C]

(t-butyldimethylsilyl group),

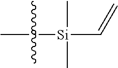

(vinyldimethylsilyl group),

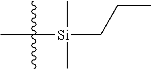 [SMILES: *[Si](C)(C)CCC]

(propyldimethylsilyl group

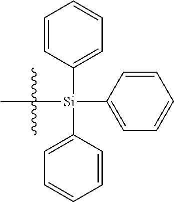 [SMILES: *[Si](c1ccccc1)(c1ccccc1)c1ccccc1]

(triphenylsilyl group),

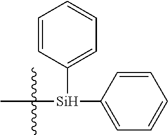

(diphenylsilyl group),

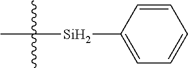

(phenylsilyl group)

In the present specification, the amine group is represented by —N(R104) (R105), and R104 and R105 are the same as or different from each other and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the phosphine oxide group is represented by —P(═O) (R106) (R107), and R106 and R107 are the same as or different from each other and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and as the alkyl group and the aryl group, the examples described above may be used. Examples of the phosphine oxide group may include a dimethylphosphine oxide group, a diphenylphosphine oxide group, a dinaphthylphosphine oxide group and the like, but are not limited thereto.

In the present specification, the descriptions on the aryl group provided above may be applied to an arylene group except that the arylene group is a divalent group.

In the present specification, a phenylene group may be selected from among the following structures, and may be further substituted with deuterium.

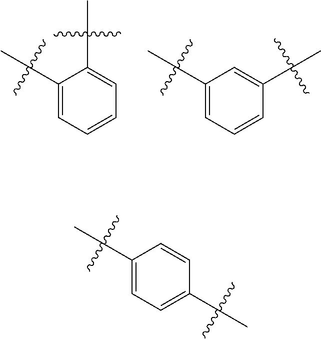 [SMILES: *c1ccccc1* ; *c1cccc(*)c1 ; *c1ccc(*)cc1]

In the present specification, the descriptions on the heteroaryl group provided above may be applied to a heteroarylene group except that the heteroarylene group is a divalent group.

One embodiment of the present specification provides a heterocyclic compound of Chemical Formula 1.

In one embodiment of the present specification, R1 to R10 are the same as or different from each other and each independently hydrogen; deuterium; a halogen group; a cyano group; —Si(R11) (R12) (R13); —N(R14) (R15); a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and R11 to R15 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In one embodiment of the present specification, R1 to R10 are the same as or different from each other and each independently hydrogen; deuterium; —N(R14) (R15); or a substituted or unsubstituted C6 to C60 aryl group, and R14 and R15 are each independently a substituted or unsubstituted C6 to C60 aryl group; or a substituted or unsubstituted C2 to C60 heteroaryl group.

In one embodiment of the present specification, R1 to R10 are the same as or different from each other and each independently hydrogen; deuterium; —N(R14) (R15); or a substituted or unsubstituted C6 to C30 aryl group, and R14 and R15 are each independently a substituted or unsubstituted C6 to C30 aryl group; or a substituted or unsubstituted C2 to C30 heteroaryl group.

In one embodiment of the present specification, R1 to R10 are the same as or different from each other and each independently hydrogen; deuterium; —N(R14) (R15); or a substituted or unsubstituted C6 to C20 aryl group, and R14 and R15 are each independently a substituted or unsubstituted C6 to C20 aryl group; or a substituted or unsubstituted C2 to C20 heteroaryl group.

In one embodiment of the present specification, R1 to R10 are the same as or different from each other and each independently hydrogen; deuterium; —N(R14) (R15); or a substituted or unsubstituted C6 to C20 aryl group, and R14 and R15 are each independently a substituted or unsubstituted C6 to C20 tricyclic or lower aryl group; or a C2 to C20 heteroaryl group substituted or unsubstituted and including 0 or S.

In one embodiment of the present specification, at least one of R1 to R10 is -(L)l-N(R24) (R25), and at least one of the rest of R1 to R10 is a substituted or unsubstituted C6 to C60 aryl group.

In one embodiment of the present specification, at least one of R1 to R10 is -(L)l-N(R24) (R25), and at least one of the rest of R1 to R10 is a substituted or unsubstituted C6 to C30 aryl group.

In one embodiment of the present specification, at least one of R1 to R10 is -(L)l-N(R24) (R25), and at least one of the rest of R1 to R10 is a substituted or unsubstituted C6 to C20 aryl group.

In one embodiment of the present specification, at least one of R1 to R10 is -(L)l-N(R24) (R25), and at least one of the rest of R1 to R10 may be a substituted or unsubstituted phenyl group; a substituted or unsubstituted biphenyl group; a substituted or unsubstituted ter-phenyl group; a substituted or unsubstituted naphthyl group; a substituted or unsubstituted phenanthrene group; a substituted or unsubstituted pyrene group; or a substituted or unsubstituted triphenylene group.

In one embodiment of the present specification, at least one of R1 to R10 is -(L)l-N(R24) (R25), and at least one of the rest of R1 to R10 is a C6 to C60 aryl group unsubstituted or substituted with deuterium.

In one embodiment of the present specification, at least one of R1 to R10 is -(L)l-N(R24) (R25), and at least one of the rest of R1 to R10 is a C6 to C30 aryl group unsubstituted or substituted with deuterium.

In one embodiment of the present specification, at least one of R1 to R10 is -(L)l-N(R24) (R25), and at least one of the rest of R1 to R10 is a C6 to C20 aryl group unsubstituted or substituted with deuterium.

In one embodiment of the present specification, at least one of R1 to R10 is -(L)l-N(R24) (R25), and at least one of the rest of R1 to R10 may be a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; a pyrene group unsubstituted or substituted with deuterium; or a triphenylene group unsubstituted or substituted with deuterium.

In one embodiment of the present specification, at least one of R1 to R10 is -(L)l-N(R24) (R25), and R24 and R25 are the same as or different from each other and each independently a substituted or unsubstituted C6 to C60 aryl group; or a substituted or unsubstituted C2 to C60 heteroaryl group.

In one embodiment of the present specification, at least one of R1 to R10 is -(L)l-N(R24) (R25), and R24 and R25 are the same as or different from each other and each independently a substituted or unsubstituted C6 to C30 aryl group; or a substituted or unsubstituted C2 to C30 heteroaryl group.

In one embodiment of the present specification, at least one of R1 to R10 is -(L)l-N(R24) (R25), and R24 and R25 are the same as or different from each other and each independently a substituted or unsubstituted C6 to C20 aryl group; or a substituted or unsubstituted C2 to C20 heteroaryl group.

In one embodiment of the present specification, at least one of R1 to R10 is -(L)l-N(R24) (R25), and R24 and R25 are the same as or different from each other and each independently a substituted or unsubstituted C6 to C20 tricyclic or lower aryl group; a C2 to C20 heteroaryl group substituted or unsubstituted and including 0 or S; or a C2 to C20 heteroaryl group substituted or unsubstituted and including a C═N bond.

In one embodiment of the present specification, at least one of R1 to R10 is -(L)l-N(R24) (R25), and R24 and R25 are the same as or different from each other and each independently a substituted or unsubstituted C6 to C20 tricyclic or lower aryl group; or a C2 to C20 heteroaryl group substituted or unsubstituted and including 0 or S.

In one embodiment of the present specification, at least one of R1 to R10 is -(L)l-N(R24) (R25), and R24 and R25 are the same as or different from each other and may be each independently a substituted or unsubstituted phenyl group; a substituted or unsubstituted biphenyl group; a substituted or unsubstituted ter-phenyl group; a substituted or unsubstituted naphthyl group; a substituted or unsubstituted phenanthrene group; a substituted or unsubstituted fluorene group; a substituted or unsubstituted dibenzofuran group; or a substituted or unsubstituted dibenzothiophene group.

In one embodiment of the present specification, at least one of R1 to R10 is -(L)l-N(R24) (R25), and R24 and R25 are the same as or different from each other and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; a fluorene group unsubstituted or substituted with one or more substituents selected from among deuterium, an alkyl group, and an aryl group unsubstituted or substituted with deuterium; a dibenzofuran group unsubstituted or substituted with one or more substituents selected from among deuterium and an aryl group; or a dibenzothiophene group unsubstituted or substituted with one or more substituents selected from among deuterium and an aryl group.

In one embodiment of the present specification, at least one of R1 to R10 is -(L)l-N(R24) (R25), and R24 and R25 are the same as or different from each other and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; a fluorene group unsubstituted or substituted with one or more substituents selected from among deuterium, an alkyl group and an aryl group; a dibenzofuran group unsubstituted or substituted with one or more substituents selected from among deuterium and an aryl group; or a dibenzothiophene group unsubstituted or substituted with one or more substituents selected from among deuterium and an aryl group.

In one embodiment of the present specification, at least one of R24 and R25 may be a substituted or unsubstituted C10 to C20 tricyclic or lower aryl group; or a C2 to C20 heteroaryl group substituted or unsubstituted and including 0 or S.

In one embodiment of the present specification, at least one of R24 and R25 may be a substituted or unsubstituted biphenyl group; a substituted or unsubstituted ter-phenyl group; a substituted or unsubstituted naphthyl group; a substituted or unsubstituted phenanthrene group; a substituted or unsubstituted fluorene group; a substituted or unsubstituted dibenzofuran group; or a substituted or unsubstituted dibenzothiophene group.

In one embodiment of the present specification, -(L)l-N(R24) (R25) may be represented by any one of the following Chemical Formulae N-1 to N-3.

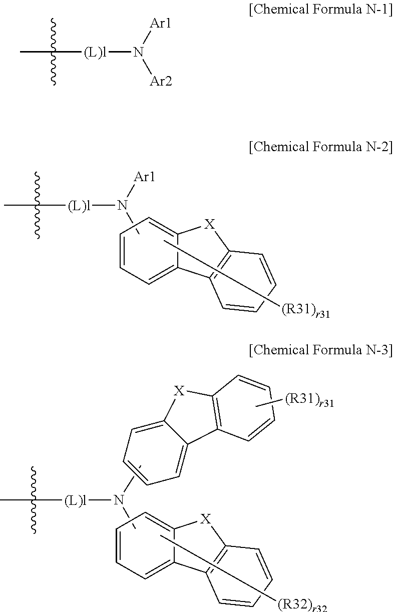

In Chemical Formulae N-1 to N-3,
L and 1 have the same definitions as in Chemical Formula 1,
X and Y are each independently 0; or S,
Ar1 and Ar2 are the same as or different from each other, and each independently a substituted or unsubstituted C6 to C60 aryl group,
R31 and R32 are each independently hydrogen; deuterium; or a substituted or unsubstituted C6 to C30 aryl group, and
r31 and r32 are each an integer of 0 to 7, and when r31 and r32 are each 2 or greater, substituents in the parentheses are the same as or different from each other.

In one embodiment of the present specification, L is a direct bond; a substituted or unsubstituted C6 to C60 arylene group; or a substituted or unsubstituted C2 to C60 heteroarylene group.

In one embodiment of the present specification, L is a direct bond; or a substituted or unsubstituted C6 to C60 arylene group.

In one embodiment of the present specification, L is a direct bond; or a substituted or unsubstituted C6 to C30 arylene group.

In one embodiment of the present specification, L is a direct bond; or a substituted or unsubstituted C6 to C15 arylene group.

In one embodiment of the present specification, L may be a direct bond; or a substituted or unsubstituted phenylene group.

In one embodiment of the present specification, L may be a direct bond; or a phenylene group unsubstituted or substituted with deuterium.

In one embodiment of the present specification, 1 is an integer of 1 to 3.

In one embodiment of the present specification, 1 may be 1.

In one embodiment of the present specification, Ar1 and Ar2 in Chemical Formula N-1 are the same as or different from each other, and each independently a substituted or unsubstituted C6 to C60 aryl group.

In one embodiment of the present specification, Ar1 and Ar2 in Chemical Formula N-1 are the same as or different from each other, and each independently a substituted or unsubstituted C6 to C30 aryl group.

In one embodiment of the present specification, Ar1 and Ar2 in Chemical Formula N-1 are the same as or different from each other, and each independently a substituted or unsubstituted C6 to C20 aryl group.

In one embodiment of the present specification, Ar1 and Ar2 in Chemical Formula N-1 are the same as or different from each other, and may be each independently a substituted or unsubstituted C6 to C20 tricyclic or lower aryl group.

In one embodiment of the present specification, Ar1 and Ar2 in Chemical Formula N-1 are the same as or different from each other, and may be each independently a substituted or unsubstituted phenyl group; a substituted or unsubstituted biphenyl group; a substituted or unsubstituted ter-phenyl group; a substituted or unsubstituted naphthyl group; a substituted or unsubstituted phenanthrene group; or a substituted or unsubstituted fluorene group.

In one embodiment of the present specification, Ar1 and Ar2 in Chemical Formula N-1 are the same as or different from each other, and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; or a fluorene group unsubstituted or substituted with one or more substituents selected from among deuterium, an alkyl group, and an aryl group unsubstituted or substituted with deuterium.

In one embodiment of the present specification, Ar1 and Ar2 in Chemical Formula N-1 are the same as or different from each other, and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; or a fluorene group unsubstituted or substituted with one or more substituents selected from among deuterium, an alkyl group and an aryl group.

In one embodiment of the present specification, Ar2 in Chemical Formula N-1 may be a substituted or unsubstituted C10 to C20 tricyclic or lower aryl group.

In one embodiment of the present specification, Ar2 in Chemical Formula N-1 may be a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; or a fluorene group unsubstituted or substituted with one or more substituents selected from among deuterium, an alkyl group, and an aryl group unsubstituted or substituted with deuterium.

In one embodiment of the present specification, Ar2 in Chemical Formula N-1 may be a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; or a fluorene group unsubstituted or substituted with one or more substituents selected from among deuterium, an alkyl group and an aryl group.

In one embodiment of the present specification, descriptions on Ar1 in Chemical Formula N-2 are the same as the descriptions on Ar1 in Chemical Formula N-1.

In one embodiment of the present specification, R31 in Chemical Formula N-2 is hydrogen; deuterium; or a substituted or unsubstituted C6 to C30 aryl group.

In one embodiment of the present specification, R31 in Chemical Formula N-2 is hydrogen; deuterium; or a substituted or unsubstituted phenyl group.

In one embodiment of the present specification, R31 in Chemical Formula N-2 is hydrogen; deuterium; or a phenyl group unsubstituted or substituted with deuterium.

In one embodiment of the present specification, descriptions on R31 in Chemical Formula N-3 are the same as the descriptions on R31 in Chemical Formula N-2.

In one embodiment of the present specification, R32 in Chemical Formula N-3 is hydrogen; deuterium; or a substituted or unsubstituted C6 to C30 aryl group.

In one embodiment of the present specification, R32 in Chemical Formula N-3 is hydrogen; deuterium; or a substituted or unsubstituted phenyl group.

In one embodiment of the present specification, R32 in Chemical Formula N-3 is hydrogen; deuterium; or a phenyl group unsubstituted or substituted with deuterium.

In one embodiment of the present specification, X and Y in Chemical Formulae N-2 and N-3 are each independently 0; or S.

In one embodiment of the present specification, X and Y in Chemical Formulae N-2 and N-3 are different from each other.

In one embodiment of the present specification, when X is O in Chemical Formula N-3, Y is S.

In one embodiment of the present specification, when X is S in Chemical Formula N-3, Y is O.

In one embodiment of the present specification, any one of R1 to R10 is -(L)l-N(R24) (R25), another one of R1 to R10 is a C6 to C60 aryl group unsubstituted or substituted with one or more substituents selected from among deuterium, an alkyl group, and an aryl group unsubstituted or substituted with deuterium, and the rest of R1 to R10 are hydrogen; or deuterium.

In one embodiment of the present specification, any one of R1 to R10 is -(L)l-N(R24) (R25), another one of R1 to R10 is a C6 to C30 aryl group unsubstituted or substituted with one or more substituents selected from among deuterium, an alkyl group, and an aryl group unsubstituted or substituted with deuterium, and the rest of R1 to R10 are hydrogen; or deuterium.

In one embodiment of the present specification, any one of R1 to R10 is -(L)l-N(R24) (R25), another one of R1 to R10 is a C6 to C60 aryl group unsubstituted or substituted with deuterium, and the rest of R1 to R10 are hydrogen; or deuterium.

In one embodiment of the present specification, any one of R1 to R10 is -(L)l-N(R24) (R25), another one of R1 to R10 is a C6 to C30 aryl group unsubstituted or substituted with deuterium, and the rest of R1 to R10 are hydrogen; or deuterium.

In one embodiment of the present specification, any one of R1 to R10 is -(L)l-N(R24) (R25), another one of R1 to R10 is a C6 to C20 aryl group unsubstituted or substituted with deuterium, and the rest of R1 to R10 are hydrogen; or deuterium.

In one embodiment of the present specification, Chemical Formula 1 may be represented by any one of the following Chemical Formulae 1-1 to 1-4.

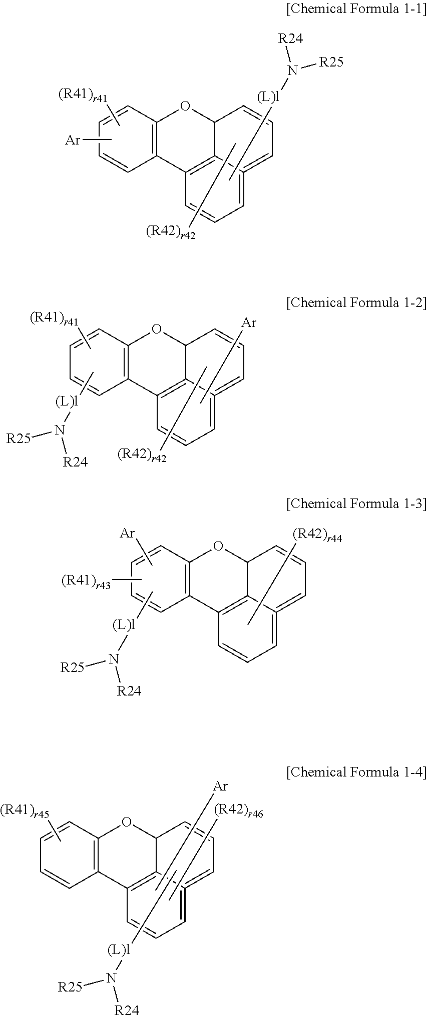

In Chemical Formulae 1-1 to 1-4,
L, 1, R24 and R25 have the same definitions as in Chemical Formula 1,
Ar is a substituted or unsubstituted C6 to C60 aryl group,
R41 and R42 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
r41 is an integer of 0 to 3, r42 is an integer of 0 to 5, r43 is an integer of 0 to 2, r44 is an integer of 0 to 6, and r45 and r46 are each an integer of 0 to 4, and
when r41, r42 and r44 to r46 are 2 or greater or r43 is 2, substituents in the parentheses are the same as or different from each other.

In one embodiment of the present specification, Ar is a substituted or unsubstituted C6 to C60 aryl group.

In one embodiment of the present specification, Ar is a substituted or unsubstituted C6 to C30 aryl group.

In one embodiment of the present specification, Ar is a substituted or unsubstituted C6 to C20 aryl group.

In one embodiment of the present specification, Ar may be a substituted or unsubstituted phenyl group; a substituted or unsubstituted biphenyl group; a substituted or unsubstituted ter-phenyl group; a substituted or unsubstituted naphthyl group; a substituted or unsubstituted phenanthrene group; a substituted or unsubstituted pyrene group; or a substituted or unsubstituted triphenylene group.

In one embodiment of the present specification, Ar is a C6 to C60 aryl group unsubstituted or substituted with deuterium.

In one embodiment of the present specification, Ar is a C6 to C30 aryl group unsubstituted or substituted with deuterium.

In one embodiment of the present specification, Ar is a C6 to C20 aryl group unsubstituted or substituted with deuterium.

In one embodiment of the present specification, Ar may be a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; a phenanthrene group unsubstituted or substituted with deuterium; a pyrene group unsubstituted or substituted with deuterium; or a triphenylene group unsubstituted or substituted with deuterium.

In one embodiment of the present specification, R41 and R42 are each independently hydrogen; deuterium; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In one embodiment of the present specification, R41 and R42 are each independently hydrogen; deuterium; a substituted or unsubstituted C6 to C30 aryl group; or a substituted or unsubstituted C2 to C30 heteroaryl group.

In one embodiment of the present specification, R41 and R42 are each independently hydrogen; deuterium; a substituted or unsubstituted C6 to C20 aryl group; or a substituted or unsubstituted C2 to C20 heteroaryl group.

In one embodiment of the present specification, R41 and R42 are each independently hydrogen; or deuterium.

In one embodiment of the present specification, Chemical Formula 1 may be represented by Chemical Formula 1-1.

In one embodiment of the present specification, Chemical Formula 1 may be represented by Chemical Formula 1-2.

In one embodiment of the present specification, Chemical Formula 1 may be represented by Chemical Formula 1-3.

In one embodiment of the present specification, Chemical Formula 1 may be represented by Chemical Formula 1-4.

In one embodiment of the present specification, R1 is -(L)l-N(R24) (R25), and R2 is a substituted or unsubstituted C6 to C60 aryl group.

In one embodiment of the present specification, R1 is -(L)l-N(R24) (R25), and R3 is a substituted or unsubstituted C6 to C60 aryl group.

In one embodiment of the present specification, R1 is -(L)l-N(R24) (R25), and R4 is a substituted or unsubstituted C6 to C60 aryl group.

In one embodiment of the present specification, R1 is -(L)l-N(R24) (R25), and R5 is a substituted or unsubstituted C6 to C60 aryl group.

In one embodiment of the present specification, R1 is -(L)l-N(R24) (R25), and R6 is a substituted or unsubstituted C6 to C60 aryl group.

In one embodiment of the present specification, R1 is -(L) l-N(R24) (R25), and R7 is a substituted or unsubstituted C6 to C60 aryl group.

In one embodiment of the present specification, R1 is -(L) l-N(R24) (R25), and R8 is a substituted or unsubstituted C6 to C60 aryl group.

In one embodiment of the present specification, R1 is -(L) l-N(R24) (R25), and R9 is a substituted or unsubstituted C6 to C60 aryl group.

In one embodiment of the present specification, R1 is -(L) l-N(R24) (R25), and R10 is a substituted or unsubstituted C6 to C60 aryl group.

In one embodiment of the present specification, R2 is -(L) l-N(R24) (R25), and R1 is a substituted or unsubstituted C6 to C60 aryl group.

In one embodiment of the present specification, R2 is -(L) l-N(R24) (R25), and R3 is a substituted or unsubstituted C6 to C60 aryl group.

In one embodiment of the present specification, R2 is -(L) l-N(R24) (R25), and R4 is a substituted or unsubstituted C6 to C60 aryl group.

In one embodiment of the present specification, R2 is -(L) l-N(R24) (R25), and R5 is a substituted or unsubstituted C6 to C60 aryl group.

In one embodiment of the present specification, R2 is -(L) l-N(R24) (R25), and R6 is a substituted or unsubstituted C6 to C60 aryl group.

In one embodiment of the present specification, R2 is -(L) l-N(R24) (R25), and R7 is a substituted or unsubstituted C6 to C60 aryl group.

In one embodiment of the present specification, R2 is -(L) l-N(R24) (R25), and R8 is a substituted or unsubstituted C6 to C60 aryl group.

In one embodiment of the present specification, R2 is -(L) l-N(R24) (R25), and R9 is a substituted or unsubstituted C6 to C60 aryl group.

In one embodiment of the present specification, R2 is -(L) l-N(R24) (R25), and R10 is a substituted or unsubstituted C6 to C60 aryl group.

In one embodiment of the present specification, R3 is -(L) l-N(R24) (R25), and R1 is a substituted or unsubstituted C6 to C60 aryl group.

In one embodiment of the present specification, R3 is -(L) l-N(R24) (R25), and R2 is a substituted or unsubstituted C6 to C60 aryl group.

In one embodiment of the present specification, R3 is -(L) l-N(R24) (R25), and R4 is a substituted or unsubstituted C6 to C60 aryl group.

In one embodiment of the present specification, R3 is -(L) l-N(R24) (R25), and R5 is a substituted or unsubstituted C6 to C60 aryl group.

In one embodiment of the present specification, R3 is -(L) l-N(R24) (R25), and R6 is a substituted or unsubstituted C6 to C60 aryl group.

In one embodiment of the present specification, R3 is -(L) l-N(R24) (R25), and R7 is a substituted or unsubstituted C6 to C60 aryl group.

In one embodiment of the present specification, R3 is -(L) l-N(R24) (R25), and R8 is a substituted or unsubstituted C6 to C60 aryl group.

In one embodiment of the present specification, R3 is -(L) l-N(R24) (R25), and R9 is a substituted or unsubstituted C6 to C60 aryl group.

In one embodiment of the present specification, R3 is -(L) l-N(R24) (R25), and R10 is a substituted or unsubstituted C6 to C60 aryl group.

In one embodiment of the present specification, R4 is -(L) l-N(R24) (R25), and R1 is a substituted or unsubstituted C6 to C60 aryl group.

In one embodiment of the present specification, R4 is -(L) l-N(R24) (R25), and R2 is a substituted or unsubstituted C6 to C60 aryl group.

In one embodiment of the present specification, R4 is -(L) l-N(R24) (R25), and R3 is a substituted or unsubstituted C6 to C60 aryl group.

In one embodiment of the present specification, R4 is -(L) l-N(R24) (R25), and R5 is a substituted or unsubstituted C6 to C60 aryl group.

In one embodiment of the present specification, R4 is -(L) l-N(R24) (R25), and R6 is a substituted or unsubstituted C6 to C60 aryl group.

In one embodiment of the present specification, R4 is -(L) l-N(R24) (R25), and R7 is a substituted or unsubstituted C6 to C60 aryl group.

In one embodiment of the present specification, R4 is -(L) l-N(R24) (R25), and R8 is a substituted or unsubstituted C6 to C60 aryl group.

In one embodiment of the present specification, R4 is -(L) l-N(R24) (R25), and R9 is a substituted or unsubstituted C6 to C60 aryl group.

In one embodiment of the present specification, R4 is -(L) l-N(R24) (R25), and R10 is a substituted or unsubstituted C6 to C60 aryl group.

In one embodiment of the present specification, R5 is -(L) l-N(R24) (R25), and R1 is a substituted or unsubstituted C6 to C60 aryl group.

In one embodiment of the present specification, R5 is -(L) l-N(R24) (R25), and R2 is a substituted or unsubstituted C6 to C60 aryl group.

In one embodiment of the present specification, R5 is -(L) l-N(R24) (R25), and R3 is a substituted or unsubstituted C6 to C60 aryl group.

In one embodiment of the present specification, R5 is -(L) l-N(R24) (R25), and R4 is a substituted or unsubstituted C6 to C60 aryl group.

In one embodiment of the present specification, R5 is -(L) l-N(R24) (R25), and R6 is a substituted or unsubstituted C6 to C60 aryl group.

In one embodiment of the present specification, R5 is -(L) l-N(R24) (R25), and R7 is a substituted or unsubstituted C6 to C60 aryl group.

In one embodiment of the present specification, R5 is -(L) l-N(R24) (R25), and R8 is a substituted or unsubstituted C6 to C60 aryl group.

In one embodiment of the present specification, R5 is -(L) l-N(R24) (R25), and R9 is a substituted or unsubstituted C6 to C60 aryl group.

In one embodiment of the present specification, R5 is -(L) l-N(R24) (R25), and R10 is a substituted or unsubstituted C6 to C60 aryl group.

In one embodiment of the present specification, R6 is -(L) l-N(R24) (R25), and R1 is a substituted or unsubstituted C6 to C60 aryl group.

In one embodiment of the present specification, R6 is -(L) l-N(R24) (R25), and R2 is a substituted or unsubstituted C6 to C60 aryl group.

In one embodiment of the present specification, R6 is -(L) l-N(R24) (R25), and R3 is a substituted or unsubstituted C6 to C60 aryl group.

In one embodiment of the present specification, R6 is -(L) l-N(R24) (R25), and R4 is a substituted or unsubstituted C6 to C60 aryl group.

In one embodiment of the present specification, R6 is -(L) l-N(R24) (R25), and R5 is a substituted or unsubstituted C6 to C60 aryl group.

In one embodiment of the present specification, R6 is -(L) l-N(R24) (R25), and R7 is a substituted or unsubstituted C6 to C60 aryl group.

In one embodiment of the present specification, R6 is -(L) l-N(R24) (R25), and R8 is a substituted or unsubstituted C6 to C60 aryl group.

In one embodiment of the present specification, R6 is -(L) l-N(R24) (R25), and R9 is a substituted or unsubstituted C6 to C60 aryl group.

In one embodiment of the present specification, R6 is -(L) l-N(R24) (R25), and R10 is a substituted or unsubstituted C6 to C60 aryl group.

In one embodiment of the present specification, R7 is -(L) l-N(R24) (R25), and R1 is a substituted or unsubstituted C6 to C60 aryl group.

In one embodiment of the present specification, R7 is -(L) l-N(R24) (R25), and R2 is a substituted or unsubstituted C6 to C60 aryl group.

In one embodiment of the present specification, R7 is -(L) l-N(R24) (R25), and R3 is a substituted or unsubstituted C6 to C60 aryl group.

In one embodiment of the present specification, R7 is -(L) l-N(R24) (R25), and R4 is a substituted or unsubstituted C6 to C60 aryl group.

In one embodiment of the present specification, R7 is -(L) l-N(R24) (R25), and R5 is a substituted or unsubstituted C6 to C60 aryl group.

In one embodiment of the present specification, R7 is -(L) l-N(R24) (R25), and R6 is a substituted or unsubstituted C6 to C60 aryl group.

In one embodiment of the present specification, R7 is -(L) l-N(R24) (R25), and R8 is a substituted or unsubstituted C6 to C60 aryl group.

In one embodiment of the present specification, R7 is -(L) l-N(R24) (R25), and R9 is a substituted or unsubstituted C6 to C60 aryl group.

In one embodiment of the present specification, R7 is -(L) l-N(R24) (R25), and R10 is a substituted or unsubstituted C6 to C60 aryl group.

In one embodiment of the present specification, R8 is -(L) l-N(R24) (R25), and R1 is a substituted or unsubstituted C6 to C60 aryl group.

In one embodiment of the present specification, R8 is -(L) l-N(R24) (R25), and R2 is a substituted or unsubstituted C6 to C60 aryl group.

In one embodiment of the present specification, R8 is -(L) l-N(R24) (R25), and R3 is a substituted or unsubstituted C6 to C60 aryl group.

In one embodiment of the present specification, R8 is -(L) l-N(R24) (R25), and R4 is a substituted or unsubstituted C6 to C60 aryl group.

In one embodiment of the present specification, R8 is -(L) l-N(R24) (R25), and R5 is a substituted or unsubstituted C6 to C60 aryl group.

In one embodiment of the present specification, R8 is -(L) l-N(R24) (R25), and R6 is a substituted or unsubstituted C6 to C60 aryl group.

In one embodiment of the present specification, R8 is -(L) l-N(R24) (R25), and R7 is a substituted or unsubstituted C6 to C60 aryl group.

In one embodiment of the present specification, R8 is -(L) l-N(R24) (R25), and R9 is a substituted or unsubstituted C6 to C60 aryl group.

In one embodiment of the present specification, R8 is -(L) l-N(R24) (R25), and R10 is a substituted or unsubstituted C6 to C60 aryl group.

In one embodiment of the present specification, R9 is -(L) l-N(R24) (R25), and R1 is a substituted or unsubstituted C6 to C60 aryl group.

In one embodiment of the present specification, R9 is -(L) l-N(R24) (R25), and R2 is a substituted or unsubstituted C6 to C60 aryl group.

In one embodiment of the present specification, R9 is -(L) l-N(R24) (R25), and R3 is a substituted or unsubstituted C6 to C60 aryl group.

In one embodiment of the present specification, R9 is -(L) l-N(R24) (R25), and R4 is a substituted or unsubstituted C6 to C60 aryl group.

In one embodiment of the present specification, R9 is -(L) l-N(R24) (R25), and R5 is a substituted or unsubstituted C6 to C60 aryl group.

In one embodiment of the present specification, R9 is -(L) l-N(R24) (R25), and R6 is a substituted or unsubstituted C6 to C60 aryl group.

In one embodiment of the present specification, R9 is -(L) l-N(R24) (R25), and R7 is a substituted or unsubstituted C6 to C60 aryl group.

In one embodiment of the present specification, R9 is -(L) l-N(R24) (R25), and R8 is a substituted or unsubstituted C6 to C60 aryl group.

In one embodiment of the present specification, R9 is -(L) l-N(R24) (R25), and R10 is a substituted or unsubstituted C6 to C60 aryl group.

In one embodiment of the present specification, R10 is -(L) l-N(R24) (R25), and R1 is a substituted or unsubstituted C6 to C60 aryl group.

In one embodiment of the present specification, R10 is -(L) l-N(R24) (R25), and R2 is a substituted or unsubstituted C6 to C60 aryl group.

In one embodiment of the present specification, R10 is -(L) l-N(R24) (R25), and R3 is a substituted or unsubstituted C6 to C60 aryl group.

In one embodiment of the present specification, R10 is -(L) l-N(R24) (R25), and R4 is a substituted or unsubstituted C6 to C60 aryl group.

In one embodiment of the present specification, R10 is -(L) l-N(R24) (R25), and R5 is a substituted or unsubstituted C6 to C60 aryl group.

In one embodiment of the present specification, R10 is -(L) l-N(R24) (R25), and R6 is a substituted or unsubstituted C6 to C60 aryl group.

In one embodiment of the present specification, R10 is -(L) l-N(R24) (R25), and R7 is a substituted or unsubstituted C6 to C60 aryl group.

In one embodiment of the present specification, R10 is -(L)l-N(R24) (R25), and R8 is a substituted or unsubstituted C6 to C60 aryl group.

In one embodiment of the present specification, R10 is -(L)l-N(R24) (R25), and R9 is a substituted or unsubstituted C6 to C60 aryl group.

In one embodiment of the present specification, Chemical Formula 1 may have a deuterium content of 0% to 100%.

In one embodiment of the present specification, Chemical Formula 1 may have a deuterium content of 0%.

In one embodiment of the present specification, Chemical Formula 1 may have a deuterium content of greater than 0% and less than or equal to 100%.

In one embodiment of the present specification, Chemical Formula 1 may have a deuterium content of 10% to 100%.

In one embodiment of the present specification, Chemical Formula 1 may have a deuterium content of 20% to 100%, 30% to 100%, or 40% to 100%.

In one embodiment of the present specification, Chemical Formula 1 may have a deuterium content of either 0%, or 10% to 100%.

In one embodiment of the present application, the deuterium content in the heterocyclic compound of Chemical Formula 1 satisfies the above-mentioned range, and although the compound not including deuterium and the compound including deuterium have almost similar photochemical properties, the material including deuterium tends to be packed with narrower intermolecular distances when deposited on a thin film.

Accordingly, when manufacturing an EOD (electron only device) and a HOD (hole only device) and checking voltage-dependent current density, it is identified that, among the heterocyclic compounds of Chemical Formula 1 according to the present application, the compound including deuterium exhibits far more balanced charge transfer properties compared to the compound not including deuterium.

In addition, when examining the thin film surface using an atomic force microscope (AFM), it is identified that the thin film manufactured using the compound including deuterium is deposited as a more uniform surface with no aggregated places.

In addition, single bond dissociation energy of carbon and deuterium is higher than single bond dissociation energy of carbon and hydrogen, and as the deuterium content satisfies the above-mentioned range in the heterocyclic compound of Chemical Formula 1 according to the present application, an effect of improving a device lifetime is obtained by increasing stability of the whole molecule.

In one embodiment of the present specification, Chemical Formula 1 may be represented by any one of the following compounds, but is not limited thereto.

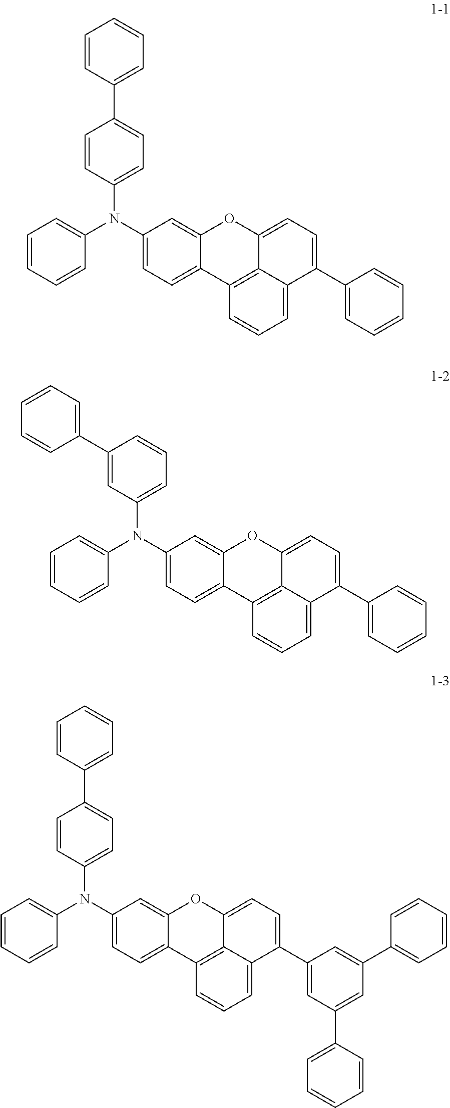 [SMILES: c1ccc(-c2ccc(N(c3ccccc3)c3ccc4c(c3)Oc3ccc(-c5ccccc5)c5cccc-4c35)cc2)cc1 ; c1ccc(-c2cccc(N(c3ccccc3)c3ccc4c(c3)Oc3ccc(-c5ccccc5)c5cccc-4c35)c2)cc1 ; c1ccc(-c2ccc(N(c3ccccc3)c3ccc4c(c3)Oc3ccc(-c5cc(-c6ccccc6)cc(-c6ccccc6)c5)c5cccc-4c35)cc2)cc1]

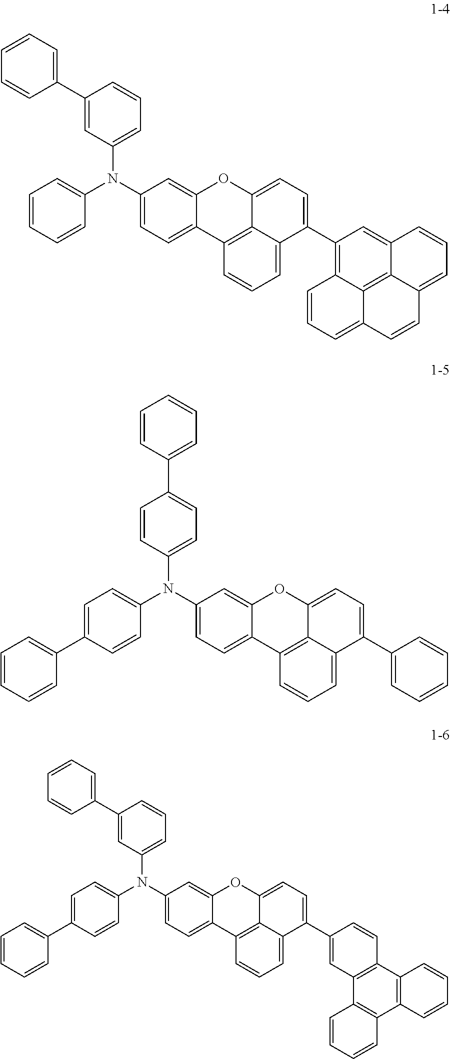 [SMILES: c1ccc(-c2cccc(N(c3ccccc3)c3ccc4c(c3)Oc3ccc(-c5cc6cccc7ccc8cccc5c8c76)c5cccc-4c35)c2)cc1 ; c1ccc(-c2ccc(N(c3ccc(-c4ccccc4)cc3)c3ccc4c(c3)Oc3ccc(-c5ccccc5)c5cccc-4c35)cc2)cc1 ; c1ccc(-c2ccc(N(c3cccc(-c4ccccc4)c3)c3ccc4c(c3)Oc3ccc(-c5ccc6c7ccccc7c7ccccc7c6c5)c5cccc-4c35)cc2)cc1]

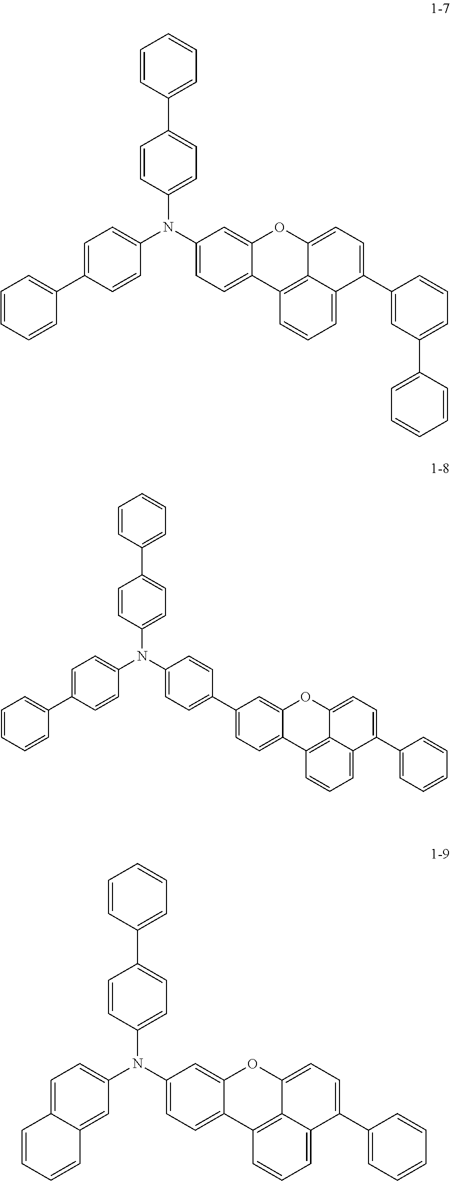 [SMILES: c1ccc(-c2ccc(N(c3ccc(-c4ccccc4)cc3)c3ccc4c(c3)Oc3ccc(-c5cccc(-c6ccccc6)c5)c5cccc-4c35)cc2)cc1 ; c1ccc(-c2ccc(N(c3ccc(-c4ccccc4)cc3)c3ccc(-c4ccc5c(c4)Oc4ccc(-c6ccccc6)c6cccc-5c46)cc3)cc2)cc1 ; c1ccc(-c2ccc(N(c3ccc4c(c3)Oc3ccc(-c5ccccc5)c5cccc-4c35)c3ccc4ccccc4c3)cc2)cc1]

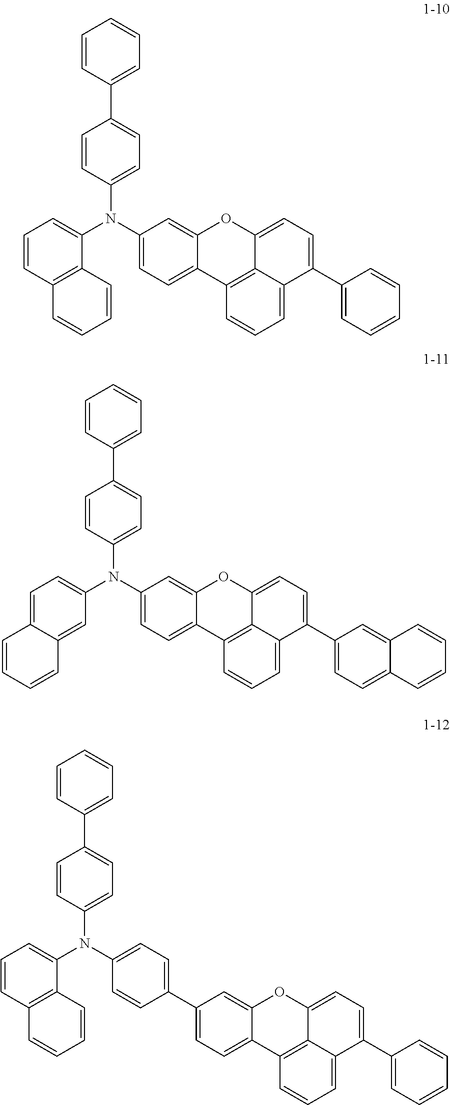 [SMILES: c1ccc(-c2ccc(N(c3ccc4c(c3)Oc3ccc(-c5ccccc5)c5cccc-4c35)c3cccc4ccccc34)cc2)cc1 ; c1ccc(-c2ccc(N(c3ccc4c(c3)Oc3ccc(-c5ccc6ccccc6c5)c5cccc-4c35)c3ccc4ccccc4c3)cc2)cc1 ; c1ccc(-c2ccc(N(c3ccc(-c4ccc5c(c4)Oc4ccc(-c6ccccc6)c6cccc-5c46)cc3)c3cccc4ccccc34)cc2)cc1]

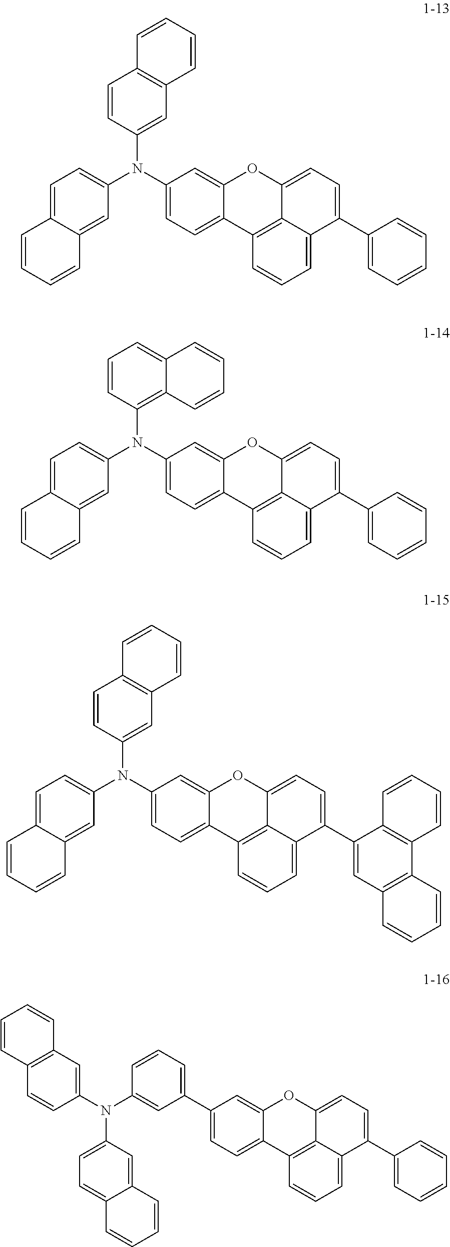 [SMILES: c1ccc(-c2ccc3c4c(cccc24)-c2ccc(N(c4ccc5ccccc5c4)c4ccc5ccccc5c4)cc2O3)cc1 ; c1ccc(-c2ccc3c4c(cccc24)-c2ccc(N(c4ccc5ccccc5c4)c4cccc5ccccc45)cc2O3)cc1 ; c1ccc2cc(N(c3ccc4c(c3)Oc3ccc(-c5cc6ccccc6c6ccccc56)c5cccc-4c35)c3ccc4ccccc4c3)ccc2c1 ; c1ccc(-c2ccc3c4c(cccc24)-c2ccc(-c4cccc(N(c5ccc6ccccc6c5)c5ccc6ccccc6c5)c4)cc2O3)cc1]

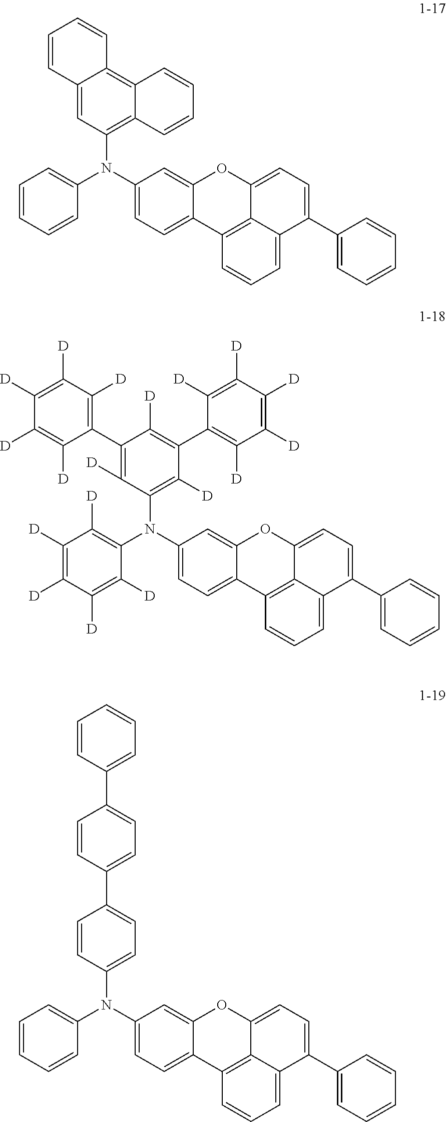 [SMILES: c1ccc(-c2ccc3c4c(cccc24)-c2ccc(N(c4ccccc4)c4cc5ccccc5c5ccccc45)cc2O3)cc1 ; [2H]c1c([2H])c([2H])c(-c2c([2H])c(-c3c([2H])c([2H])c([2H])c([2H])c3[2H])c([2H])c(N(c3ccc4c(c3)Oc3ccc(-c5ccccc5)c5cccc-4c35)c3c([2H])c([2H])c([2H])c([2H])c3[2H])c2[2H])c([2H])c1[2H] ; c1ccc(-c2ccc(-c3ccc(N(c4ccccc4)c4ccc5c(c4)Oc4ccc(-c6ccccc6)c6cccc-5c46)cc3)cc2)cc1]

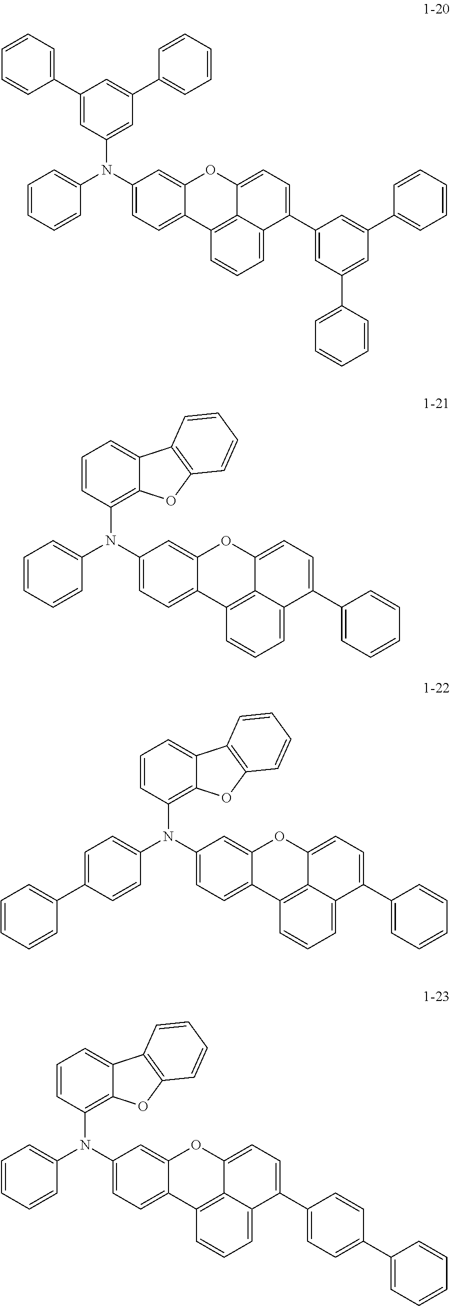 [SMILES: c1ccc(-c2cc(-c3ccccc3)cc(-c3ccc4c5c(cccc35)-c3ccc(N(c5ccccc5)c5cc(-c6ccccc6)cc(-c6ccccc6)c5)cc3O4)c2)cc1 ; c1ccc(-c2ccc3c4c(cccc24)-c2ccc(N(c4ccccc4)c4cccc5c4oc4ccccc45)cc2O3)cc1 ; c1ccc(-c2ccc(N(c3ccc4c(c3)Oc3ccc(-c5ccccc5)c5cccc-4c35)c3cccc4c3oc3ccccc34)cc2)cc1 ; c1ccc(-c2ccc(-c3ccc4c5c(cccc35)-c3ccc(N(c5ccccc5)c5cccc6c5oc5ccccc56)cc3O4)cc2)cc1]

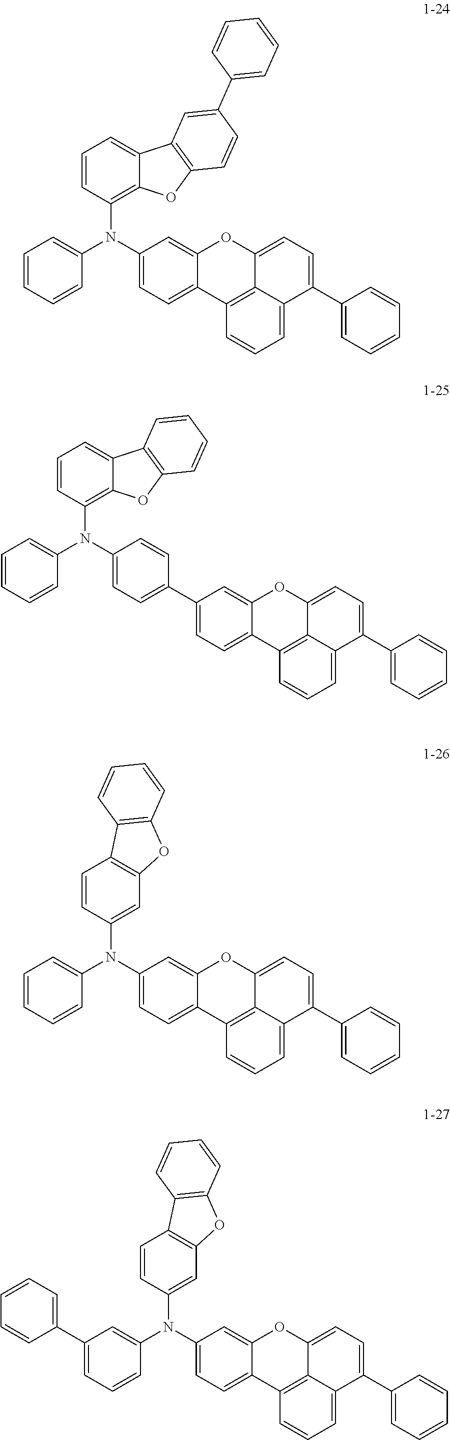 [SMILES: c1ccc(-c2ccc3oc4c(N(c5ccccc5)c5ccc6c(c5)Oc5ccc(-c7ccccc7)c7cccc-6c57)cccc4c3c2)cc1 ; c1ccc(-c2ccc3c4c(cccc24)-c2ccc(-c4ccc(N(c5ccccc5)c5cccc6c5oc5ccccc56)cc4)cc2O3)cc1 ; c1ccc(-c2ccc3c4c(cccc24)-c2ccc(N(c4ccccc4)c4ccc5c(c4)oc4ccccc45)cc2O3)cc1 ; c1ccc(-c2cccc(N(c3ccc4c(c3)Oc3ccc(-c5ccccc5)c5cccc-4c35)c3ccc4c(c3)oc3ccccc34)c2)cc1]

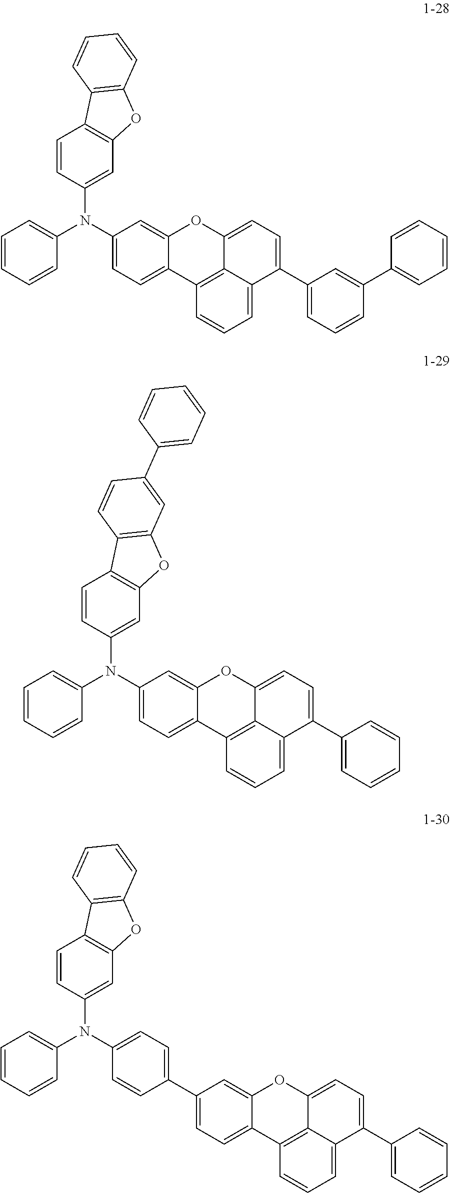 [SMILES: c1ccc(-c2cccc(-c3ccc4c5c(cccc35)-c3ccc(N(c5ccccc5)c5ccc6c(c5)oc5ccccc56)cc3O4)c2)cc1 ; c1ccc(-c2ccc3c(c2)oc2cc(N(c4ccccc4)c4ccc5c(c4)Oc4ccc(-c6ccccc6)c6cccc-5c46)ccc23)cc1 ; c1ccc(-c2ccc3c4c(cccc24)-c2ccc(-c4ccc(N(c5ccccc5)c5ccc6c(c5)oc5ccccc56)cc4)cc2O3)cc1]

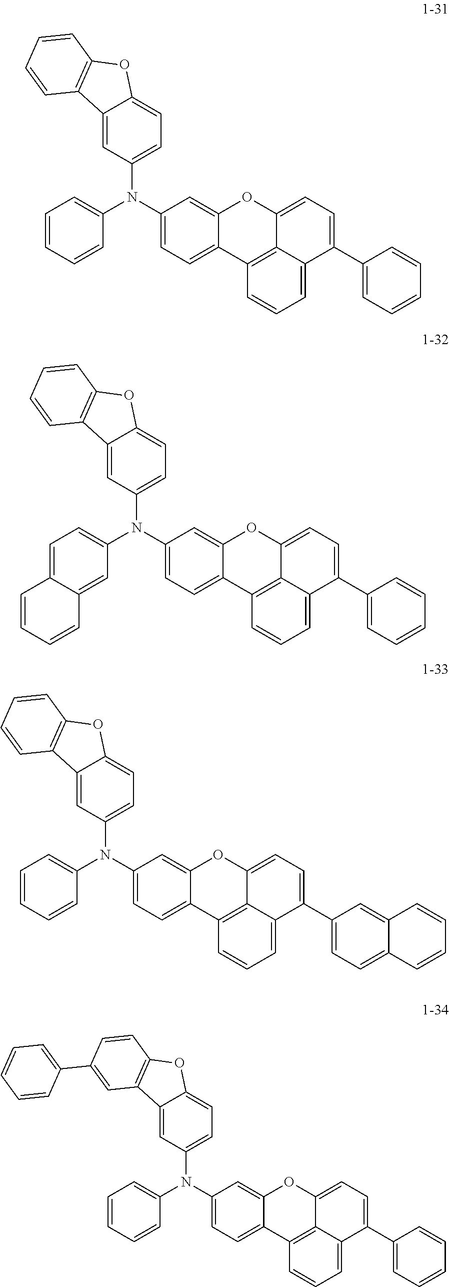 [SMILES: c1ccc(-c2ccc3c4c(cccc24)-c2ccc(N(c4ccccc4)c4ccc5oc6ccccc6c5c4)cc2O3)cc1 ; c1ccc(-c2ccc3c4c(cccc24)-c2ccc(N(c4ccc5ccccc5c4)c4ccc5oc6ccccc6c5c4)cc2O3)cc1 ; c1ccc(N(c2ccc3c(c2)Oc2ccc(-c4ccc5ccccc5c4)c4cccc-3c24)c2ccc3oc4ccccc4c3c2)cc1 ; c1ccc(-c2ccc3oc4ccc(N(c5ccccc5)c5ccc6c(c5)Oc5ccc(-c7ccccc7)c7cccc-6c57)cc4c3c2)cc1]

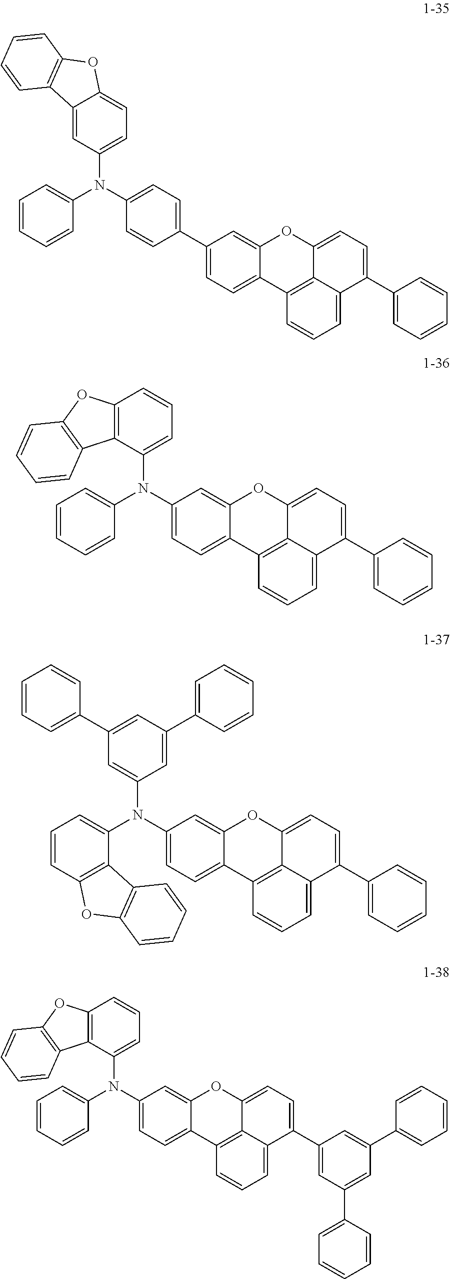 [SMILES: c1ccc(-c2ccc3c4c(cccc24)-c2ccc(-c4ccc(N(c5ccccc5)c5ccc6oc7ccccc7c6c5)cc4)cc2O3)cc1 ; c1ccc(-c2ccc3c4c(cccc24)-c2ccc(N(c4ccccc4)c4cccc5oc6ccccc6c45)cc2O3)cc1 ; c1ccc(-c2cc(-c3ccccc3)cc(N(c3ccc4c(c3)Oc3ccc(-c5ccccc5)c5cccc-4c35)c3cccc4oc5ccccc5c34)c2)cc1 ; c1ccc(-c2cc(-c3ccccc3)cc(-c3ccc4c5c(cccc35)-c3ccc(N(c5ccccc5)c5cccc6oc7ccccc7c56)cc3O4)c2)cc1]

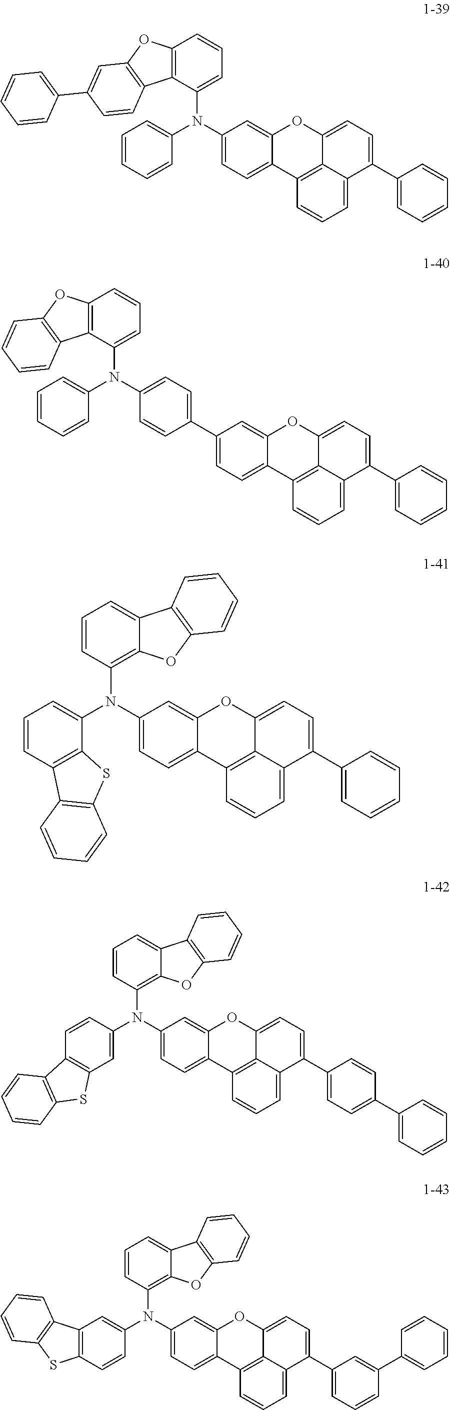 [SMILES: c1ccc(-c2ccc3c(c2)oc2cccc(N(c4ccccc4)c4ccc5c(c4)Oc4ccc(-c6ccccc6)c6cccc-5c46)c23)cc1 ; c1ccc(-c2ccc3c4c(cccc24)-c2ccc(-c4ccc(N(c5ccccc5)c5cccc6oc7ccccc7c56)cc4)cc2O3)cc1 ; c1ccc(-c2ccc3c4c(cccc24)-c2ccc(N(c4cccc5c4oc4ccccc45)c4cccc5c4sc4ccccc45)cc2O3)cc1 ; c1ccc(-c2ccc(-c3ccc4c5c(cccc35)-c3ccc(N(c5ccc6c(c5)sc5ccccc56)c5cccc6c5oc5ccccc56)cc3O4)cc2)cc1 ; c1ccc(-c2cccc(-c3ccc4c5c(cccc35)-c3ccc(N(c5ccc6sc7ccccc7c6c5)c5cccc6c5oc5ccccc56)cc3O4)c2)cc1]

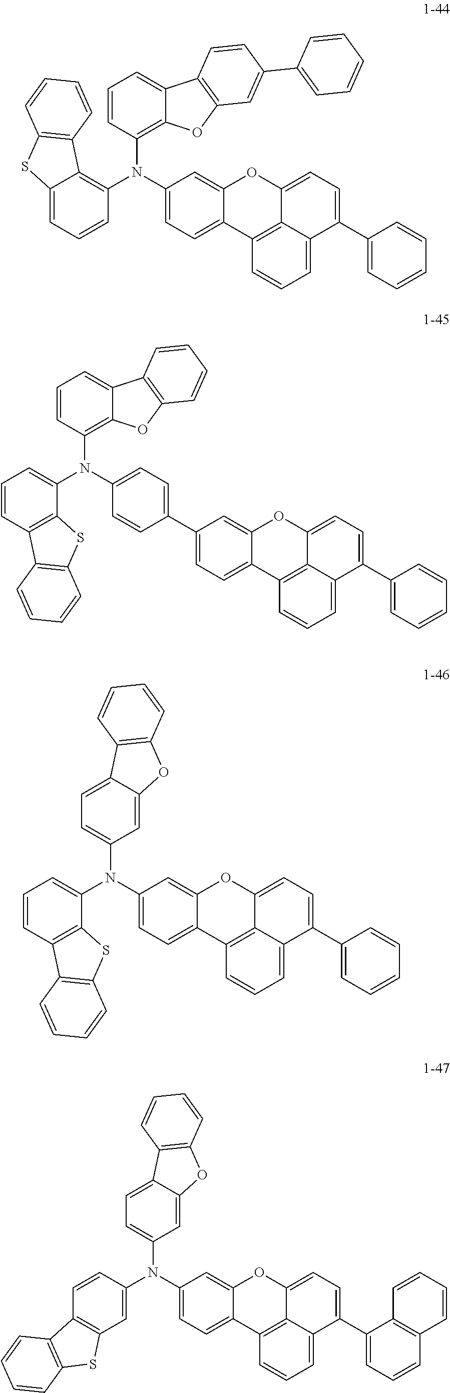 [SMILES: c1ccc(-c2ccc3c(c2)oc2c(N(c4ccc5c(c4)Oc4ccc(-c6ccccc6)c6cccc-5c46)c4cccc5sc6ccccc6c45)cccc23)cc1 ; c1ccc(-c2ccc3c4c(cccc24)-c2ccc(-c4ccc(N(c5cccc6c5oc5ccccc56)c5cccc6c5sc5ccccc56)cc4)cc2O3)cc1 ; c1ccc(-c2ccc3c4c(cccc24)-c2ccc(N(c4ccc5c(c4)oc4ccccc45)c4cccc5c4sc4ccccc45)cc2O3)cc1 ; c1ccc2c(-c3ccc4c5c(cccc35)-c3ccc(N(c5ccc6c(c5)oc5ccccc56)c5ccc6c(c5)sc5ccccc56)cc3O4)cccc2c1]

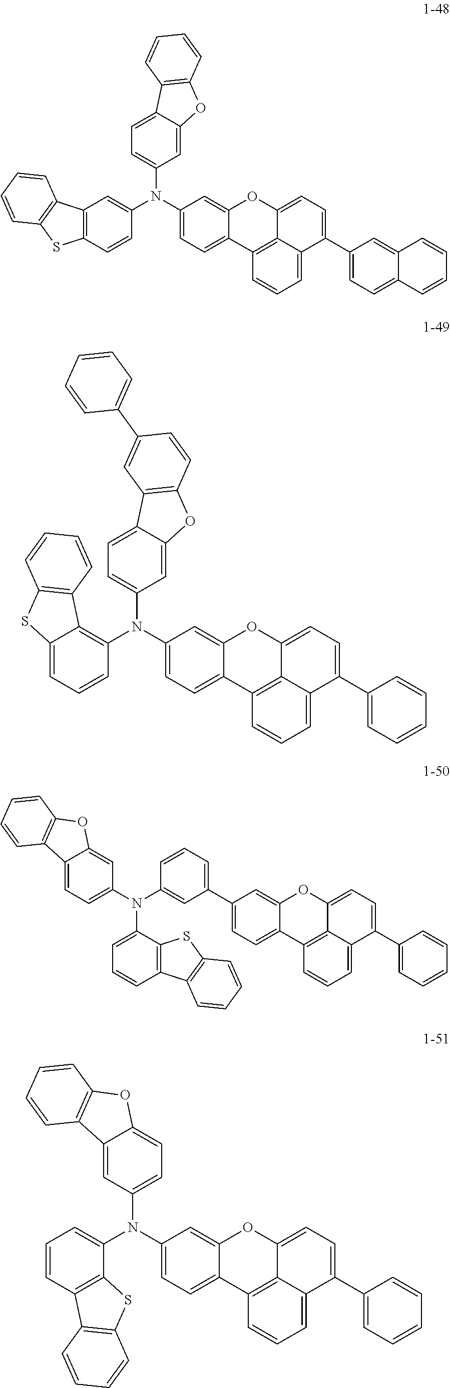 [SMILES: c1ccc2cc(-c3ccc4c5c(cccc35)-c3ccc(N(c5ccc6c(c5)oc5ccccc56)c5ccc6sc7ccccc7c6c5)cc3O4)ccc2c1 ; c1ccc(-c2ccc3oc4cc(N(c5ccc6c(c5)Oc5ccc(-c7ccccc7)c7cccc-6c57)c5cccc6sc7ccccc7c56)ccc4c3c2)cc1 ; c1ccc(-c2ccc3c4c(cccc24)-c2ccc(-c4cccc(N(c5ccc6c(c5)oc5ccccc56)c5cccc6c5sc5ccccc56)c4)cc2O3)cc1 ; c1ccc(-c2ccc3c4c(cccc24)-c2ccc(N(c4ccc5oc6ccccc6c5c4)c4cccc5c4sc4ccccc45)cc2O3)cc1]

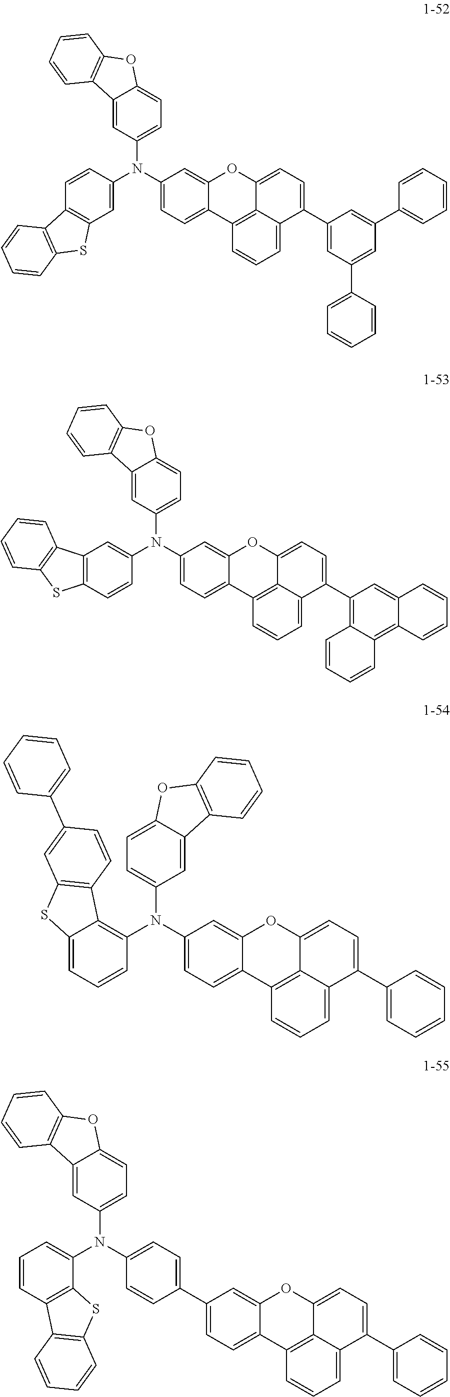 [SMILES: c1ccc(-c2cc(-c3ccccc3)cc(-c3ccc4c5c(cccc35)-c3ccc(N(c5ccc6c(c5)sc5ccccc56)c5ccc6oc7ccccc7c6c5)cc3O4)c2)cc1 ; c1ccc2c(c1)cc(-c1ccc3c4c(cccc14)-c1ccc(N(c4ccc5oc6ccccc6c5c4)c4ccc5sc6ccccc6c5c4)cc1O3)c1ccccc12 ; c1ccc(-c2ccc3c(c2)sc2cccc(N(c4ccc5c(c4)Oc4ccc(-c6ccccc6)c6cccc-5c46)c4ccc5oc6ccccc6c5c4)c23)cc1 ; c1ccc(-c2ccc3c4c(cccc24)-c2ccc(-c4ccc(N(c5ccc6oc7ccccc7c6c5)c5cccc6c5sc5ccccc56)cc4)cc2O3)cc1]

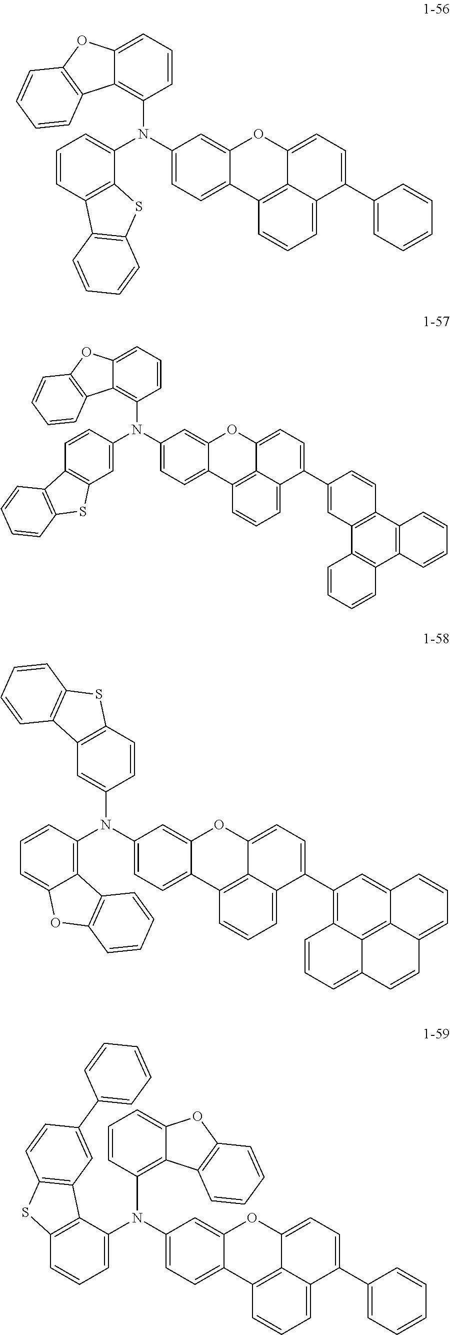 [SMILES: c1ccc(-c2ccc3c4c(cccc24)-c2ccc(N(c4cccc5c4sc4ccccc45)c4cccc5oc6ccccc6c45)cc2O3)cc1 ; c1ccc2c(c1)oc1cccc(N(c3ccc4c(c3)Oc3ccc(-c5ccc6c7ccccc7c7ccccc7c6c5)c5cccc-4c35)c3ccc4c(c3)sc3ccccc34)c12 ; c1cc2ccc3cccc4c(-c5ccc6c7c(cccc57)-c5ccc(N(c7ccc8sc9ccccc9c8c7)c7cccc8oc9ccccc9c78)cc5O6)cc(c1)c2c34 ; c1ccc(-c2ccc3sc4cccc(N(c5ccc6c(c5)Oc5ccc(-c7ccccc7)c7cccc-6c57)c5cccc6oc7ccccc7c56)c4c3c2)cc1]

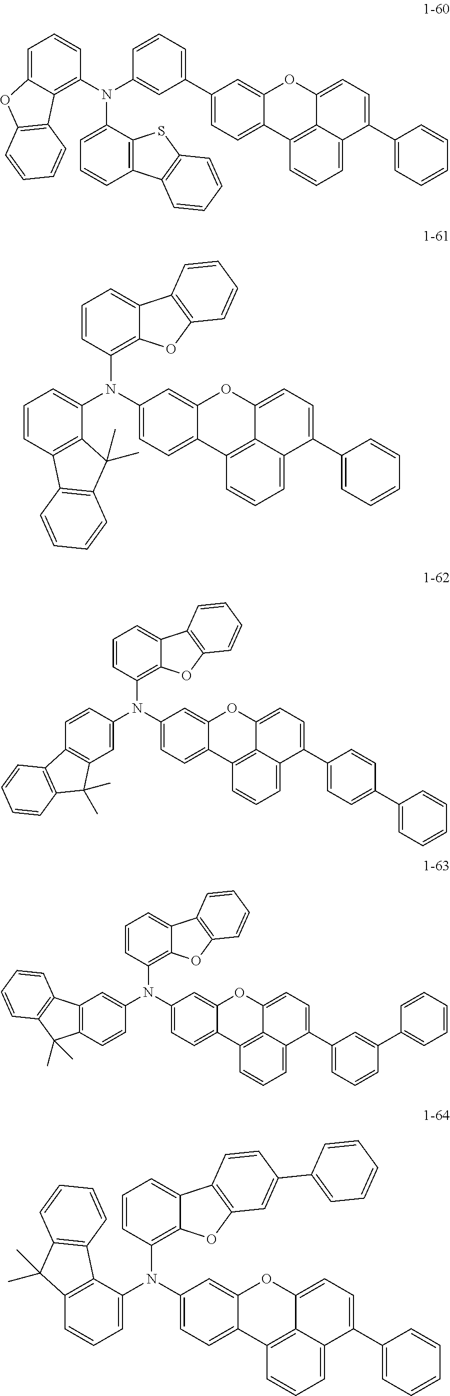 [SMILES: c1ccc(-c2ccc3c4c(cccc24)-c2ccc(-c4cccc(N(c5cccc6c5sc5ccccc56)c5cccc6oc7ccccc7c56)c4)cc2O3)cc1 ; CC1(C)c2ccccc2-c2cccc(N(c3ccc4c(c3)Oc3ccc(-c5ccccc5)c5cccc-4c35)c3cccc4c3oc3ccccc34)c21 ; CC1(C)c2ccccc2-c2ccc(N(c3ccc4c(c3)Oc3ccc(-c5ccc(-c6ccccc6)cc5)c5cccc-4c35)c3cccc4c3oc3ccccc34)cc21 ; CC1(C)c2ccccc2-c2cc(N(c3ccc4c(c3)Oc3ccc(-c5cccc(-c6ccccc6)c5)c5cccc-4c35)c3cccc4c3oc3ccccc34)ccc21 ; CC1(C)c2ccccc2-c2c(N(c3ccc4c(c3)Oc3ccc(-c5ccccc5)c5cccc-4c35)c3cccc4c3oc3cc(-c5ccccc5)ccc34)cccc21]

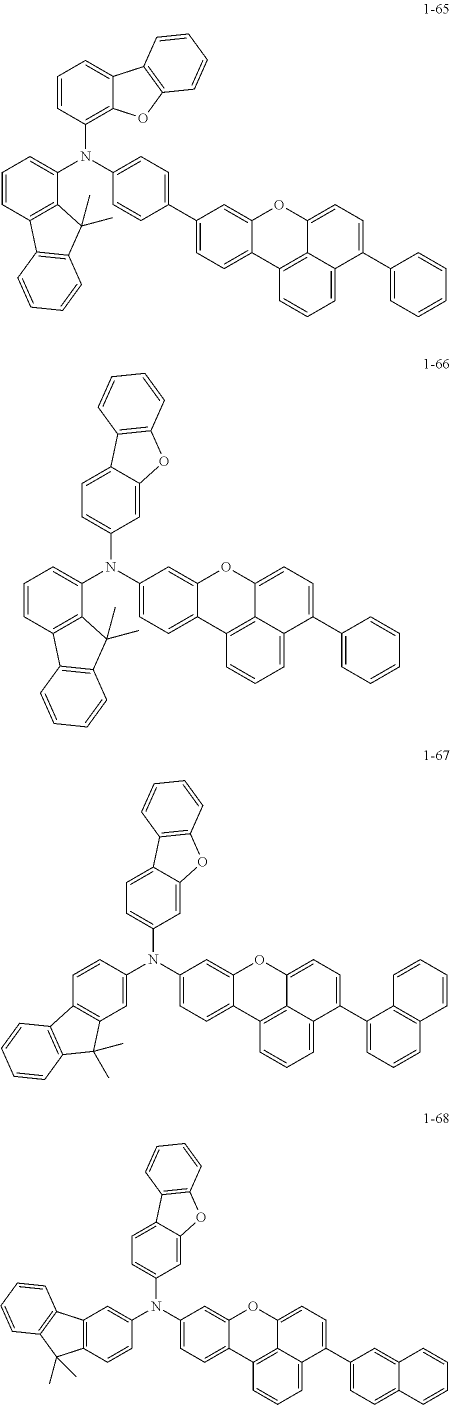 [SMILES: CC1(C)c2ccccc2-c2cccc(N(c3ccc(-c4ccc5c(c4)Oc4ccc(-c6ccccc6)c6cccc-5c46)cc3)c3cccc4c3oc3ccccc34)c21 ; CC1(C)c2ccccc2-c2cccc(N(c3ccc4c(c3)Oc3ccc(-c5ccccc5)c5cccc-4c35)c3ccc4c(c3)oc3ccccc34)c21 ; CC1(C)c2ccccc2-c2ccc(N(c3ccc4c(c3)Oc3ccc(-c5cccc6ccccc56)c5cccc-4c35)c3ccc4c(c3)oc3ccccc34)cc21 ; CC1(C)c2ccccc2-c2cc(N(c3ccc4c(c3)Oc3ccc(-c5ccc6ccccc6c5)c5cccc-4c35)c3ccc4c(c3)oc3ccccc34)ccc21]

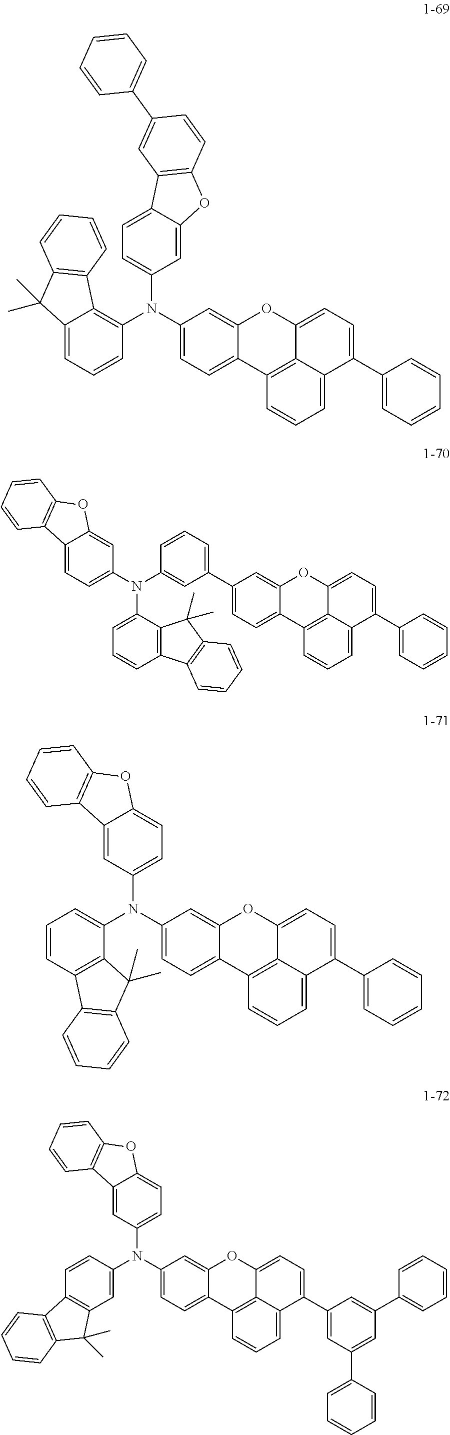 [SMILES: CC1(C)c2ccccc2-c2c(N(c3ccc4c(c3)Oc3ccc(-c5ccccc5)c5cccc-4c35)c3ccc4c(c3)oc3ccc(-c5ccccc5)cc34)cccc21 ; CC1(C)c2ccccc2-c2cccc(N(c3cccc(-c4ccc5c(c4)Oc4ccc(-c6ccccc6)c6cccc-5c46)c3)c3ccc4c(c3)oc3ccccc34)c21 ; CC1(C)c2ccccc2-c2cccc(N(c3ccc4c(c3)Oc3ccc(-c5ccccc5)c5cccc-4c35)c3ccc4oc5ccccc5c4c3)c21 ; CC1(C)c2ccccc2-c2ccc(N(c3ccc4c(c3)Oc3ccc(-c5cc(-c6ccccc6)cc(-c6ccccc6)c5)c5cccc-4c35)c3ccc4oc5ccccc5c4c3)cc21]

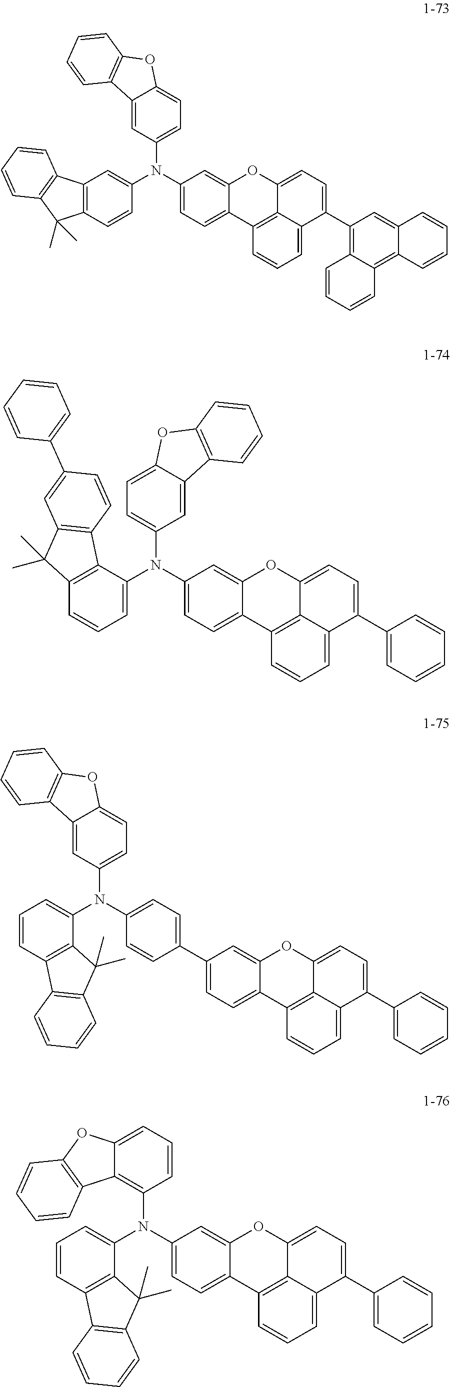 [SMILES: CC1(C)c2ccccc2-c2cc(N(c3ccc4c(c3)Oc3ccc(-c5cc6ccccc6c6ccccc56)c5cccc-4c35)c3ccc4oc5ccccc5c4c3)ccc21 ; CC1(C)c2cc(-c3ccccc3)ccc2-c2c(N(c3ccc4c(c3)Oc3ccc(-c5ccccc5)c5cccc-4c35)c3ccc4oc5ccccc5c4c3)cccc21 ; CC1(C)c2ccccc2-c2cccc(N(c3ccc(-c4ccc5c(c4)Oc4ccc(-c6ccccc6)c6cccc-5c46)cc3)c3ccc4oc5ccccc5c4c3)c21 ; CC1(C)c2ccccc2-c2cccc(N(c3ccc4c(c3)Oc3ccc(-c5ccccc5)c5cccc-4c35)c3cccc4oc5ccccc5c34)c21]

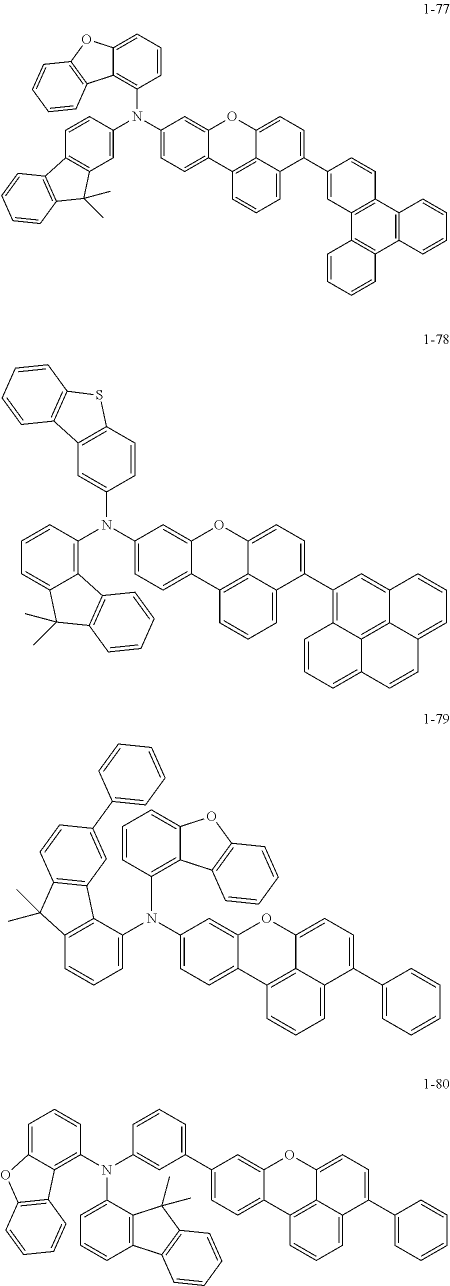 [SMILES: CC1(C)c2ccccc2-c2ccc(N(c3ccc4c(c3)Oc3ccc(-c5ccc6c7ccccc7c7ccccc7c6c5)c5cccc-4c35)c3cccc4oc5ccccc5c34)cc21 ; CC1(C)c2ccccc2-c2c(N(c3ccc4c(c3)Oc3ccc(-c5cc6cccc7ccc8cccc5c8c76)c5cccc-4c35)c3ccc4sc5ccccc5c4c3)cccc21 ; CC1(C)c2ccc(-c3ccccc3)cc2-c2c(N(c3ccc4c(c3)Oc3ccc(-c5ccccc5)c5cccc-4c35)c3cccc4oc5ccccc5c34)cccc21 ; CC1(C)c2ccccc2-c2cccc(N(c3cccc(-c4ccc5c(c4)Oc4ccc(-c6ccccc6)c6cccc-5c46)c3)c3cccc4oc5ccccc5c34)c21]

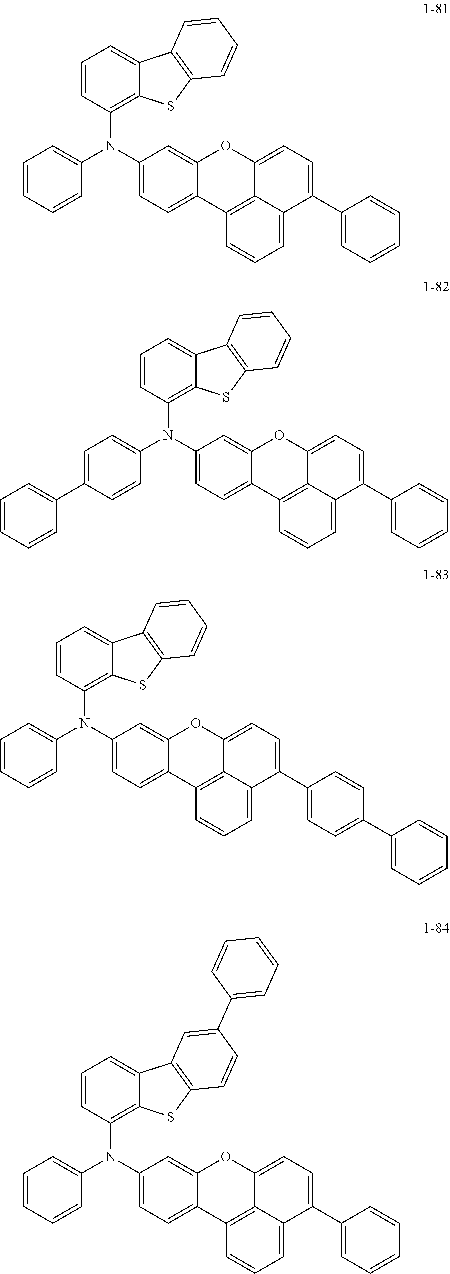 [SMILES: c1ccc(-c2ccc3c4c(cccc24)-c2ccc(N(c4ccccc4)c4cccc5c4sc4ccccc45)cc2O3)cc1 ; c1ccc(-c2ccc(N(c3ccc4c(c3)Oc3ccc(-c5ccccc5)c5cccc-4c35)c3cccc4c3sc3ccccc34)cc2)cc1 ; c1ccc(-c2ccc(-c3ccc4c5c(cccc35)-c3ccc(N(c5ccccc5)c5cccc6c5sc5ccccc56)cc3O4)cc2)cc1 ; c1ccc(-c2ccc3sc4c(N(c5ccccc5)c5ccc6c(c5)Oc5ccc(-c7ccccc7)c7cccc-6c57)cccc4c3c2)cc1]

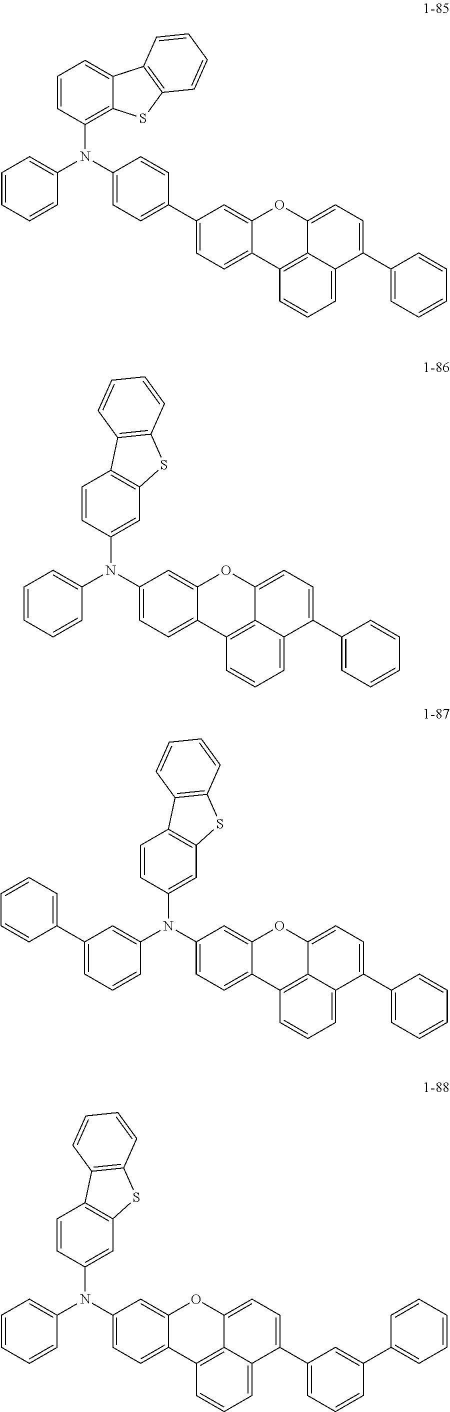 [SMILES: c1ccc(-c2ccc3c4c(cccc24)-c2ccc(-c4ccc(N(c5ccccc5)c5cccc6c5sc5ccccc56)cc4)cc2O3)cc1 ; c1ccc(-c2ccc3c4c(cccc24)-c2ccc(N(c4ccccc4)c4ccc5c(c4)sc4ccccc45)cc2O3)cc1 ; c1ccc(-c2cccc(N(c3ccc4c(c3)Oc3ccc(-c5ccccc5)c5cccc-4c35)c3ccc4c(c3)sc3ccccc34)c2)cc1 ; c1ccc(-c2cccc(-c3ccc4c5c(cccc35)-c3ccc(N(c5ccccc5)c5ccc6c(c5)sc5ccccc56)cc3O4)c2)cc1]

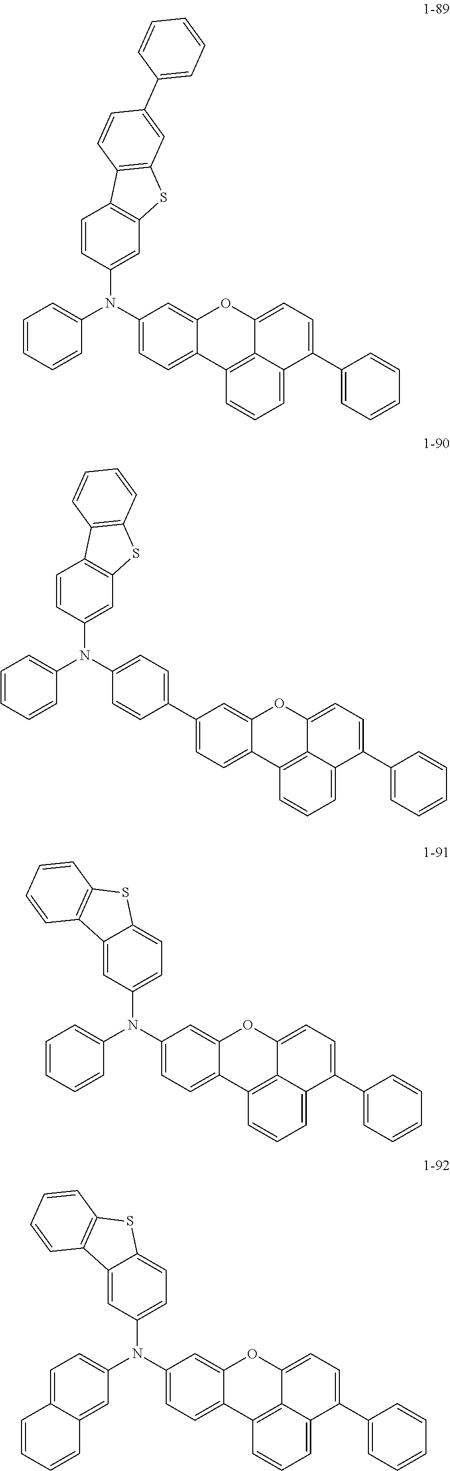 [SMILES: c1ccc(-c2ccc3c(c2)sc2cc(N(c4ccccc4)c4ccc5c(c4)Oc4ccc(-c6ccccc6)c6cccc-5c46)ccc23)cc1 ; c1ccc(-c2ccc3c4c(cccc24)-c2ccc(-c4ccc(N(c5ccccc5)c5ccc6c(c5)sc5ccccc56)cc4)cc2O3)cc1 ; c1ccc(-c2ccc3c4c(cccc24)-c2ccc(N(c4ccccc4)c4ccc5sc6ccccc6c5c4)cc2O3)cc1 ; c1ccc(-c2ccc3c4c(cccc24)-c2ccc(N(c4ccc5ccccc5c4)c4ccc5sc6ccccc6c5c4)cc2O3)cc1]

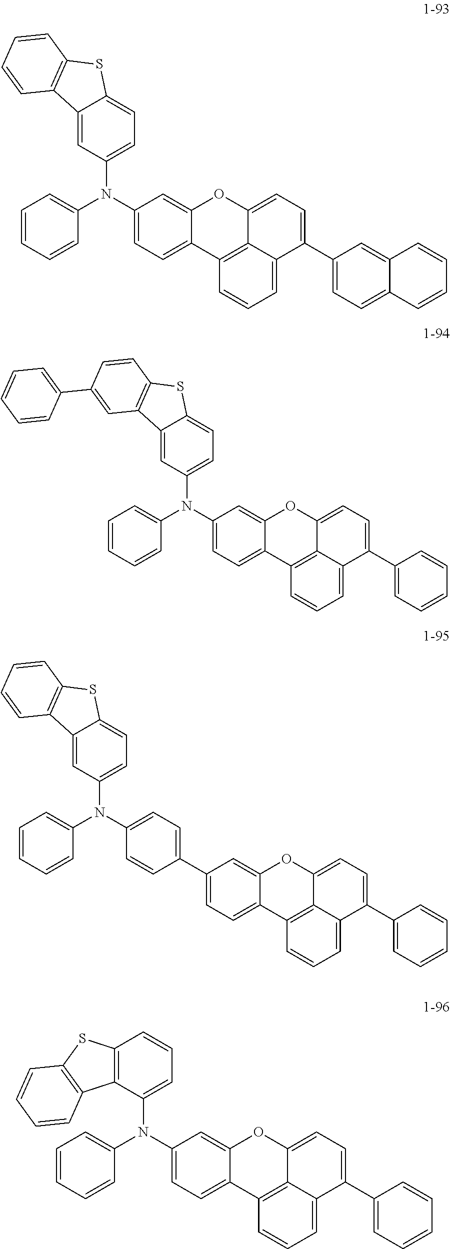 [SMILES: c1ccc(N(c2ccc3c(c2)Oc2ccc(-c4ccc5ccccc5c4)c4cccc-3c24)c2ccc3sc4ccccc4c3c2)cc1 ; c1ccc(-c2ccc3sc4ccc(N(c5ccccc5)c5ccc6c(c5)Oc5ccc(-c7ccccc7)c7cccc-6c57)cc4c3c2)cc1 ; c1ccc(-c2ccc3c4c(cccc24)-c2ccc(-c4ccc(N(c5ccccc5)c5ccc6sc7ccccc7c6c5)cc4)cc2O3)cc1 ; c1ccc(-c2ccc3c4c(cccc24)-c2ccc(N(c4ccccc4)c4cccc5sc6ccccc6c45)cc2O3)cc1]

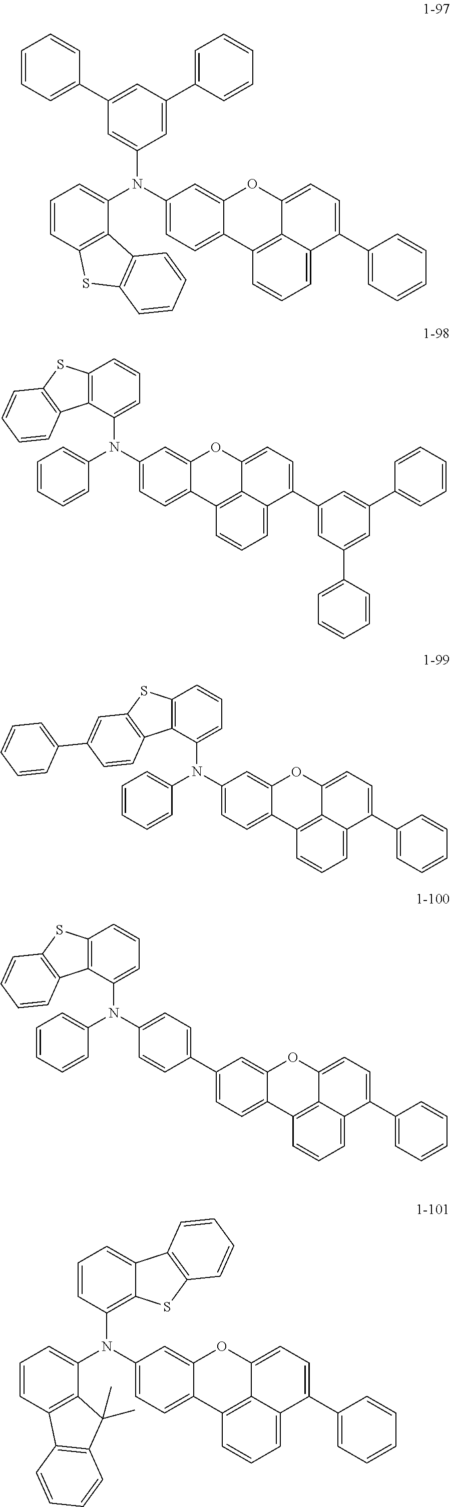 [SMILES: c1ccc(-c2cc(-c3ccccc3)cc(N(c3ccc4c(c3)Oc3ccc(-c5ccccc5)c5cccc-4c35)c3cccc4sc5ccccc5c34)c2)cc1 ; c1ccc(-c2cc(-c3ccccc3)cc(-c3ccc4c5c(cccc35)-c3ccc(N(c5ccccc5)c5cccc6sc7ccccc7c56)cc3O4)c2)cc1 ; c1ccc(-c2ccc3c(c2)sc2cccc(N(c4ccccc4)c4ccc5c(c4)Oc4ccc(-c6ccccc6)c6cccc-5c46)c23)cc1 ; c1ccc(-c2ccc3c4c(cccc24)-c2ccc(-c4ccc(N(c5ccccc5)c5cccc6sc7ccccc7c56)cc4)cc2O3)cc1 ; CC1(C)c2ccccc2-c2cccc(N(c3ccc4c(c3)Oc3ccc(-c5ccccc5)c5cccc-4c35)c3cccc4c3sc3ccccc34)c21]

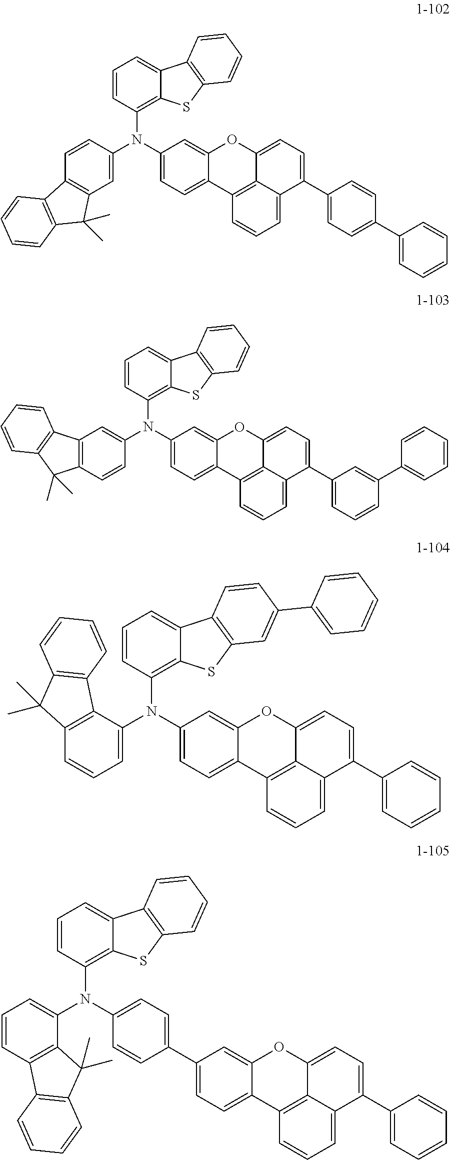 [SMILES: CC1(C)c2ccccc2-c2ccc(N(c3ccc4c(c3)Oc3ccc(-c5ccc(-c6ccccc6)cc5)c5cccc-4c35)c3cccc4c3sc3ccccc34)cc21 ; CC1(C)c2ccccc2-c2cc(N(c3ccc4c(c3)Oc3ccc(-c5cccc(-c6ccccc6)c5)c5cccc-4c35)c3cccc4c3sc3ccccc34)ccc21 ; CC1(C)c2ccccc2-c2c(N(c3ccc4c(c3)Oc3ccc(-c5ccccc5)c5cccc-4c35)c3cccc4c3sc3cc(-c5ccccc5)ccc34)cccc21 ; CC1(C)c2ccccc2-c2cccc(N(c3ccc(-c4ccc5c(c4)Oc4ccc(-c6ccccc6)c6cccc-5c46)cc3)c3cccc4c3sc3ccccc34)c21]

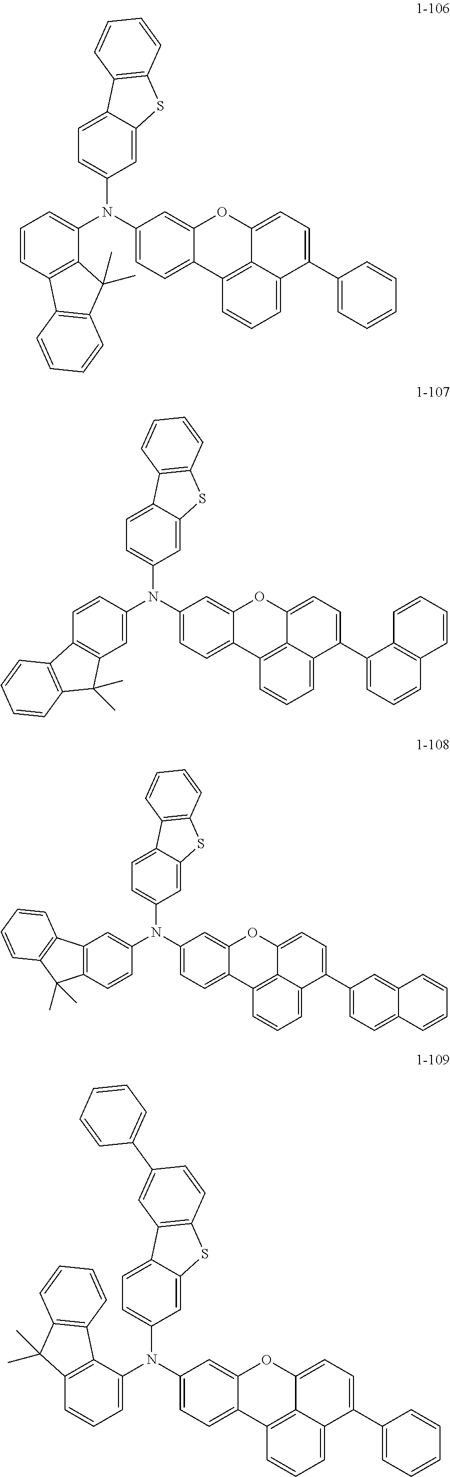 [SMILES: CC1(C)c2ccccc2-c2cccc(N(c3ccc4c(c3)Oc3ccc(-c5ccccc5)c5cccc-4c35)c3ccc4c(c3)sc3ccccc34)c21 ; CC1(C)c2ccccc2-c2ccc(N(c3ccc4c(c3)Oc3ccc(-c5cccc6ccccc56)c5cccc-4c35)c3ccc4c(c3)sc3ccccc34)cc21 ; CC1(C)c2ccccc2-c2cc(N(c3ccc4c(c3)Oc3ccc(-c5ccc6ccccc6c5)c5cccc-4c35)c3ccc4c(c3)sc3ccccc34)ccc21 ; CC1(C)c2ccccc2-c2c(N(c3ccc4c(c3)Oc3ccc(-c5ccccc5)c5cccc-4c35)c3ccc4c(c3)sc3ccc(-c5ccccc5)cc34)cccc21]

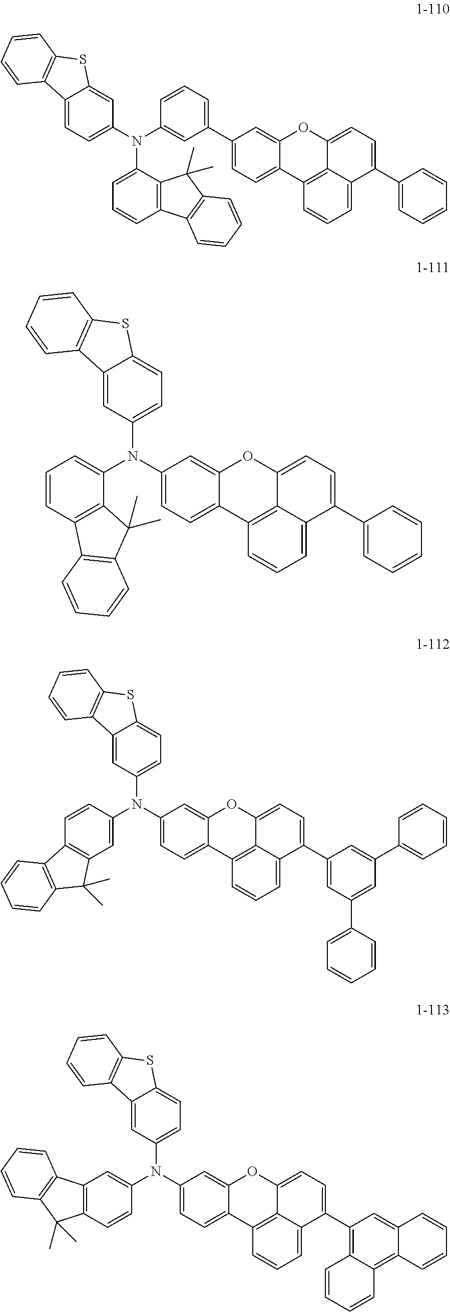 [SMILES: CC1(C)c2ccccc2-c2cccc(N(c3cccc(-c4ccc5c(c4)Oc4ccc(-c6ccccc6)c6cccc-5c46)c3)c3ccc4c(c3)sc3ccccc34)c21 ; CC1(C)c2ccccc2-c2cccc(N(c3ccc4c(c3)Oc3ccc(-c5ccccc5)c5cccc-4c35)c3ccc4sc5ccccc5c4c3)c21 ; CC1(C)c2ccccc2-c2ccc(N(c3ccc4c(c3)Oc3ccc(-c5cc(-c6ccccc6)cc(-c6ccccc6)c5)c5cccc-4c35)c3ccc4sc5ccccc5c4c3)cc21 ; CC1(C)c2ccccc2-c2cc(N(c3ccc4c(c3)Oc3ccc(-c5cc6ccccc6c6ccccc56)c5cccc-4c35)c3ccc4sc5ccccc5c4c3)ccc21]

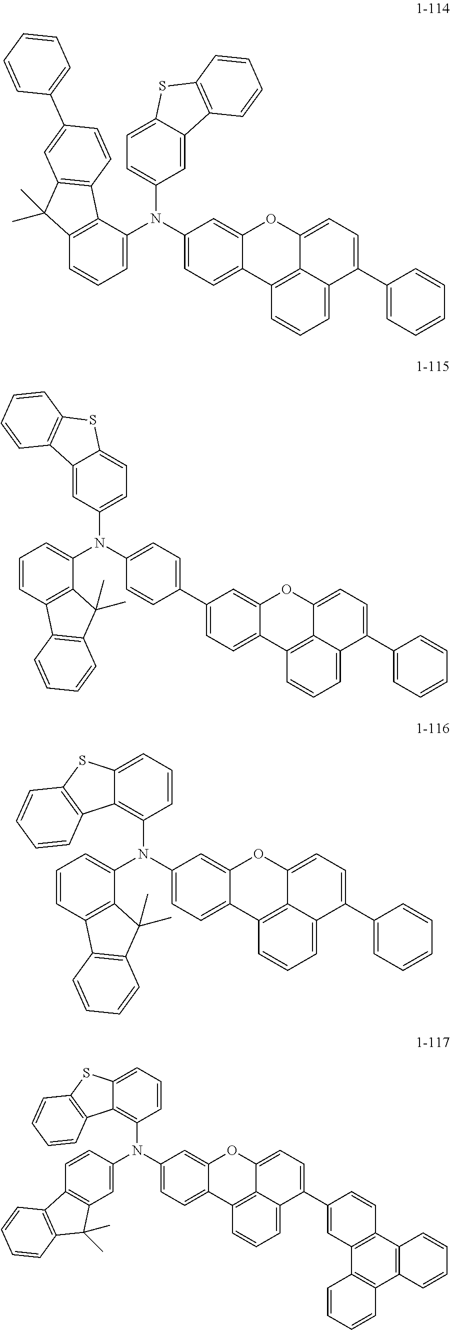 [SMILES: CC1(C)c2cc(-c3ccccc3)ccc2-c2c(N(c3ccc4c(c3)Oc3ccc(-c5ccccc5)c5cccc-4c35)c3ccc4sc5ccccc5c4c3)cccc21 ; CC1(C)c2ccccc2-c2cccc(N(c3ccc(-c4ccc5c(c4)Oc4ccc(-c6ccccc6)c6cccc-5c46)cc3)c3ccc4sc5ccccc5c4c3)c21 ; CC1(C)c2ccccc2-c2cccc(N(c3ccc4c(c3)Oc3ccc(-c5ccccc5)c5cccc-4c35)c3cccc4sc5ccccc5c34)c21 ; CC1(C)c2ccccc2-c2ccc(N(c3ccc4c(c3)Oc3ccc(-c5ccc6c7ccccc7c7ccccc7c6c5)c5cccc-4c35)c3cccc4sc5ccccc5c34)cc21]

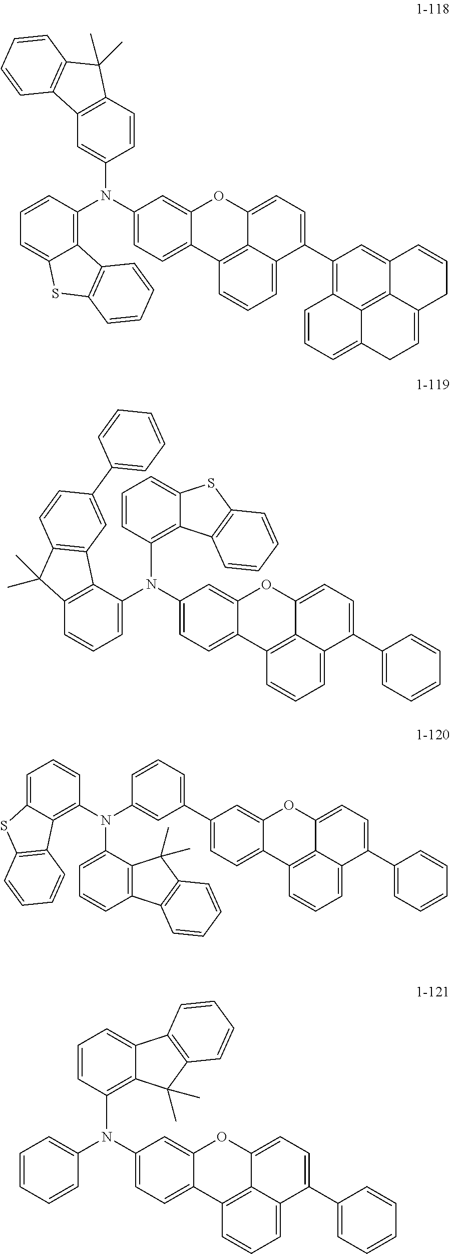 [SMILES: CC1(C)c2ccccc2-c2cc(N(c3ccc4c(c3)Oc3ccc(-c5cc6c7c8c(cccc58)CC=C7CC=C6)c5cccc-4c35)c3cccc4sc5ccccc5c34)ccc21 ; CC1(C)c2ccc(-c3ccccc3)cc2-c2c(N(c3ccc4c(c3)Oc3ccc(-c5ccccc5)c5cccc-4c35)c3cccc4sc5ccccc5c34)cccc21 ; CC1(C)c2ccccc2-c2cccc(N(c3cccc(-c4ccc5c(c4)Oc4ccc(-c6ccccc6)c6cccc-5c46)c3)c3cccc4sc5ccccc5c34)c21 ; CC1(C)c2ccccc2-c2cccc(N(c3ccccc3)c3ccc4c(c3)Oc3ccc(-c5ccccc5)c5cccc-4c35)c21]

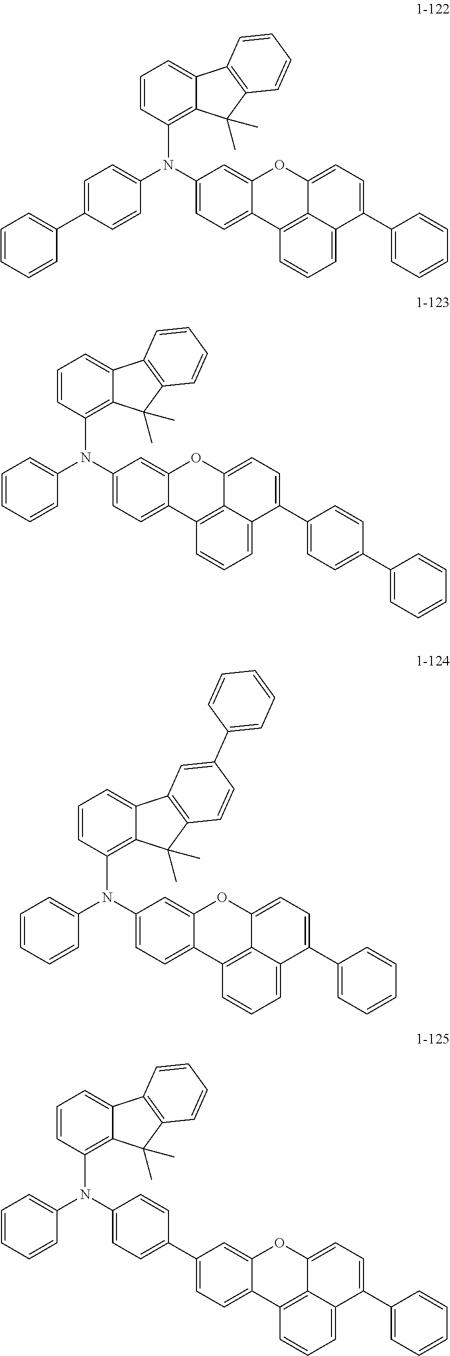 [SMILES: CC1(C)c2ccccc2-c2cccc(N(c3ccc(-c4ccccc4)cc3)c3ccc4c(c3)Oc3ccc(-c5ccccc5)c5cccc-4c35)c21 ; CC1(C)c2ccccc2-c2cccc(N(c3ccccc3)c3ccc4c(c3)Oc3ccc(-c5ccc(-c6ccccc6)cc5)c5cccc-4c35)c21 ; CC1(C)c2ccc(-c3ccccc3)cc2-c2cccc(N(c3ccccc3)c3ccc4c(c3)Oc3ccc(-c5ccccc5)c5cccc-4c35)c21 ; CC1(C)c2ccccc2-c2cccc(N(c3ccccc3)c3ccc(-c4ccc5c(c4)Oc4ccc(-c6ccccc6)c6cccc-5c46)cc3)c21]

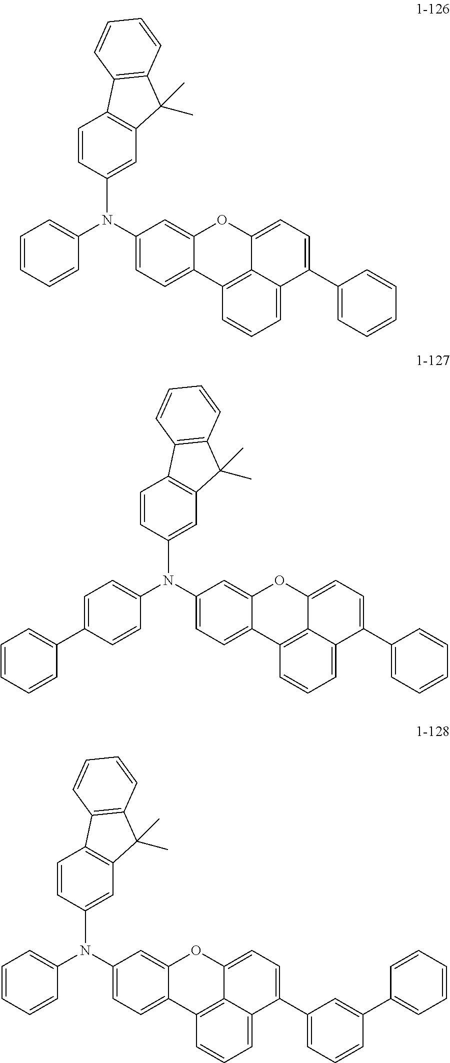 [SMILES: CC1(C)c2ccccc2-c2ccc(N(c3ccccc3)c3ccc4c(c3)Oc3ccc(-c5ccccc5)c5cccc-4c35)cc21 ; CC1(C)c2ccccc2-c2ccc(N(c3ccc(-c4ccccc4)cc3)c3ccc4c(c3)Oc3ccc(-c5ccccc5)c5cccc-4c35)cc21 ; CC1(C)c2ccccc2-c2ccc(N(c3ccccc3)c3ccc4c(c3)Oc3ccc(-c5cccc(-c6ccccc6)c5)c5cccc-4c35)cc21]

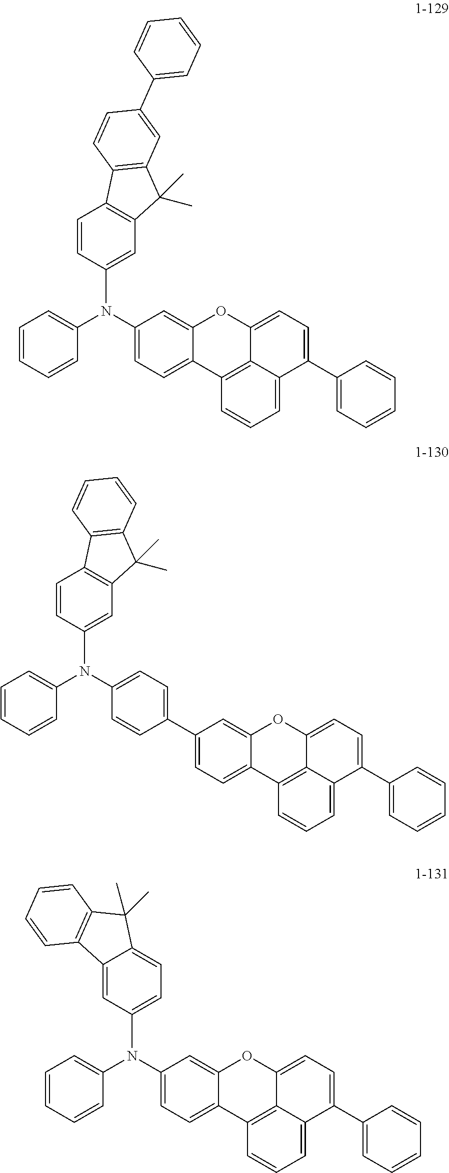 [SMILES: CC1(C)c2cc(-c3ccccc3)ccc2-c2ccc(N(c3ccccc3)c3ccc4c(c3)Oc3ccc(-c5ccccc5)c5cccc-4c35)cc21 ; CC1(C)c2ccccc2-c2ccc(N(c3ccccc3)c3ccc(-c4ccc5c(c4)Oc4ccc(-c6ccccc6)c6cccc-5c46)cc3)cc21 ; CC1(C)c2ccccc2-c2cc(N(c3ccccc3)c3ccc4c(c3)Oc3ccc(-c5ccccc5)c5cccc-4c35)ccc21]

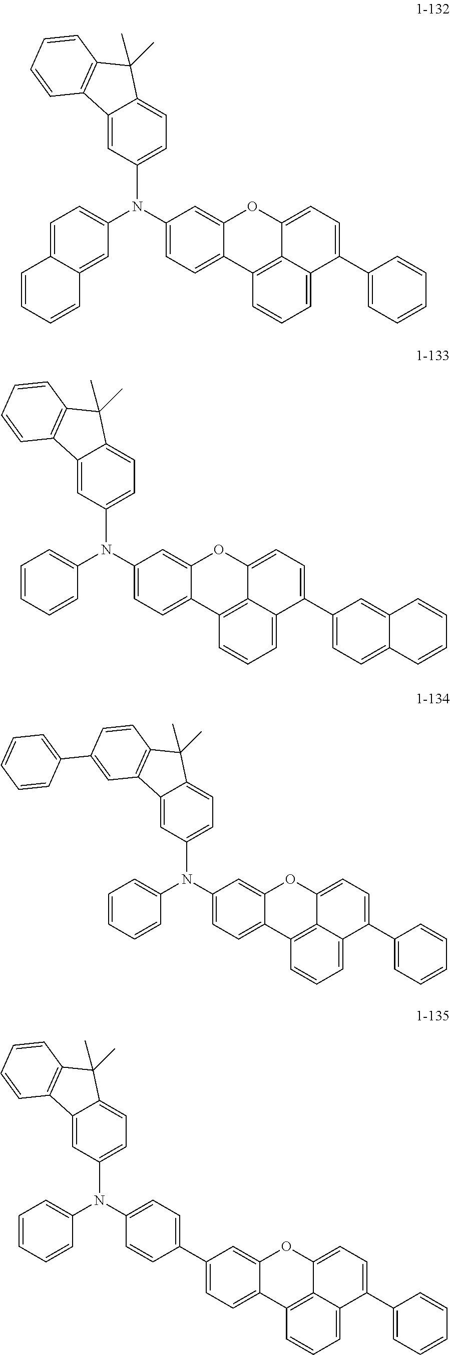 [SMILES: CC1(C)c2ccccc2-c2cc(N(c3ccc4c(c3)Oc3ccc(-c5ccccc5)c5cccc-4c35)c3ccc4ccccc4c3)ccc21 ; CC1(C)c2ccccc2-c2cc(N(c3ccccc3)c3ccc4c(c3)Oc3ccc(-c5ccc6ccccc6c5)c5cccc-4c35)ccc21 ; CC1(C)c2ccc(-c3ccccc3)cc2-c2cc(N(c3ccccc3)c3ccc4c(c3)Oc3ccc(-c5ccccc5)c5cccc-4c35)ccc21 ; CC1(C)c2ccccc2-c2cc(N(c3ccccc3)c3ccc(-c4ccc5c(c4)Oc4ccc(-c6ccccc6)c6cccc-5c46)cc3)ccc21]

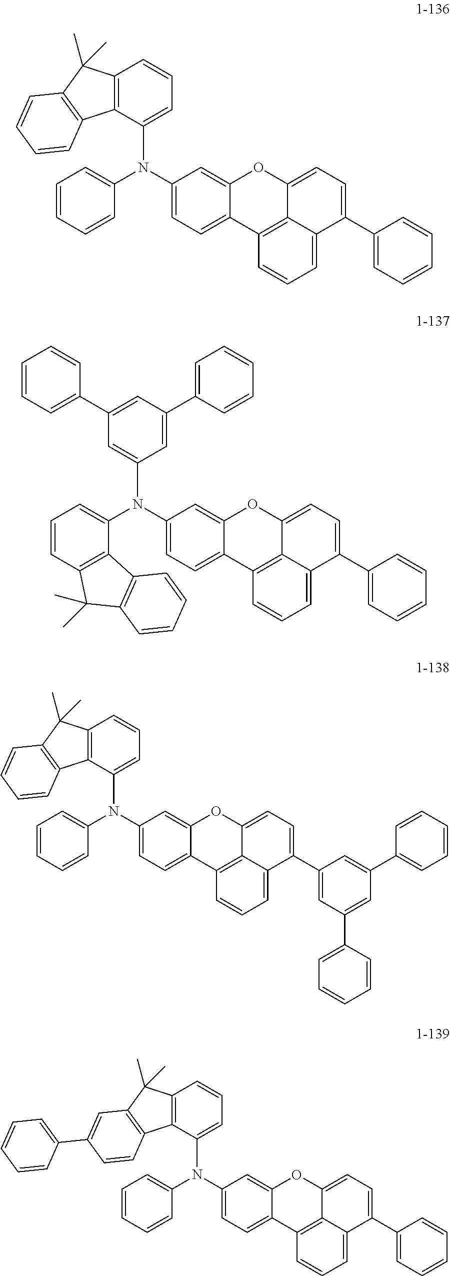 [SMILES: CC1(C)c2ccccc2-c2c(N(c3ccccc3)c3ccc4c(c3)Oc3ccc(-c5ccccc5)c5cccc-4c35)cccc21 ; CC1(C)c2ccccc2-c2c(N(c3cc(-c4ccccc4)cc(-c4ccccc4)c3)c3ccc4c(c3)Oc3ccc(-c5ccccc5)c5cccc-4c35)cccc21 ; CC1(C)c2ccccc2-c2c(N(c3ccccc3)c3ccc4c(c3)Oc3ccc(-c5cc(-c6ccccc6)cc(-c6ccccc6)c5)c5cccc-4c35)cccc21 ; CC1(C)c2cc(-c3ccccc3)ccc2-c2c(N(c3ccccc3)c3ccc4c(c3)Oc3ccc(-c5ccccc5)c5cccc-4c35)cccc21]

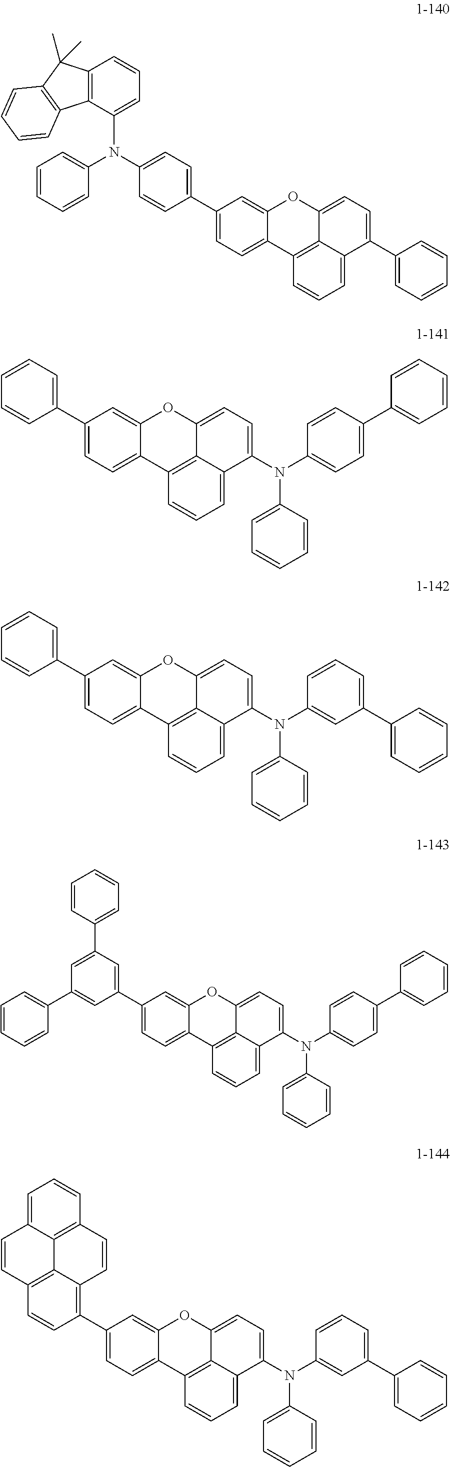 [SMILES: CC1(C)c2ccccc2-c2c(N(c3ccccc3)c3ccc(-c4ccc5c(c4)Oc4ccc(-c6ccccc6)c6cccc-5c46)cc3)cccc21 ; c1ccc(-c2ccc(N(c3ccccc3)c3ccc4c5c(cccc35)-c3ccc(-c5ccccc5)cc3O4)cc2)cc1 ; c1ccc(-c2cccc(N(c3ccccc3)c3ccc4c5c(cccc35)-c3ccc(-c5ccccc5)cc3O4)c2)cc1 ; c1ccc(-c2ccc(N(c3ccccc3)c3ccc4c5c(cccc35)-c3ccc(-c5cc(-c6ccccc6)cc(-c6ccccc6)c5)cc3O4)cc2)cc1 ; c1ccc(-c2cccc(N(c3ccccc3)c3ccc4c5c(cccc35)-c3ccc(-c5ccc6ccc7cccc8ccc5c6c78)cc3O4)c2)cc1]

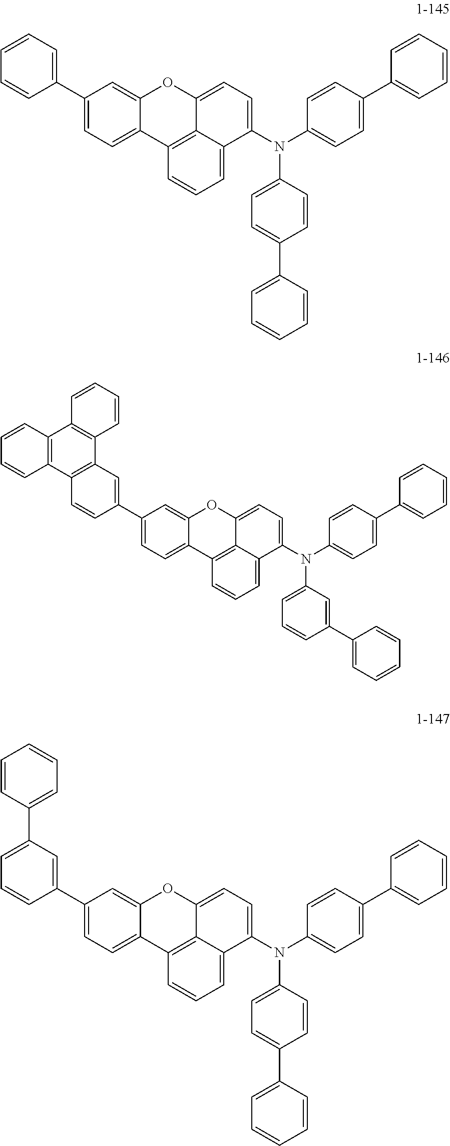 [SMILES: c1ccc(-c2ccc(N(c3ccc(-c4ccccc4)cc3)c3ccc4c5c(cccc35)-c3ccc(-c5ccccc5)cc3O4)cc2)cc1 ; c1ccc(-c2ccc(N(c3cccc(-c4ccccc4)c3)c3ccc4c5c(cccc35)-c3ccc(-c5ccc6c7ccccc7c7ccccc7c6c5)cc3O4)cc2)cc1 ; c1ccc(-c2ccc(N(c3ccc(-c4ccccc4)cc3)c3ccc4c5c(cccc35)-c3ccc(-c5cccc(-c6ccccc6)c5)cc3O4)cc2)cc1]

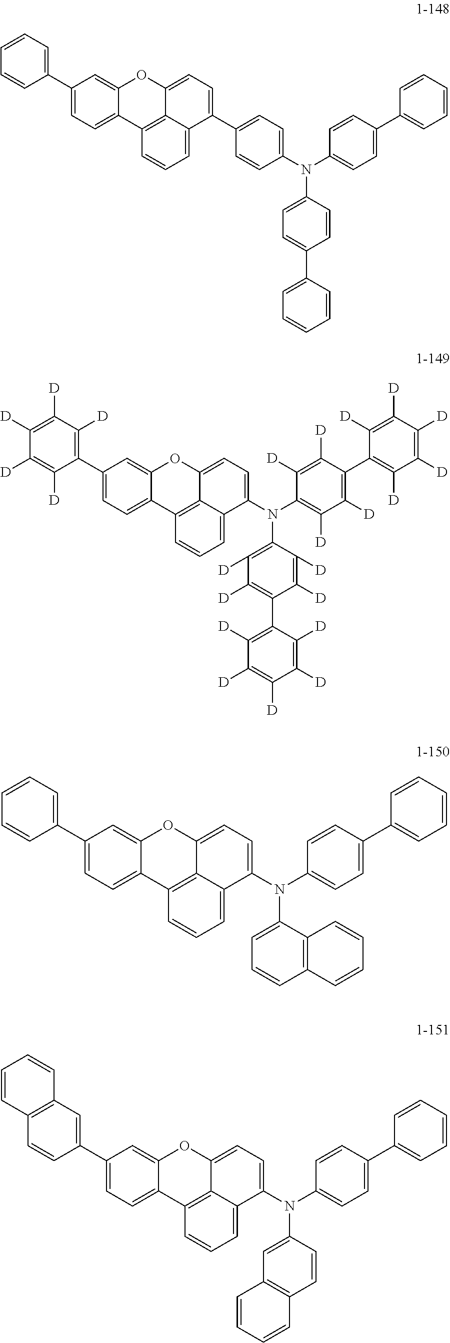 [SMILES: c1ccc(-c2ccc(N(c3ccc(-c4ccccc4)cc3)c3ccc(-c4ccc5c6c(cccc46)-c4ccc(-c6ccccc6)cc4O5)cc3)cc2)cc1 ; [2H]c1c([2H])c([2H])c(-c2ccc3c(c2)Oc2ccc(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])c4cccc-3c24)c([2H])c1[2H] ; c1ccc(-c2ccc(N(c3cccc4ccccc34)c3ccc4c5c(cccc35)-c3ccc(-c5ccccc5)cc3O4)cc2)cc1 ; c1ccc(-c2ccc(N(c3ccc4ccccc4c3)c3ccc4c5c(cccc35)-c3ccc(-c5ccc6ccccc6c5)cc3O4)cc2)cc1]

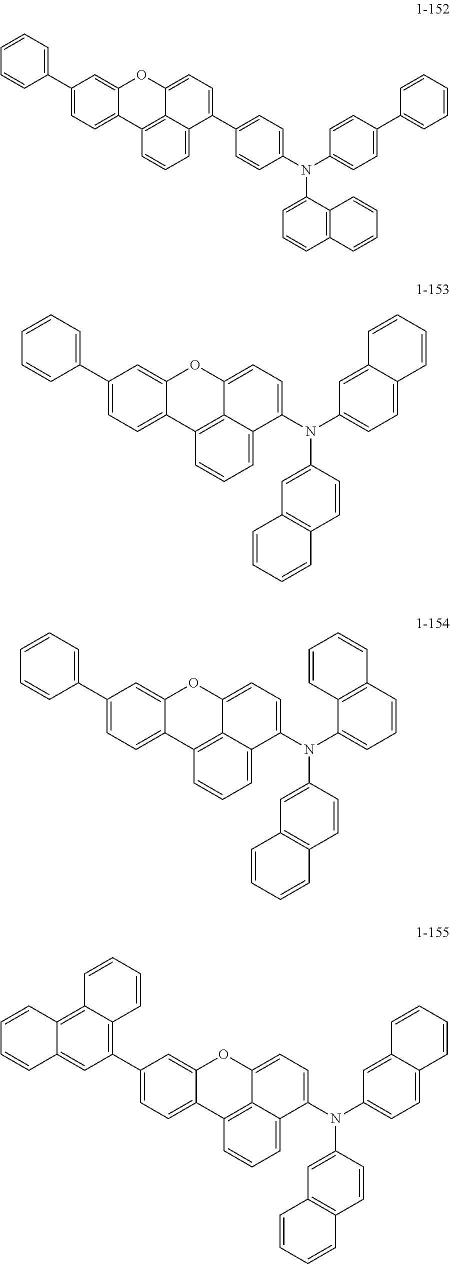 [SMILES: c1ccc(-c2ccc(N(c3ccc(-c4ccc5c6c(cccc46)-c4ccc(-c6ccccc6)cc4O5)cc3)c3cccc4ccccc34)cc2)cc1 ; c1ccc(-c2ccc3c(c2)Oc2ccc(N(c4ccc5ccccc5c4)c4ccc5ccccc5c4)c4cccc-3c24)cc1 ; c1ccc(-c2ccc3c(c2)Oc2ccc(N(c4ccc5ccccc5c4)c4cccc5ccccc45)c4cccc-3c24)cc1 ; c1ccc2cc(N(c3ccc4ccccc4c3)c3ccc4c5c(cccc35)-c3ccc(-c5cc6ccccc6c6ccccc56)cc3O4)ccc2c1]

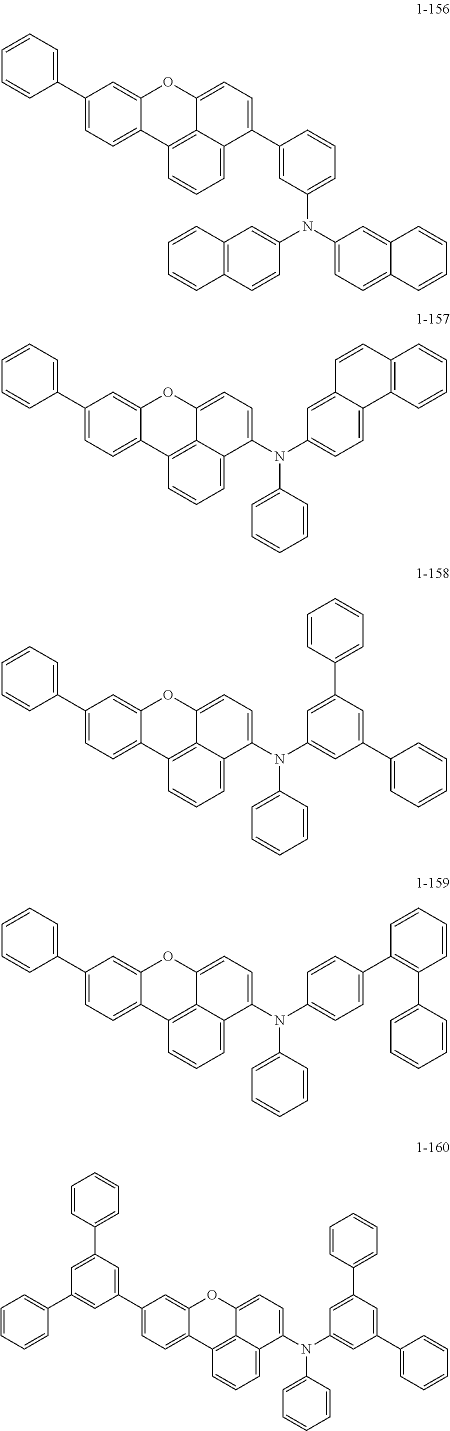 [SMILES: c1ccc(-c2ccc3c(c2)Oc2ccc(-c4cccc(N(c5ccc6ccccc6c5)c5ccc6ccccc6c5)c4)c4cccc-3c24)cc1 ; c1ccc(-c2ccc3c(c2)Oc2ccc(N(c4ccccc4)c4ccc5c(ccc6ccccc65)c4)c4cccc-3c24)cc1 ; c1ccc(-c2cc(-c3ccccc3)cc(N(c3ccccc3)c3ccc4c5c(cccc35)-c3ccc(-c5ccccc5)cc3O4)c2)cc1 ; c1ccc(-c2ccc3c(c2)Oc2ccc(N(c4ccccc4)c4ccc(-c5ccccc5-c5ccccc5)cc4)c4cccc-3c24)cc1 ; c1ccc(-c2cc(-c3ccccc3)cc(-c3ccc4c(c3)Oc3ccc(N(c5ccccc5)c5cc(-c6ccccc6)cc(-c6ccccc6)c5)c5cccc-4c35)c2)cc1]

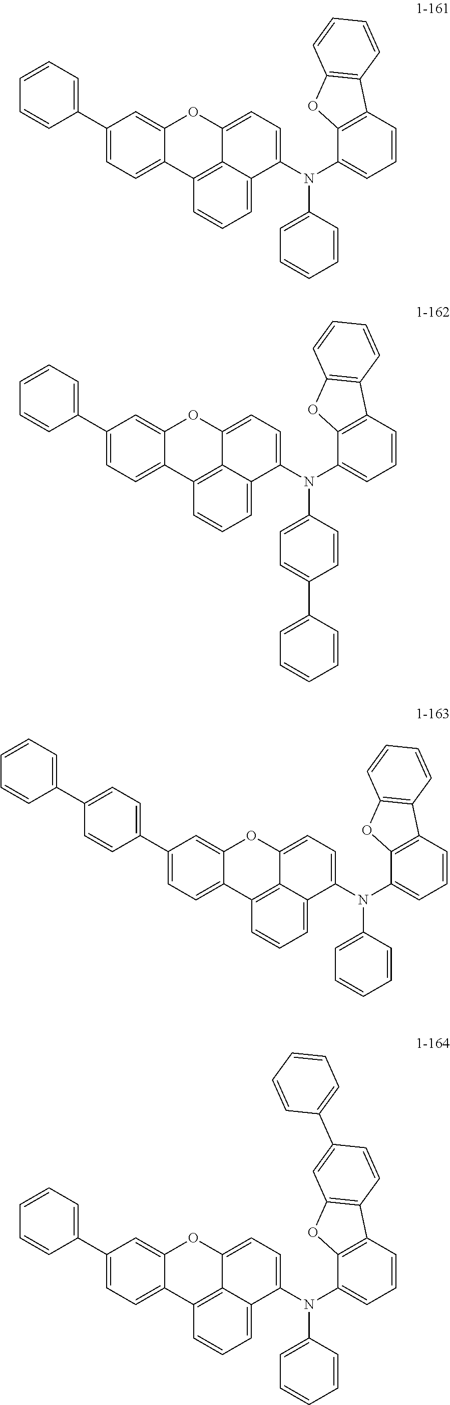 [SMILES: c1ccc(-c2ccc3c(c2)Oc2ccc(N(c4ccccc4)c4cccc5c4oc4ccccc45)c4cccc-3c24)cc1 ; c1ccc(-c2ccc(N(c3ccc4c5c(cccc35)-c3ccc(-c5ccccc5)cc3O4)c3cccc4c3oc3ccccc34)cc2)cc1 ; c1ccc(-c2ccc(-c3ccc4c(c3)Oc3ccc(N(c5ccccc5)c5cccc6c5oc5ccccc56)c5cccc-4c35)cc2)cc1 ; c1ccc(-c2ccc3c(c2)Oc2ccc(N(c4ccccc4)c4cccc5c4oc4cc(-c6ccccc6)ccc45)c4cccc-3c24)cc1]

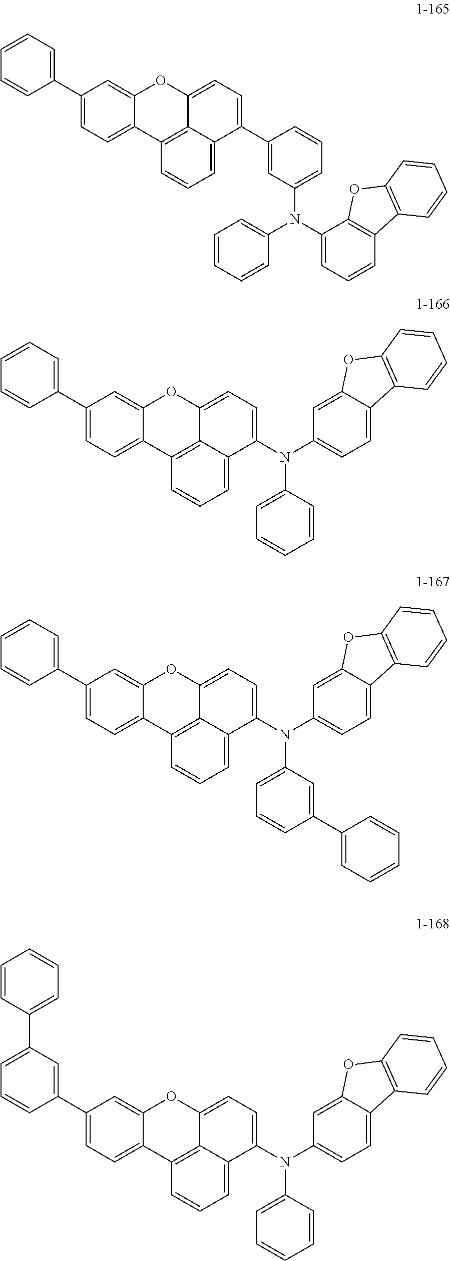 [SMILES: c1ccc(-c2ccc3c(c2)Oc2ccc(-c4cccc(N(c5ccccc5)c5cccc6c5oc5ccccc56)c4)c4cccc-3c24)cc1 ; c1ccc(-c2ccc3c(c2)Oc2ccc(N(c4ccccc4)c4ccc5c(c4)oc4ccccc45)c4cccc-3c24)cc1 ; c1ccc(-c2cccc(N(c3ccc4c(c3)oc3ccccc34)c3ccc4c5c(cccc35)-c3ccc(-c5ccccc5)cc3O4)c2)cc1 ; c1ccc(-c2cccc(-c3ccc4c(c3)Oc3ccc(N(c5ccccc5)c5ccc6c(c5)oc5ccccc56)c5cccc-4c35)c2)cc1]

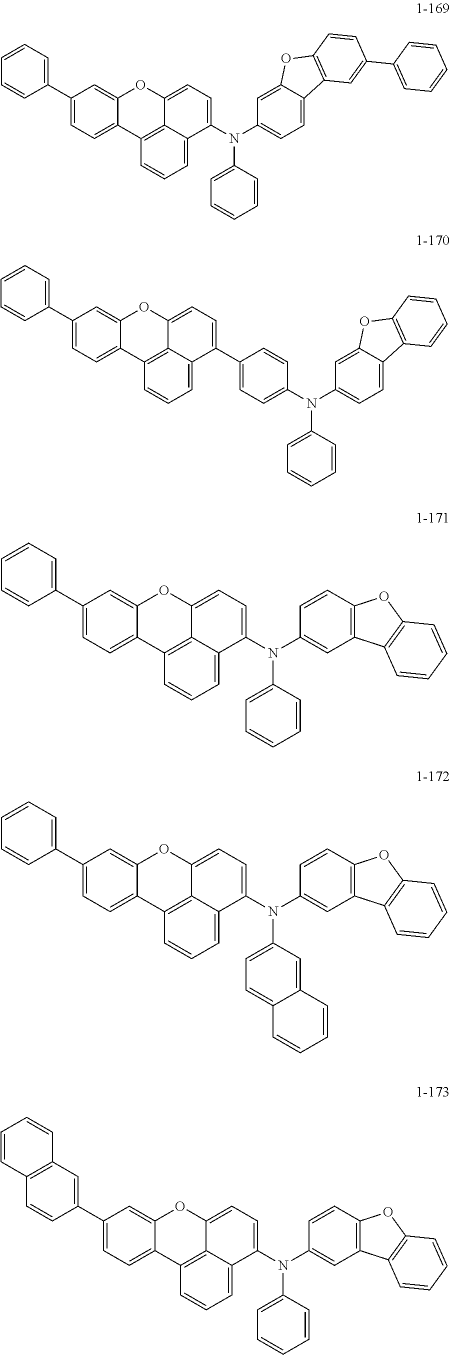 [SMILES: c1ccc(-c2ccc3c(c2)Oc2ccc(N(c4ccccc4)c4ccc5c(c4)oc4ccc(-c6ccccc6)cc45)c4cccc-3c24)cc1 ; c1ccc(-c2ccc3c(c2)Oc2ccc(-c4ccc(N(c5ccccc5)c5ccc6c(c5)oc5ccccc56)cc4)c4cccc-3c24)cc1 ; c1ccc(-c2ccc3c(c2)Oc2ccc(N(c4ccccc4)c4ccc5oc6ccccc6c5c4)c4cccc-3c24)cc1 ; c1ccc(-c2ccc3c(c2)Oc2ccc(N(c4ccc5ccccc5c4)c4ccc5oc6ccccc6c5c4)c4cccc-3c24)cc1 ; c1ccc(N(c2ccc3oc4ccccc4c3c2)c2ccc3c4c(cccc24)-c2ccc(-c4ccc5ccccc5c4)cc2O3)cc1]

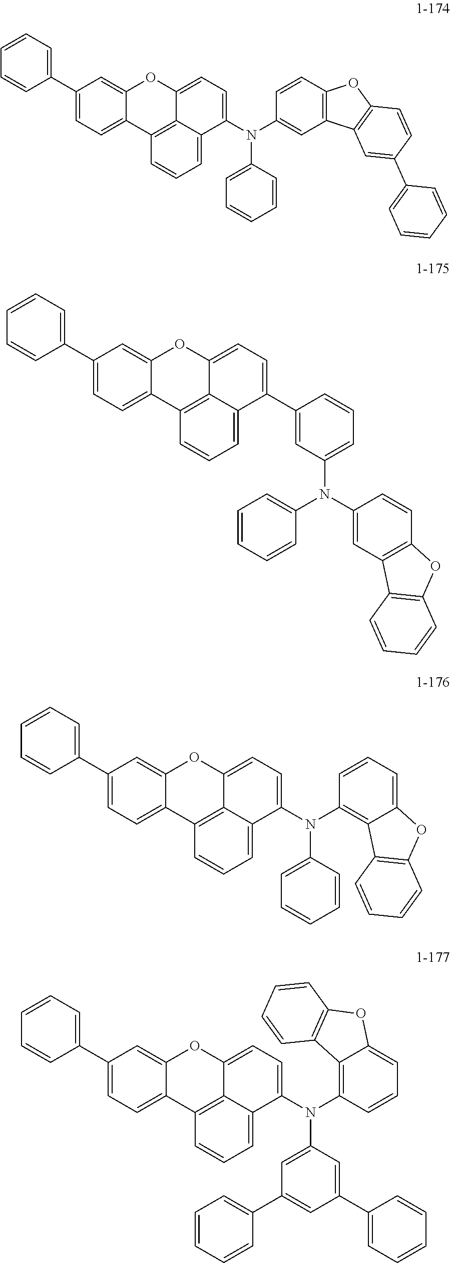 [SMILES: c1ccc(-c2ccc3c(c2)Oc2ccc(N(c4ccccc4)c4ccc5oc6ccc(-c7ccccc7)cc6c5c4)c4cccc-3c24)cc1 ; c1ccc(-c2ccc3c(c2)Oc2ccc(-c4cccc(N(c5ccccc5)c5ccc6oc7ccccc7c6c5)c4)c4cccc-3c24)cc1 ; c1ccc(-c2ccc3c(c2)Oc2ccc(N(c4ccccc4)c4cccc5oc6ccccc6c45)c4cccc-3c24)cc1 ; c1ccc(-c2cc(-c3ccccc3)cc(N(c3ccc4c5c(cccc35)-c3ccc(-c5ccccc5)cc3O4)c3cccc4oc5ccccc5c34)c2)cc1]

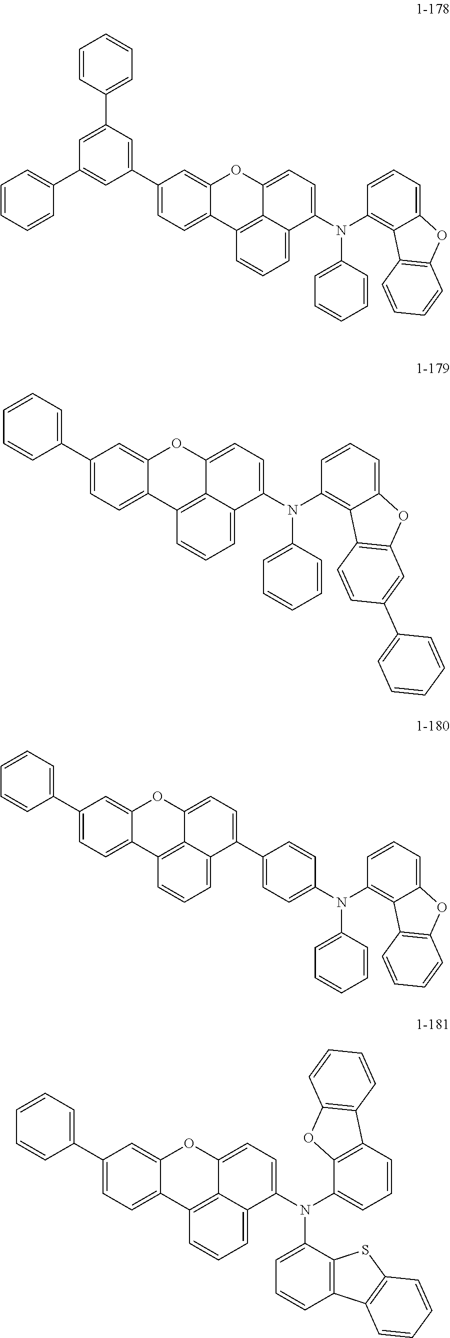 [SMILES: c1ccc(-c2cc(-c3ccccc3)cc(-c3ccc4c(c3)Oc3ccc(N(c5ccccc5)c5cccc6oc7ccccc7c56)c5cccc-4c35)c2)cc1 ; c1ccc(-c2ccc3c(c2)Oc2ccc(N(c4ccccc4)c4cccc5oc6cc(-c7ccccc7)ccc6c45)c4cccc-3c24)cc1 ; c1ccc(-c2ccc3c(c2)Oc2ccc(-c4ccc(N(c5ccccc5)c5cccc6oc7ccccc7c56)cc4)c4cccc-3c24)cc1 ; c1ccc(-c2ccc3c(c2)Oc2ccc(N(c4cccc5c4oc4ccccc45)c4cccc5c4sc4ccccc45)c4cccc-3c24)cc1]

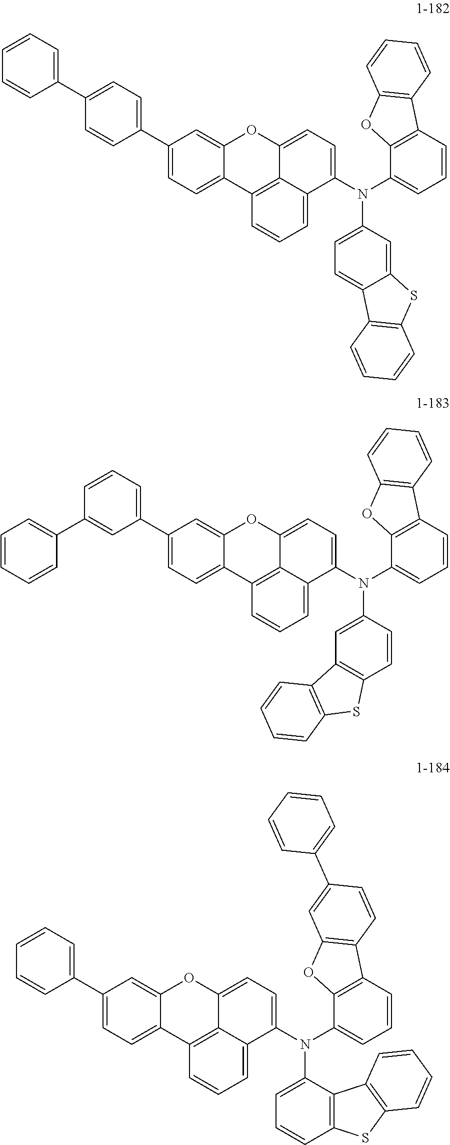 [SMILES: c1ccc(-c2ccc(-c3ccc4c(c3)Oc3ccc(N(c5ccc6c(c5)sc5ccccc56)c5cccc6c5oc5ccccc56)c5cccc-4c35)cc2)cc1 ; c1ccc(-c2cccc(-c3ccc4c(c3)Oc3ccc(N(c5ccc6sc7ccccc7c6c5)c5cccc6c5oc5ccccc56)c5cccc-4c35)c2)cc1 ; c1ccc(-c2ccc3c(c2)Oc2ccc(N(c4cccc5c4oc4cc(-c6ccccc6)ccc45)c4cccc5sc6ccccc6c45)c4cccc-3c24)cc1]

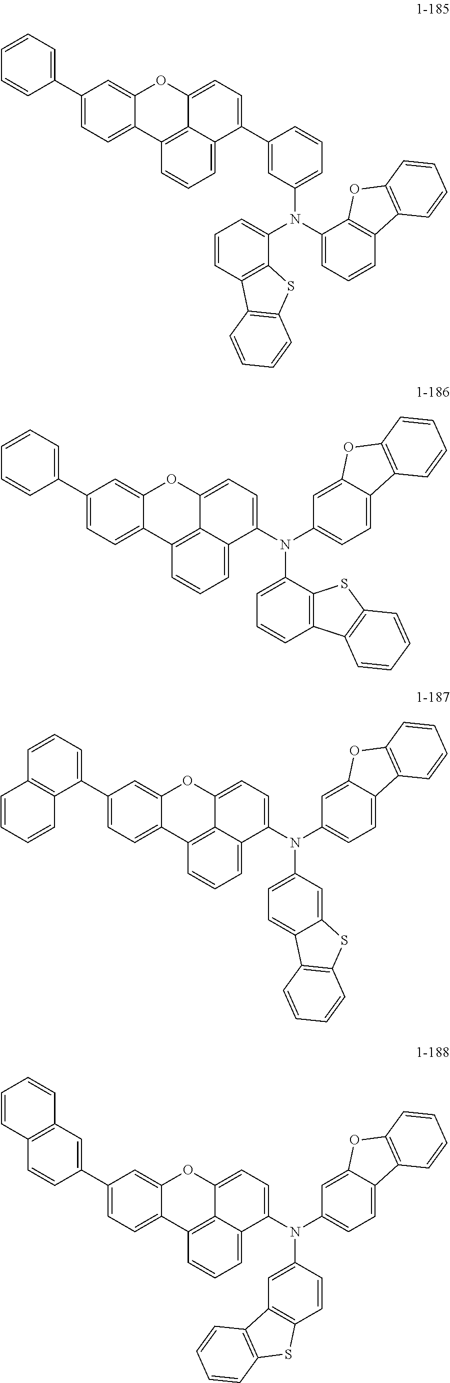 [SMILES: c1ccc(-c2ccc3c(c2)Oc2ccc(-c4cccc(N(c5cccc6c5oc5ccccc56)c5cccc6c5sc5ccccc56)c4)c4cccc-3c24)cc1 ; c1ccc(-c2ccc3c(c2)Oc2ccc(N(c4ccc5c(c4)oc4ccccc45)c4cccc5c4sc4ccccc45)c4cccc-3c24)cc1 ; c1ccc2c(-c3ccc4c(c3)Oc3ccc(N(c5ccc6c(c5)oc5ccccc56)c5ccc6c(c5)sc5ccccc56)c5cccc-4c35)cccc2c1 ; c1ccc2cc(-c3ccc4c(c3)Oc3ccc(N(c5ccc6c(c5)oc5ccccc56)c5ccc6sc7ccccc7c6c5)c5cccc-4c35)ccc2c1]

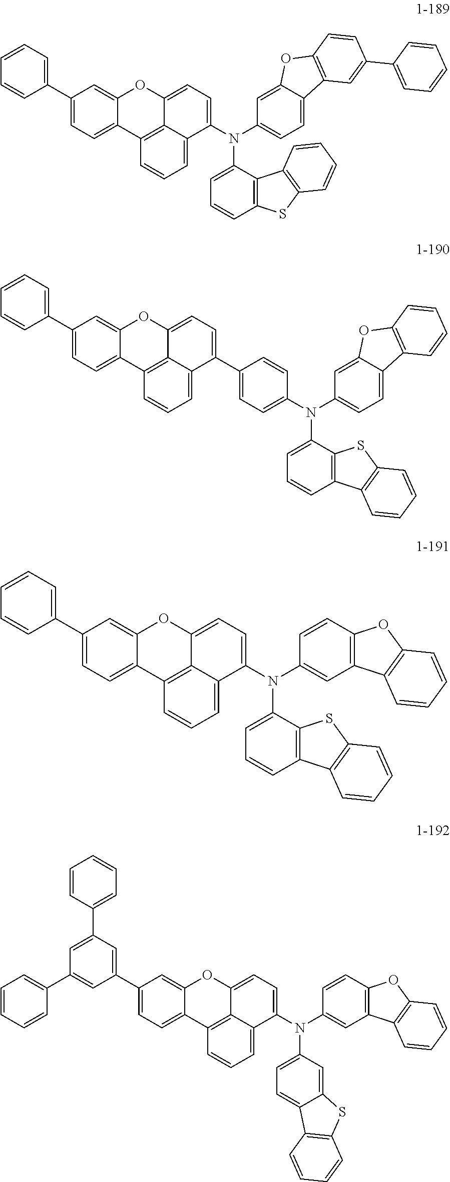 [SMILES: c1ccc(-c2ccc3c(c2)Oc2ccc(N(c4ccc5c(c4)oc4ccc(-c6ccccc6)cc45)c4cccc5sc6ccccc6c45)c4cccc-3c24)cc1 ; c1ccc(-c2ccc3c(c2)Oc2ccc(-c4ccc(N(c5ccc6c(c5)oc5ccccc56)c5cccc6c5sc5ccccc56)cc4)c4cccc-3c24)cc1 ; c1ccc(-c2ccc3c(c2)Oc2ccc(N(c4ccc5oc6ccccc6c5c4)c4cccc5c4sc4ccccc45)c4cccc-3c24)cc1 ; c1ccc(-c2cc(-c3ccccc3)cc(-c3ccc4c(c3)Oc3ccc(N(c5ccc6c(c5)sc5ccccc56)c5ccc6oc7ccccc7c6c5)c5cccc-4c35)c2)cc1]

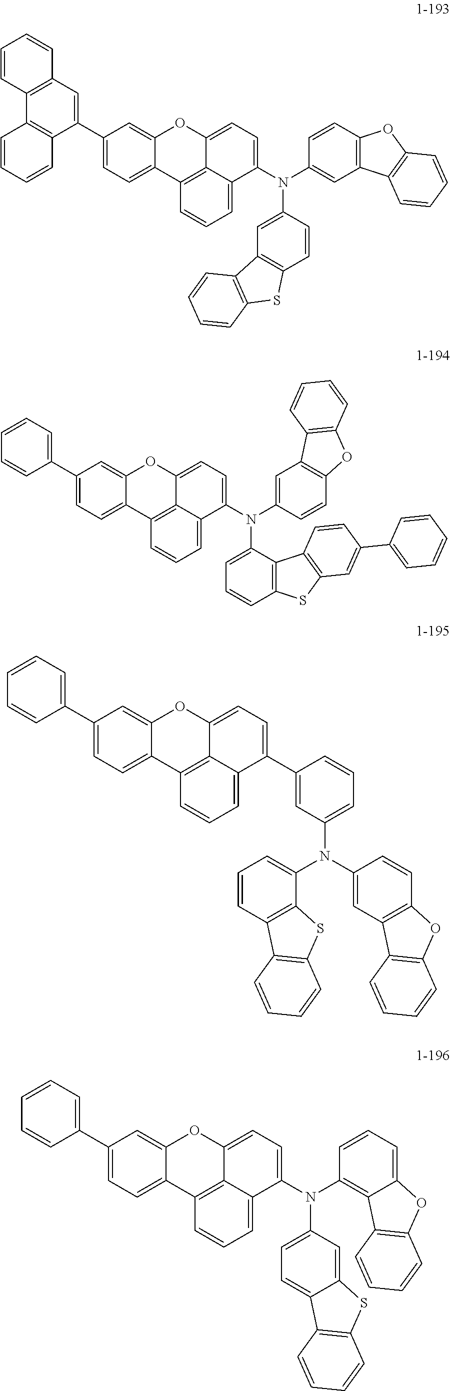 [SMILES: c1ccc2c(c1)cc(-c1ccc3c(c1)Oc1ccc(N(c4ccc5oc6ccccc6c5c4)c4ccc5sc6ccccc6c5c4)c4cccc-3c14)c1ccccc12 ; c1ccc(-c2ccc3c(c2)Oc2ccc(N(c4ccc5oc6ccccc6c5c4)c4cccc5sc6cc(-c7ccccc7)ccc6c45)c4cccc-3c24)cc1 ; c1ccc(-c2ccc3c(c2)Oc2ccc(-c4cccc(N(c5ccc6oc7ccccc7c6c5)c5cccc6c5sc5ccccc56)c4)c4cccc-3c24)cc1 ; c1ccc(-c2ccc3c(c2)Oc2ccc(N(c4ccc5c(c4)sc4ccccc45)c4cccc5oc6ccccc6c45)c4cccc-3c24)cc1]

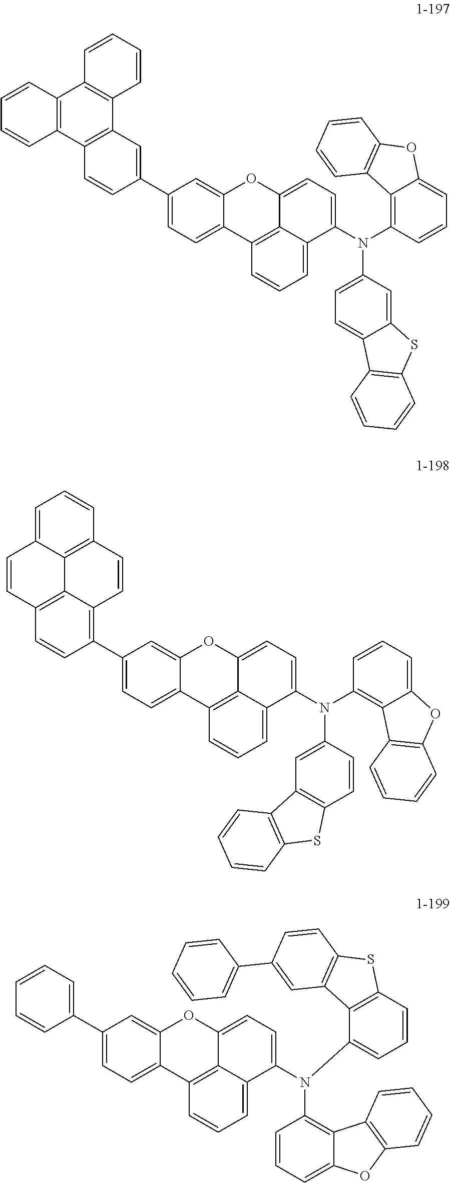 [SMILES: c1ccc2c(c1)oc1cccc(N(c3ccc4c(c3)sc3ccccc34)c3ccc4c5c(cccc35)-c3ccc(-c5ccc6c7ccccc7c7ccccc7c6c5)cc3O4)c12 ; c1cc2ccc3ccc(-c4ccc5c(c4)Oc4ccc(N(c6ccc7sc8ccccc8c7c6)c6cccc7oc8ccccc8c67)c6cccc-5c46)c4ccc(c1)c2c34 ; c1ccc(-c2ccc3c(c2)Oc2ccc(N(c4cccc5oc6ccccc6c45)c4cccc5sc6ccc(-c7ccccc7)cc6c45)c4cccc-3c24)cc1]

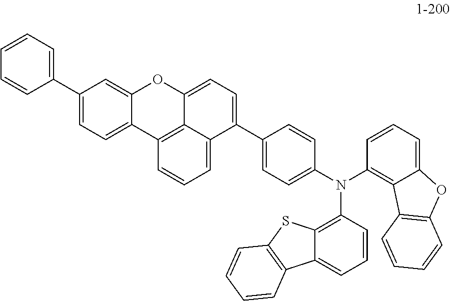 [SMILES: c1ccc(-c2ccc3c(c2)Oc2ccc(-c4ccc(N(c5cccc6c5sc5ccccc56)c5cccc6oc7ccccc7c56)cc4)c4cccc-3c24)cc1]

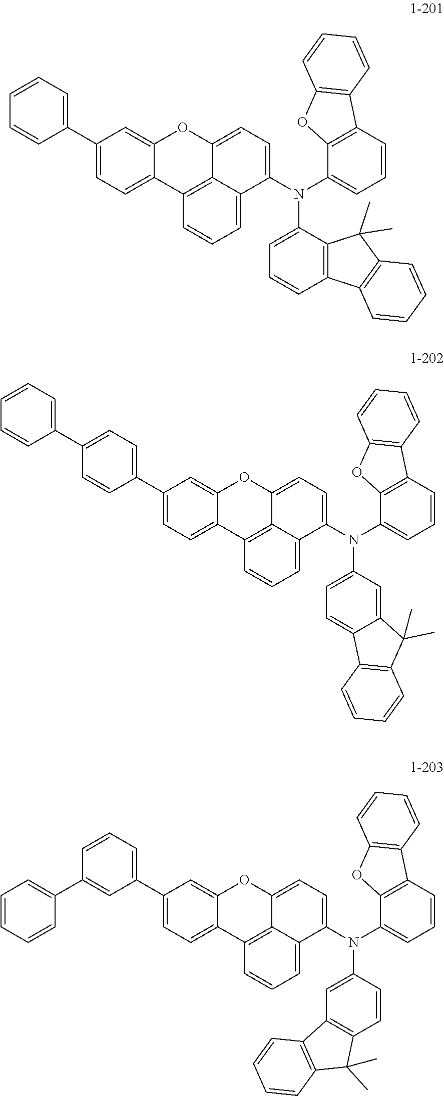 [SMILES: CC1(C)c2ccccc2-c2cccc(N(c3ccc4c5c(cccc35)-c3ccc(-c5ccccc5)cc3O4)c3cccc4c3oc3ccccc34)c21 ; CC1(C)c2ccccc2-c2ccc(N(c3ccc4c5c(cccc35)-c3ccc(-c5ccc(-c6ccccc6)cc5)cc3O4)c3cccc4c3oc3ccccc34)cc21 ; CC1(C)c2ccccc2-c2cc(N(c3ccc4c5c(cccc35)-c3ccc(-c5cccc(-c6ccccc6)c5)cc3O4)c3cccc4c3oc3ccccc34)ccc21]

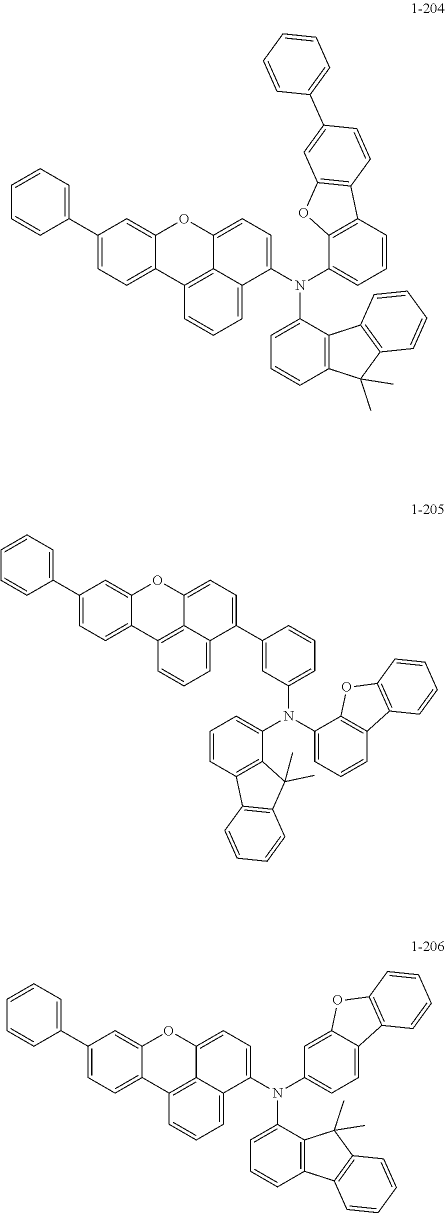

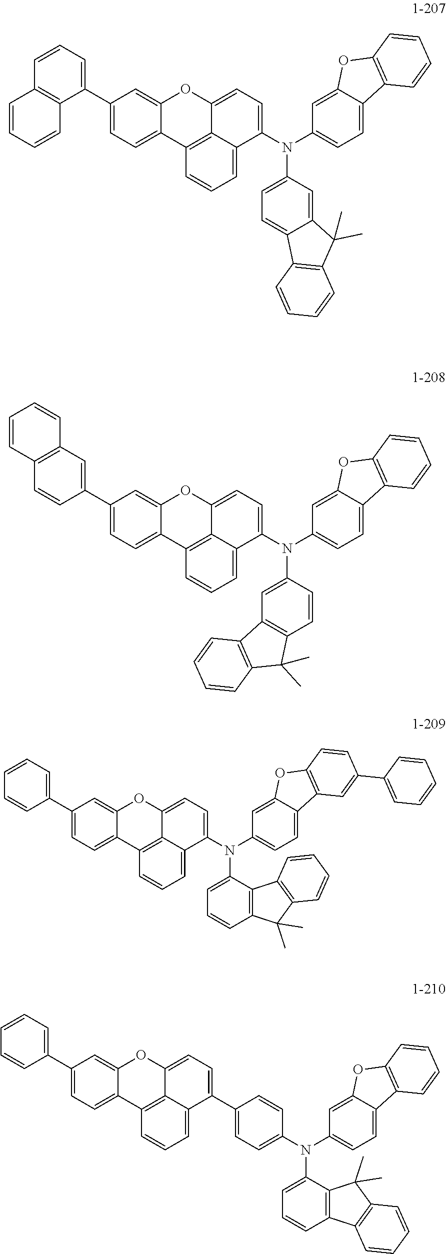 [SMILES: CC1(C)c2ccccc2-c2ccc(N(c3ccc4c(c3)oc3ccccc34)c3ccc4c5c(cccc35)-c3ccc(-c5cccc6ccccc56)cc3O4)cc21 ; CC1(C)c2ccccc2-c2cc(N(c3ccc4c(c3)oc3ccccc34)c3ccc4c5c(cccc35)-c3ccc(-c5ccc6ccccc6c5)cc3O4)ccc21 ; CC1(C)c2ccccc2-c2c(N(c3ccc4c(c3)oc3ccc(-c5ccccc5)cc34)c3ccc4c5c(cccc35)-c3ccc(-c5ccccc5)cc3O4)cccc21 ; CC1(C)c2ccccc2-c2cccc(N(c3ccc(-c4ccc5c6c(cccc46)-c4ccc(-c6ccccc6)cc4O5)cc3)c3ccc4c(c3)oc3ccccc34)c21]

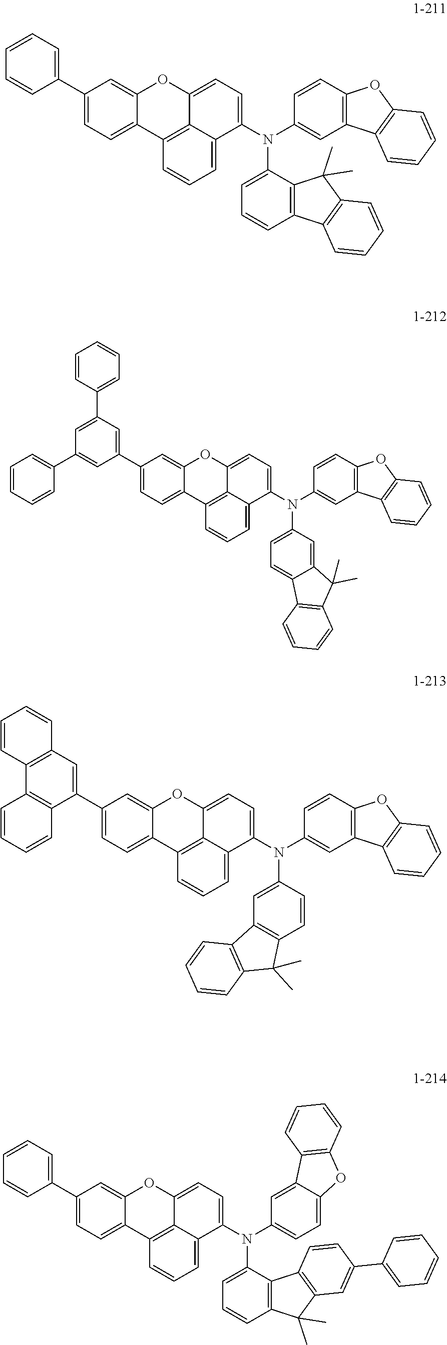 [SMILES: CC1(C)c2ccccc2-c2cccc(N(c3ccc4oc5ccccc5c4c3)c3ccc4c5c(cccc35)-c3ccc(-c5ccccc5)cc3O4)c21 ; CC1(C)c2ccccc2-c2ccc(N(c3ccc4oc5ccccc5c4c3)c3ccc4c5c(cccc35)-c3ccc(-c5cc(-c6ccccc6)cc(-c6ccccc6)c5)cc3O4)cc21 ; CC1(C)c2ccccc2-c2cc(N(c3ccc4oc5ccccc5c4c3)c3ccc4c5c(cccc35)-c3ccc(-c5cc6ccccc6c6ccccc56)cc3O4)ccc21 ; CC1(C)c2cc(-c3ccccc3)ccc2-c2c(N(c3ccc4oc5ccccc5c4c3)c3ccc4c5c(cccc35)-c3ccc(-c5ccccc5)cc3O4)cccc21]

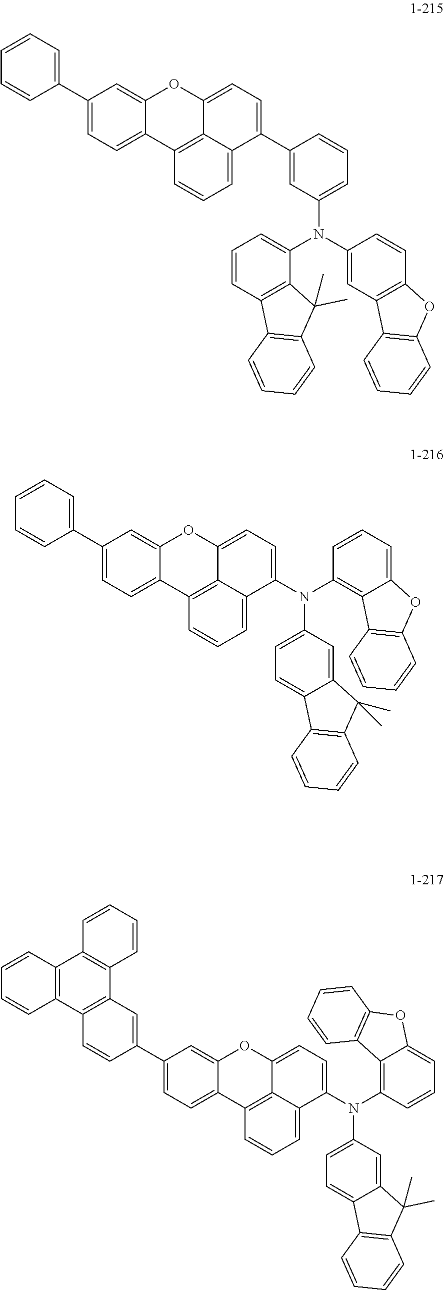

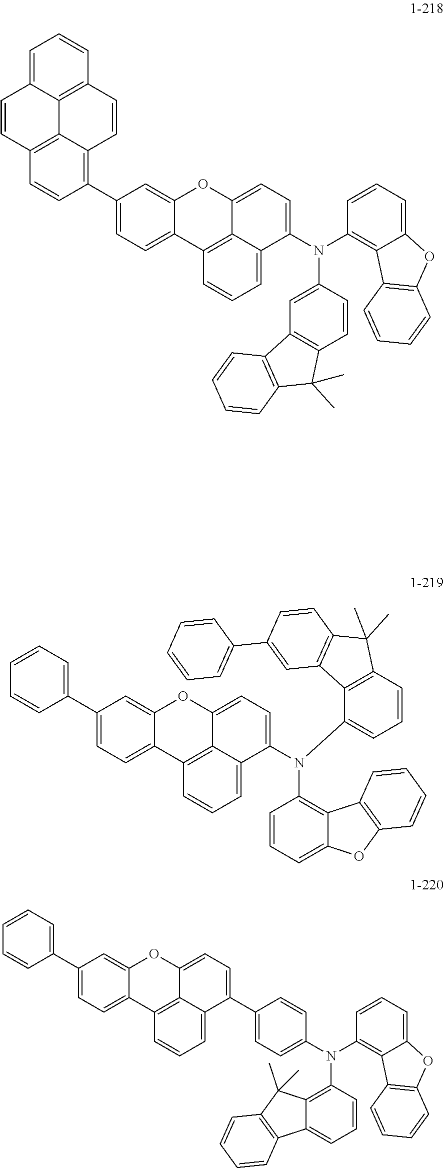 [SMILES: CC1(C)c2ccccc2-c2cc(N(c3ccc4c5c(cccc35)-c3ccc(-c5ccc6ccc7cccc8ccc5c6c78)cc3O4)c3cccc4oc5ccccc5c34)ccc21 ; CC1(C)c2ccc(-c3ccccc3)cc2-c2c(N(c3ccc4c5c(cccc35)-c3ccc(-c5ccccc5)cc3O4)c3cccc4oc5ccccc5c34)cccc21 ; CC1(C)c2ccccc2-c2cccc(N(c3ccc(-c4ccc5c6c(cccc46)-c4ccc(-c6ccccc6)cc4O5)cc3)c3cccc4oc5ccccc5c34)c21]

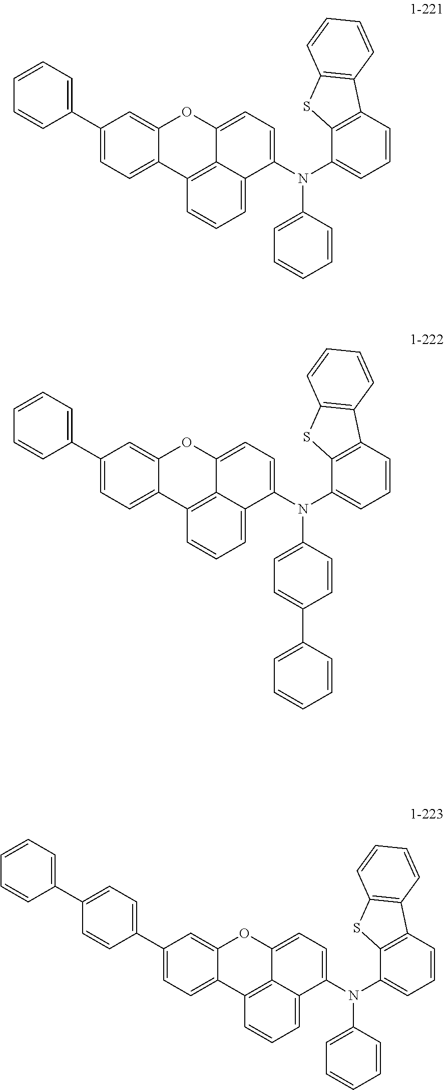 [SMILES: c1ccc(-c2ccc3c(c2)Oc2ccc(N(c4ccccc4)c4cccc5c4sc4ccccc45)c4cccc-3c24)cc1 ; c1ccc(-c2ccc(N(c3ccc4c5c(cccc35)-c3ccc(-c5ccccc5)cc3O4)c3cccc4c3sc3ccccc34)cc2)cc1 ; c1ccc(-c2ccc(-c3ccc4c(c3)Oc3ccc(N(c5ccccc5)c5cccc6c5sc5ccccc56)c5cccc-4c35)cc2)cc1]

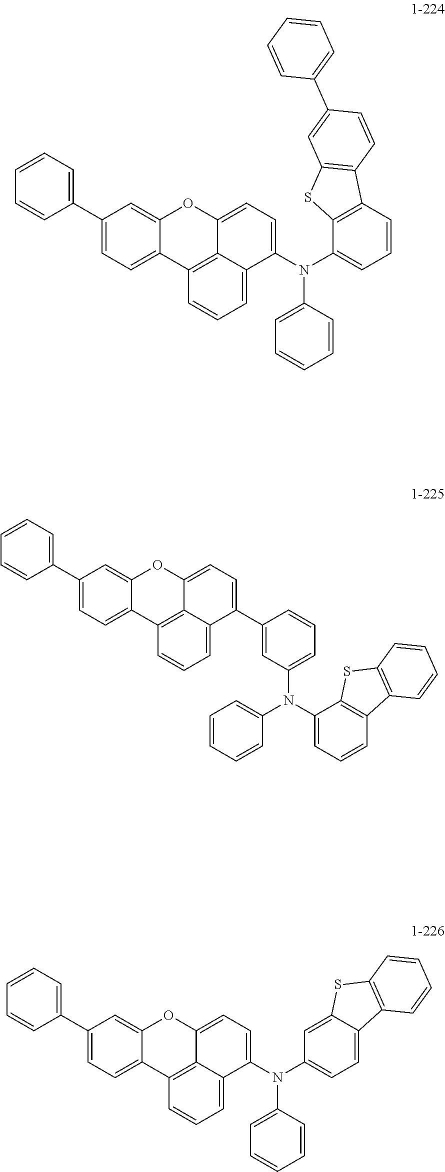

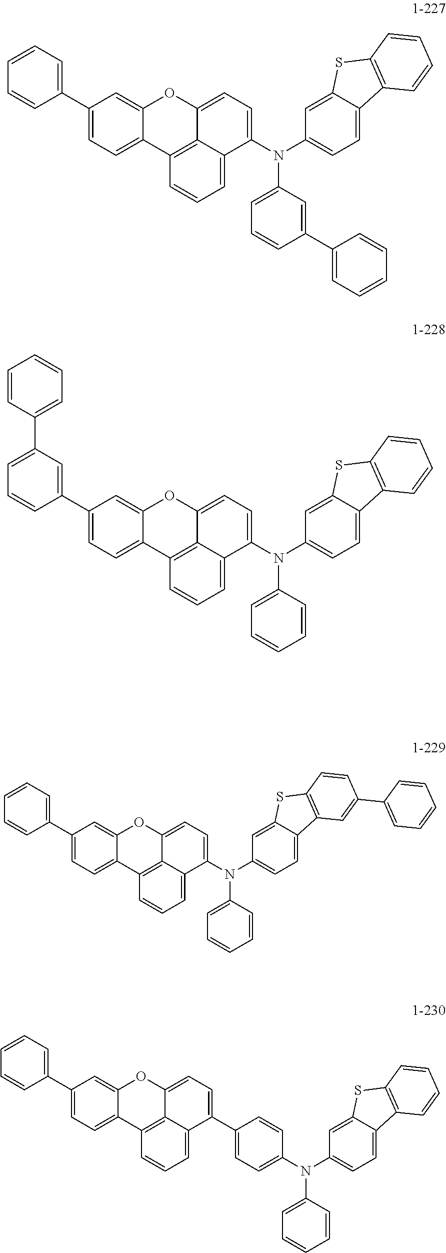 [SMILES: c1ccc(-c2cccc(N(c3ccc4c(c3)sc3ccccc34)c3ccc4c5c(cccc35)-c3ccc(-c5ccccc5)cc3O4)c2)cc1 ; c1ccc(-c2cccc(-c3ccc4c(c3)Oc3ccc(N(c5ccccc5)c5ccc6c(c5)sc5ccccc56)c5cccc-4c35)c2)cc1 ; c1ccc(-c2ccc3c(c2)Oc2ccc(N(c4ccccc4)c4ccc5c(c4)sc4ccc(-c6ccccc6)cc45)c4cccc-3c24)cc1 ; c1ccc(-c2ccc3c(c2)Oc2ccc(-c4ccc(N(c5ccccc5)c5ccc6c(c5)sc5ccccc56)cc4)c4cccc-3c24)cc1]

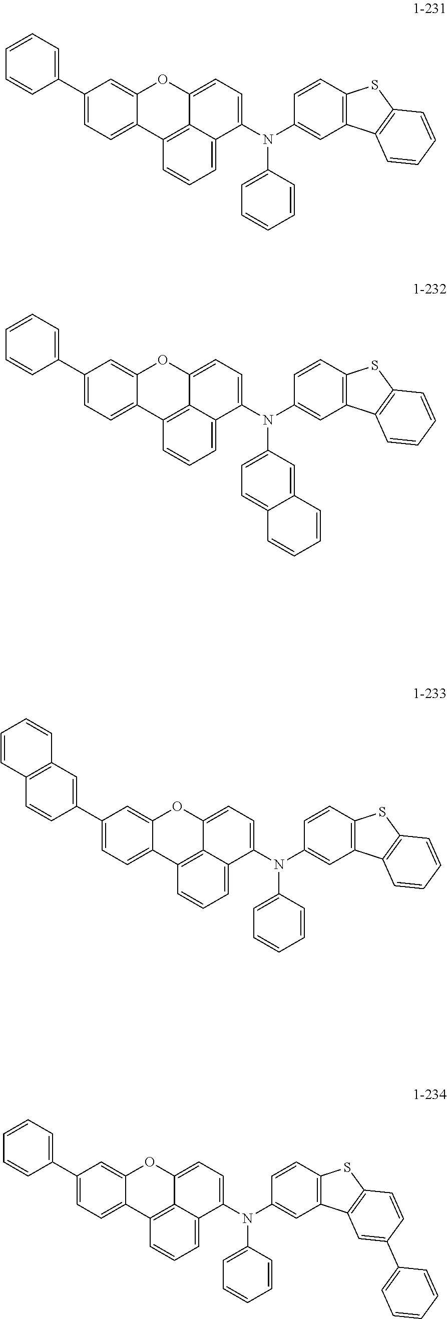 [SMILES: c1ccc(-c2ccc3c(c2)Oc2ccc(N(c4ccccc4)c4ccc5sc6ccccc6c5c4)c4cccc-3c24)cc1 ; c1ccc(-c2ccc3c(c2)Oc2ccc(N(c4ccc5ccccc5c4)c4ccc5sc6ccccc6c5c4)c4cccc-3c24)cc1 ; c1ccc(N(c2ccc3sc4ccccc4c3c2)c2ccc3c4c(cccc24)-c2ccc(-c4ccc5ccccc5c4)cc2O3)cc1 ; c1ccc(-c2ccc3c(c2)Oc2ccc(N(c4ccccc4)c4ccc5sc6ccc(-c7ccccc7)cc6c5c4)c4cccc-3c24)cc1]

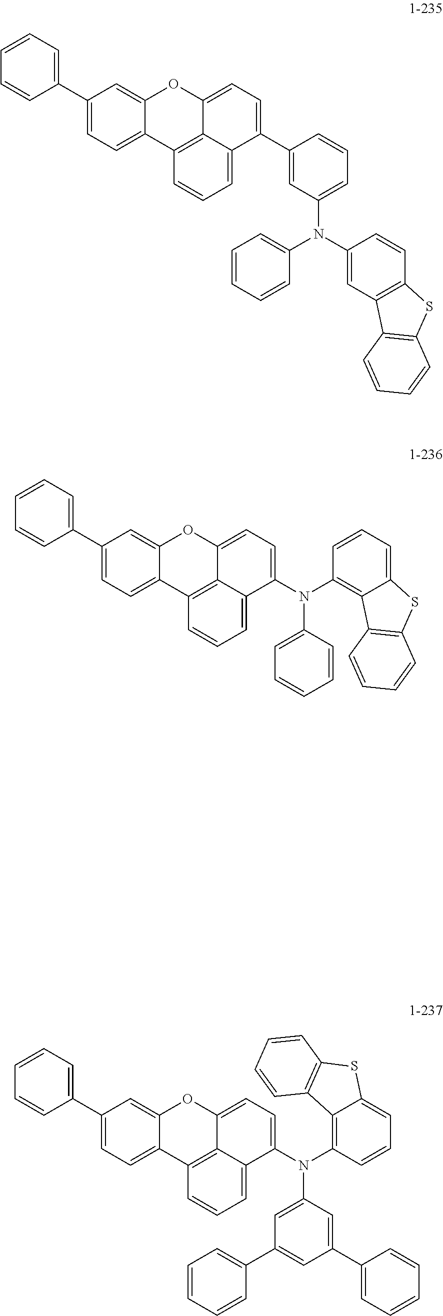

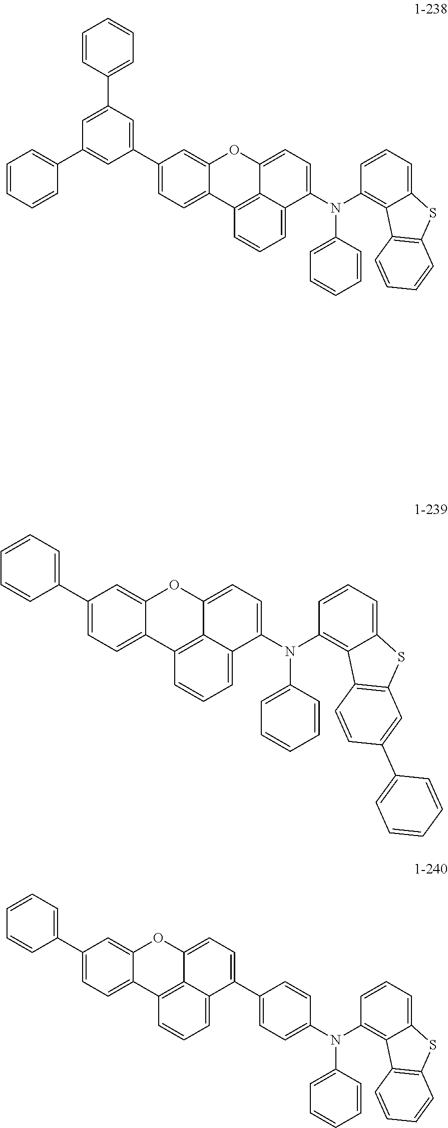 [SMILES: c1ccc(-c2cc(-c3ccccc3)cc(-c3ccc4c(c3)Oc3ccc(N(c5ccccc5)c5cccc6sc7ccccc7c56)c5cccc-4c35)c2)cc1 ; c1ccc(-c2ccc3c(c2)Oc2ccc(N(c4ccccc4)c4cccc5sc6cc(-c7ccccc7)ccc6c45)c4cccc-3c24)cc1 ; c1ccc(-c2ccc3c(c2)Oc2ccc(-c4ccc(N(c5ccccc5)c5cccc6sc7ccccc7c56)cc4)c4cccc-3c24)cc1]

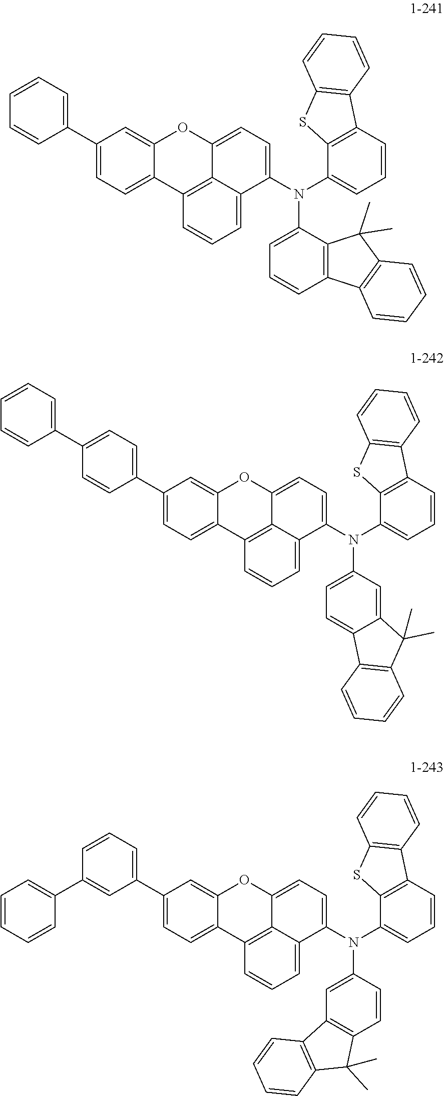 [SMILES: CC1(C)c2ccccc2-c2cccc(N(c3ccc4c5c(cccc35)-c3ccc(-c5ccccc5)cc3O4)c3cccc4c3sc3ccccc34)c21 ; CC1(C)c2ccccc2-c2ccc(N(c3ccc4c5c(cccc35)-c3ccc(-c5ccc(-c6ccccc6)cc5)cc3O4)c3cccc4c3sc3ccccc34)cc21 ; CC1(C)c2ccccc2-c2cc(N(c3ccc4c5c(cccc35)-c3ccc(-c5cccc(-c6ccccc6)c5)cc3O4)c3cccc4c3sc3ccccc34)ccc21]

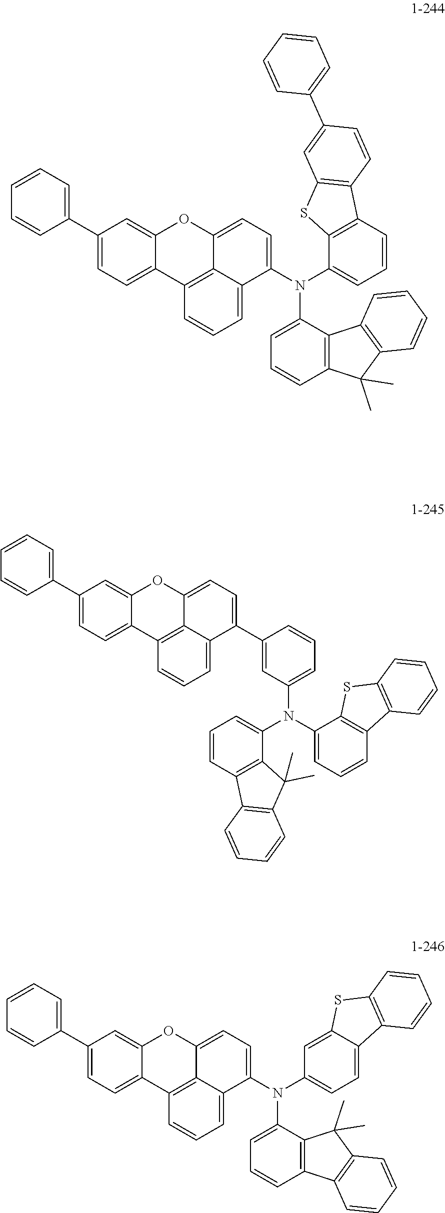

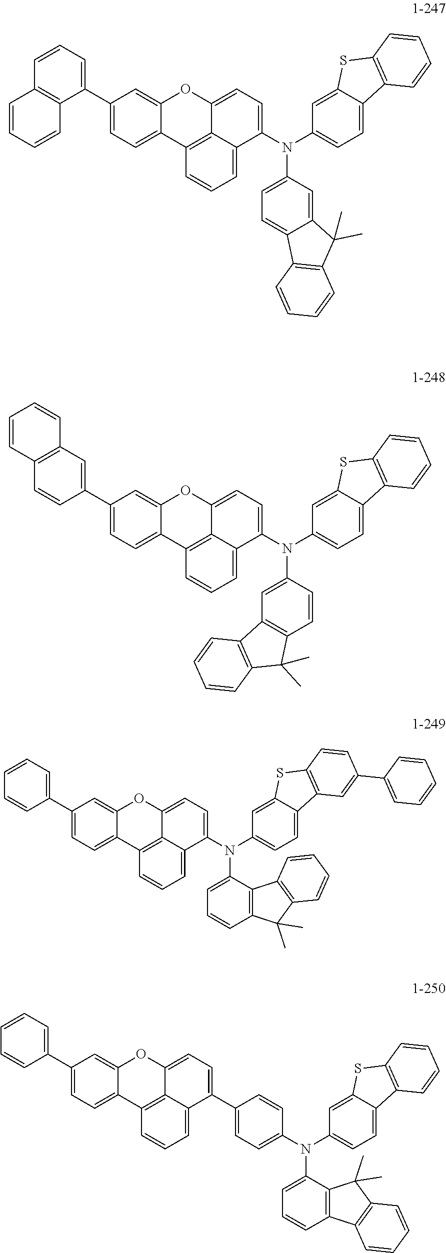 [SMILES: CC1(C)c2ccccc2-c2ccc(N(c3ccc4c(c3)sc3ccccc34)c3ccc4c5c(cccc35)-c3ccc(-c5cccc6ccccc56)cc3O4)cc21 ; CC1(C)c2ccccc2-c2cc(N(c3ccc4c(c3)sc3ccccc34)c3ccc4c5c(cccc35)-c3ccc(-c5ccc6ccccc6c5)cc3O4)ccc21 ; CC1(C)c2ccccc2-c2c(N(c3ccc4c(c3)sc3ccc(-c5ccccc5)cc34)c3ccc4c5c(cccc35)-c3ccc(-c5ccccc5)cc3O4)cccc21 ; CC1(C)c2ccccc2-c2cccc(N(c3ccc(-c4ccc5c6c(cccc46)-c4ccc(-c6ccccc6)cc4O5)cc3)c3ccc4c(c3)sc3ccccc34)c21]

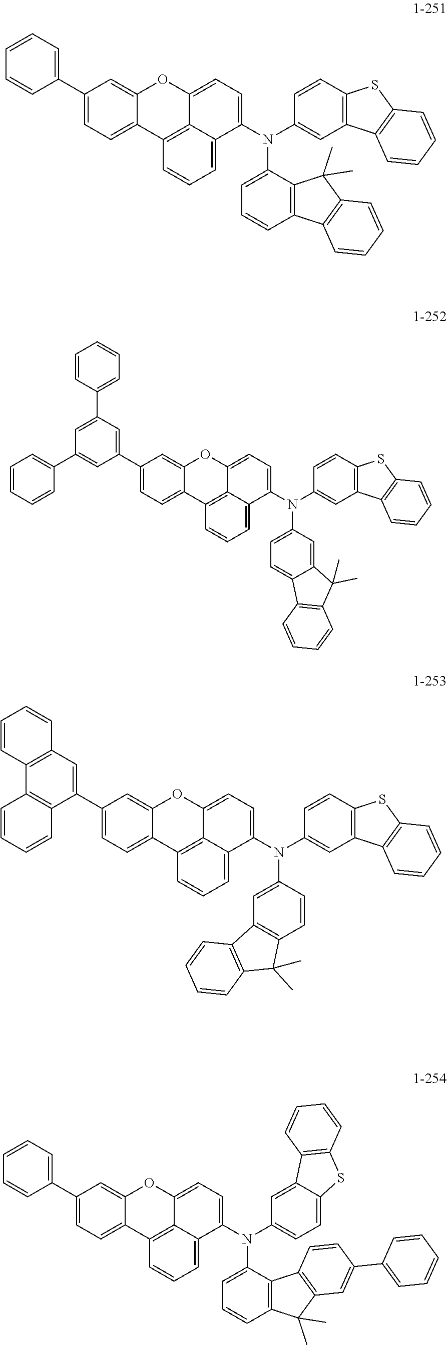 [SMILES: CC1(C)c2ccccc2-c2cccc(N(c3ccc4sc5ccccc5c4c3)c3ccc4c5c(cccc35)-c3ccc(-c5ccccc5)cc3O4)c21 ; CC1(C)c2ccccc2-c2ccc(N(c3ccc4sc5ccccc5c4c3)c3ccc4c5c(cccc35)-c3ccc(-c5cc(-c6ccccc6)cc(-c6ccccc6)c5)cc3O4)cc21 ; CC1(C)c2ccccc2-c2cc(N(c3ccc4sc5ccccc5c4c3)c3ccc4c5c(cccc35)-c3ccc(-c5cc6ccccc6c6ccccc56)cc3O4)ccc21 ; CC1(C)c2cc(-c3ccccc3)ccc2-c2c(N(c3ccc4sc5ccccc5c4c3)c3ccc4c5c(cccc35)-c3ccc(-c5ccccc5)cc3O4)cccc21]

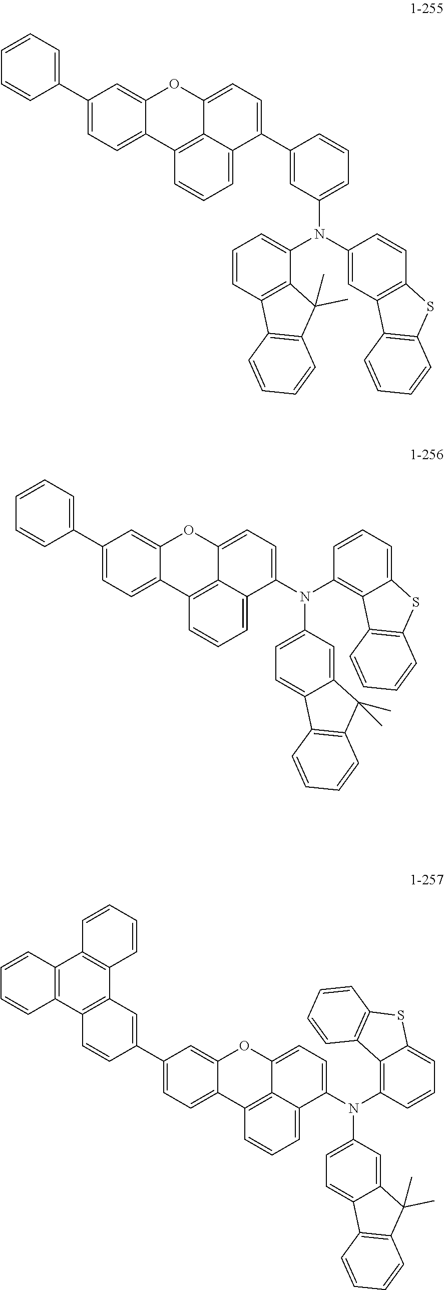

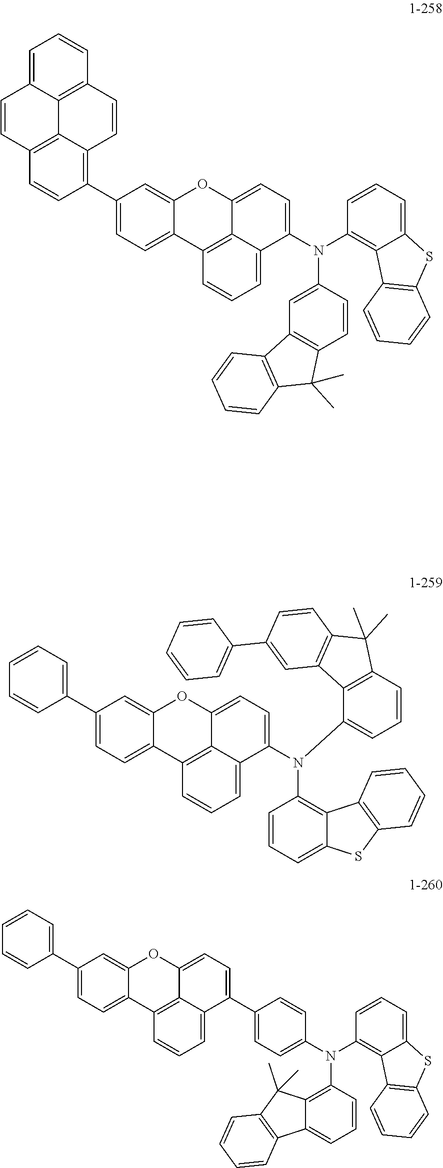 [SMILES: CC1(C)c2ccccc2-c2cc(N(c3ccc4c5c(cccc35)-c3ccc(-c5ccc6ccc7cccc8ccc5c6c78)cc3O4)c3cccc4sc5ccccc5c34)ccc21 ; CC1(C)c2ccc(-c3ccccc3)cc2-c2c(N(c3ccc4c5c(cccc35)-c3ccc(-c5ccccc5)cc3O4)c3cccc4sc5ccccc5c34)cccc21 ; CC1(C)c2ccccc2-c2cccc(N(c3ccc(-c4ccc5c6c(cccc46)-c4ccc(-c6ccccc6)cc4O5)cc3)c3cccc4sc5ccccc5c34)c21]

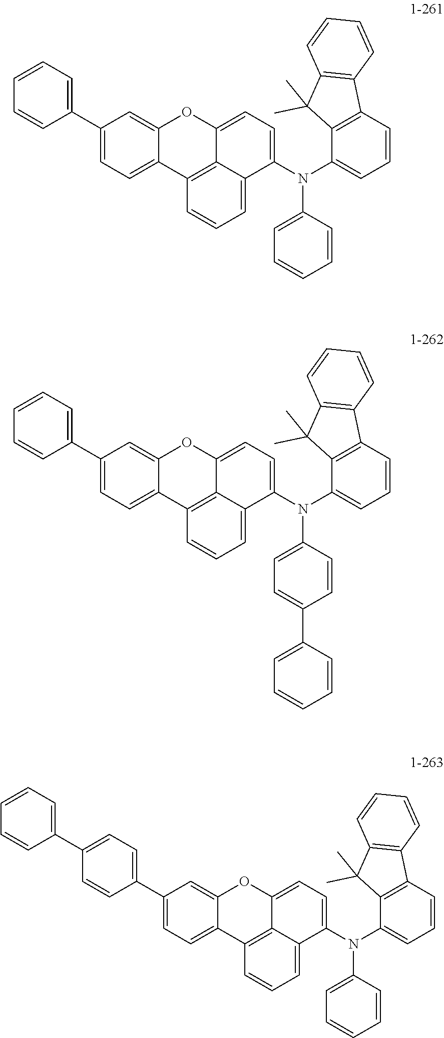 [SMILES: CC1(C)c2ccccc2-c2cccc(N(c3ccccc3)c3ccc4c5c(cccc35)-c3ccc(-c5ccccc5)cc3O4)c21 ; CC1(C)c2ccccc2-c2cccc(N(c3ccc(-c4ccccc4)cc3)c3ccc4c5c(cccc35)-c3ccc(-c5ccccc5)cc3O4)c21 ; CC1(C)c2ccccc2-c2cccc(N(c3ccccc3)c3ccc4c5c(cccc35)-c3ccc(-c5ccc(-c6ccccc6)cc5)cc3O4)c21]

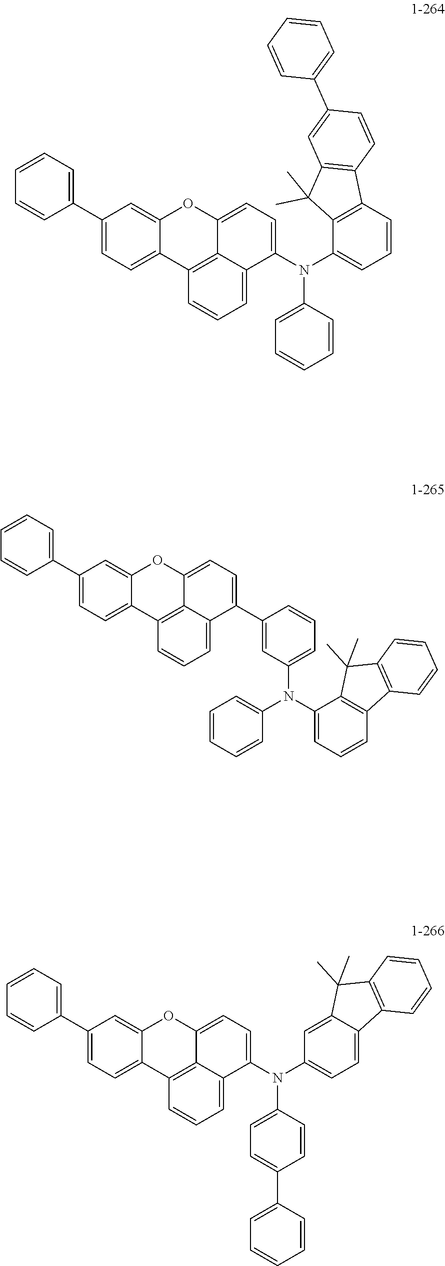

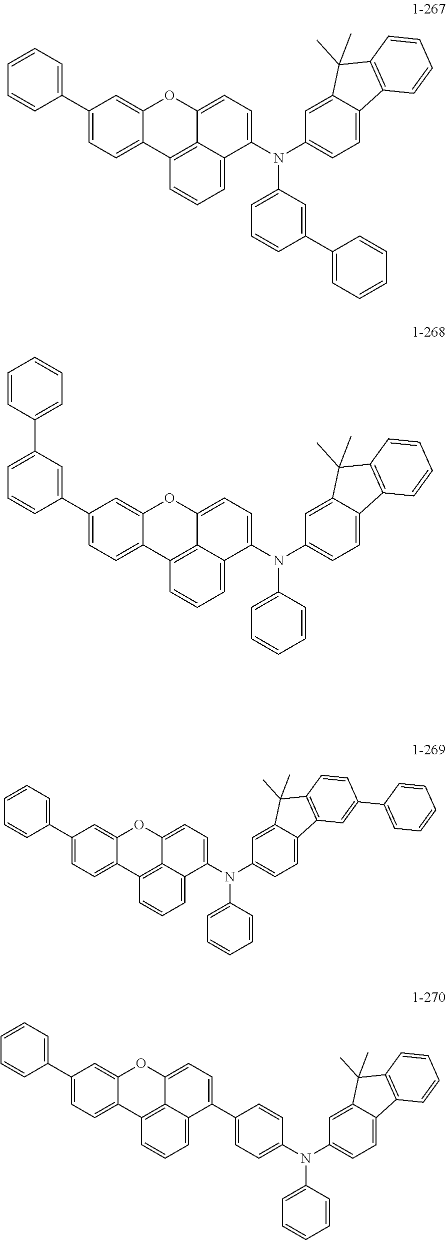 [SMILES: CC1(C)c2ccccc2-c2ccc(N(c3cccc(-c4ccccc4)c3)c3ccc4c5c(cccc35)-c3ccc(-c5ccccc5)cc3O4)cc21 ; CC1(C)c2ccccc2-c2ccc(N(c3ccccc3)c3ccc4c5c(cccc35)-c3ccc(-c5cccc(-c6ccccc6)c5)cc3O4)cc21 ; CC1(C)c2ccc(-c3ccccc3)cc2-c2ccc(N(c3ccccc3)c3ccc4c5c(cccc35)-c3ccc(-c5ccccc5)cc3O4)cc21 ; CC1(C)c2ccccc2-c2ccc(N(c3ccccc3)c3ccc(-c4ccc5c6c(cccc46)-c4ccc(-c6ccccc6)cc4O5)cc3)cc21]

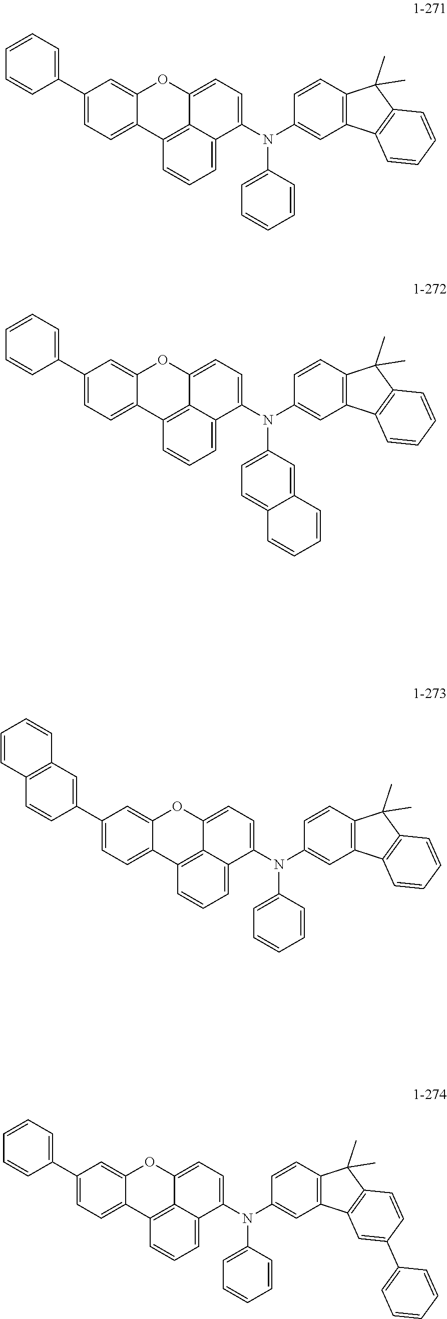 [SMILES: CC1(C)c2ccccc2-c2cc(N(c3ccccc3)c3ccc4c5c(cccc35)-c3ccc(-c5ccccc5)cc3O4)ccc21 ; CC1(C)c2ccccc2-c2cc(N(c3ccc4ccccc4c3)c3ccc4c5c(cccc35)-c3ccc(-c5ccccc5)cc3O4)ccc21 ; CC1(C)c2ccccc2-c2cc(N(c3ccccc3)c3ccc4c5c(cccc35)-c3ccc(-c5ccc6ccccc6c5)cc3O4)ccc21 ; CC1(C)c2ccc(-c3ccccc3)cc2-c2cc(N(c3ccccc3)c3ccc4c5c(cccc35)-c3ccc(-c5ccccc5)cc3O4)ccc21]

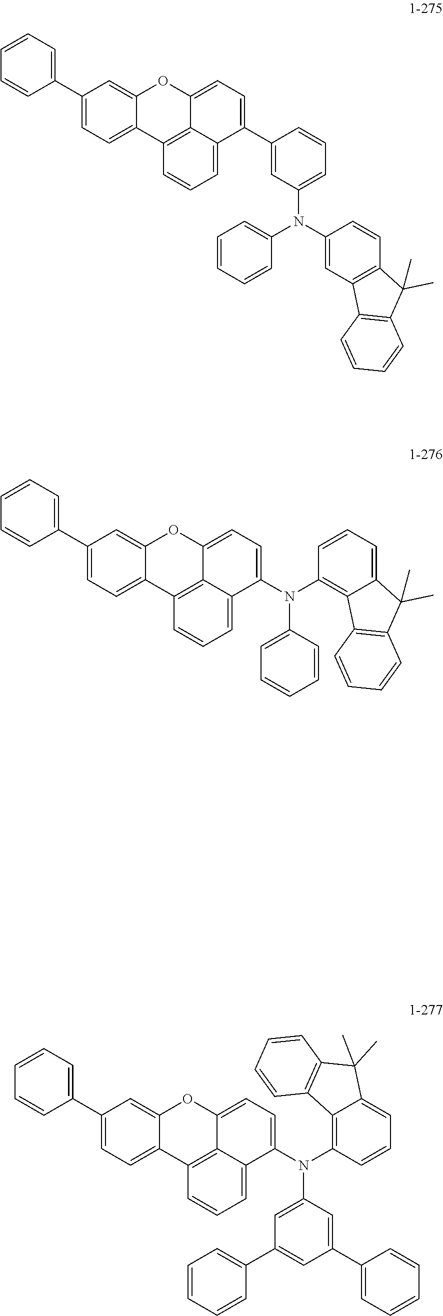

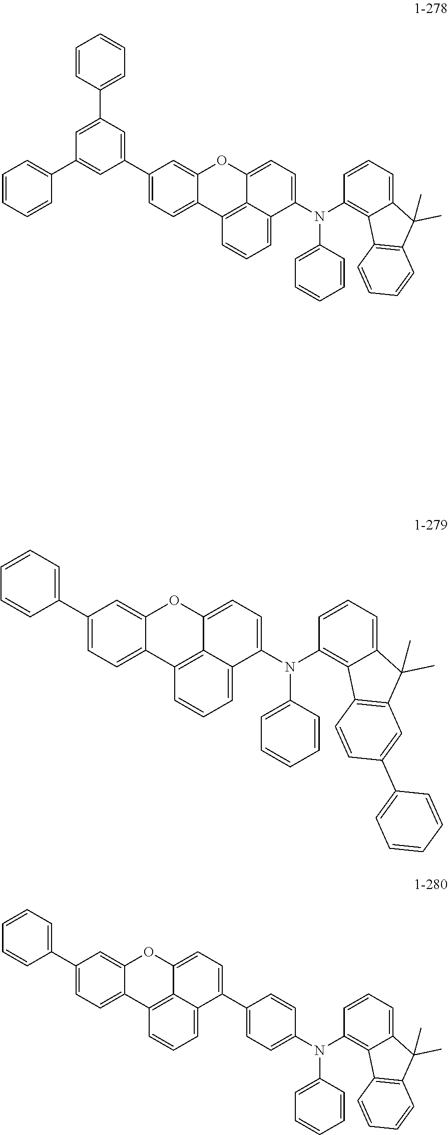 [SMILES: CC1(C)c2ccccc2-c2c(N(c3ccccc3)c3ccc4c5c(cccc35)-c3ccc(-c5cc(-c6ccccc6)cc(-c6ccccc6)c5)cc3O4)cccc21 ; CC1(C)c2cc(-c3ccccc3)ccc2-c2c(N(c3ccccc3)c3ccc4c5c(cccc35)-c3ccc(-c5ccccc5)cc3O4)cccc21 ; CC1(C)c2ccccc2-c2c(N(c3ccccc3)c3ccc(-c4ccc5c6c(cccc46)-c4ccc(-c6ccccc6)cc4O5)cc3)cccc21]

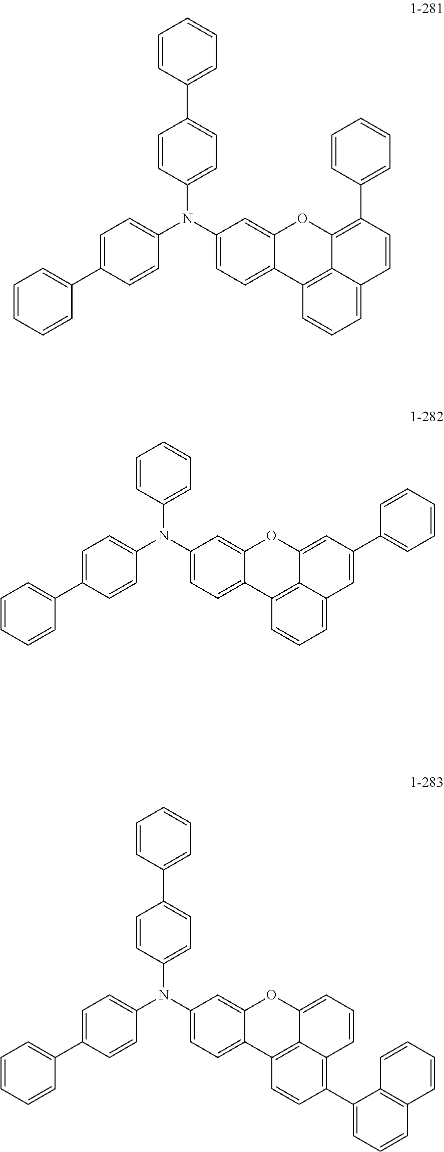 [SMILES: c1ccc(-c2ccc(N(c3ccc(-c4ccccc4)cc3)c3ccc4c(c3)Oc3c(-c5ccccc5)ccc5cccc-4c35)cc2)cc1 ; c1ccc(-c2ccc(N(c3ccccc3)c3ccc4c(c3)Oc3cc(-c5ccccc5)cc5cccc-4c35)cc2)cc1 ; c1ccc(-c2ccc(N(c3ccc(-c4ccccc4)cc3)c3ccc4c(c3)Oc3cccc5c(-c6cccc7ccccc67)ccc-4c35)cc2)cc1]

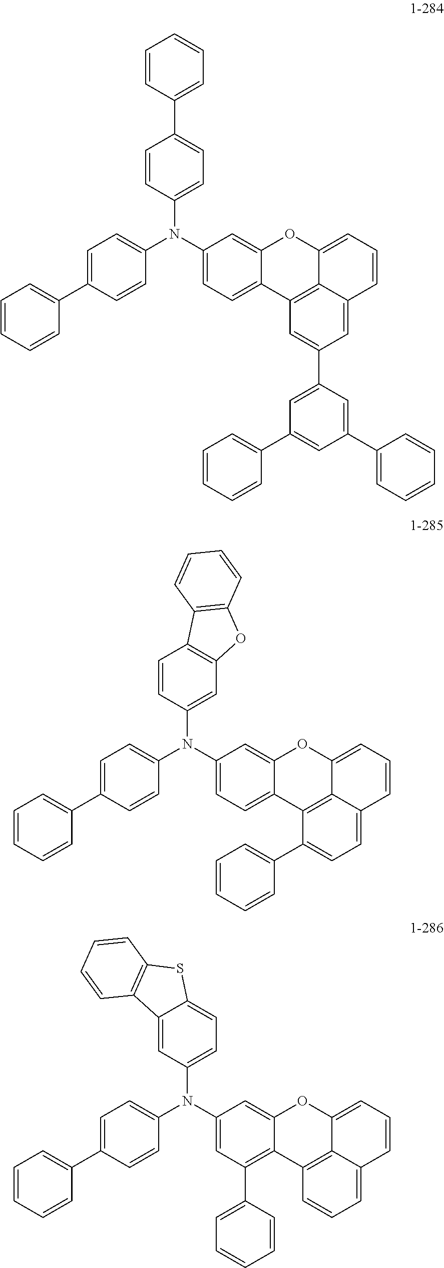 [SMILES: c1ccc(-c2ccc(N(c3ccc(-c4ccccc4)cc3)c3ccc4c(c3)Oc3cccc5cc(-c6cc(-c7ccccc7)cc(-c7ccccc7)c6)cc-4c35)cc2)cc1 ; c1ccc(-c2ccc(N(c3ccc4c(c3)Oc3cccc5ccc(-c6ccccc6)c-4c35)c3ccc4c(c3)oc3ccccc34)cc2)cc1 ; c1ccc(-c2ccc(N(c3cc4c(c(-c5ccccc5)c3)-c3cccc5cccc(c35)O4)c3ccc4sc5ccccc5c4c3)cc2)cc1]

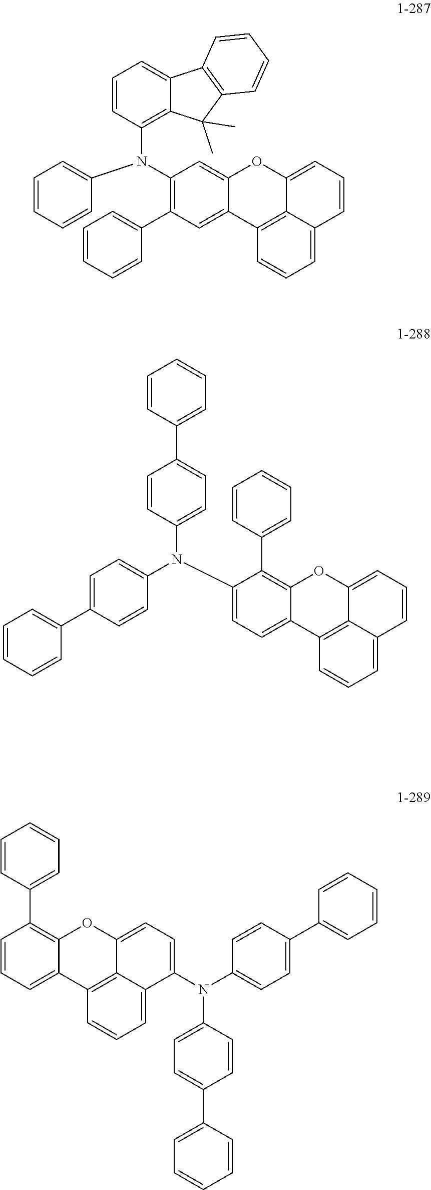 [SMILES: CC1(C)c2ccccc2-c2cccc(N(c3ccccc3)c3cc4c(cc3-c3ccccc3)-c3cccc5cccc(c35)O4)c21 ; c1ccc(-c2ccc(N(c3ccc(-c4ccccc4)cc3)c3ccc4c(c3-c3ccccc3)Oc3cccc5cccc-4c35)cc2)cc1 ; c1ccc(-c2ccc(N(c3ccc(-c4ccccc4)cc3)c3ccc4c5c(cccc35)-c3cccc(-c5ccccc5)c3O4)cc2)cc1]

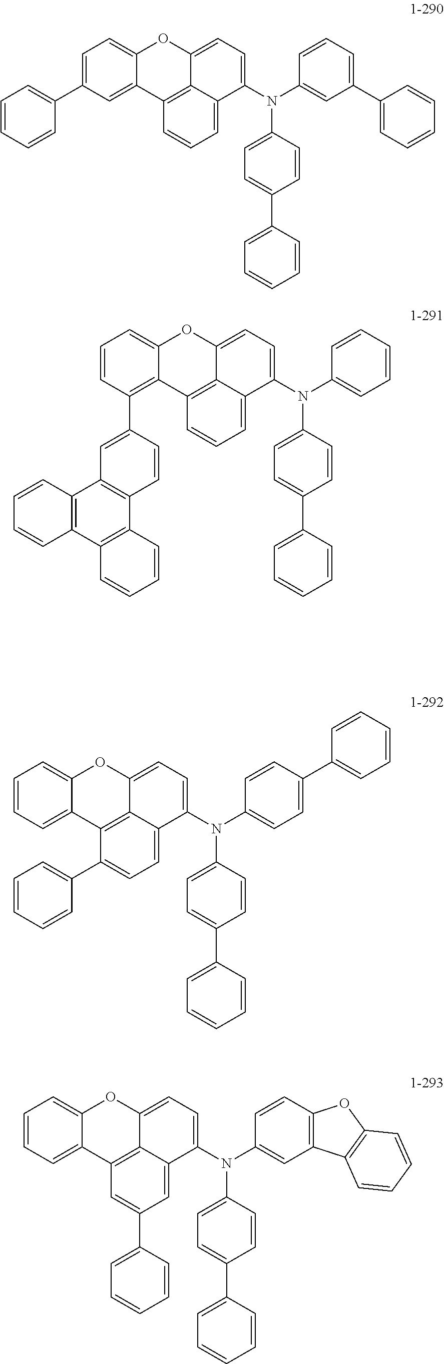 [SMILES: c1ccc(-c2ccc(N(c3cccc(-c4ccccc4)c3)c3ccc4c5c(cccc35)-c3cc(-c5ccccc5)ccc3O4)cc2)cc1 ; c1ccc(-c2ccc(N(c3ccccc3)c3ccc4c5c(cccc35)-c3c(cccc3-c3ccc5c6ccccc6c6ccccc6c5c3)O4)cc2)cc1 ; c1ccc(-c2ccc(N(c3ccc(-c4ccccc4)cc3)c3ccc4c5c(c(-c6ccccc6)ccc35)-c3ccccc3O4)cc2)cc1 ; c1ccc(-c2ccc(N(c3ccc4oc5ccccc5c4c3)c3ccc4c5c(cc(-c6ccccc6)cc35)-c3ccccc3O4)cc2)cc1]

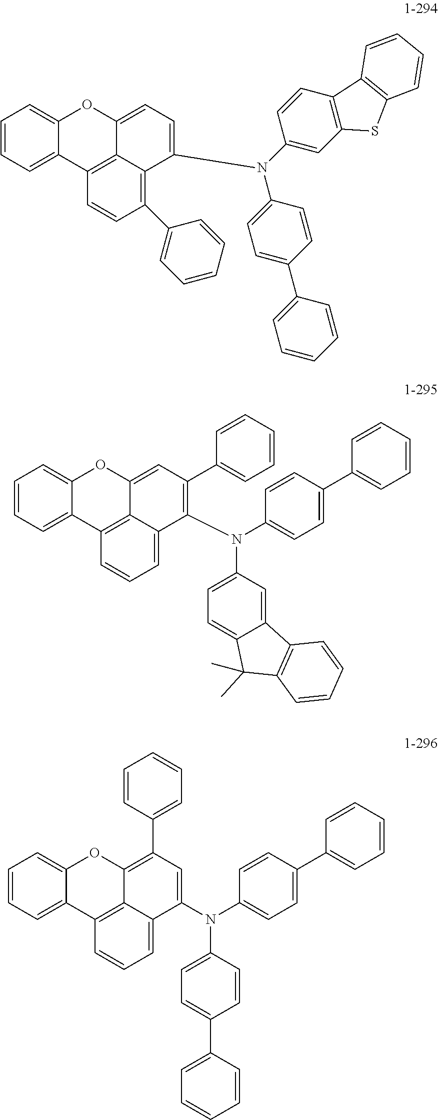 [SMILES: c1ccc(-c2ccc(N(c3ccc4c(c3)sc3ccccc34)c3ccc4c5c(ccc(-c6ccccc6)c35)-c3ccccc3O4)cc2)cc1 ; CC1(C)c2ccccc2-c2cc(N(c3ccc(-c4ccccc4)cc3)c3c(-c4ccccc4)cc4c5c(cccc35)-c3ccccc3O4)ccc21 ; c1ccc(-c2ccc(N(c3ccc(-c4ccccc4)cc3)c3cc(-c4ccccc4)c4c5c(cccc35)-c3ccccc3O4)cc2)cc1]

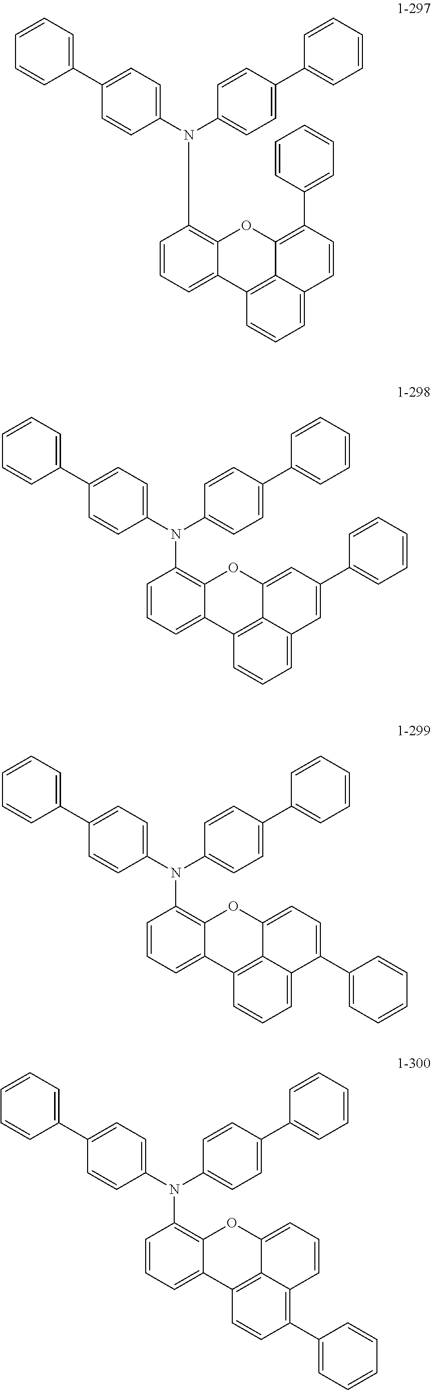 [SMILES: c1ccc(-c2ccc(N(c3ccc(-c4ccccc4)cc3)c3cccc4c3Oc3c(-c5ccccc5)ccc5cccc-4c35)cc2)cc1 ; c1ccc(-c2ccc(N(c3ccc(-c4ccccc4)cc3)c3cccc4c3Oc3cc(-c5ccccc5)cc5cccc-4c35)cc2)cc1 ; c1ccc(-c2ccc(N(c3ccc(-c4ccccc4)cc3)c3cccc4c3Oc3ccc(-c5ccccc5)c5cccc-4c35)cc2)cc1 ; c1ccc(-c2ccc(N(c3ccc(-c4ccccc4)cc3)c3cccc4c3Oc3cccc5c(-c6ccccc6)ccc-4c35)cc2)cc1]

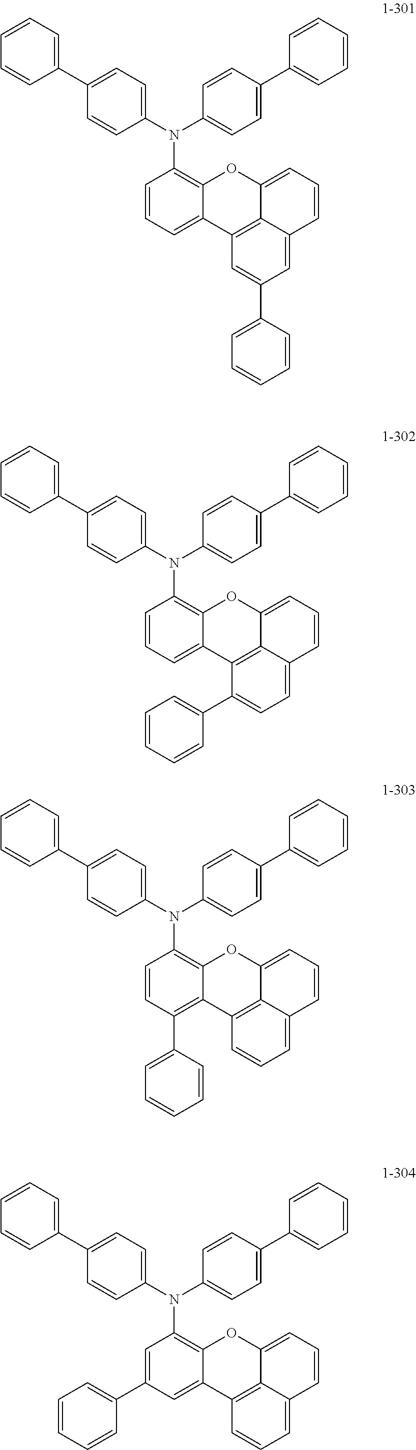 [SMILES: c1ccc(-c2ccc(N(c3ccc(-c4ccccc4)cc3)c3cccc4c3Oc3cccc5cc(-c6ccccc6)cc-4c35)cc2)cc1 ; c1ccc(-c2ccc(N(c3ccc(-c4ccccc4)cc3)c3cccc4c3Oc3cccc5ccc(-c6ccccc6)c-4c35)cc2)cc1 ; c1ccc(-c2ccc(N(c3ccc(-c4ccccc4)cc3)c3ccc(-c4ccccc4)c4c3Oc3cccc5cccc-4c35)cc2)cc1 ; c1ccc(-c2ccc(N(c3ccc(-c4ccccc4)cc3)c3cc(-c4ccccc4)cc4c3Oc3cccc5cccc-4c35)cc2)cc1]

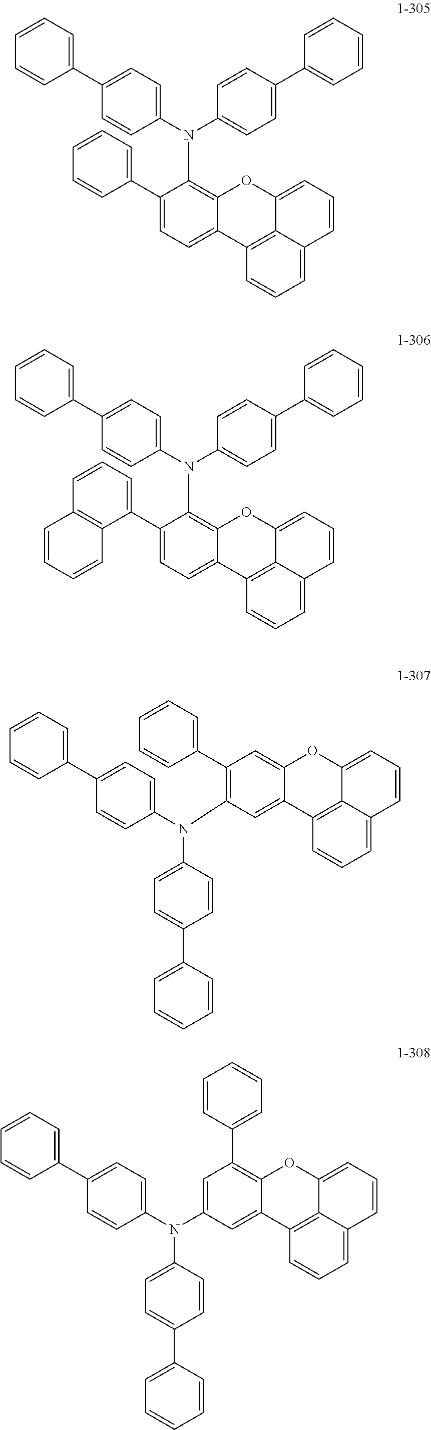 [SMILES: c1ccc(-c2ccc(N(c3ccc(-c4ccccc4)cc3)c3c(-c4ccccc4)ccc4c3Oc3cccc5cccc-4c35)cc2)cc1 ; c1ccc(-c2ccc(N(c3ccc(-c4ccccc4)cc3)c3c(-c4cccc5ccccc45)ccc4c3Oc3cccc5cccc-4c35)cc2)cc1 ; c1ccc(-c2ccc(N(c3ccc(-c4ccccc4)cc3)c3cc4c(cc3-c3ccccc3)Oc3cccc5cccc-4c35)cc2)cc1 ; c1ccc(-c2ccc(N(c3ccc(-c4ccccc4)cc3)c3cc(-c4ccccc4)c4c(c3)-c3cccc5cccc(c35)O4)cc2)cc1]

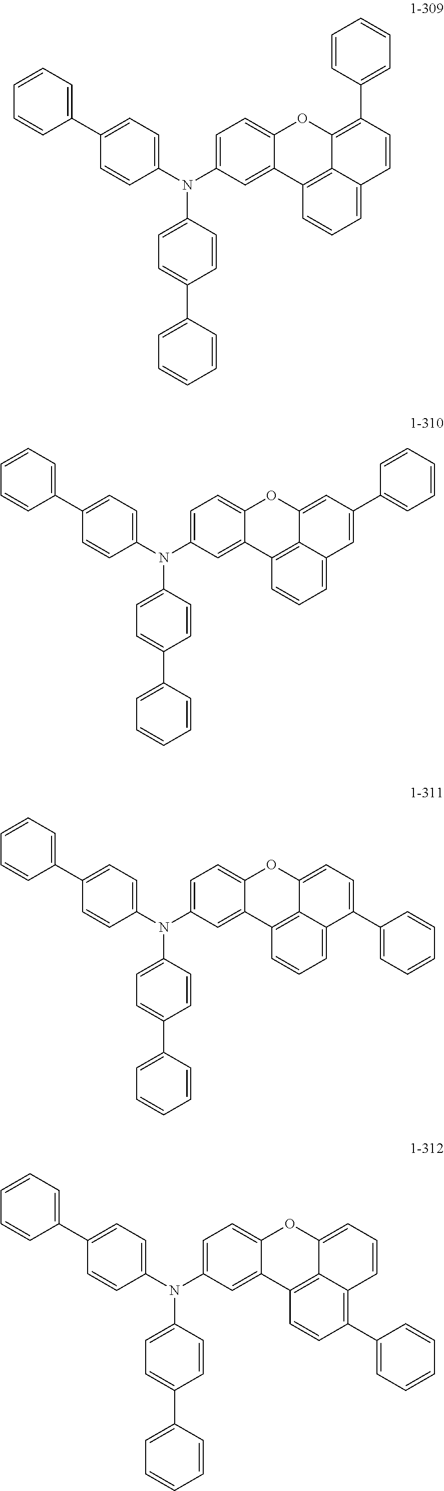 [SMILES: c1ccc(-c2ccc(N(c3ccc(-c4ccccc4)cc3)c3ccc4c(c3)-c3cccc5ccc(-c6ccccc6)c(c35)O4)cc2)cc1 ; c1ccc(-c2ccc(N(c3ccc(-c4ccccc4)cc3)c3ccc4c(c3)-c3cccc5cc(-c6ccccc6)cc(c35)O4)cc2)cc1 ; c1ccc(-c2ccc(N(c3ccc(-c4ccccc4)cc3)c3ccc4c(c3)-c3cccc5c(-c6ccccc6)ccc(c35)O4)cc2)cc1 ; c1ccc(-c2ccc(N(c3ccc(-c4ccccc4)cc3)c3ccc4c(c3)-c3ccc(-c5ccccc5)c5cccc(c35)O4)cc2)cc1]

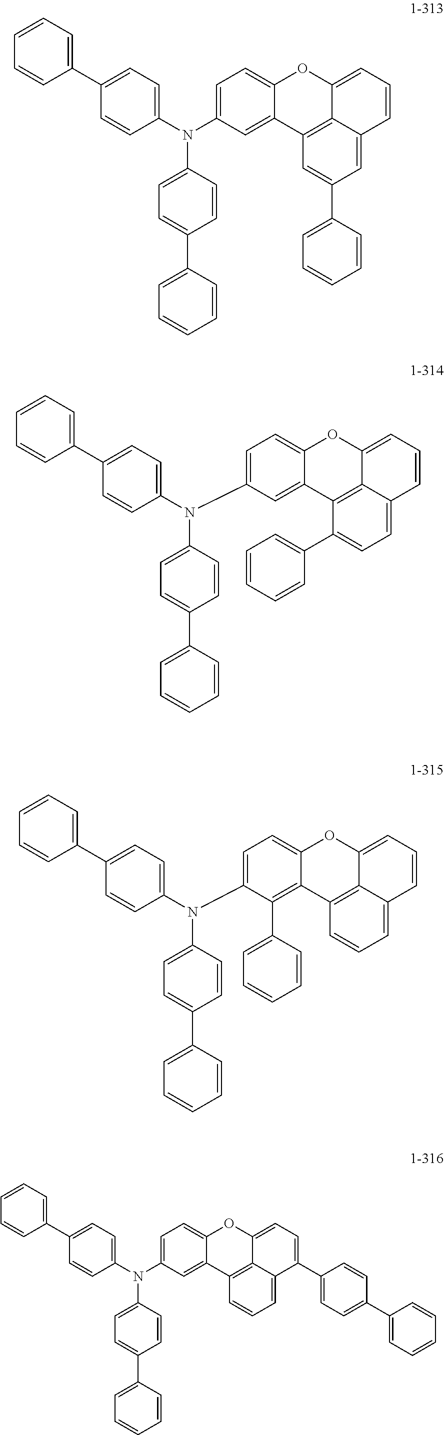 [SMILES: c1ccc(-c2ccc(N(c3ccc(-c4ccccc4)cc3)c3ccc4c(c3)-c3cc(-c5ccccc5)cc5cccc(c35)O4)cc2)cc1 ; c1ccc(-c2ccc(N(c3ccc(-c4ccccc4)cc3)c3ccc4c(c3)-c3c(-c5ccccc5)ccc5cccc(c35)O4)cc2)cc1 ; c1ccc(-c2ccc(N(c3ccc(-c4ccccc4)cc3)c3ccc4c(c3-c3ccccc3)-c3cccc5cccc(c35)O4)cc2)cc1 ; c1ccc(-c2ccc(-c3ccc4c5c(cccc35)-c3cc(N(c5ccc(-c6ccccc6)cc5)c5ccc(-c6ccccc6)cc5)ccc3O4)cc2)cc1]

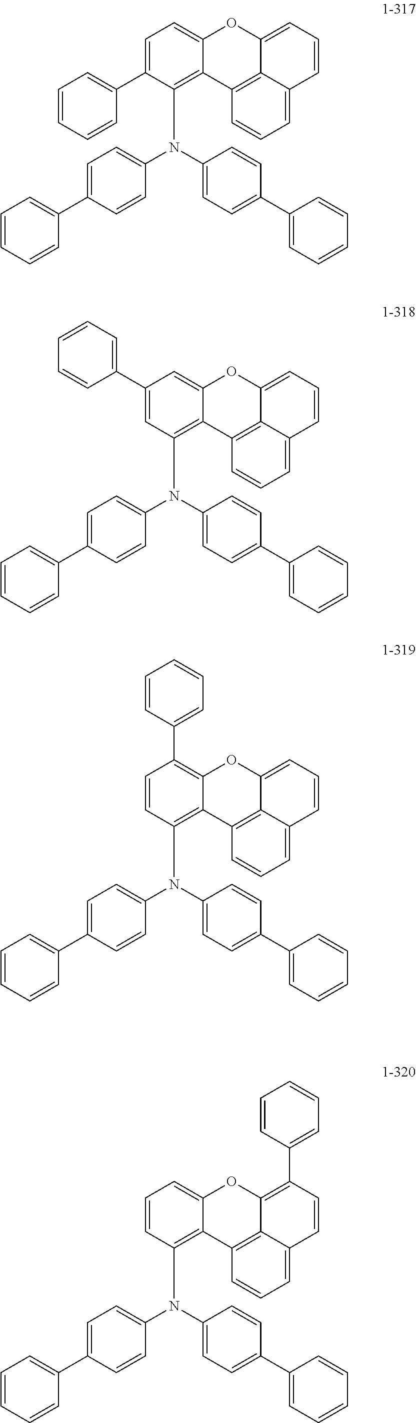 [SMILES: c1ccc(-c2ccc(N(c3ccc(-c4ccccc4)cc3)c3c(-c4ccccc4)ccc4c3-c3cccc5cccc(c35)O4)cc2)cc1 ; c1ccc(-c2ccc(N(c3ccc(-c4ccccc4)cc3)c3cc(-c4ccccc4)cc4c3-c3cccc5cccc(c35)O4)cc2)cc1 ; c1ccc(-c2ccc(N(c3ccc(-c4ccccc4)cc3)c3ccc(-c4ccccc4)c4c3-c3cccc5cccc(c35)O4)cc2)cc1 ; c1ccc(-c2ccc(N(c3ccc(-c4ccccc4)cc3)c3cccc4c3-c3cccc5ccc(-c6ccccc6)c(c35)O4)cc2)cc1]

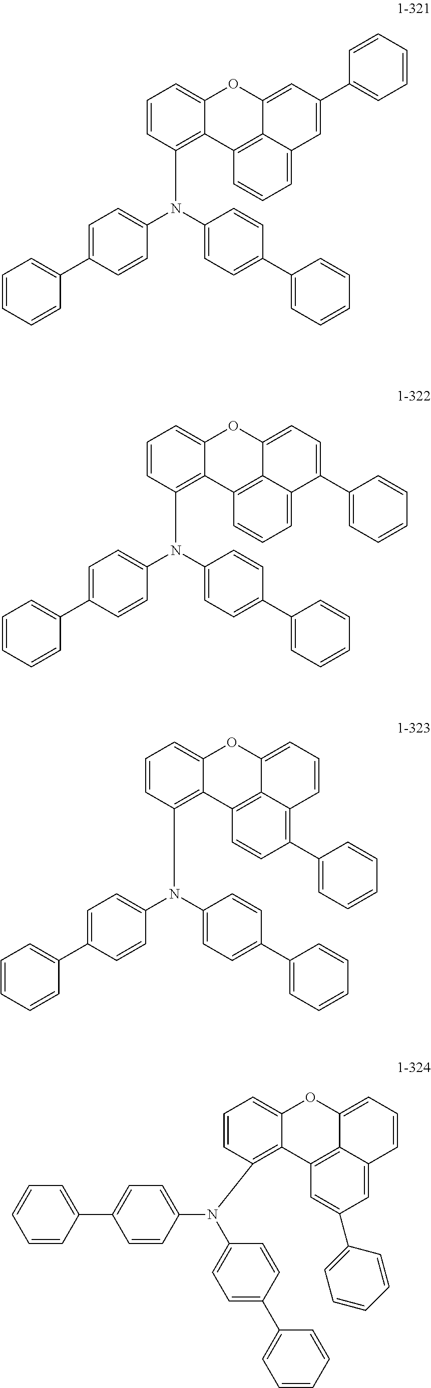 [SMILES: c1ccc(-c2ccc(N(c3ccc(-c4ccccc4)cc3)c3cccc4c3-c3cccc5cc(-c6ccccc6)cc(c35)O4)cc2)cc1 ; c1ccc(-c2ccc(N(c3ccc(-c4ccccc4)cc3)c3cccc4c3-c3cccc5c(-c6ccccc6)ccc(c35)O4)cc2)cc1 ; c1ccc(-c2ccc(N(c3ccc(-c4ccccc4)cc3)c3cccc4c3-c3ccc(-c5ccccc5)c5cccc(c35)O4)cc2)cc1 ; c1ccc(-c2ccc(N(c3ccc(-c4ccccc4)cc3)c3cccc4c3-c3cc(-c5ccccc5)cc5cccc(c35)O4)cc2)cc1]

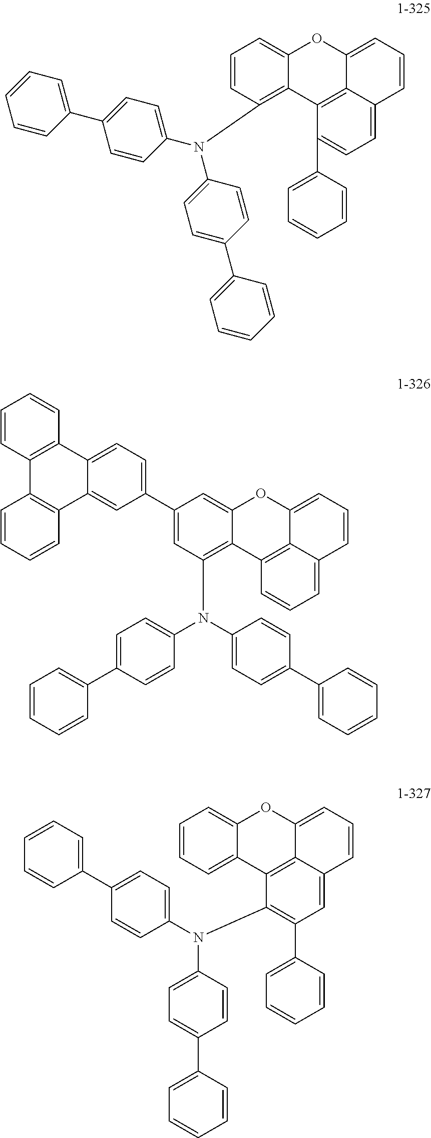 [SMILES: c1ccc(-c2ccc(N(c3ccc(-c4ccccc4)cc3)c3cccc4c3-c3c(-c5ccccc5)ccc5cccc(c35)O4)cc2)cc1 ; c1ccc(-c2ccc(N(c3ccc(-c4ccccc4)cc3)c3cc(-c4ccc5c6ccccc6c6ccccc6c5c4)cc4c3-c3cccc5cccc(c35)O4)cc2)cc1 ; c1ccc(-c2ccc(N(c3ccc(-c4ccccc4)cc3)c3c(-c4ccccc4)cc4cccc5c4c3-c3ccccc3O5)cc2)cc1]

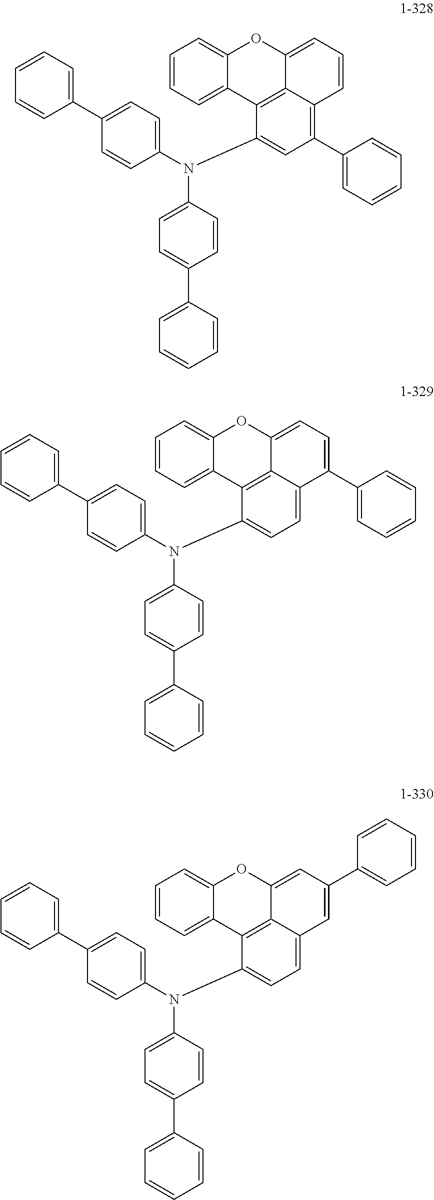 [SMILES: c1ccc(-c2ccc(N(c3ccc(-c4ccccc4)cc3)c3cc(-c4ccccc4)c4cccc5c4c3-c3ccccc3O5)cc2)cc1 ; c1ccc(-c2ccc(N(c3ccc(-c4ccccc4)cc3)c3ccc4c(-c5ccccc5)ccc5c4c3-c3ccccc3O5)cc2)cc1 ; c1ccc(-c2ccc(N(c3ccc(-c4ccccc4)cc3)c3ccc4cc(-c5ccccc5)cc5c4c3-c3ccccc3O5)cc2)cc1]

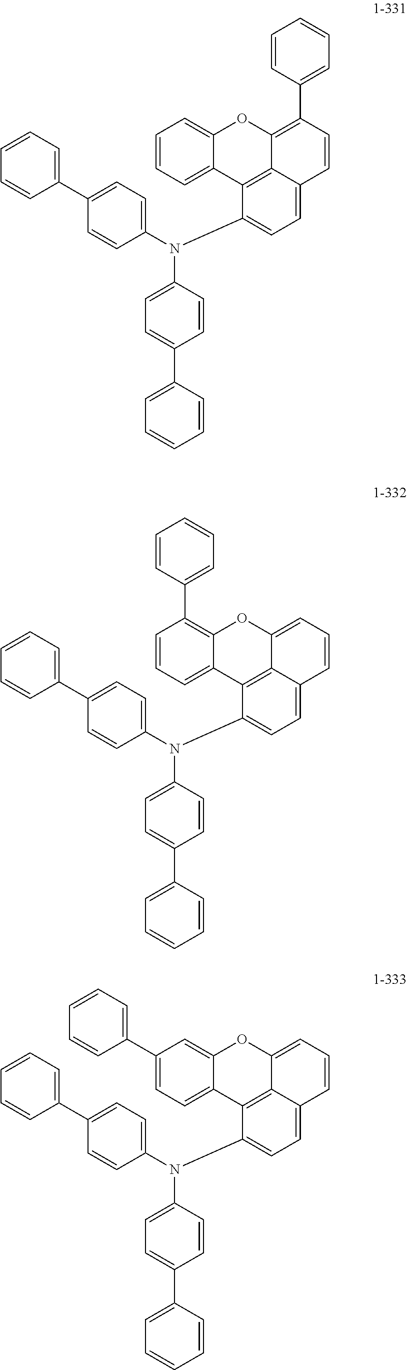 [SMILES: c1ccc(-c2ccc(N(c3ccc(-c4ccccc4)cc3)c3ccc4ccc(-c5ccccc5)c5c4c3-c3ccccc3O5)cc2)cc1 ; c1ccc(-c2ccc(N(c3ccc(-c4ccccc4)cc3)c3ccc4cccc5c4c3-c3cccc(-c4ccccc4)c3O5)cc2)cc1 ; c1ccc(-c2ccc(N(c3ccc(-c4ccccc4)cc3)c3ccc4cccc5c4c3-c3ccc(-c4ccccc4)cc3O5)cc2)cc1]

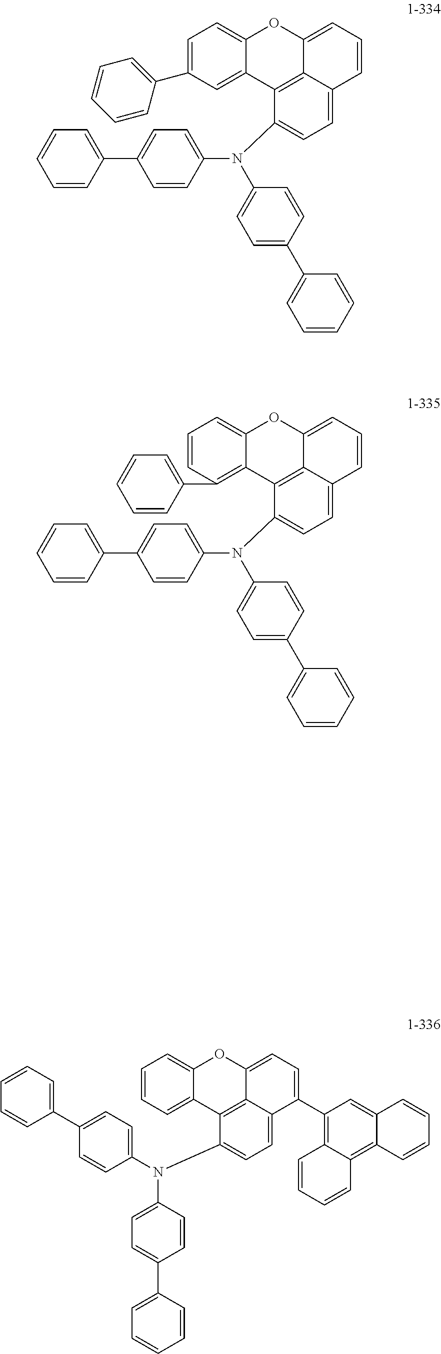 [SMILES: c1ccc(-c2ccc(N(c3ccc(-c4ccccc4)cc3)c3ccc4cccc5c4c3-c3cc(-c4ccccc4)ccc3O5)cc2)cc1 ; c1ccc(-c2ccc(N(c3ccc(-c4ccccc4)cc3)c3ccc4cccc5c4c3-c3c(cccc3-c3ccccc3)O5)cc2)cc1 ; c1ccc(-c2ccc(N(c3ccc(-c4ccccc4)cc3)c3ccc4c(-c5cc6ccccc6c6ccccc56)ccc5c4c3-c3ccccc3O5)cc2)cc1]

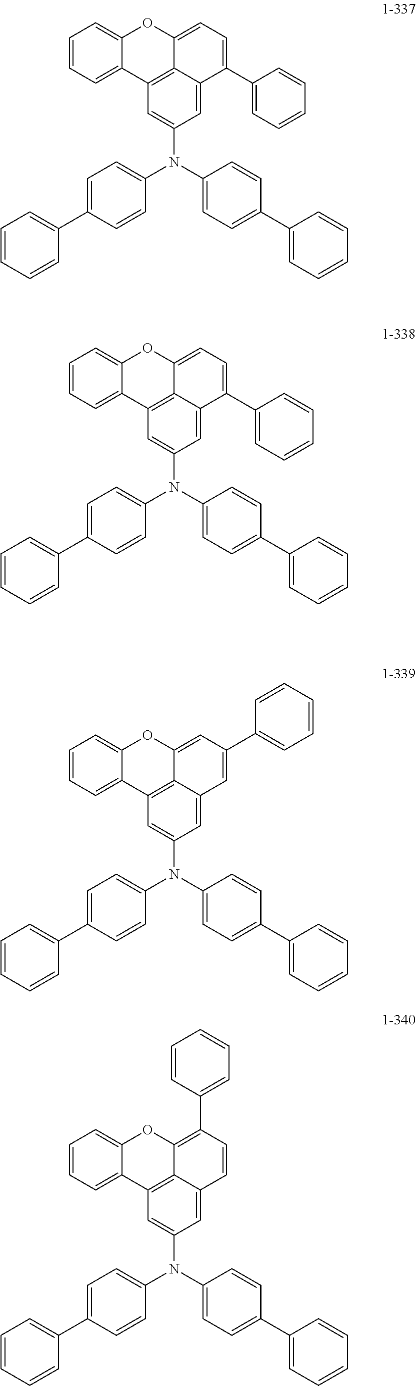 [SMILES: c1ccc(-c2ccc(N(c3ccc(-c4ccccc4)cc3)c3cc4c5c(ccc(-c6ccccc6)c5c3)Oc3ccccc3-4)cc2)cc1 ; c1ccc(-c2ccc(N(c3ccc(-c4ccccc4)cc3)c3cc4c5c(ccc(-c6ccccc6)c5c3)Oc3ccccc3-4)cc2)cc1 ; c1ccc(-c2ccc(N(c3ccc(-c4ccccc4)cc3)c3cc4c5c(cc(-c6ccccc6)cc5c3)Oc3ccccc3-4)cc2)cc1 ; c1ccc(-c2ccc(N(c3ccc(-c4ccccc4)cc3)c3cc4c5c(c(-c6ccccc6)ccc5c3)Oc3ccccc3-4)cc2)cc1]

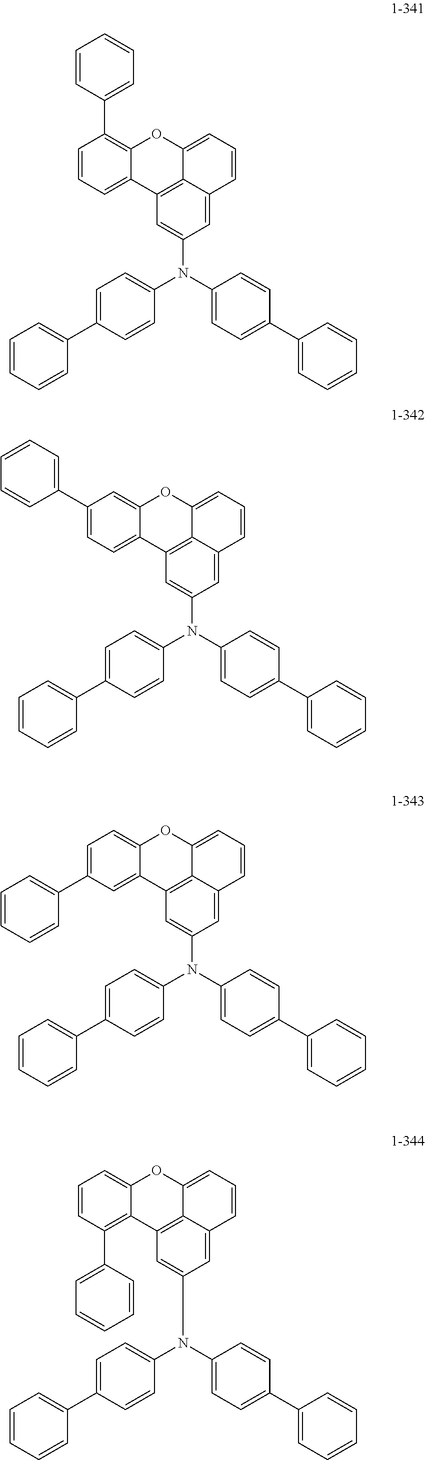 [SMILES: c1ccc(-c2ccc(N(c3ccc(-c4ccccc4)cc3)c3cc4c5c(cccc5c3)Oc3c(-c5ccccc5)cccc3-4)cc2)cc1 ; c1ccc(-c2ccc(N(c3ccc(-c4ccccc4)cc3)c3cc4c5c(cccc5c3)Oc3cc(-c5ccccc5)ccc3-4)cc2)cc1 ; c1ccc(-c2ccc(N(c3ccc(-c4ccccc4)cc3)c3cc4c5c(cccc5c3)Oc3ccc(-c5ccccc5)cc3-4)cc2)cc1 ; c1ccc(-c2ccc(N(c3ccc(-c4ccccc4)cc3)c3cc4c5c(cccc5c3)Oc3cccc(-c5ccccc5)c3-4)cc2)cc1]

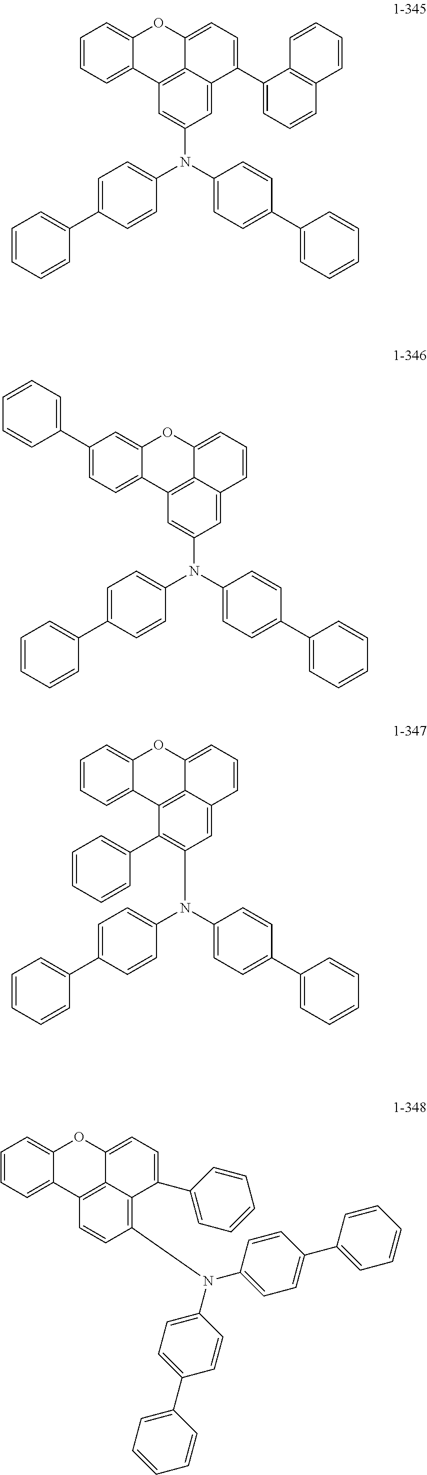 [SMILES: c1ccc(-c2ccc(N(c3ccc(-c4ccccc4)cc3)c3cc4c5c(ccc(-c6cccc7ccccc67)c5c3)Oc3ccccc3-4)cc2)cc1 ; c1ccc(-c2ccc(N(c3ccc(-c4ccccc4)cc3)c3cc4c5c(cccc5c3)Oc3cc(-c5ccccc5)ccc3-4)cc2)cc1 ; c1ccc(-c2ccc(N(c3ccc(-c4ccccc4)cc3)c3cc4cccc5c4c(c3-c3ccccc3)-c3ccccc3O5)cc2)cc1 ; c1ccc(-c2ccc(N(c3ccc(-c4ccccc4)cc3)c3ccc4c5c(ccc(-c6ccccc6)c35)Oc3ccccc3-4)cc2)cc1]

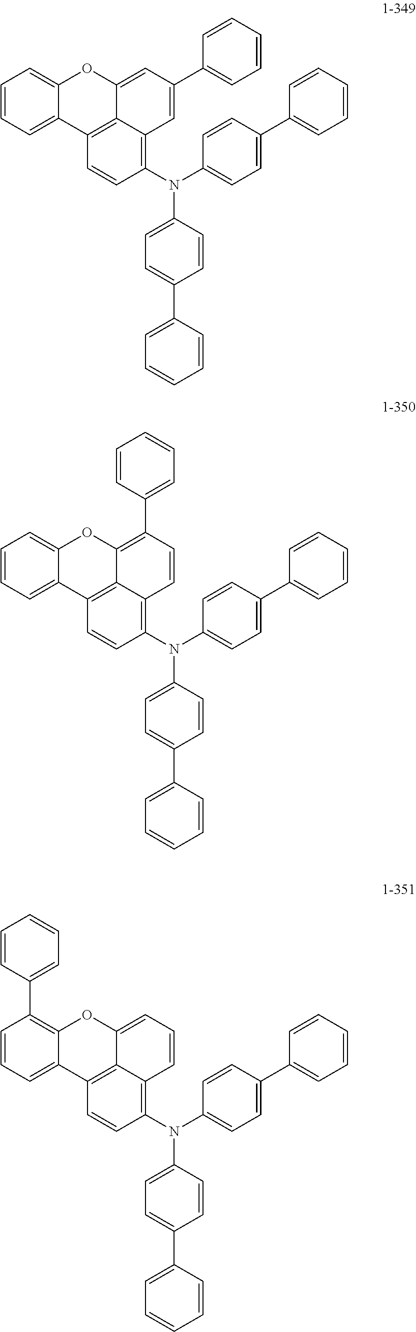 [SMILES: c1ccc(-c2ccc(N(c3ccc(-c4ccccc4)cc3)c3ccc4c5c(cc(-c6ccccc6)cc35)Oc3ccccc3-4)cc2)cc1 ; c1ccc(-c2ccc(N(c3ccc(-c4ccccc4)cc3)c3ccc4c5c(c(-c6ccccc6)ccc35)Oc3ccccc3-4)cc2)cc1 ; c1ccc(-c2ccc(N(c3ccc(-c4ccccc4)cc3)c3ccc4c5c(cccc35)Oc3c(-c5ccccc5)cccc3-4)cc2)cc1]

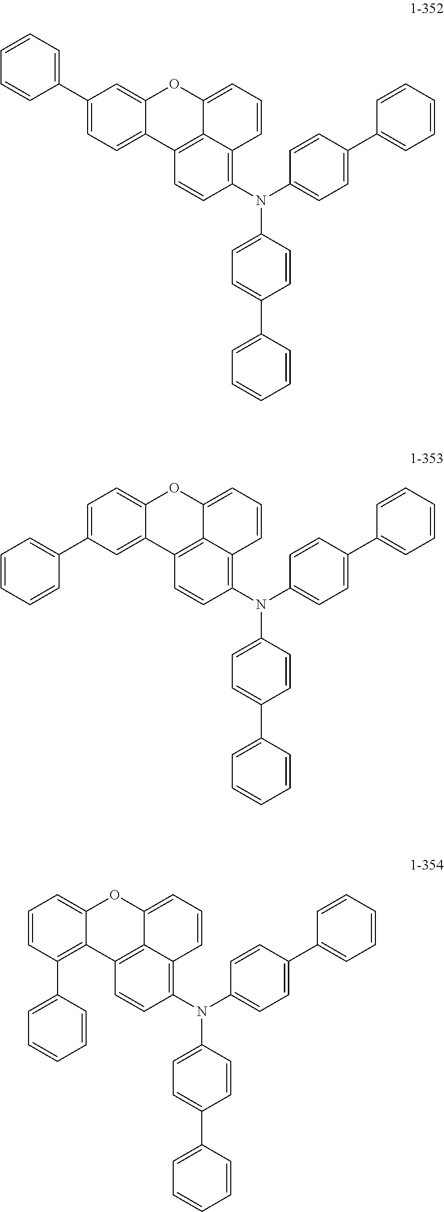 [SMILES: c1ccc(-c2ccc(N(c3ccc(-c4ccccc4)cc3)c3ccc4c5c(cccc35)Oc3cc(-c5ccccc5)ccc3-4)cc2)cc1 ; c1ccc(-c2ccc(N(c3ccc(-c4ccccc4)cc3)c3ccc4c5c(cccc35)Oc3ccc(-c5ccccc5)cc3-4)cc2)cc1 ; c1ccc(-c2ccc(N(c3ccc(-c4ccccc4)cc3)c3ccc4c5c(cccc35)Oc3cccc(-c5ccccc5)c3-4)cc2)cc1]

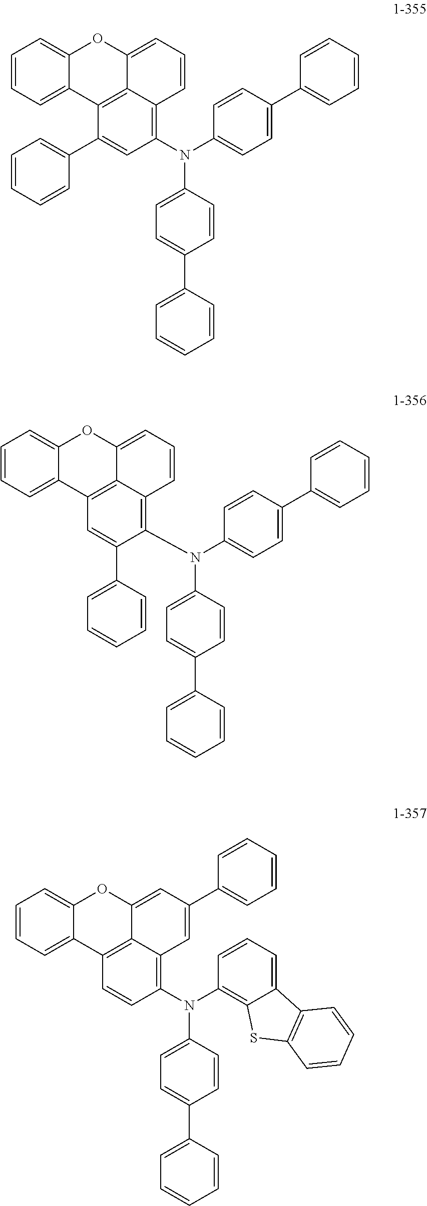 [SMILES: c1ccc(-c2ccc(N(c3ccc(-c4ccccc4)cc3)c3cc(-c4ccccc4)c4c5c(cccc35)Oc3ccccc3-4)cc2)cc1 ; c1ccc(-c2ccc(N(c3ccc(-c4ccccc4)cc3)c3c(-c4ccccc4)cc4c5c(cccc35)Oc3ccccc3-4)cc2)cc1 ; c1ccc(-c2ccc(N(c3ccc4c5c(cc(-c6ccccc6)cc35)Oc3ccccc3-4)c3cccc4c3sc3ccccc34)cc2)cc1]

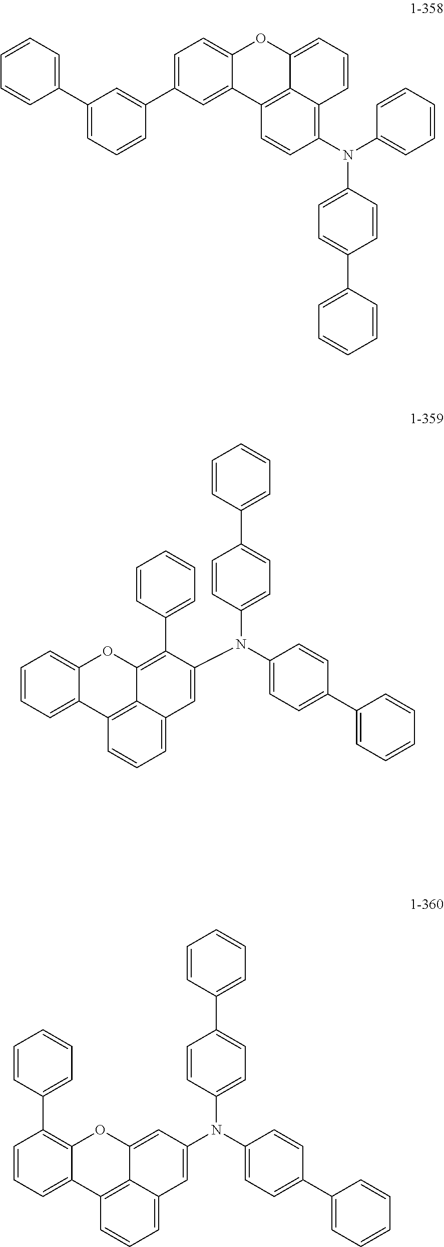 [SMILES: c1ccc(-c2ccc(N(c3ccccc3)c3ccc4c5c(cccc35)Oc3ccc(-c5cccc(-c6ccccc6)c5)cc3-4)cc2)cc1 ; c1ccc(-c2ccc(N(c3ccc(-c4ccccc4)cc3)c3cc4cccc5c4c(c3-c3ccccc3)Oc3ccccc3-5)cc2)cc1 ; c1ccc(-c2ccc(N(c3ccc(-c4ccccc4)cc3)c3cc4c5c(cccc5c3)-c3cccc(-c5ccccc5)c3O4)cc2)cc1]

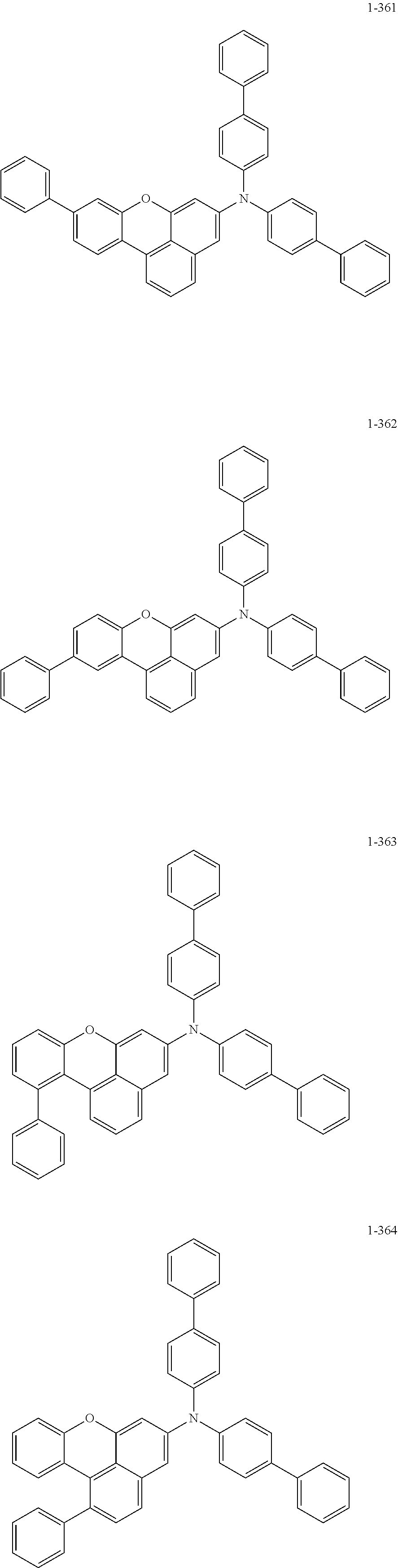 [SMILES: c1ccc(-c2ccc(N(c3ccc(-c4ccccc4)cc3)c3cc4c5c(cccc5c3)-c3ccc(-c5ccccc5)cc3O4)cc2)cc1 ; c1ccc(-c2ccc(N(c3ccc(-c4ccccc4)cc3)c3cc4c5c(cccc5c3)-c3cc(-c5ccccc5)ccc3O4)cc2)cc1 ; c1ccc(-c2ccc(N(c3ccc(-c4ccccc4)cc3)c3cc4c5c(cccc5c3)-c3c(cccc3-c3ccccc3)O4)cc2)cc1 ; c1ccc(-c2ccc(N(c3ccc(-c4ccccc4)cc3)c3cc4c5c(c(-c6ccccc6)ccc5c3)-c3ccccc3O4)cc2)cc1]

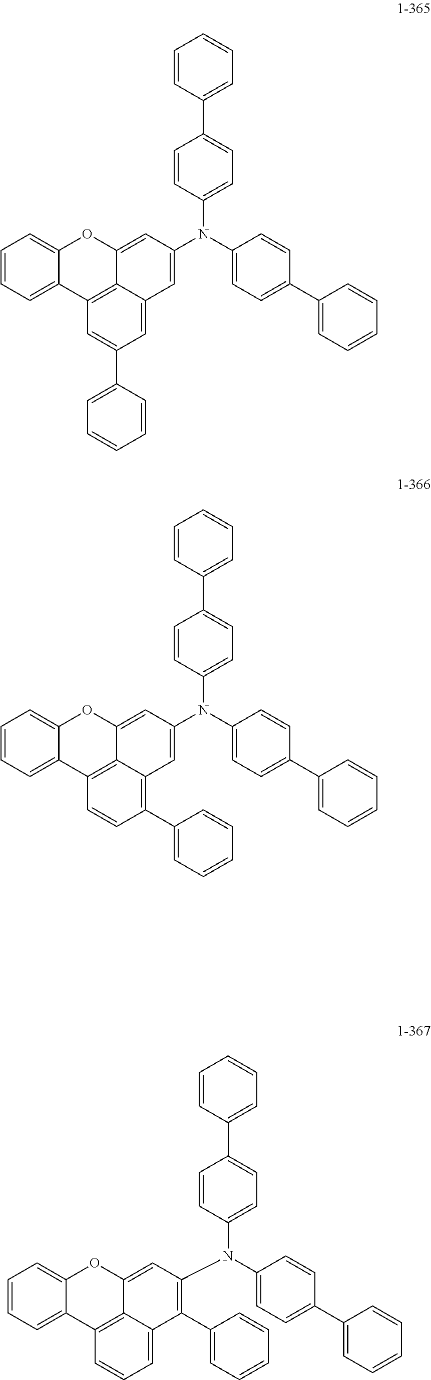 [SMILES: c1ccc(-c2ccc(N(c3ccc(-c4ccccc4)cc3)c3cc4c5c(cc(-c6ccccc6)cc5c3)-c3ccccc3O4)cc2)cc1 ; c1ccc(-c2ccc(N(c3ccc(-c4ccccc4)cc3)c3cc4c5c(ccc(-c6ccccc6)c5c3)-c3ccccc3O4)cc2)cc1 ; c1ccc(-c2ccc(N(c3ccc(-c4ccccc4)cc3)c3cc4c5c(cccc5c3-c3ccccc3)-c3ccccc3O4)cc2)cc1]

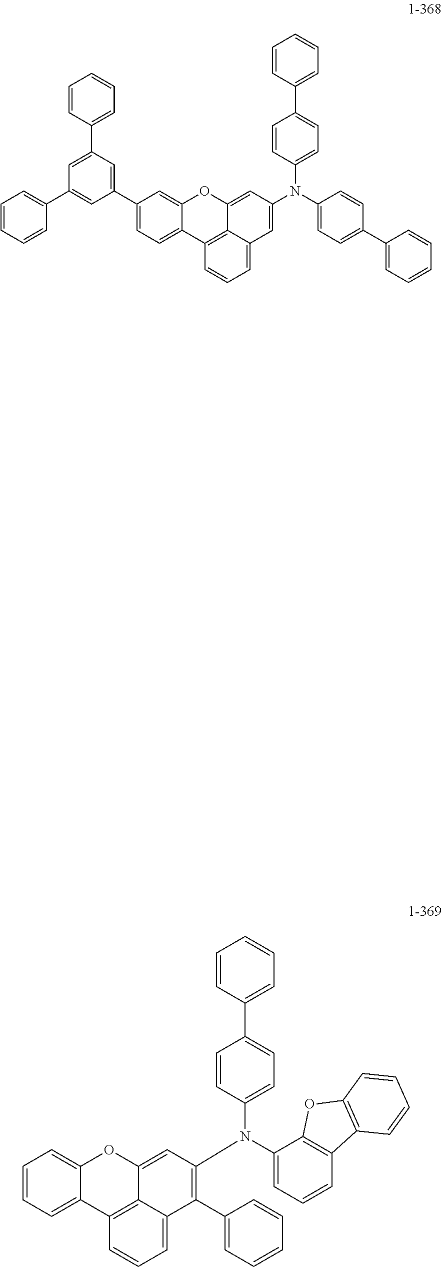 [SMILES: c1ccc(-c2ccc(N(c3ccc(-c4ccccc4)cc3)c3cc4c5c(cccc5c3)-c3ccc(-c5cc(-c6ccccc6)cc(-c6ccccc6)c5)cc3O4)cc2)cc1 ; c1ccc(-c2ccc(N(c3cc4c5c(cccc5c3-c3ccccc3)-c3ccccc3O4)c3cccc4c3oc3ccccc34)cc2)cc1]

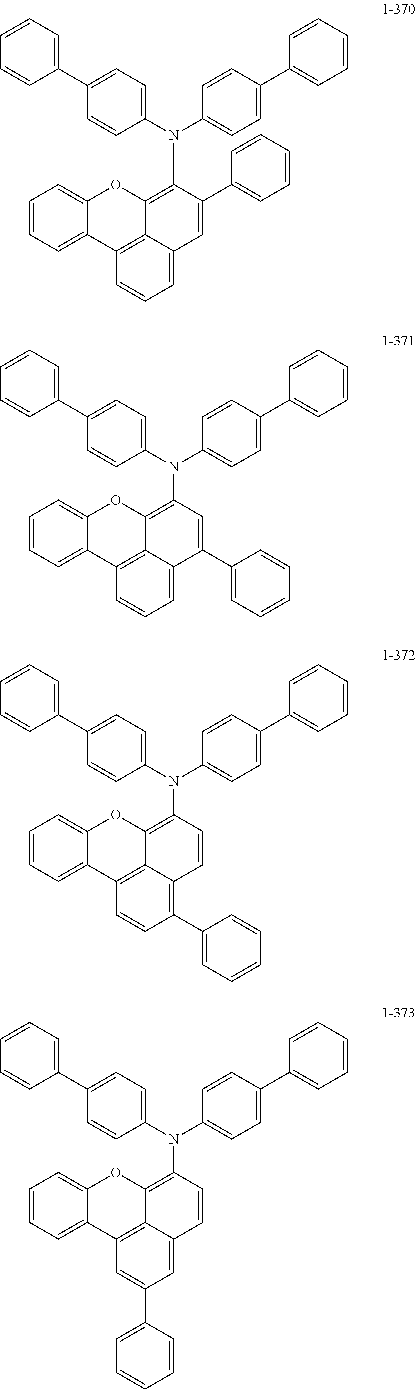 [SMILES: c1ccc(-c2ccc(N(c3ccc(-c4ccccc4)cc3)c3c(-c4ccccc4)cc4cccc5c4c3Oc3ccccc3-5)cc2)cc1 ; c1ccc(-c2ccc(N(c3ccc(-c4ccccc4)cc3)c3cc(-c4ccccc4)c4cccc5c4c3Oc3ccccc3-5)cc2)cc1 ; c1ccc(-c2ccc(N(c3ccc(-c4ccccc4)cc3)c3ccc4c(-c5ccccc5)ccc5c4c3Oc3ccccc3-5)cc2)cc1 ; c1ccc(-c2ccc(N(c3ccc(-c4ccccc4)cc3)c3ccc4cc(-c5ccccc5)cc5c4c3Oc3ccccc3-5)cc2)cc1]

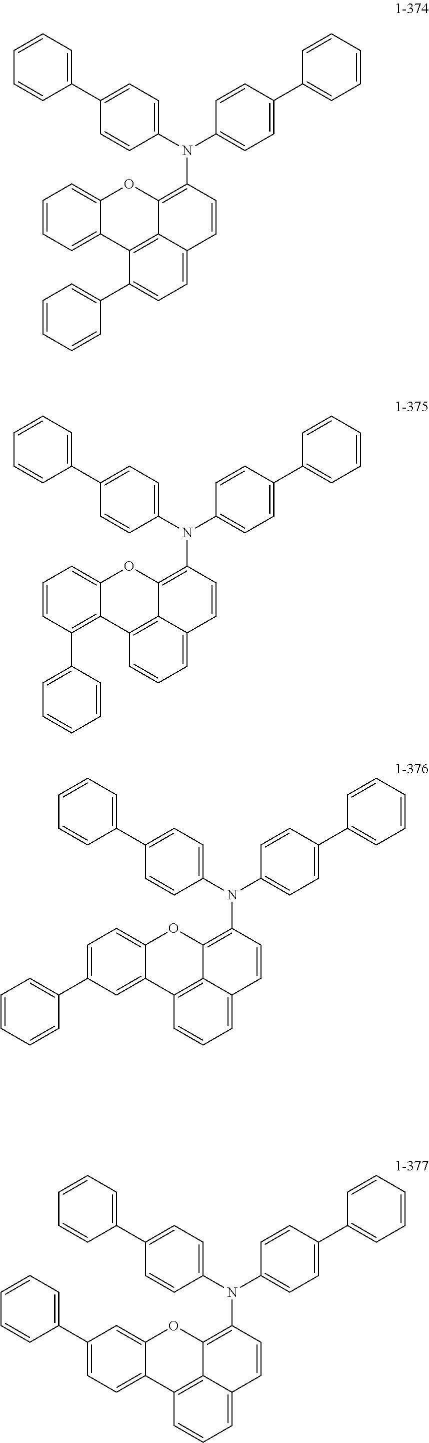 [SMILES: c1ccc(-c2ccc(N(c3ccc(-c4ccccc4)cc3)c3ccc4ccc(-c5ccccc5)c5c4c3Oc3ccccc3-5)cc2)cc1 ; c1ccc(-c2ccc(N(c3ccc(-c4ccccc4)cc3)c3ccc4cccc5c4c3Oc3cccc(-c4ccccc4)c3-5)cc2)cc1 ; c1ccc(-c2ccc(N(c3ccc(-c4ccccc4)cc3)c3ccc4cccc5c4c3Oc3ccc(-c4ccccc4)cc3-5)cc2)cc1 ; c1ccc(-c2ccc(N(c3ccc(-c4ccccc4)cc3)c3ccc4cccc5c4c3Oc3cc(-c4ccccc4)ccc3-5)cc2)cc1]

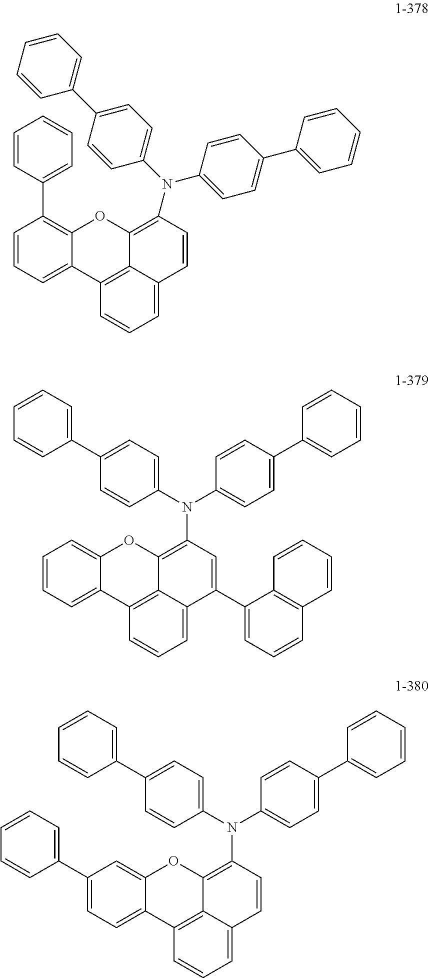 [SMILES: c1ccc(-c2ccc(N(c3ccc(-c4ccccc4)cc3)c3ccc4cccc5c4c3Oc3c(-c4ccccc4)cccc3-5)cc2)cc1 ; c1ccc(-c2ccc(N(c3ccc(-c4ccccc4)cc3)c3cc(-c4cccc5ccccc45)c4cccc5c4c3Oc3ccccc3-5)cc2)cc1 ; c1ccc(-c2ccc(N(c3ccc(-c4ccccc4)cc3)c3ccc4cccc5c4c3Oc3cc(-c4ccccc4)ccc3-5)cc2)cc1]

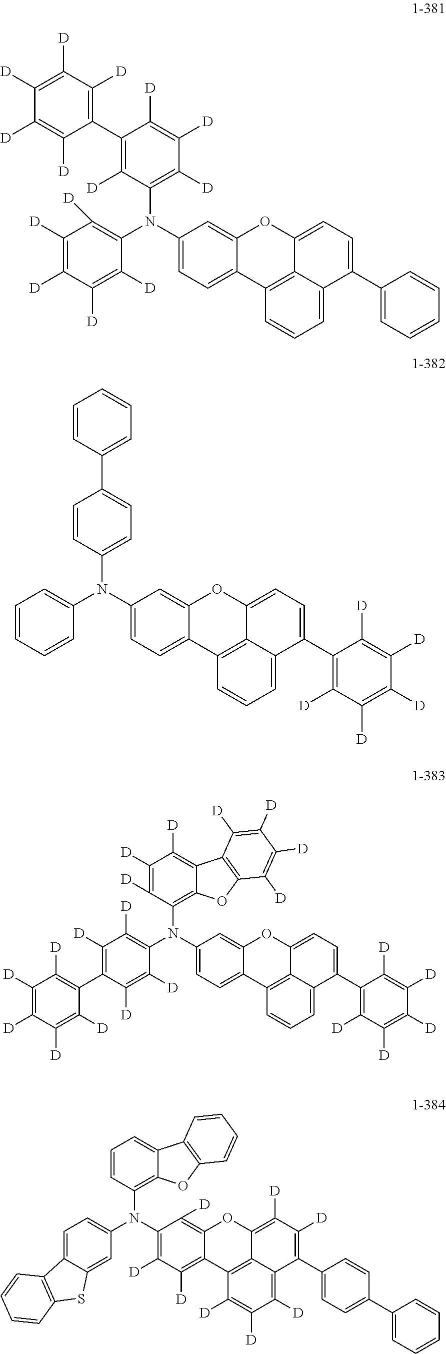

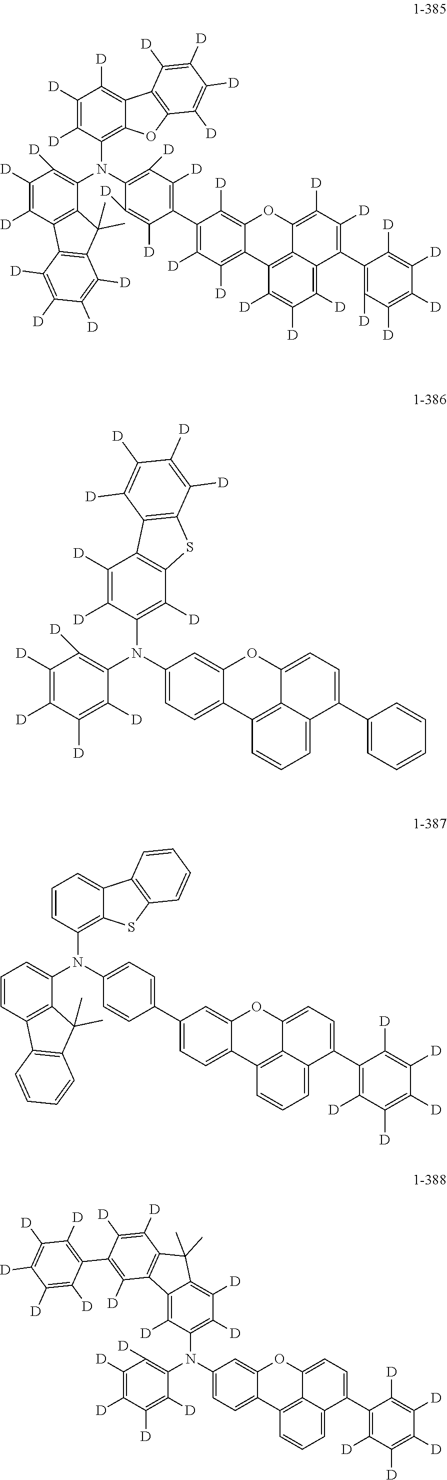

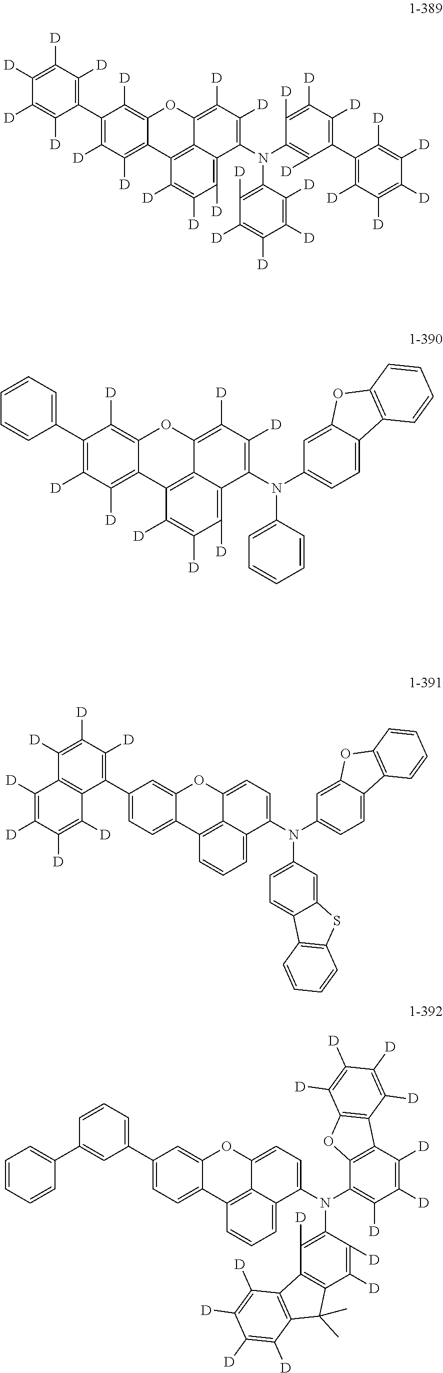

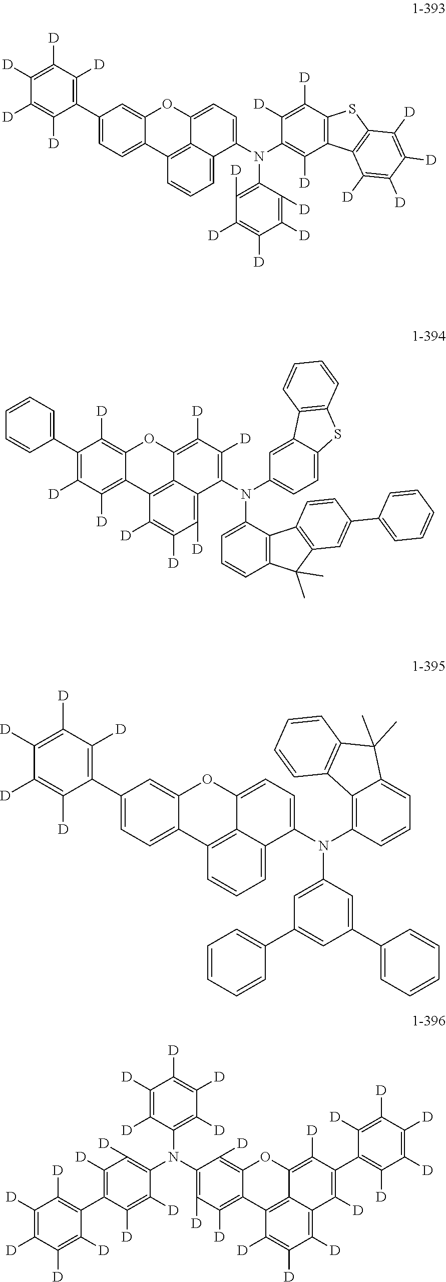

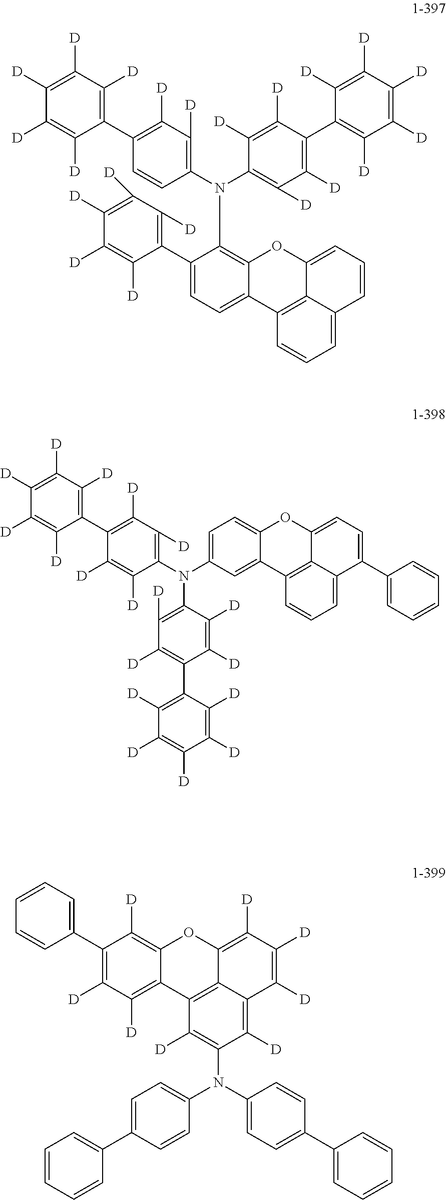

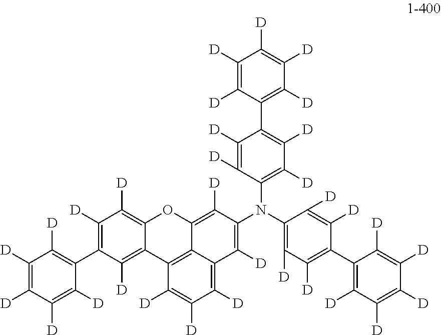 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c5c(c([2H])c([2H])c([2H])c5c3[2H])-c3c([2H])c(-c5c([2H])c([2H])c([2H])c([2H])c5[2H])c([2H])c([2H])c3O4)c([2H])c2[2H])c([2H])c1[2H]]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

One embodiment of the present specification provides an organic light emitting device including a first electrode; a second electrode; and one or more organic material layers provided between the first electrode and the second electrode, wherein one or more layers of the organic material layers include the heterocyclic compound of Chemical Formula 1.

In one embodiment of the present specification, the first electrode may be an anode, and the second electrode may be a cathode.

In another embodiment of the present specification, the first electrode may be a cathode, and the second electrode may be an anode.

In one embodiment of the present specification, the organic light emitting device may be a blue organic light emitting device, and the heterocyclic compound of Chemical Formula 1 may be used as a material of the blue organic light emitting device. For example, the heterocyclic compound of Chemical Formula 1 may be included in a hole transfer layer or an electron blocking layer of the blue organic light emitting device.

In one embodiment of the present specification, the organic light emitting device may be a green organic light emitting device, and the heterocyclic compound of Chemical Formula 1 may be used as a material of the green organic light emitting device. For example, the heterocyclic compound of Chemical Formula 1 may be included in a hole transfer layer or an electron blocking layer of the green organic light emitting device.

In one embodiment of the present specification, the organic light emitting device may be a red organic light emitting device, and the heterocyclic compound of Chemical Formula 1 may be used as a material of the red organic light emitting device. For example, the heterocyclic compound of Chemical Formula 1 may be included in a hole transfer layer or an electron blocking layer of the red organic light emitting device.

The organic light emitting device of the present specification may be manufactured using common organic light emitting device manufacturing methods and materials except that one or more organic material layers are formed using the heterocyclic compound described above.

The heterocyclic compound may be formed into an organic material layer using a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present specification may be formed in a single layer structure, but may be formed in a multilayer structure in which two or more organic material layers are laminated. For example, the organic light emitting device of the present disclosure may have a structure including a hole injection layer, a hole transfer layer, a light emitting layer, an electron transfer layer, an electron injection layer and the like as the organic material layer. However, the structure of the organic light emitting device is not limited thereto, and may include a smaller number of organic material layers.

In the organic light emitting device of the present specification, the organic material layer includes a hole transfer layer, and the hole transfer layer may include the heterocyclic compound of Chemical Formula 1.

In the organic light emitting device of the present specification, the organic material layer includes an electron blocking layer, and the electron blocking layer may include the heterocyclic compound of Chemical Formula 1.

The organic light emitting device of the present disclosure may further include one, two or more layers selected from the group consisting of a light emitting layer, a hole injection layer, a hole transfer layer, an electron injection layer, an electron transfer layer, an electron blocking layer and a hole blocking layer.

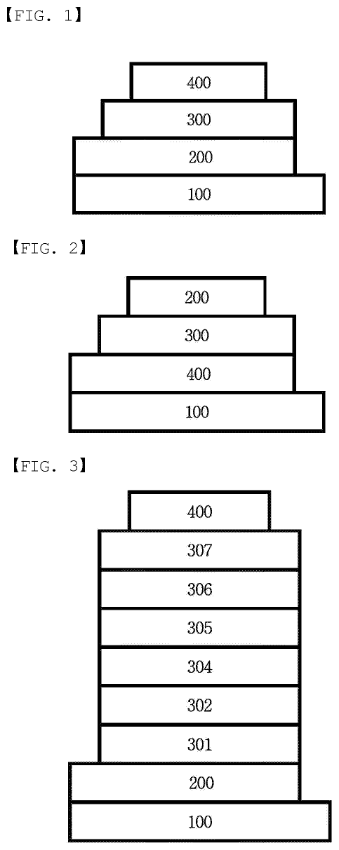

FIG. 1 to FIG. 4 illustrate a lamination order of electrodes and organic material layers of the organic light emitting device according to one embodiment of the present specification. However, the scope of the present application is not limited to these diagrams, and structures of organic light emitting devices known in the art may also be used in the present application.

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

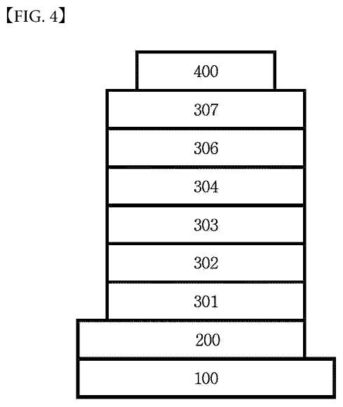

FIG. 3 and FIG. 4 illustrate cases of the organic material layer being a multilayer. The organic light emitting device according to FIG. 3 includes a hole injection layer (301), a hole transfer layer (302), a light emitting layer (304), a hole blocking layer (305), an electron transfer layer (306) and an electron injection layer (307), and the organic light emitting device according to FIG. 4 includes a hole injection layer (301), a hole transfer layer (302), an electron blocking layer (303), a light emitting layer (304), an electron transfer layer (306) and an electron injection layer (307). However, the scope of the present application is not limited to such a lamination structure, and as necessary, some layers may not be included, or other functional layers may be further added.

The organic material layer including the heterocyclic compound of Chemical Formula 1 may further include other materials as necessary.

In the organic light emitting device according to one embodiment of the present specification, materials other than the heterocyclic compound of Chemical Formula 1 are illustrated below, however, these are for illustrative purposes only and not for limiting the scope of the present application, and these materials may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris (4-carbazoyl-9-ylphenyl) amine (TCTA), 4,4′,4″-tri [phenyl (m-tolyl) amino]triphenylamine (m-MTDATA), 1,3,5-tris [4-(3-methylphenylphenylamino) phenyl]benzene (m-MTDAPB) or 4,4′,4″-tris [2-naphthyl (phenyl) amino]triphenylamine (2-TNATA) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used. For example, N,N′-di (1-naphthyl)-N,N′-diphenyl-(1,1′-biphenyl)-4,4′-diamine (NPB) may be used.

As the hole blocking material, BCP (bathocuproine) may be used, however, the hole blocking material is not limited thereto.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials. For example, tris (8-hydroxyquinolinato) aluminum (Alq₃) may be used.

As examples of the electron injection material, LiF is typically used in the art, however, the present application is not limited thereto.

As the light emitting material, red, green or blue light emitting materials may be used, and as necessary, two or more light emitting materials may be mixed and used. Herein, the two or more light emitting materials may be deposited with individual sources of supply or pre-mixed and deposited with one source of supply when used. In addition, fluorescent materials may also be used as the light emitting material, however, phosphorescent materials may also be used. As the light emitting material, materials emitting light by bonding holes and electrons injected from an anode and a cathode, respectively, may be used alone, however, materials having a host material and a dopant material involving together in light emission may also be used.

When mixing light emitting material hosts, same series hosts may be mixed, or different series hosts may be mixed. For example, any two or more types of materials among N-type host materials or P-type host materials may be selected and used as a host material of a light emitting layer.

The organic light emitting device according to one embodiment of the present specification may be a top-emission type, a bottom-emission type or a dual-emission type depending on the materials used.

The compound according to one embodiment of the present specification may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### <Preparation Example 1> Preparation of Intermediate A

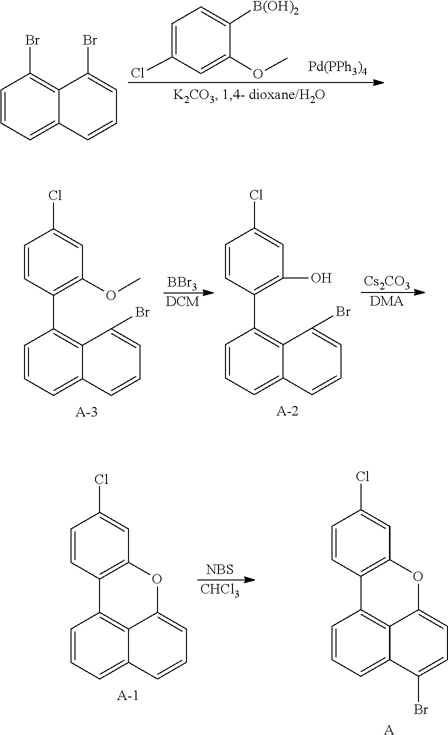

### 1) Preparation of Intermediate A-3

1,8-Dibromonaphthalene (20.0 g, 69.9 mM), (4-chloro-2-methoxyphenyl)boronic acid (13.0 g, 69.9 mM), Pd(PPh₃)₄ (tetrakis(triphenylphosphine)palladium(0)) (4.0 g, 3.5 mM) and K₂CO₃ (19.3 g, 140.0 mM) were dissolved in 1,4-dioxane/water (1,4-dioxane/H₂O) (200 mL/40 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:6) to obtain Intermediate A-3 (17.0 g, 70%).

The Hex means hexane.

### 2) Preparation of Intermediate A-2

Intermediate A-3 (17.0 g, 48.9 mM) and BBr₃ (boron tribromide) (13.9 mL, 146.7 mM) were dissolved in DCM (200 mL), and reacted for 6 hours at room temperature. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:1), and recrystallized with methanol to obtain Intermediate A-2 (14.7 g, 90%).

### 3) Preparation of Intermediate A-1

Intermediate A-2 (14.7 g, 44.1 mM) and Cs₂CO₃ (28.7 g, 88.2 mM) were dissolved in N,N-dimethylacetamide (DMA) (150 mL), and refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:1), and recrystallized with methanol to obtain Intermediate A-1 (9.5 g, 85%).

### 4) Preparation of Intermediate A

Intermediate A-1 (9.5 g, 37.6 mM) and n-bromosuccinimide (NBS) (7.4 g, 41.4 mM) were dissolved in CHCl₃ (chloroform) (100 mL), and refluxed for 24 hours. After the reaction was completed, the result was recrystallized with methanol to obtain Intermediate A (10.1 g, 81%).

Compounds including a benzo[kl]xanthene structure used for synthesizing the compounds of the present disclosure other than Intermediate A of Preparation Example 1, for example, Intermediate A1 of the following Table 2, may be synthesized using techniques known in the art.

### <Preparation Example 2> Preparation of Compound 1-1

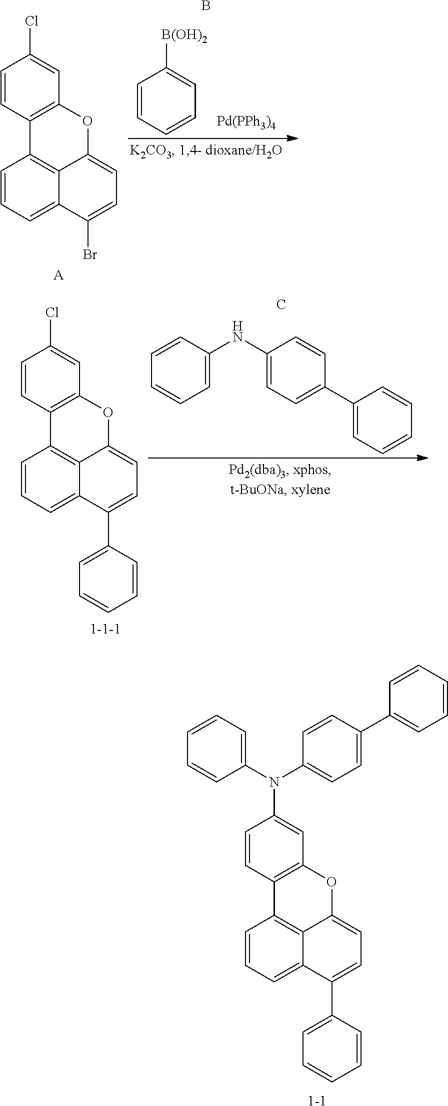

### 1) Preparation of Intermediate 1-1-1

Intermediate A (10.0 g, 30.2 mM) of Preparation Example 1, phenylboronic acid (5.5 g, 45.3 mM), Pd(PPh₃)₄ (1.7 g, 1.5 mM) and K₂CO₃ (8.3 g, 60.4 mM) were dissolved in 1,4-dioxane/water (100 mL/20 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain Intermediate 1-1-1 (9.0 g, 91%).

### 2) Preparation of Compound 1-1

Intermediate 1-1-1 (9.0 g, 27.4 mM), N-phenyl-[1,1′-biphenyl]-4-amine (6.7 g, 27.4 mM), Pd₂(dba)₃ (tris(dibenzylideneacetone)dipalladium(0)) (1.3 g, 1.4 mM), XPhos (2-dicyclohexylphosphino-2′,4′,6′-triisopropylbiphenyl) (1.3 g, 2.7 mM) and t-BuONa (sodium tert-butoxide) (3.9 g, 41.1 mM) were dissolved in xylene (100 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:4) to obtain target Compound 1-1 (12.1 g, 82%).

Target Compound A of the following Table 1 was synthesized in the same manner as in Preparation Example 2 except that Compound B of the following Table 1 was used instead of phenylboronic acid, and Compound C of the following Table 1 was used instead of N-phenyl-[1,1′-biphenyl]-4-amine.

TABLE 1

| Compound | | | Target Compound | |
|---|---|---|---|---|
| No. | Compound B | Compound C | A | Yield |
| 1-3 | 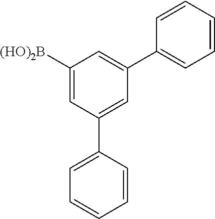 [SMILES: OB(O)c1cc(-c2ccccc2)cc(-c2ccccc2)c1] | 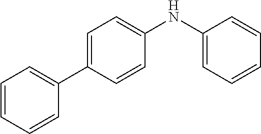 [SMILES: c1ccc(Nc2ccc(-c3ccccc3)cc2)cc1] | 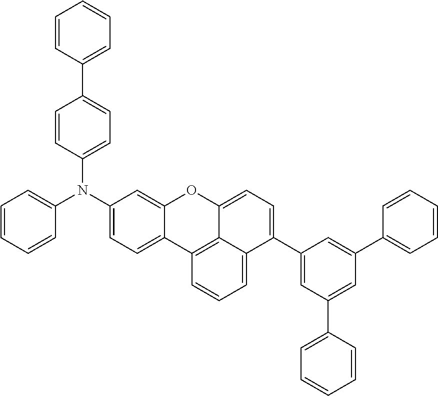 | 74% |
| 1-5 | 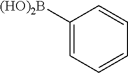 | 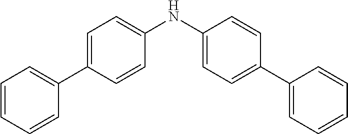 [SMILES: c1ccc(-c2ccc(Nc3ccc(-c4ccccc4)cc3)cc2)cc1] | 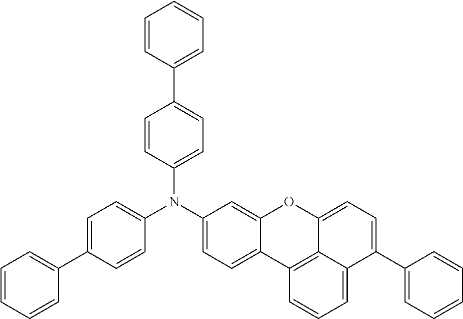 | 73% |
| 1-13 | 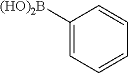 | 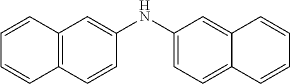 [SMILES: c1ccc2cc(Nc3ccc4ccccc4c3)ccc2c1] | 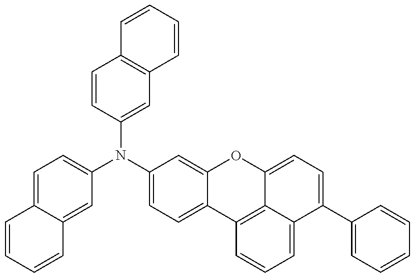 | 71% |
| 1-18 | 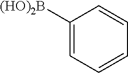 | 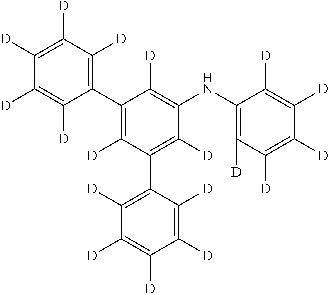 [SMILES: [2H]c1c([2H])c([2H])c(Nc2c([2H])c(-c3c([2H])c([2H])c([2H])c([2H])c3[2H])c([2H])c(-c3c([2H])c([2H])c([2H])c([2H])c3[2H])c2[2H])c([2H])c1[2H]] | 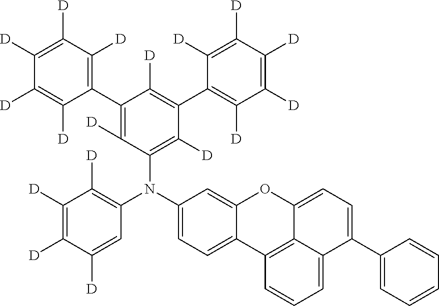 | 62% |
| 1-21 | 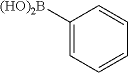 | 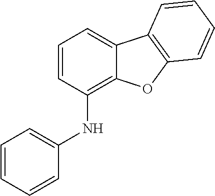 [SMILES: c1ccc(Nc2cccc3c2oc2ccccc23)cc1] | 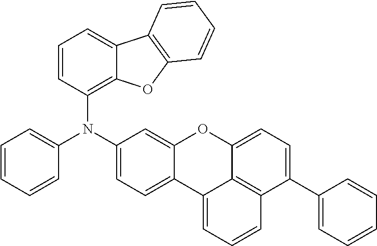 | 73% |
| 1-23 | 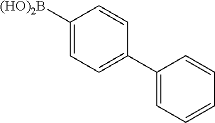 [SMILES: OB(O)c1ccc(-c2ccccc2)cc1] | 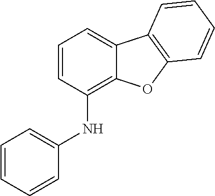 [SMILES: c1ccc(Nc2cccc3c2oc2ccccc23)cc1] | 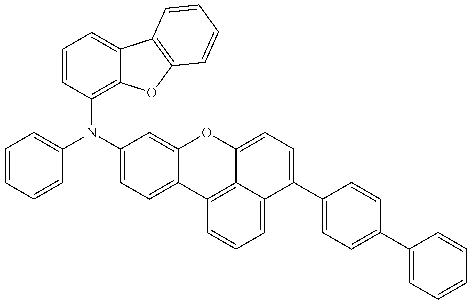 | 70% |
| 1-34 | 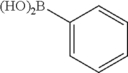 | 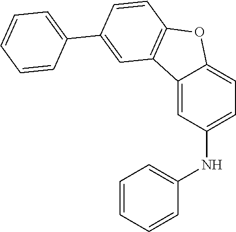 [SMILES: c1ccc(Nc2ccc3oc4ccc(-c5ccccc5)cc4c3c2)cc1] | 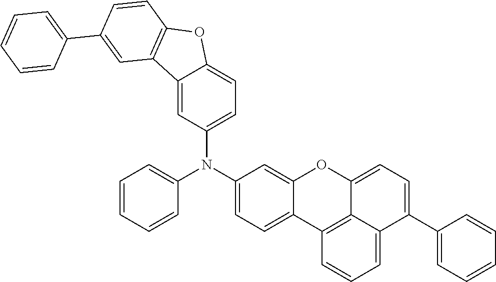 | 69% |
| 1-41 | 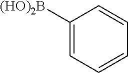 | 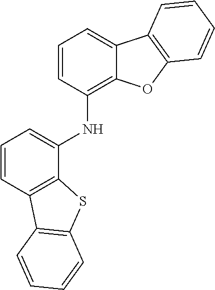 [SMILES: c1ccc2c(c1)oc1c(Nc3cccc4c3sc3ccccc34)cccc12] | 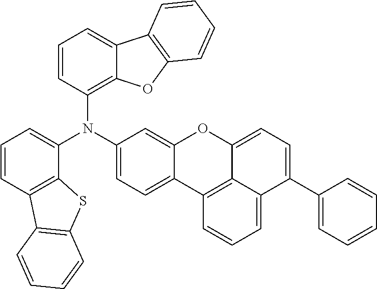 | 75% |
| 1-47 | 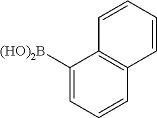 [SMILES: OB(O)c1cccc2ccccc12] | 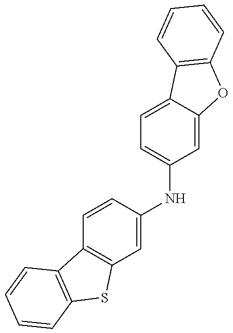 [SMILES: c1ccc2c(c1)oc1cc(Nc3ccc4c(c3)sc3ccccc34)ccc12] | 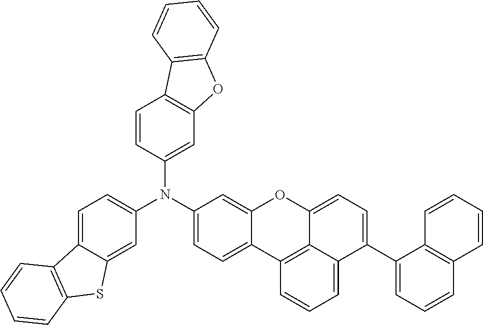 | 74% |
| 1-61 | 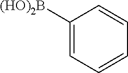 | 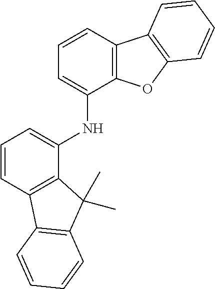 [SMILES: CC1(C)c2ccccc2-c2cccc(Nc3cccc4c3oc3ccccc34)c21] | 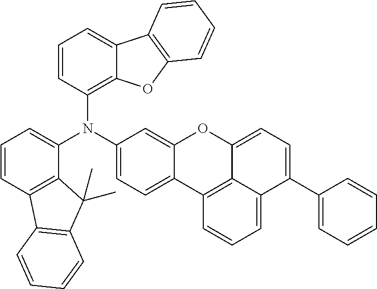 | 72% |
| 1-72 | 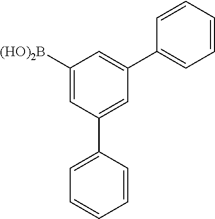 [SMILES: OB(O)c1cc(-c2ccccc2)cc(-c2ccccc2)c1] | 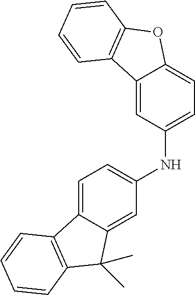 [SMILES: CC1(C)c2ccccc2-c2ccc(Nc3ccc4oc5ccccc5c4c3)cc21] | 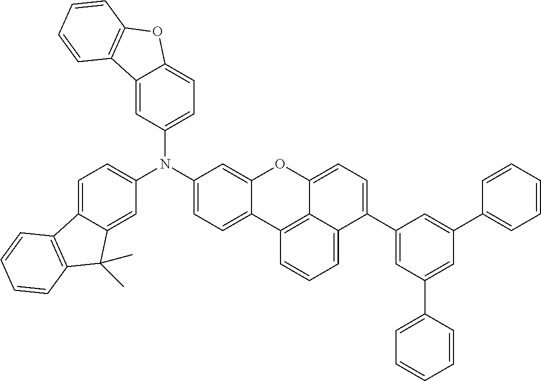 | 77% |
| 1-81 | 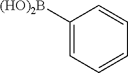 | 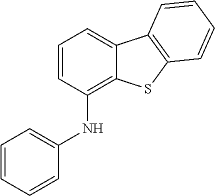 [SMILES: c1ccc(Nc2cccc3c2sc2ccccc23)cc1] | 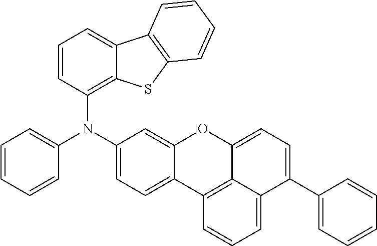 | 75% |
| 1-87 | 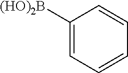 | 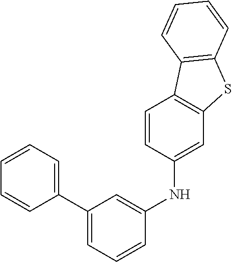 [SMILES: c1ccc(-c2cccc(Nc3ccc4c(c3)sc3ccccc34)c2)cc1] | 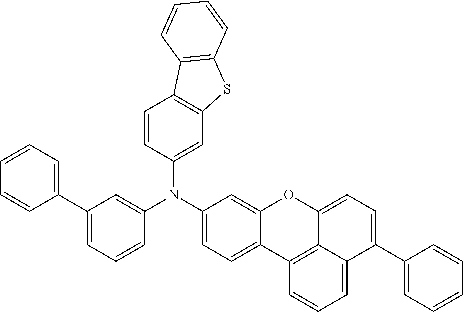 | 79% |
| 1-93 | 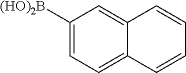 [SMILES: OB(O)c1ccc2ccccc2c1] | 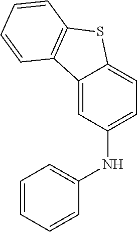 [SMILES: c1ccc(Nc2ccc3sc4ccccc4c3c2)cc1] | 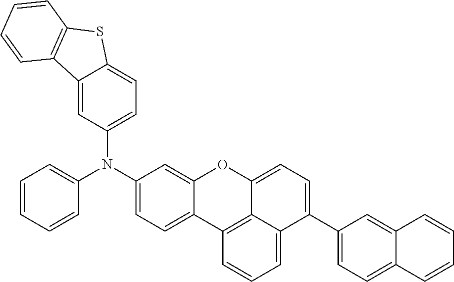 | 72% |
| 1-101 | 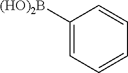 | 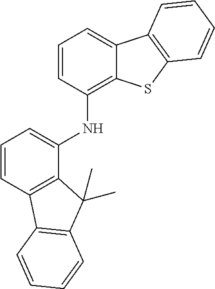 [SMILES: CC1(C)c2ccccc2-c2cccc(Nc3cccc4c3sc3ccccc34)c21] | 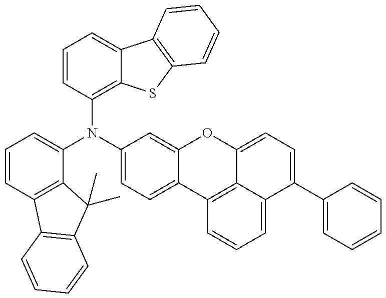 | 77% |
| 1-111 | 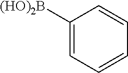 | 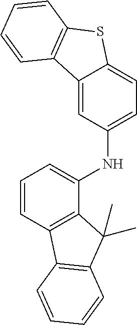 [SMILES: CC1(C)c2ccccc2-c2cccc(Nc3ccc4sc5ccccc5c4c3)c21] | 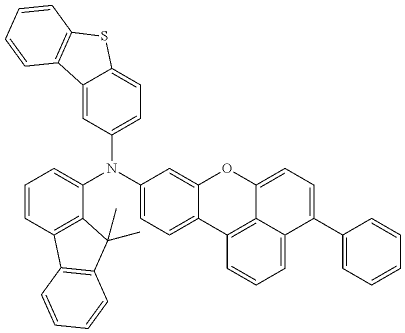 | 75% |
| 1-121 | 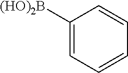 | 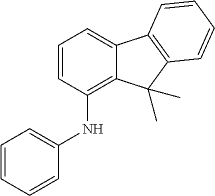 [SMILES: CC1(C)c2ccccc2-c2cccc(Nc3ccccc3)c21] | 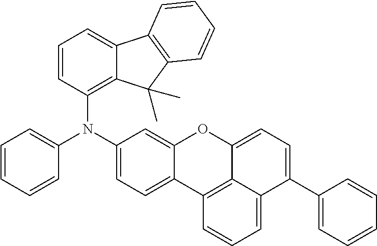 | 73% |
| 1-127 | 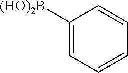 | 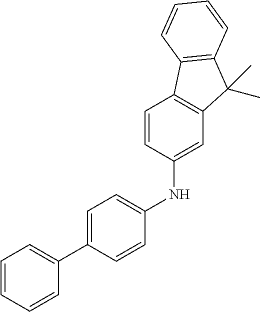 [SMILES: CC1(C)c2ccccc2-c2ccc(Nc3ccc(-c4ccccc4)cc3)cc21] | 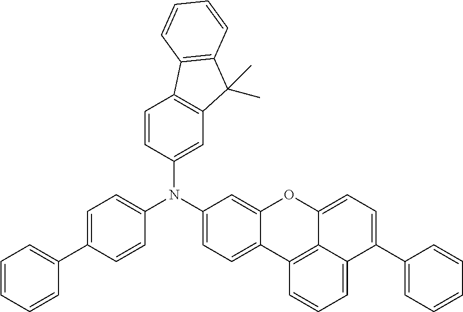 | 72% |
| 1-129 | 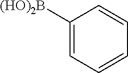 | 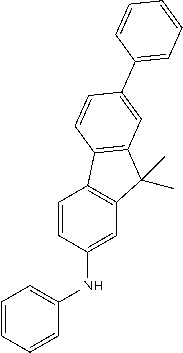 [SMILES: CC1(C)c2cc(Nc3ccccc3)ccc2-c2ccc(-c3ccccc3)cc21] | 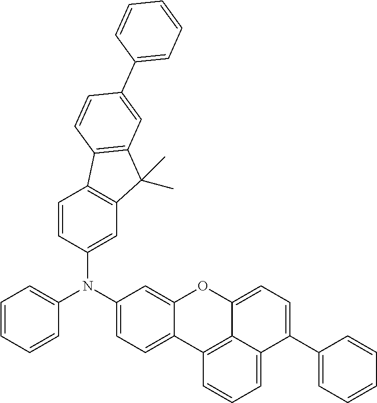 | 73% |

Target Compound A of the following Table 2 was synthesized in the same manner as in Preparation Example 2 except that Intermediate A1 of the following Table 2 was used instead of Intermediate A, and Compound C of the following Table 2 was used instead of N-phenyl-[1,1′-biphenyl]-4-amine.

TABLE 2

| Com- | | | | |
|---|---|---|---|---|
| pound | Intermediate | | Target Compound | |
| No. | A1 | Compound C | A | Yield |
| 1-281 | 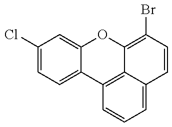 [SMILES: Clc1ccc2c(c1)Oc1c(Br)ccc3cccc-2c13] | 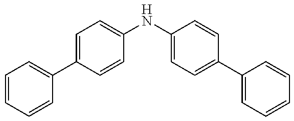 [SMILES: c1ccc(-c2ccc(Nc3ccc(-c4ccccc4)cc3)cc2)cc1] | 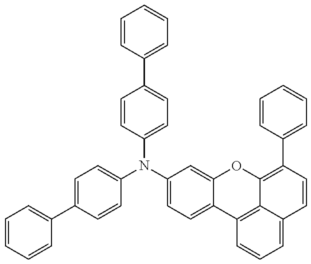 [SMILES: c1ccc(-c2ccc(N(c3ccc(-c4ccccc4)cc3)c3ccc4c(c3)Oc3c(-c5ccccc5)ccc5cccc-4c35)cc2)cc1] | 71% |
| 1-292 | 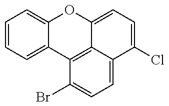 [SMILES: Clc1ccc2c3c(c(Br)ccc13)-c1ccccc1O2] | 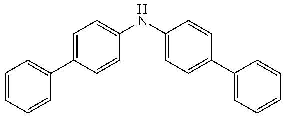 [SMILES: c1ccc(-c2ccc(Nc3ccc(-c4ccccc4)cc3)cc2)cc1] | 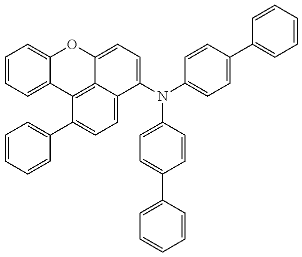 [SMILES: c1ccc(-c2ccc(N(c3ccc(-c4ccccc4)cc3)c3ccc4c5c(c(-c6ccccc6)ccc35)-c3ccccc3O4)cc2)cc1] | 68% |
| 1-298 | 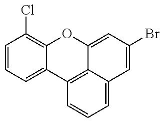 [SMILES: Clc1cccc2c1Oc1cc(Br)cc3cccc-2c13] | 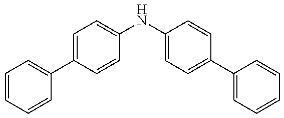 [SMILES: c1ccc(-c2ccc(Nc3ccc(-c4ccccc4)cc3)cc2)cc1] | 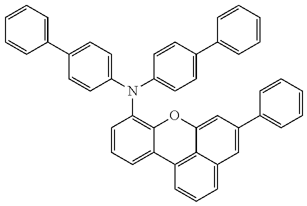 [SMILES: c1ccc(-c2ccc(N(c3ccc(-c4ccccc4)cc3)c3cccc4c3Oc3cc(-c5ccccc5)cc5cccc-4c35)cc2)cc1] | 73% |
| 1-308 | 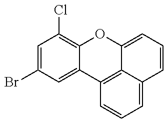 [SMILES: Clc1cc(Br)cc2c1Oc1cccc3cccc-2c13] | 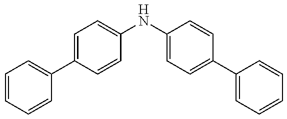 [SMILES: c1ccc(-c2ccc(Nc3ccc(-c4ccccc4)cc3)cc2)cc1] | 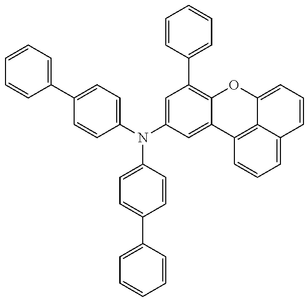 [SMILES: c1ccc(-c2ccc(N(c3ccc(-c4ccccc4)cc3)c3cc(-c4ccccc4)c4c(c3)-c3cccc5cccc(c35)O4)cc2)cc1] | 75% |
| 1-318 | 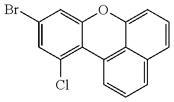 [SMILES: Clc1cc(Br)cc2c1-c1cccc3cccc(c13)O2] | 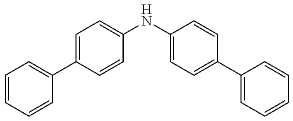 [SMILES: c1ccc(-c2ccc(Nc3ccc(-c4ccccc4)cc3)cc2)cc1] | 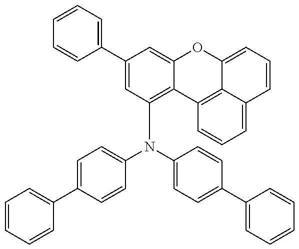 [SMILES: c1ccc(-c2ccc(N(c3ccc(-c4ccccc4)cc3)c3cc(-c4ccccc4)cc4c3-c3cccc5cccc(c35)O4)cc2)cc1] | 70% |
| 1-332 | 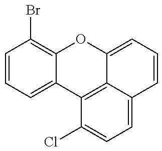 [SMILES: Clc1ccc2cccc3c2c1-c1cccc(Br)c1O3] | 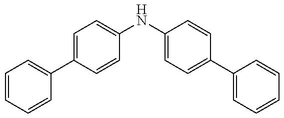 [SMILES: c1ccc(-c2ccc(Nc3ccc(-c4ccccc4)cc3)cc2)cc1] | 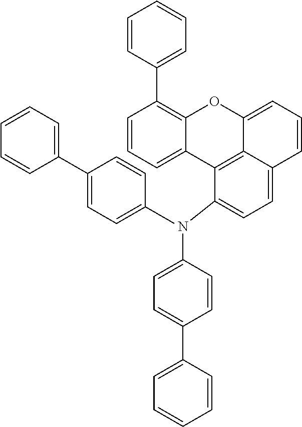 | 76% |
| 1-341 | 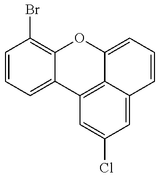 [SMILES: Clc1cc2c3c(cccc3c1)Oc1c(Br)cccc1-2] | 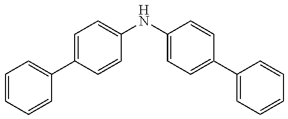 [SMILES: c1ccc(-c2ccc(Nc3ccc(-c4ccccc4)cc3)cc2)cc1] | 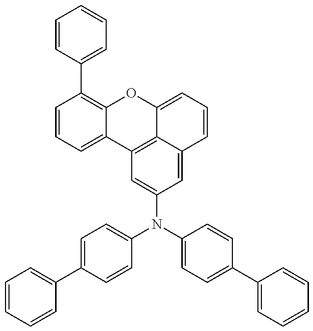 [SMILES: c1ccc(-c2ccc(N(c3ccc(-c4ccccc4)cc3)c3cc4c5c(cccc5c3)Oc3c(-c5ccccc5)cccc3-4)cc2)cc1] | 72% |
| 1-349 | 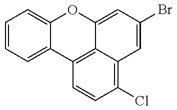 [SMILES: Clc1ccc2c3c(cc(Br)cc13)Oc1ccccc1-2] | 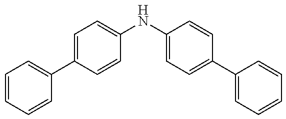 [SMILES: c1ccc(-c2ccc(Nc3ccc(-c4ccccc4)cc3)cc2)cc1] | 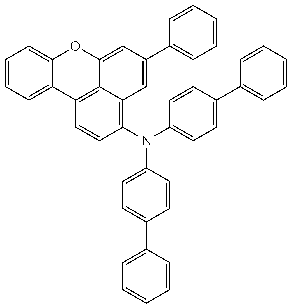 [SMILES: c1ccc(-c2ccc(N(c3ccc(-c4ccccc4)cc3)c3ccc4c5c(cc(-c6ccccc6)cc35)Oc3ccccc3-4)cc2)cc1] | 74% |
| 1-359 | 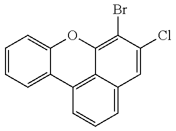 [SMILES: Clc1cc2cccc3c2c(c1Br)Oc1ccccc1-3] | 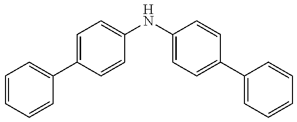 [SMILES: c1ccc(-c2ccc(Nc3ccc(-c4ccccc4)cc3)cc2)cc1] | 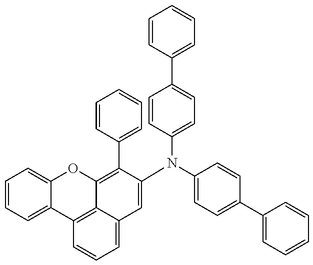 [SMILES: c1ccc(-c2ccc(N(c3ccc(-c4ccccc4)cc3)c3cc4cccc5c4c(c3-c3ccccc3)Oc3ccccc3-5)cc2)cc1] | 75% |
| 1-374 | 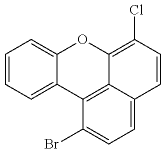 [SMILES: Clc1ccc2ccc(Br)c3c2c1Oc1ccccc1-3] | 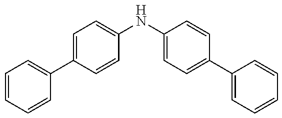 [SMILES: c1ccc(-c2ccc(Nc3ccc(-c4ccccc4)cc3)cc2)cc1] | 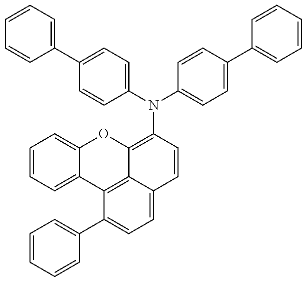 [SMILES: c1ccc(-c2ccc(N(c3ccc(-c4ccccc4)cc3)c3ccc4ccc(-c5ccccc5)c5c4c3Oc3ccccc3-5)cc2)cc1] | 70% |

### <Preparation Example 3> Preparation of Compound 1-8

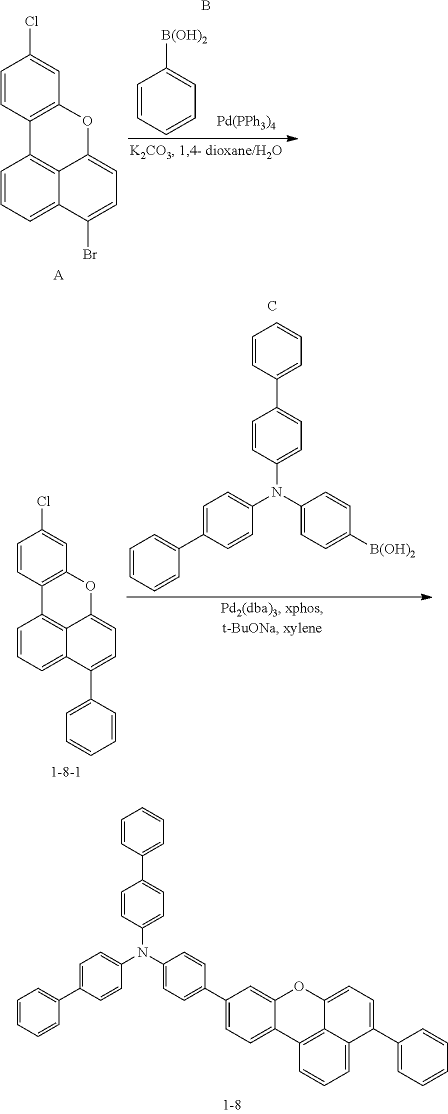

### 1) Preparation of Intermediate 1-8-1

Intermediate A (10.0 g, 30.2 mM) of Preparation Example 1, phenylboronic acid (5.5 g, 45.3 mM), Pd(PPh₃)₄(1.7 g, 1.5 mM) and K₂CO₃ (8.3 g, 60.4 mM) were dissolved in 1,4-dioxane/water (100 mL/20 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain Intermediate 1-8-1 (9.0 g, 91%).

### 2) Preparation of Compound 1-8

Intermediate 1-8-1 (9.0 g, 27.4 mM), (4-(di([1,1′-biphenyl]-4-yl)amino)phenyl)boronic acid (12.1 g, 27.4 mM), Pd₂(dba)₃ (1.3 g, 1.4 mM), XPhos (1.3 g, 2.7 mM) and K₂CO₃ (11.4 g, 82.2 mM) were dissolved in 1,4-dioxane/water (100 mL/20 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3) to obtain target Compound 1-8 (15.7 g, 83%).

Target Compound A of the following Table 3 was synthesized in the same manner as in Preparation Example 3 except that Compound C of the following Table 3 was used instead of (4-(di([1,1′-biphenyl]-4-yl)amino)phenyl)boronic acid.

TABLE 3

| Com- | | | |
|---|---|---|---|
| pound | | Target Compound | |
| No. | Compound C | A | Yield |
| 1-60 | 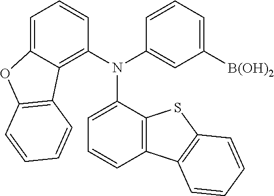 [SMILES: OB(O)c1cccc(N(c2cccc3c2sc2ccccc23)c2cccc3oc4ccccc4c23)c1] | 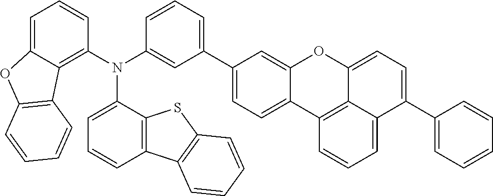 [SMILES: c1ccc(-c2ccc3c4c(cccc24)-c2ccc(-c4cccc(N(c5cccc6c5sc5ccccc56)c5cccc6oc7ccccc7c56)c4)cc2O3)cc1] | 77% |
| 1-80 | 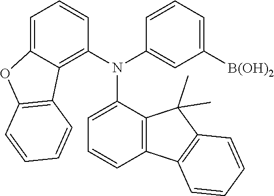 [SMILES: CC1(C)c2ccccc2-c2cccc(N(c3cccc(B(O)O)c3)c3cccc4oc5ccccc5c34)c21] | 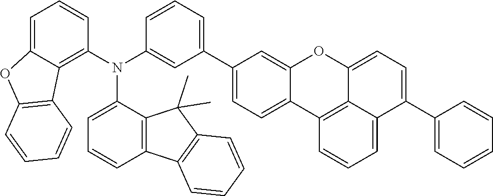 [SMILES: CC1(C)c2ccccc2-c2cccc(N(c3cccc(-c4ccc5c(c4)Oc4ccc(-c6ccccc6)c6cccc-5c46)c3)c3cccc4oc5ccccc5c34)c21] | 72% |
| 1-120 | 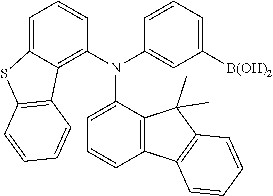 [SMILES: CC1(C)c2ccccc2-c2cccc(N(c3cccc(B(O)O)c3)c3cccc4sc5ccccc5c34)c21] | 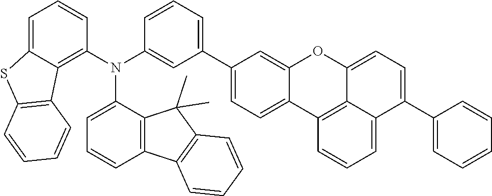 [SMILES: CC1(C)c2ccccc2-c2cccc(N(c3cccc(-c4ccc5c(c4)Oc4ccc(-c6ccccc6)c6cccc-5c46)c3)c3cccc4sc5ccccc5c34)c21] | 76% |
| 1-140 | 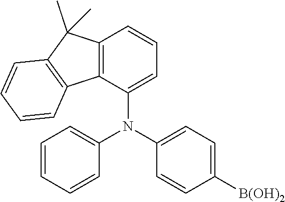 [SMILES: CC1(C)c2ccccc2-c2c(N(c3ccccc3)c3ccc(B(O)O)cc3)cccc21] | 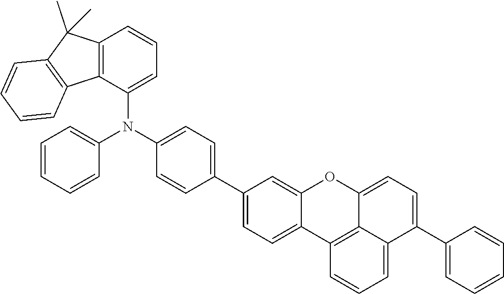 [SMILES: CC1(C)c2ccccc2-c2c(N(c3ccccc3)c3ccc(-c4ccc5c(c4)Oc4ccc(-c6ccccc6)c6cccc-5c46)cc3)cccc21] | 74% |

### <Preparation Example 4> Preparation of Compound 1-141

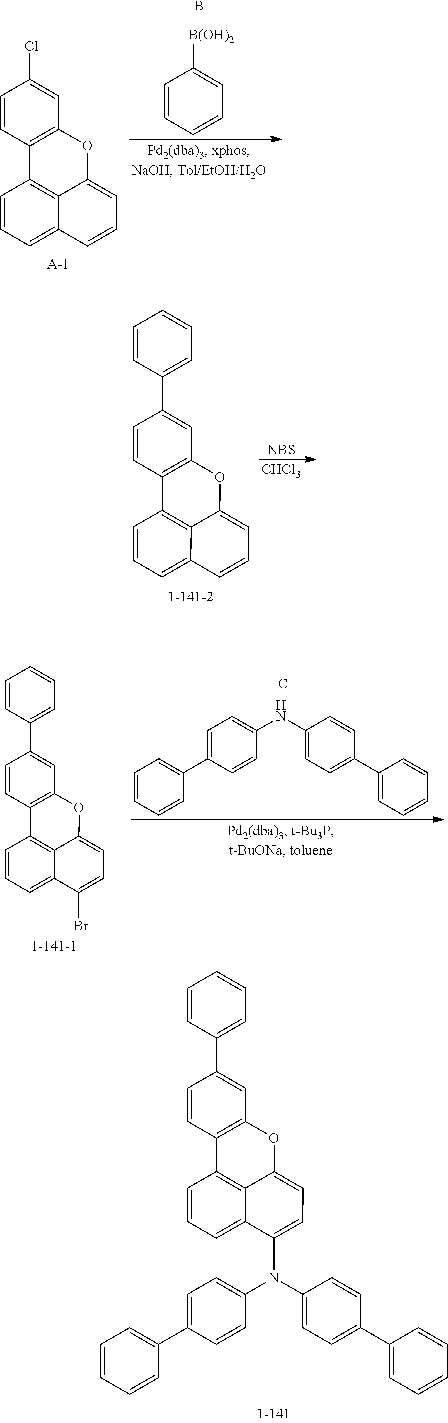

### 1) Preparation of Intermediate 1-141-2

Intermediate A-1 (10.0 g, 39.6 mM) of Preparation Example 1, phenylboronic acid (7.2 g, 59.4 mM), Pd₂(dba)₃ (1.8 g, 2.0 mM), XPhos (2.8 g, 5.9 mM) and NaOH (3.2 g, 79.2 mM) were dissolved in toluene/ethanol/water (toluene/EtOH/H₂O) (100 mL/20 mL/20 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:5), and recrystallized with methanol to obtain Intermediate 1-141-2 (8.2 g, 70%).

### 2) Preparation of Intermediate 1-141-1

Intermediate 1-141-2 (8.2 g, 27.9 mM) and NBS (5.5 g, 30.7 mM) were dissolved in CHCl₃ (100 mL), and refluxed for 24 hours. After the reaction was completed, the result was recrystallized with methanol to obtain Intermediate 1-141-1 (8.2 g, 79%).

### 3) Preparation of Compound 1-141

Intermediate 1-141-1 (8.2 g, 22.0 mM), di([1,1′-biphenyl]-4-yl)amine (7.1 g, 22.0 mM), Pd₂(dba)₃ (1.0 g, 1.4 mM), t-Bu₃P (tri-tert-butylphosphine) (1.0 mL, 2.2 mM) and t-BuONa (4.2 g, 44.0 mM) were dissolved in toluene (100 mL), and refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:7) to obtain target Compound 1-141 (11.2 g, 83%).

Target Compound A of the following Table 4 was synthesized in the same manner as in Preparation Example 4 except that Compound B of the following Table 4 was used instead of phenylboronic acid, and Compound C of the following Table 4 was used instead of di([1,1′-biphenyl]-4-yl)amine.

TABLE 4

| Com- | | | | |
|---|---|---|---|---|
| pound | | | | |
| No. | Compound B | Compound C | Target Compound A | Yield |
| 1-145 | 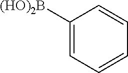 | 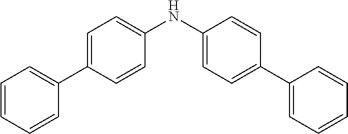 [SMILES: c1ccc(-c2ccc(Nc3ccc(-c4ccccc4)cc3)cc2)cc1] | 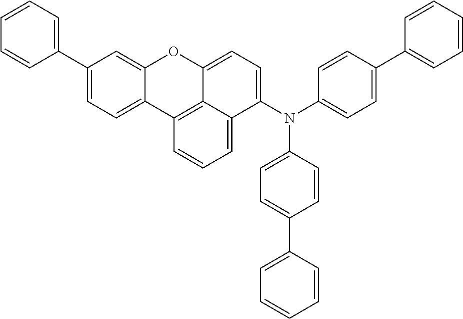 [SMILES: c1ccc(-c2ccc(N(c3ccc(-c4ccccc4)cc3)c3ccc4c5c(cccc35)-c3ccc(-c5ccccc5)cc3O4)cc2)cc1] | 45% |
| 1-149 | 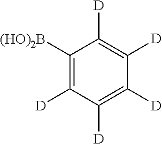 [SMILES: [2H]c1c([2H])c([2H])c(B(O)O)c([2H])c1[2H]] | 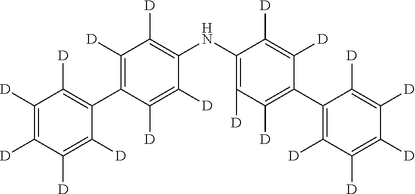 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 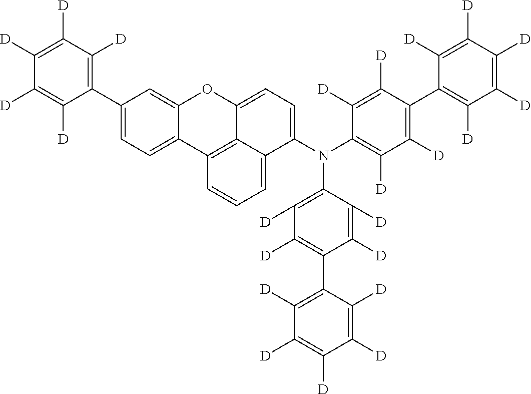 [SMILES: [2H]c1c([2H])c([2H])c(-c2ccc3c(c2)Oc2ccc(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])c4cccc-3c24)c([2H])c1[2H]] | 37% |
| 1-157 | 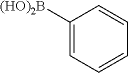 | 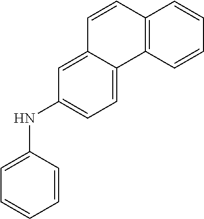 [SMILES: c1ccc(Nc2ccc3c(ccc4ccccc43)c2)cc1] | 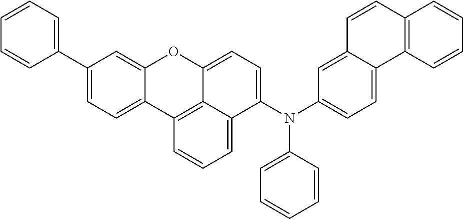 [SMILES: c1ccc(-c2ccc3c(c2)Oc2ccc(N(c4ccccc4)c4ccc5c(ccc6ccccc65)c4)c4cccc-3c24)cc1] | 40% |
| 1-160 | 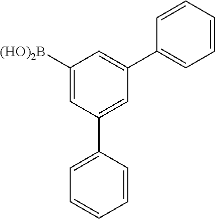 [SMILES: OB(O)c1cc(-c2ccccc2)cc(-c2ccccc2)c1] | 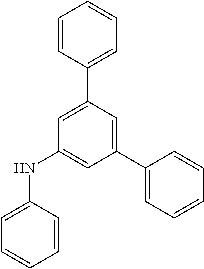 [SMILES: c1ccc(Nc2cc(-c3ccccc3)cc(-c3ccccc3)c2)cc1] | 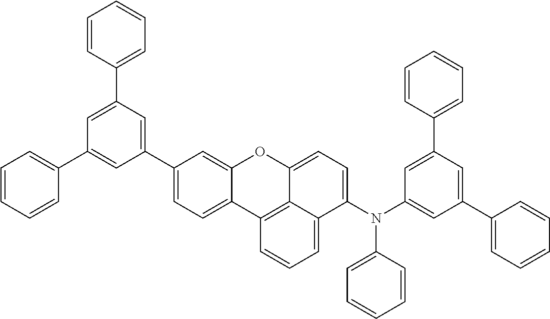 [SMILES: c1ccc(-c2cc(-c3ccccc3)cc(-c3ccc4c(c3)Oc3ccc(N(c5ccccc5)c5cc(-c6ccccc6)cc(-c6ccccc6)c5)c5cccc-4c35)c2)cc1] | 42% |
| 1-161 | 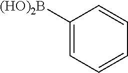 | 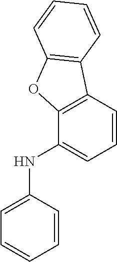 [SMILES: c1ccc(Nc2cccc3c2oc2ccccc23)cc1] | 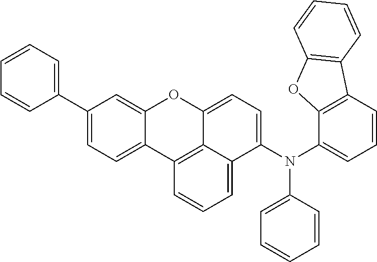 [SMILES: c1ccc(-c2ccc3c(c2)Oc2ccc(N(c4ccccc4)c4cccc5c4oc4ccccc45)c4cccc-3c24)cc1] | 46% |
| 1-169 | 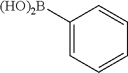 | 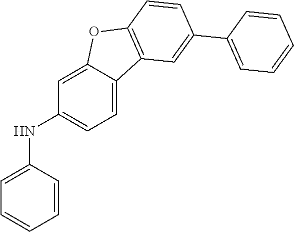 [SMILES: c1ccc(Nc2ccc3c(c2)oc2ccc(-c4ccccc4)cc23)cc1] | 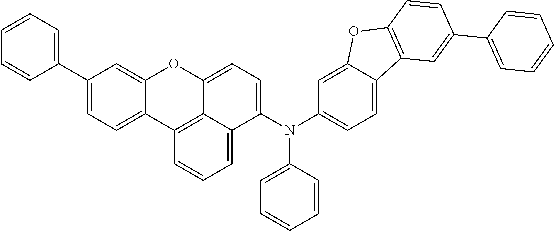 [SMILES: c1ccc(-c2ccc3c(c2)Oc2ccc(N(c4ccccc4)c4ccc5c(c4)oc4ccc(-c6ccccc6)cc45)c4cccc-3c24)cc1] | 46% |
| 1-181 | 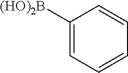 | 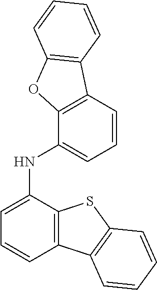 [SMILES: c1ccc2c(c1)oc1c(Nc3cccc4c3sc3ccccc34)cccc12] | 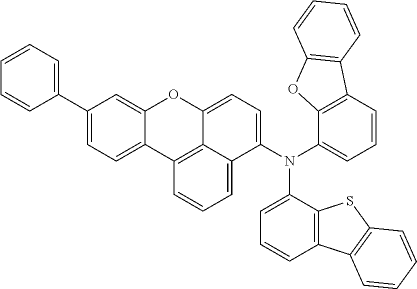 [SMILES: c1ccc(-c2ccc3c(c2)Oc2ccc(N(c4cccc5c4oc4ccccc45)c4cccc5c4sc4ccccc45)c4cccc-3c24)cc1] | 42% |
| 1-186 | 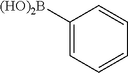 | 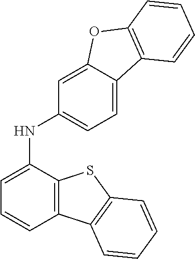 [SMILES: c1ccc2c(c1)oc1cc(Nc3cccc4c3sc3ccccc34)ccc12] | 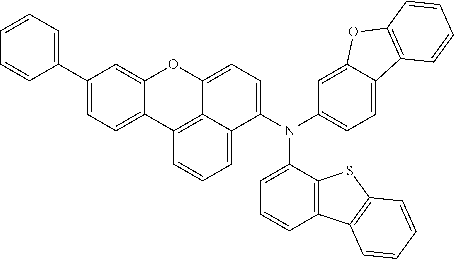 [SMILES: c1ccc(-c2ccc3c(c2)Oc2ccc(N(c4ccc5c(c4)oc4ccccc45)c4cccc5c4sc4ccccc45)c4cccc-3c24)cc1] | 48% |
| 1-192 | 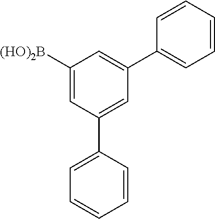 [SMILES: OB(O)c1cc(-c2ccccc2)cc(-c2ccccc2)c1] | 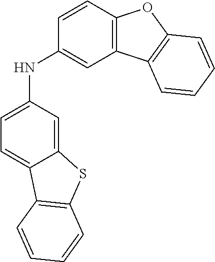 [SMILES: c1ccc2c(c1)oc1ccc(Nc3ccc4c(c3)sc3ccccc34)cc12] | 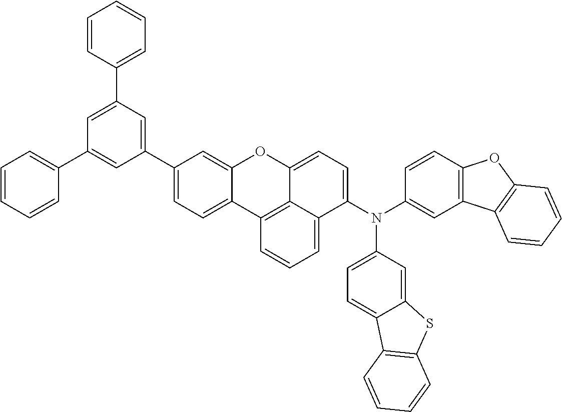 [SMILES: c1ccc(-c2cc(-c3ccccc3)cc(-c3ccc4c(c3)Oc3ccc(N(c5ccc6c(c5)sc5ccccc56)c5ccc6oc7ccccc7c6c5)c5cccc-4c35)c2)cc1] | 45% |
| 1-201 | 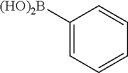 | 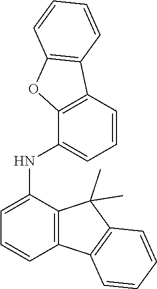 [SMILES: CC1(C)c2ccccc2-c2cccc(Nc3cccc4c3oc3ccccc34)c21] | 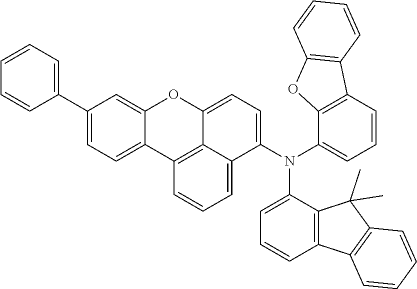 [SMILES: CC1(C)c2ccccc2-c2cccc(N(c3ccc4c5c(cccc35)-c3ccc(-c5ccccc5)cc3O4)c3cccc4c3oc3ccccc34)c21] | 39% |
| 1-207 | 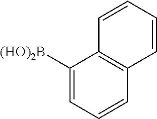 [SMILES: OB(O)c1cccc2ccccc12] | 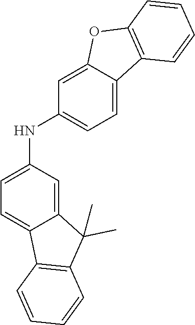 [SMILES: CC1(C)c2ccccc2-c2ccc(Nc3ccc4c(c3)oc3ccccc34)cc21] | 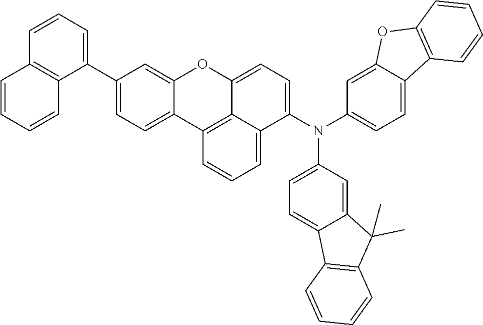 [SMILES: CC1(C)c2ccccc2-c2ccc(N(c3ccc4c(c3)oc3ccccc34)c3ccc4c5c(cccc35)-c3ccc(-c5cccc6ccccc56)cc3O4)cc21] | 43% |
| 1-217 | 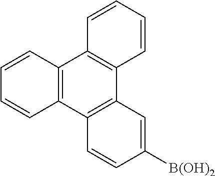 [SMILES: OB(O)c1ccc2c3ccccc3c3ccccc3c2c1] | 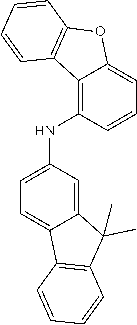 [SMILES: CC1(C)c2ccccc2-c2ccc(Nc3cccc4oc5ccccc5c34)cc21] | 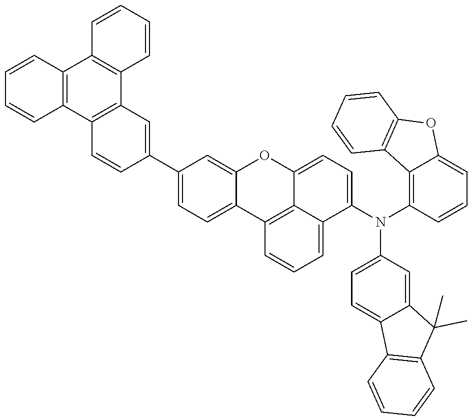 [SMILES: CC1(C)c2ccccc2-c2ccc(N(c3ccc4c5c(cccc35)-c3ccc(-c5ccc6c7ccccc7c7ccccc7c6c5)cc3O4)c3cccc4oc5ccccc5c34)cc21] | 44% |
| 1-221 | 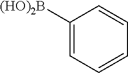 | 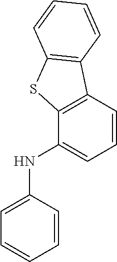 [SMILES: c1ccc(Nc2cccc3c2sc2ccccc23)cc1] | 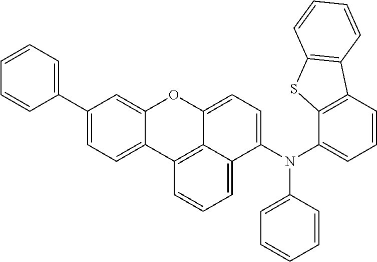 [SMILES: c1ccc(-c2ccc3c(c2)Oc2ccc(N(c4ccccc4)c4cccc5c4sc4ccccc45)c4cccc-3c24)cc1] | 44% |
| 1-234 | 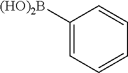 | 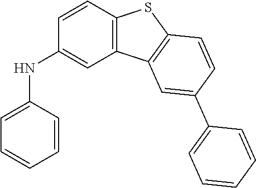 [SMILES: c1ccc(Nc2ccc3sc4ccc(-c5ccccc5)cc4c3c2)cc1] | 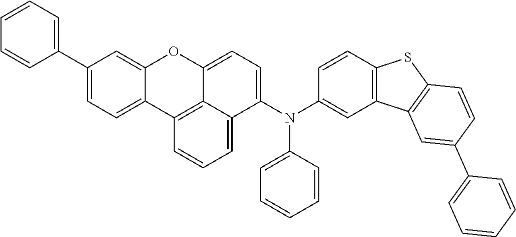 [SMILES: c1ccc(-c2ccc3c(c2)Oc2ccc(N(c4ccccc4)c4ccc5sc6ccc(-c7ccccc7)cc6c5c4)c4cccc-3c24)cc1] | 46% |
| 1-241 | 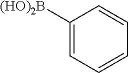 | 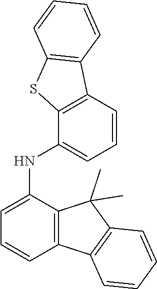 [SMILES: CC1(C)c2ccccc2-c2cccc(Nc3cccc4c3sc3ccccc34)c21] | 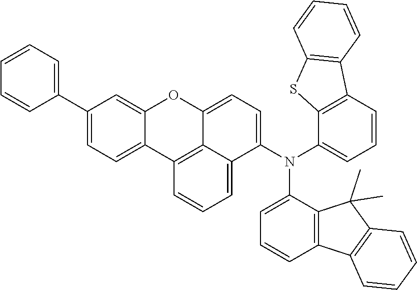 [SMILES: CC1(C)c2ccccc2-c2cccc(N(c3ccc4c5c(cccc35)-c3ccc(-c5ccccc5)cc3O4)c3cccc4c3sc3ccccc34)c21] | 47% |
| 1-242 | 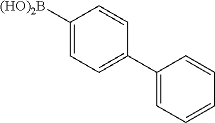 [SMILES: OB(O)c1ccc(-c2ccccc2)cc1] | 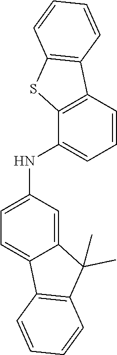 [SMILES: CC1(C)c2ccccc2-c2ccc(Nc3cccc4c3sc3ccccc34)cc21] | 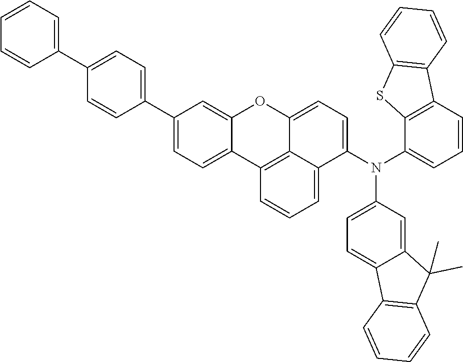 [SMILES: CC1(C)c2ccccc2-c2ccc(N(c3ccc4c5c(cccc35)-c3ccc(-c5ccc(-c6ccccc6)cc5)cc3O4)c3cccc4c3sc3ccccc34)cc21] | 49% |
| 1-247 | 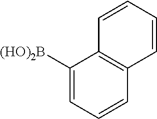 [SMILES: OB(O)c1cccc2ccccc12] | 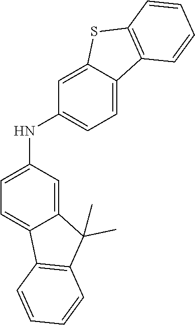 [SMILES: CC1(C)c2ccccc2-c2ccc(Nc3ccc4c(c3)sc3ccccc34)cc21] | 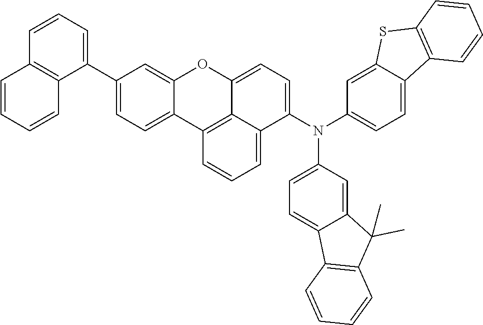 [SMILES: CC1(C)c2ccccc2-c2ccc(N(c3ccc4c(c3)sc3ccccc34)c3ccc4c5c(cccc35)-c3ccc(-c5cccc6ccccc56)cc3O4)cc21] | 48% |
| 1-261 | 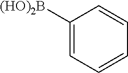 | 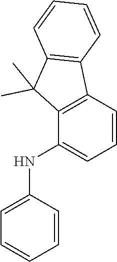 [SMILES: CC1(C)c2ccccc2-c2cccc(Nc3ccccc3)c21] | 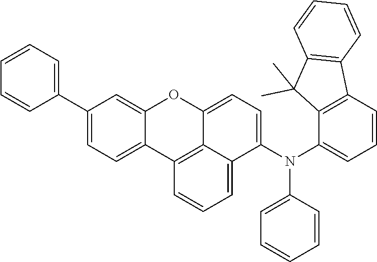 [SMILES: CC1(C)c2ccccc2-c2cccc(N(c3ccccc3)c3ccc4c5c(cccc35)-c3ccc(-c5ccccc5)cc3O4)c21] | 50% |
| 1-266 | 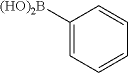 | 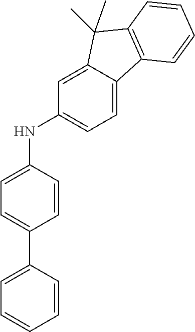 [SMILES: CC1(C)c2ccccc2-c2ccc(Nc3ccc(-c4ccccc4)cc3)cc21] | 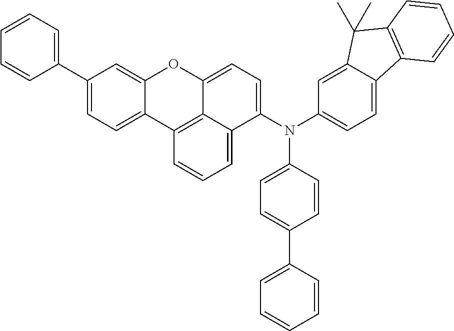 [SMILES: CC1(C)c2ccccc2-c2ccc(N(c3ccc(-c4ccccc4)cc3)c3ccc4c5c(cccc35)-c3ccc(-c5ccccc5)cc3O4)cc21] | 46% |
| 1-267 | 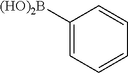 | 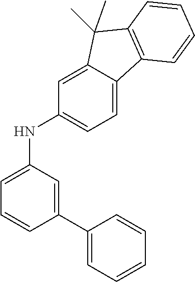 [SMILES: CC1(C)c2ccccc2-c2ccc(Nc3cccc(-c4ccccc4)c3)cc21] | 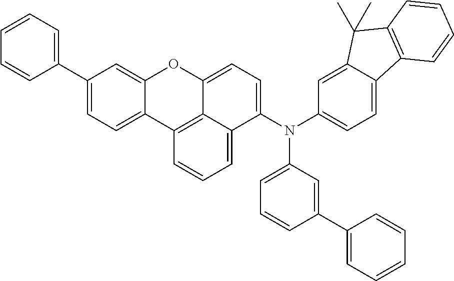 [SMILES: CC1(C)c2ccccc2-c2ccc(N(c3cccc(-c4ccccc4)c3)c3ccc4c5c(cccc35)-c3ccc(-c5ccccc5)cc3O4)cc21] | 44% |

### <Preparation Example 5> Preparation of Compound 1-148

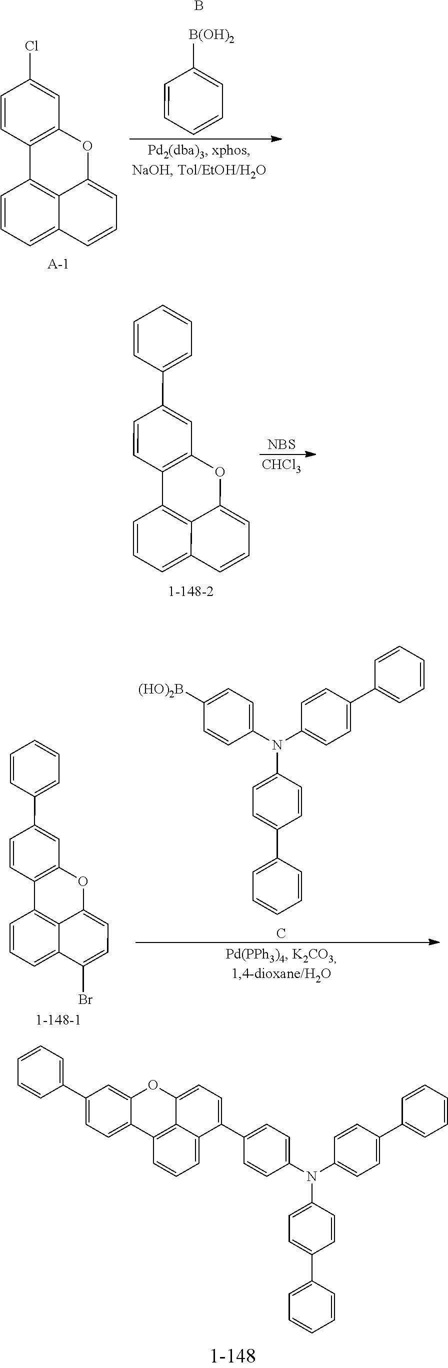

### 1) Preparation of Intermediate 1-148-2

Intermediate A-1 (10.0 g, 39.6 mM) of Preparation Example 1, phenylboronic acid (7.2 g, 59.4 mM), Pd₂(dba)₃ (1.8 g, 2.0 mM), XPhos (2.8 g, 5.9 mM) and NaOH (3.2 g, 79.2 mM) were dissolved in toluene/ethanol/water (toluene/EtOH/H₂O) (100 mL/20 mL/20 mL), and refluxed for 6 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:5), and recrystallized with methanol to obtain Intermediate 1-148-2 (8.2 g, 70%).

### 2) Preparation of Intermediate 1-148-1

Intermediate 1-148-2 (8.2 g, 27.9 mM) and NBS (5.5 g, 30.7 mM) were dissolved in CHCl₃ (100 mL), and refluxed for 24 hours. After the reaction was completed, the result was recrystallized with methanol to obtain Intermediate 1-148-1 (8.2 g, 79%).

### 3) Preparation of Compound 1-148

Intermediate 1-148-1 (8.2 g, 22.0 mM), (4-(di([1,1′-biphenyl]-4-yl)amino)phenyl)boronic acid (9.7 g, 22.0 mM), Pd(PPh₃)₄(1.3 g, 1.1 mM) and K₂CO₃ (6.1 g, 44.0 mM) were dissolved in 1,4-dioxane/water (1,4-dioxane/H₂O) (100 mL/20 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:4) to obtain target Compound 1-148 (13.5 g, 89%).

Target Compound A of the following Table 5 was synthesized in the same manner as in Preparation Example 5 except that Compound C of the following Table 5 was used instead of (4-(di([1,1′-biphenyl]-4-yl)amino)phenyl)boronic acid.

TABLE 5

| Compound | | Target Compound | |
|---|---|---|---|
| No. | Compound C | A | Yield |
| 1-180 | 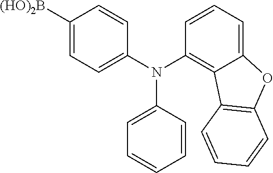 [SMILES: OB(O)c1ccc(N(c2ccccc2)c2cccc3oc4ccccc4c23)cc1] | 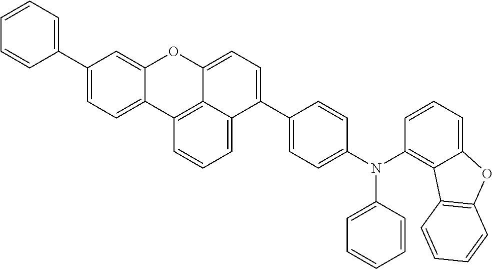 | 57% |
| 1-225 | 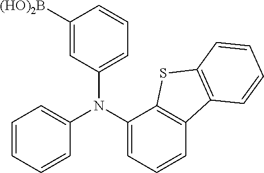 [SMILES: OB(O)c1cccc(N(c2ccccc2)c2cccc3c2sc2ccccc23)c1] | 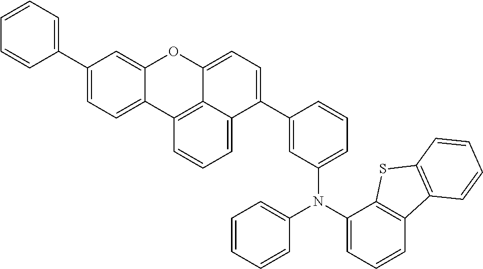 | 55% |
| 1-275 | 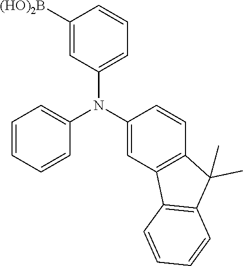 [SMILES: CC1(C)c2ccccc2-c2cc(N(c3ccccc3)c3cccc(B(O)O)c3)ccc21] | 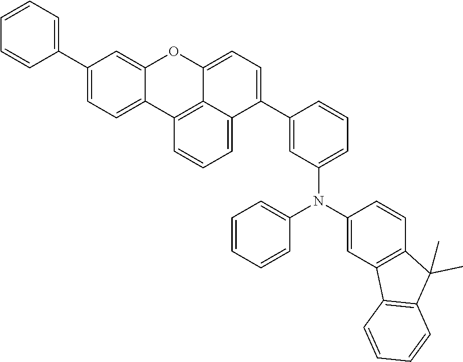 | 51% |

The rest of the compounds other than the compounds described in Table 1 to Table 5 were also prepared in the same manner as in the methods described in the preparation examples described above. Synthesis identification results for the compounds prepared above are shown in the following Table 6 and Table 7. Table 6 shows measurement values of ¹H NMR (CDCl₃, 200 Mz), and Table 7 shows measurement values of FD-mass spectrometry (FD-MS: field desorption mass spectrometry).

TABLE 6

| Compound | |
|---|---|
| No. | ¹H NMR (CDCl₃, 200 MHZ) |
| 1-1 | δ = 8.52(1H, d), 8.48(1H, d), 7.80(1H, d), 7.79(2H, d), |
| | 7.64(1H, t), 7.41-7.54(11H, m), 7.20(2H, t), 6.81(1H, |
| | t), 6.63-6.71(5H, m), 6.41(1H, d), 6.39(1H, s) |
| 1-3 | δ = 8.52(1H, d), 8.48(1H, d), 7.80(1H, d), 6.66(3H, s), |
| | 7.64(1H, t), 7.41-7.54(18H, m), 7.20(2H, t), 6.81(1H, |
| | t), 6.63-6.71(5H, m), 6.41(1H, d), 6.39(1H, s) |
| 1-5 | δ = 8.52(1H, d), 8.48(1H, d), 7.80(1H, d), 7.79(2H, d), |
| | 7.64(1H, t), 7.41-7.54(18H, m), 6.71(1H, d), 6.69(4H, |
| | d), 6.41(1H, d), 6.39(1H, s) |
| 1-8 | δ == 8.52(1H, d), 8.48(1H, d), 7.79-7. 81(4H, m), |
| | 7.64(1H, t), 7.57(1H, s), 7.41-7.54(19H, m), 6.97(1H, |
| | d), 6.71(1H, d), 6.69(6H, d) |
| 1-13 | δ = 8.52(1H, d), 8.48(1H, d), 7.74-7.88(11H, m), |
| | 7.64(1H, t), 7.36-7.51(10H, m), 6.71(1H, d), 6.41(1H, |
| | d), 6.39(1H, s) |
| 1-18 | δ = 8.52(1H, d), 8.48(1H, d), 7.80(1H, d), 7.79(2H, d), |
| | 7.64(1H, t), 7.41-7.51(4H, m), 6.71(1H, d), 6.41(1H, |
| | d), 6.39(1H, s) |
| 1-21 | δ = 8.52(1H, d), 8.48(1H, d), 7.89(1H, d), 7.80(1H, d), |
| | 7.79(2H, d), 7.66(1H, d), 7.64(1H, t), 7.20-7.51(9H, |
| | m), 7.07(1H, t), 6.81(1H, t), 6.71(1H, d), 6.63(2H, |
| | d), 6.41(1H, d), 6.39(2H, s) |
| 1-23 | δ = 8.52(1H, d), 8.48(1H, d), 7.89(1H, d), 7.80(1H, d), |
| | 7.66(1H, d), 7.64(1H, t), 7.20-7.52(15H, m), 7.07(1H, |
| | t), 6.81(1H, t), 6.71(1H, d), 6.63(2H, d), 6.41(1H, |
| | d), 6.39(2H, s) |
| 1-34 | δ = 8.52(1H, d), 8.48(1H, d), 7.64-7.81(8H, m), 7.41- |
| | 7.52(10H, m), 7.20(2H, t), 6.81(1H, t), 6.71(1H, d), |
| | 6.63(2H, d), 6.41(1H, d), 6.39(2H, s) |
| 1-41 | δ = 8.52(1H, d), 8.48(1H, d), 8.45(1H, d), 7.98(1H, d), |
| | 7.89(1H, d), 7.79-7.81(4H, m), 6.66(1H, d), 6.64(1H, |
| | t), 7.25-7.52(10H, m), 7.07(1H, t), 6.86(1H, d), |
| | 6.71(1H, d), 6.41(1H, d), 6.39(2H, s) |
| 1-47 | δ = 8.42-8.55(5H, m), 7.98-8.08(3H, m), 7.89(1H, d), |
| | 7.80(2H, d), 7.32-7.64(12H, m), 7.06(1H, s), 6.88(1H, |
| | d), 6.71(1H, d), 6.33-6.41(3H, m) |
| 1-60 | δ = 8.45-8.52(3H, m), 7.98(1H, d), 7.79-7.89(6H, m), |
| | 7.27-7.66(12H, m), 7.13(1H, t), 7.02(1H, d), 6.97(1H, |
| | d), 6.89(2H, d), 6.88(1H, d), 6.71(1H, d), 6.59(1H, |
| | d), 6.33(1H, d) |
| 1-61 | δ = 8.52(1H, d), 8.48(1H, d), 7.79-7.89(5H, m), 7.66(1H, |
| | d), 7.64(1H, t), 7.50-7.55(4H, m), 7.23-7.41(7H, m), |
| | 7.13(1H, t), 7.07(1H, t), 6.71(1H, d), 6.39-6.48(4H, |
| | m), 1.72(6H, s) |
| 1-72 | δ = 8.52(1H, d), 8.48(1H, d), 7.89(1H, d), 7.87(1H, d), |
| | 7.80(1H, d), 7.28-7.66(24H, m), 6.75(1H, s), 6.71(1H, |
| | d), 6.58(1H, d), 6.41(1H, d), 6.39(2H, s), 1.72(6H, s) |
| 1-80 | δ = 8.52(1H, d), 8.48(1H, d), 7.79-7.89(6H, m), 7.23- |
| | 7.66(13H, m), 7.13(2H, t), 7.02(1H, d), 6.97(1H, d), |
| | 6.89(1H, d), 6.88(1H, d), 6.71(1H, d), 6.59(1H, d), |
| | 6.48(1H, d), 6.33(1H, d), 1.72(6H, s) |
| 1-81 | δ = 8.45-8.52(3H, m), 7.98(1H, d), 7.79-7.81(4H, m), |
| | 7.64(1H, t), 7.41-7.52(6H, m), 7.27(1H, t), 7.20(2H, |
| | t), 6.86(1H, d), 6.81(1H, t), 6.71(1H, d), 6.63(2H, |
| | d), 6.41(1H, d), 6.39(1H, s) |
| 1-87 | δ = 8.45-8.52(3H, m), 7.98(1H, d), 7.80(2H, d), 7.79(2H, |
| | d), 7.64(1H, t), 7.41-7.52(12H, m), 7.06(1H, s), |
| | 6.89(1H, s), 6.88(2H, d), 6.71(1H, d), 6.59(1H, d), |
| | 6.41(1H, d), 6.39(1H, s) |
| 1-93 | δ = 8.45-8.52(3H, m), 7.92-8.00(4H, m), 7.50-7.80(10H, |
| | m), 7.40(1H, s), 7.20(2H, t), 6.86(1H, d), 6.81(1H, |
| | t), 6.71(1H, d), 6.63(2H, d), 6.41(1H, d), 6.39(1H, s) |
| 1-101 | δ = 8.45-8.52(3H, m), 7.98(1H, d), 7.79-7.87(5H, m), |
| | 7.64(1H, t), 7.38-7.52(8H, m), 7.23-7.28(3H, m), |
| | 7.13(1H, t), 6.86(1H, d), 6.71(1H, d), 6.39-6.48(3H, |
| | m), 1.72(6H, s) |
| 1-111 | δ = 8.45-8.52(3H, m), 7.98(1H, d), 7.73-7.87(5H, m), |
| | 7.64(1H, t), 7.38-7.55(9H, m), 7.28(1H, t), 7.23(1H, |
| | d), 7.13(1H, t), 6.86(1H, d), 6.71(1H, d), 6.39- |
| | 6.48(3H, m), 1.72(6H, s) |
| 1-120 | δ = 8.45-8.52(3H, m), 7.98(1H, d), 7.79-7.87(5H, m), |
| | 7.25-7.64(14H, m), 7.13(1H, t), 6.97(1H, d), 6.89(1H, |
| | s), 6.88(2H, d), 6.71(1H, d), 6.59(1H, d), 6.48(1H, |
| | d), 1.72(6H, s) |
| 1-121 | δ = 8.52(1H, d), 8.48(1H, d), 7.79-7.87(4H, m), 7.64(1H, |
| | t), 7.50-7.55(4H, m), 7.41(1H, t), 7.38(1H, t), 7.13- |
| | 7.28(5H, m), 6.81(1H, t), 6.71(1H, d), 6.63(2H, d), |
| | 6.39-6.48(3H, m), 1.72(6H, s) |
| 1-127 | δ = 8.52(1H, d), 8.48(1H, d), 7.79-7.87(4H, m), 7.38- |
| | 7.64(15H, m), 7.28(1H, t), 6.69-6.75(4H, m), 6.58(1H, |
| | d), 6.41(1H, d), 6.39(1H, s), 1.72(6H, s) |
| 1-129 | δ = 8.52(1H, d), 8.48(1H, d), 7.93(1H, d), 7. 77-7.80(4H, |
| | m), 7.62-7.64(3H, m), 7.41-7.52(9H, m), 7.20(2H, t), |
| | 6.71-6.81(3H, m), 6.63(2H, d), 6.58(1H, d), 6.41(1H, |
| | d), 6.39(1H, s), 1.72(6H, s) |
| 1-140 | δ = 8.52(1H, d), 8.48(1H, d), 7. 79-7. 87(5H, m), 7.51- |
| | 7.64(7H, m), 7.41(1H, t), 7.38(1H, t), 7.28(1H, t), |
| | 7.20(2H, t), 6.91-7.03(3H, m), 6.81(1H, t), 6.63- |
| | 6.71(6H, m), 1.72(6H, s) |
| 1-141 | δ = 8.46(1H, d), 8.04(1H, d), 7.81(1H, d), 7.41- |
| | 7.63(14H, m), 7.20(2H, t), 6.97(1H, d), 6.83(1H, d), |
| | 6.81(1H, t), 6.69(2H, d), 6.63(2H, d), 6.48(1H, d) |
| 1-145 | δ = 8.46(1H, d), 8.04(1H, d), 7.81(1H, d), 7.41- |
| | 7.63(21H, m), 6.97(1H, d), 6.83(1H, d), 6.69(4H, d), |
| | 6.48(1H, d) |
| 1-148 | δ = 8.52(1H, d), 8.48(1H, d), 7.81(1H, d), 7.80(1H, d), |
| | 7.64(1H, t), 7.41-7.57(22H, m), 6.97(1H, d), 6.71(1H, |
| | d), 6.69(6H, d) |
| 1-149 | δ = 8.46(1H, d), 8.04(1H, d), 7.81(1H, d), 7.63(1H, t), |
| | 7.57(1H, s), 6.97(1H, d), 6.83(1H, d), 6.48(1H, d) |
| 1-157 | δ = 8.93(1H, d), 8.68(1H, d), 8.46(1H, d), 8.12(1H, d), |
| | 8.04(1H, d), 7.81-7.88(3H, m), 7.41-7.71(9H, m), |
| | 7.32(1H, s), 7.20(2H, t), 7.08(1H, d), 6.97(1H, d), |
| | 6.83(1H, d), 6.81(1H, t), 6.63(2H, d), 6.48(1H, d) |
| 1-160 | δ = 8.46(1H, d), 8.04(1H, d), 7.81(1H, d), 7.41- |
| | 7.66(25H, m), 7.20(2H, t), 7.06(1H, s), 6.97(1H, d), |
| | 6.81-6.85(4H, m), 6.63(2H, d), 6.48(1H, d) |
| 1-161 | δ = 8.46(1H, d), 8.04(1H, d), 7.89(1H, d), 7.81(1H, d), |
| | 7.20-7.66(13H, m), 7.07(1H, t), 6.97(1H, d), 6.83(1H, |
| | d), 6.81(1H, t), 6.63(2H, d), 6.48(1H, d), 6.39(1H, d) |
| 1-169 | δ = 8.46(1H, d), 8.04(1H, d), 7.81(2H, d), 7.72(1H, d), |
| | 7.71(1H, s), 7.41-7.64(14H, m), 7.20(2H, t), 6.97(1H, |
| | d), 6.83(1H, d), 6.81(1H, t), 6.63(2H, d), 6.48(1H, |
| | d), 6.33(1H, d) |
| 1-180 | δ = 8.52(1H, d), 8.48(1H, d), 7.89(1H, d), 7.81(1H, d), |
| | 7.80(1H, d), 7.32-7.66(12H, m), 7.20(2H, t), 7.13(1H, |
| | t), 7.02(1H, d), 6.97(1H, d), 6.81(1H, t), 6.63- |
| | 6.71(5H, m), 6.48(1H, d) |
| 1-181 | δ = 8.46(1H, d), 8.45(1H, d), 8.04(1H, d), 7.98(1H, d), |
| | 7.89(1H, d), 7.81(2H, d), 7.25-7.66(14H, m), 7.07(1H, |
| | t), 6.97(1H, d), 6.86(1H, d), 6.83(1H, d), 6.48(1H, |
| | d), 6.39(1H, d) |
| 1-186 | δ = 8.46(1H, d), 8.45(1H, d), 8.04(1H, d), 7.98(1H, d), |
| | 7.89(1H, d), 7.81(2H, d), 7.27-7.66(15H, m), 6.97(1H, |
| | d), 6.86(1H, d), 6.83(1H, d), 6.48(1H, d), 6.33(1H, d) |
| 1-192 | δ = 8.46(1H, d), 8.45(1H, d), 8.04(1H, d), 7.98(1H, d), |
| | 7.89(1H, d), 7.81(1H, d), 7.80(1H, d), 7.32-7.66(22H, |
| | m), 7.06(1H, s), 6.97(1H, d), 6.88(1H, d), 6.83(1H, |
| | d), 6.48(1H, d), 6.39(1H, d) |
| 1-201 | δ = 8.46(1H, d), 8.04(1H, d), 7.81-7.89(3H, m), 7.23- |
| | 7.66(15H, m), 7.13(1H, t), 7.07(1H, t), 6.97(1H, d), |
| | 6.83(1H, d), 6.48(2H, d), 6.39(1H, d), 1.72(6H, s) |
| 1-207 | δ = 9.15(1H, s), 8.93(2H, d), 8.46(1H, d), 8.04-8.18(5H, |
| | m), 7.81-7.89(7H, m), 7.55-7.66(5H, m), 7.28-7.38(4H, |
| | m), 7.13(1H, t), 7.02(1H, d), 6.97(1H, d), 6.89(1H, |
| | d), 6.75(1H, s), 6.58(1H, d), 6.48(1H, d), 6.33(1H, |
| | d), 1.72(6H, s) |
| 1-217 | δ = 9.15(1H, s), 8.93(2H, d), 8.46(1H, d), 8.04-8.18(5H, |
| | m), 7.81-7.89(7H, m), 7.55-7.66(5H, m), 7.28-7.38(4H, |
| | m), 7.13(1H, t), 7.02(1H, d), 6.97(1H, d), 6.83(1H, |
| | d), 6.75(1H, s), 6.58(1H, d), 6.48(1H, d), 6.33(1H, |
| | d), 1.72(6H, s) |
| 1-221 | δ = 8.46(1H, d), 8.45(1H, d), 8.04(1H, d), 7.98(1H, d), |
| | 7.81(2H, d), 7.41-7.63(9H, m), 7.27(1H, t), 7.20(2H, |
| | t), 6.97(1H, d), 6.86(1H, d), 6.83(1H, d), 6.81(1H, |
| | t), 6.63(2H, d), 6.48(1H, d) |
| 1-225 | δ = 8.45-8.52(3H, m), 7.98(1H, d), 7.81(2H, d), 7.80(1H, |
| | d), 7.41-7.64(10H, m), 7.27(1H, t), 7.20(2H, t), |
| | 6.97(1H, d), 6.81-6.89(4H, m), 6.71(1H, d), 6.63(2H, |
| | d), 6.59(1H, d) |
| 1-234 | δ = 8.46(1H, d), 8.04(1H, d), 8.00(2H, d), 7.86(1H, d), |
| | 7.81(1H, d), 7.73(1H, d), 7.40-7.63(13H, m), 7.20(2H, |
| | t), 6.97(1H, d), 6.86(1H, d), 6.83(1H, d), 6.81(1H, |
| | t), 6.63(2H, d), 6.48(1H, d) |
| 1-241 | δ = 8.46(1H, d), 8.45(1H, d), 8.04(1H, d), 7.98(1H, d), |
| | 7.87(1H, d), 7.81(2H, d), 7.38-7.63(11H, m), 7.28(1H, |
| | t), 7.27(1H, t), 7.23(1H, d), 7.13(1H, t), 6.97(1H, |
| | d), 6.86(1H, d), 6.83(1H, d), 6.48(2H, d), 1.72(6H, s) |
| 1-242 | δ = 8.46(1H, d), 8.45(1H, d), 8.04(1H, d), 7.98(1H, d), |
| | 7.87(1H, d), 7.81(2H, d), 7.38-7.63(12H, m), 7.25- |
| | 7.28(6H, m), 6.97(1H, d), 6.86(1H, d), 6.83(1H, d), |
| | 6.75(1H, s), 6.58(1H, d), 6.48(1H, d), 1.72(6H, s) |
| 1-247 | δ = 8.55(1H, d), 8.42-8.46(3H, m), 7.98-8.08(4H, m), |
| | 7.80-7.87(3H, m), 7.50-7.63(9H, m), 7.38(1H, t), |
| | 7.28(1H, t), 7.06(1H, s), 6.97(1H, d), 6.88(1H, d), |
| | 6.83(1H, d), 6.75(1H, s), 6.58(1H, d), 6.48(1H, d), |
| | 1.72(6H, s) |
| 1-261 | δ = 8.46(1H, d), 8.04(1H, d), 7.87(1H, d), 7.81(1H, d), |
| | 7.51-7.63(7H, m), 7.41(1H, t), 7.38(1H, t), 7.13- |
| | 7.28(5H, m), 6.97(1H, d), 6.83(1H, t), 6.81(1H, t), |
| | 6.63(2H, d), 6.48(2H, d), 1.72(6H, s) |
| 1-266 | δ = 8.46(1H, d), 8.04(1H, d), 7.87(1H, d), 7.81(1H, d), |
| | 7.25-7.63(17H, m), 7.28(1H, t), 6.97(1H, d), 6.83(1H, |
| | d), 6.75(1H, s), 6.69(2H, d), 6.58(1H, d), 6.48(1H, |
| | d), 1.72(6H, s) |
| 1-267 | δ = 8.46(1H, d), 8.04(1H, d), 7.87(1H, d), 7.81(1H, d), |
| | 7.25-7.63(16H, m), 7.28(1H, t), 6.97(1H, d), 6.83- |
| | 6.89(3H, m), 6.75(1H, s), 6.59(1H, d), 6.58(1H, d), |
| | 6.48(1H, d), 1.72(6H, s) |
| 1-275 | δ = 8.52(1H, d), 8.48(1H, d), 7.80-7.87(3H, m), 7.20- |
| | 7.64(14H, m), 7.04(1H, s), 6.97(1H, d), 6.81-6.89(3H, |
| | m), 6.71(1H, d), 6.63(2H, d), 6.59(1H, d), 6.48(1H, |
| | d), 1.72(6H, s) |
| 1-281 | δ = 8.48(1H, d), 8.05(1H, d), 7.80(1H, d), 7.78(1H, d), |
| | 7.64(1H, t), 7.41-7.54(20H, m), 6.69(4H, d), 6.41(1H, |
| | d), 6.39(1H, s) |
| 1-292 | δ = 8.10(1H, d), 8.03(1H, d), 7.79(2H, d), 7.75(1H, d), |
| | 7.37-7.54(18H, m), 7.23(1H, t), 7.20(1H, d), 6.83(1H, |
| | d), 6.69(4H, d), 6.48(1H, d) |
| 1-298 | δ = 8.48(1H, d), 8.05(1H, d), 7.64(1H, t), 7.41- |
| | 7.54(20H, m), 7.08-7.16(3H, m), 6.69(4H, d), 6.55(1H, |
| | d) |
| 1-308 | δ = 8.48(1H, d), 8.05(1H, d), 7.72(1H, d), 7.64(1H, t), |
| | 7.40-7.54(20H, m), 6.81(2H, s), 6.69(4H, d), 6.65(1H, |
| | d) |
| 1-318 | δ = 8.48(1H, d), 8.05(1H, d), 7.72(1H, d), 7.64(1H, t), |
| | 7.40-7.54(20H, m), 6.93(1H, s), 6.65-6.69(6H, m) |
| 1-332 | δ = 7.71-7.74(3H, m), 7.41-7.60(20H, m), 7.32-7.52(11H, |
| | m), 7.29(1H, t), 7.28(1H, d), 7.17(1H, t), 6.69(4H, |
| | d), 6.56(1H, d) |
| 1-341 | δ = 7.71(2H, d), 7.41-7.54(20H, m), 7.31(1H, t), |
| | 7.29(1H, t), 7.05(1H, s), 6.77(1H, s), 6.69(4H, d), |
| | 6.42(1H, d) |
| 1-349 | δ = 7.84(1H, d), 7.75(1H, d), 7.37-7.54(21H, m), |
| | 7.23(1H, t), 7.20(1H, t), 7.07(1H, d), 7.06(1H, s), |
| | 6.69(4H, d) |
| 1-359 | δ = 8.25(1H, d), 7.82(1H, d), 7. 75(1H, d), 7.37- |
| | 7.55(21H, m), 7.23(1H, t), 7.20(1H, d), 6.69(4H, d), |
| | 6.50(1H, d) |
| 1-374 | δ = 7.99(1H, d), 7.75-7.81(4H, m), 7.37-7.54(19H, m), |
| | 7.23(1H, t), 7.20(1H, d), 7.04(1H, d), 6.69(4H, d) |

TABLE 7

| Compound | FD-MS | Compound | FD-MS |
|---|---|---|---|
| 1-1 | m/z = 537.21 | 1-3 | m/z = 689.27 |
| | (C₄₀H₂₇NO = 537.65) | | (C₅₂H₃₅NO = 689.84) |
| 1-5 | m/z = 613.24 | 1-8 | m/z = 689.27 |
| | (C₄₆H₃₁NO = 613.74) | | (C₅₂H₃₅NO = 689.84) |
| 1-13 | m/z = 561.21 | 1-18 | m/z = 631.35 |
| | (C₄₂H₂₇NO = 561.67) | | (C₄₆H₁₃D₁₈NO = 631.86) |
| 1-21 | m/z = 551.19 | 1-23 | m/z = 627.22 |
| | (C₄₀H₂₅NO₂ = 551.63) | | (C₄₆H₂₉NO₂ = 627.73) |
| 1-34 | m/z = 627.22 | 1-41 | m/z = 657.18 |
| | (C₄₆H₂₉NO₂ = 627.73) | | (C₄₆H₂₇NO₂S = 657.78) |
| 1-47 | m/z = 707.19 | 1-60 | m/z = 733.21 |
| | (C₅₀H₂₉NO₂S = 707.84) | | (C₅₁H₃₁NO₂S = 733.87) |
| 1-61 | m/z = 667.25 | 1-72 | m/z = 819.31 |
| | (C₄₉H₃₃NO₂ = 667.79) | | (C₆₁H₄₁NO₂ = 819.98) |
| 1-80 | m/z = 743.28 | 1-81 | m/z = 567.17 |
| | (C₅₅H₃₇NO₂ = 743.89) | | (C₄₀H₂₅NOS = 567.70) |
| 1-87 | m/z = 643.20 | 1-93 | m/z = 617.18 |
| | (C₄₆H₂₉NOS = 643.79) | | (C₄₄H₂₇NOS = 617.76) |
| 1-101 | m/z = 683.23 | 1-111 | m/z = 683.23 |
| | (C₄₉H₃₃NOS = 683.86) | | (C₄₉H₃₃NOS = 683.86) |
| 1-120 | m/z = 759.26 | 1-121 | m/z = 577.24 |
| | (C₅₅H₃₇NOS = 759.95) | | (C₄₃H₃₁NO = 577.71) |
| 1-127 | m/z = 353.27 | 1-129 | m/z = 353.27 |
| | (C₄₉H₃₅NO = 653.81) | | (C₄₉H₃₅NO = 653.81) |
| 1-140 | m/z = 353.27 | 1-141 | m/z = 537.21 |
| | (C₄₉H₃₅NO = 653.81) | | (C₄₀H₂₇NO = 537.65) |
| 1-145 | m/z = 613.24 | 1-148 | m/z = 689.27 |
| | (C₄₆H₃₁NO = 613.74) | | (C₅₂H₃₅NO = 689.84) |
| 1-149 | m/z = 636.38 | 1-157 | m/z = 561.21 |
| | (C₄₆H₃D₂₃NO = 636.89) | | (C₄₂H₂₇NO = 561.67) |
| 1-160 | m/z = 765.30 | 1-161 | m/z = 551.19 |
| | (C₅₈H₃₉NO = 765.94) | | (C₄₀H₂₅NO₂ = 551.63) |
| 1-169 | m/z = 627.22 | 1-180 | m/z = 627.22 |
| | (C₄₆H₂₉NO₂ = 627.73) | | (C₄₆H₂₉NO₂ = 627.73) |
| 1-181 | m/z = 657.18 | 1-186 | m/z = 657.18 |
| | (C₄₆H₂₇NO₂S = 657.78) | | (C₄₆H₂₇NO₂S = 657.78) |
| 1-192 | m/z = 609.24 | 1-201 | m/z = 667.25 |
| | (C₅₈H₃₅NO₂S = 609.97) | | (C₄₉H₃₃NO₂ = 667.79) |
| 1-207 | m/z = 717.27 | 1-217 | m/z = 817.30 |
| | (C₅₃H₃₅NO₂ = 717.85) | | (C₆₁H₃₉NO₂ = 817.97) |
| 1-221 | m/z = 567.17 | 1-225 | m/z = 643.20 |
| | (C₄₀H₂₅NOS = 567.70) | | (C₄₆H₂₉NOS = 643.79) |
| 1-234 | m/z = 643.20 | 1-241 | m/z = 683.23 |
| | (C₄₆H₂₉NOS = 643.79) | | (C₄₉H₃₃NOS = 683.86) |
| 1-242 | m/z = 759.26 | 1-247 | m/z = 733.24 |
| | (C₅₅H₃₇NOS = 759.95) | | (C₅₃H₃₅NOS = 733.92) |
| 1-261 | m/z = 577.24 | 1-266 | m/z = 353.27 |
| | (C₄₃H₃₁NO = 577.71) | | (C₄₉H₃₅NO = 653.81) |
| 1-267 | m/z = 353.27 | 1-275 | m/z = 353.27 |
| | (C₄₉H₃₅NO = 653.81) | | (C₄₉H₃₅NO = 653.81) |
| 1-281 | m/z = 613.24 | 1-292 | m/z = 613.24 |
| | (C₄₆H₃₁NO = 613.74) | | (C₄₆H₃₁NO = 613.74) |
| 1-298 | m/z = 613.24 | 1-308 | m/z = 613.24 |
| | (C₄₆H₃₁NO = 613.74) | | (C₄₆H₃₁NO = 613.74) |
| 1-318 | m/z = 613.24 | 1-332 | m/z = 613.24 |
| | (C₄₆H₃₁NO = 613.74) | | (C₄₆H₃₁NO = 613.74) |
| 1-341 | m/z = 613.24 | 1-349 | m/z = 613.24 |
| | (C₄₆H₃₁NO = 613.74) | | (C₄₆H₃₁NO = 613.74) |
| 1-359 | m/z = 613.24 | 1-374 | m/z = 613.24 |
| | (C₄₆H₃₁NO = 613.74) | | (C₄₆H₃₁NO = 613.74) |

### Experimental Example 1

### 1) Manufacture of Organic Light Emitting Device

### Comparative Example 1

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to an apparatus for organic deposition.

The following 4,4′,4″-tris (N,N-(2-naphthyl)-phenylamino) triphenylamine (2-TNATA) was introduced to a cell in the vacuum deposition apparatus.

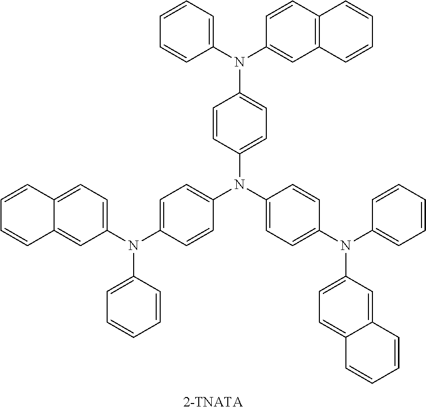 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

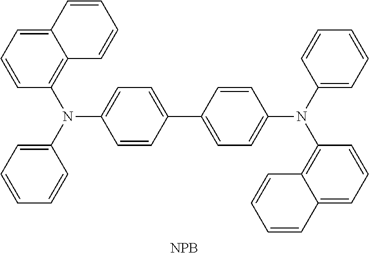 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. As the light emitting layer, a compound of 9-[4-(4,6-diphenyl-1,3,5-triazin-2-yl)phenyl]-9′-phenyl-3,3′-Bi-9H-carbazole was deposited to a thickness of 400 Å as a host, and as a green phosphorescent dopant, Ir(ppy)₃ was doped and deposited by 7%. After that, BCP was deposited to a thickness of 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (A1) cathode to a thickness of 1,200 Å, and as a result, an organic light emitting device of Comparative Example 1 was manufactured.

Meanwhile, all the organic compounds required to manufacture the organic light emitting device were vacuum sublimation purified under 10⁻⁸ torr to 10⁻⁶ torr for each material to be used in the organic light emitting device manufacture.

### Examples 1 to 60

Organic light emitting devices of Examples 1 to 60 were manufactured in the same manner as in the method for manufacturing an organic light emitting device of Comparative Example 1 except that compounds of Examples 1 to 60 of the following Table 8 were used instead of Compound NPB used when forming the hole transfer layer in Comparative Example 1.

### Comparative Examples 2 to 7

Organic light emitting devices of Comparative Examples 2 to 7 were manufactured in the same manner as in the method for manufacturing an organic light emitting device of Comparative Example 1 except that compounds of M1 to M6 of the following Table 8 were each used instead of Compound NPB used when forming the hole transfer layer in Comparative Example 1.

Herein, Compounds M1 to M6 used in Comparative Examples 2 to 7 are as follows.

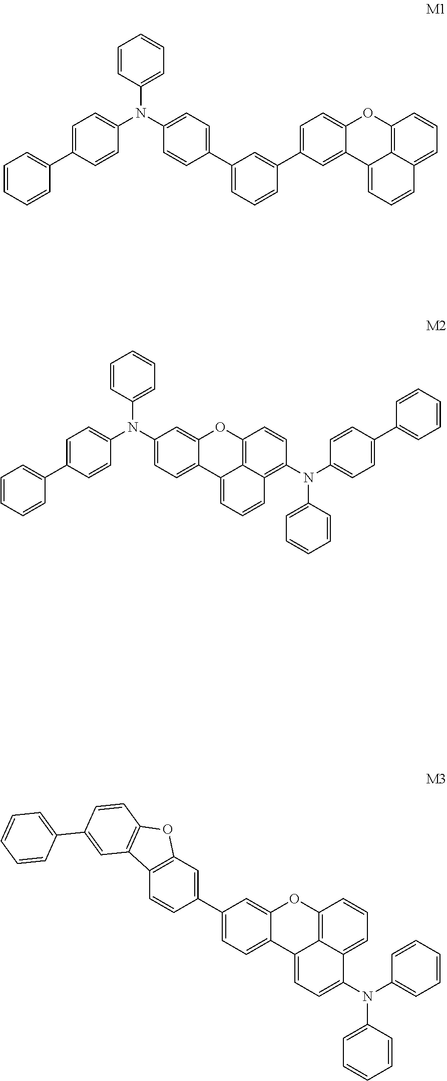 [SMILES: c1ccc(-c2ccc(N(c3ccccc3)c3ccc(-c4cccc(-c5ccc6c(c5)-c5cccc7cccc(c57)O6)c4)cc3)cc2)cc1 ; c1ccc(-c2ccc(N(c3ccccc3)c3ccc4c(c3)Oc3ccc(N(c5ccccc5)c5ccc(-c6ccccc6)cc5)c5cccc-4c35)cc2)cc1 ; c1ccc(-c2ccc3oc4cc(-c5ccc6c(c5)Oc5cccc7c(N(c8ccccc8)c8ccccc8)ccc-6c57)ccc4c3c2)cc1]

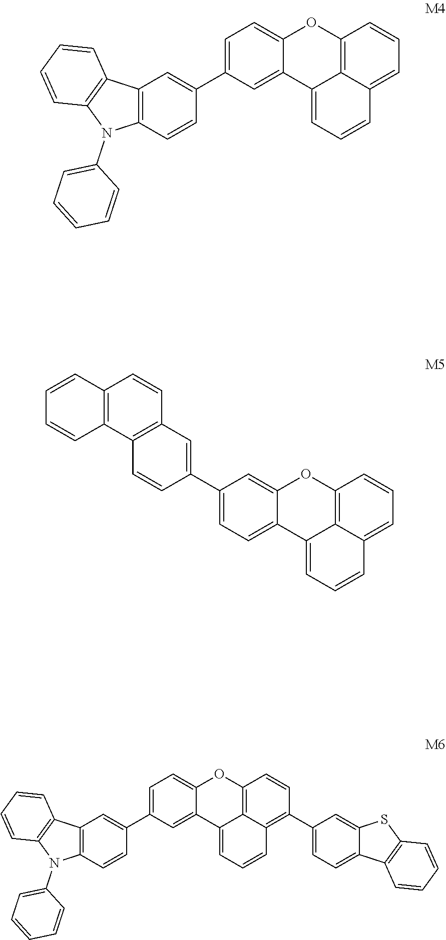 [SMILES: c1ccc(-n2c3ccccc3c3cc(-c4ccc5c(c4)-c4cccc6cccc(c46)O5)ccc32)cc1 ; c1cc2c3c(cccc3c1)-c1ccc(-c3ccc4c(ccc5ccccc54)c3)cc1O2 ; c1ccc(-n2c3ccccc3c3cc(-c4ccc5c(c4)-c4cccc6c(-c7ccc8c(c7)sc7ccccc78)ccc(c46)O5)ccc32)cc1]

### (2) Driving Voltage and Light Emission Efficiency of Organic Light Emitting Device

For each of the organic light emitting devices of Examples 1 to 60 and Comparative Examples 1 to 7 manufactured as above, electroluminescent (EL) properties were measured using M7000 manufactured by McScience Inc., and with the measurement results, a lifetime T₉₀ (unit: h, hour), time taken to become 90% with respect to initial luminance, was measured when standard luminance was 6,000 cd/m² through a lifetime measurement system (M6000) manufactured by McScience Inc.

Properties of the organic light emitting devices of the present disclosure shown as the measurement results are as shown in the following Table 8.

TABLE 8

| | | Driving | Light Emission | Lifetime |
|---|---|---|---|---|
| | Compound | Voltage (V) | Efficiency (cd/A) | (T₉₀) |
| Example 1 | 1-1 | 3.94 | 129.08 | 267 |
| Example 2 | 1-3 | 3.77 | 131.71 | 269 |
| Example 3 | 1-5 | 3.79 | 128.85 | 268 |
| Example 4 | 1-8 | 3.75 | 128.29 | 266 |
| Example 5 | 1-13 | 3.76 | 131.89 | 266 |
| Example 6 | 1-18 | 3.50 | 138.62 | 275 |
| Example 7 | 1-21 | 4.19 | 125.71 | 256 |
| Example 8 | 1-23 | 4.31 | 125.19 | 253 |
| Example 9 | 1-34 | 4.23 | 123.88 | 236 |
| Example 10 | 1-41 | 4.35 | 122.92 | 231 |
| Example 11 | 1-47 | 4.33 | 122.32 | 230 |
| Example 12 | 1-60 | 4.32 | 122.17 | 231 |
| Example 13 | 1-61 | 4.31 | 121.62 | 222 |
| Example 14 | 1-72 | 4.34 | 122.76 | 216 |
| Example 15 | 1-80 | 4.31 | 121.36 | 208 |
| Example 16 | 1-81 | 4.21 | 125.84 | 250 |
| Example 17 | 1-87 | 4.17 | 125.52 | 251 |
| Example 18 | 1-93 | 4.24 | 126.79 | 251 |
| Example 19 | 1-101 | 4.34 | 123.70 | 231 |
| Example 20 | 1-111 | 4.33 | 123.35 | 237 |
| Example 21 | 1-120 | 4.38 | 122.62 | 234 |
| Example 22 | 1-121 | 4.24 | 124.32 | 244 |
| Example 23 | 1-127 | 4.20 | 124.23 | 246 |
| Example 24 | 1-129 | 4.17 | 124.59 | 241 |
| Example 25 | 1-140 | 4.15 | 124.59 | 240 |
| Example 26 | 1-141 | 3.91 | 127.34 | 263 |
| Example 27 | 1-145 | 3.97 | 127.26 | 264 |
| Example 28 | 1-148 | 4.10 | 127.29 | 266 |
| Example 29 | 1-149 | 3.61 | 134.27 | 270 |
| Example 30 | 1-157 | 3.91 | 127.05 | 261 |
| Example 31 | 1-160 | 3.88 | 127.89 | 266 |
| Example 32 | 1-161 | 4.15 | 125.99 | 250 |
| Example 33 | 1-169 | 4.19 | 125.86 | 254 |
| Example 34 | 1-180 | 4.16 | 124.99 | 253 |
| Example 35 | 1-181 | 4.30 | 122.55 | 231 |
| Example 36 | 1-186 | 4.35 | 122.88 | 233 |
| Example 37 | 1-192 | 4.38 | 122.01 | 227 |
| Example 38 | 1-201 | 4.33 | 123.73 | 234 |
| Example 39 | 1-207 | 4.37 | 123.17 | 238 |
| Example 40 | 1-217 | 4.39 | 122.54 | 235 |
| Example 41 | 1-221 | 4.25 | 124.85 | 250 |
| Example 42 | 1-225 | 4.27 | 124.12 | 249 |
| Example 43 | 1-234 | 4.22 | 125.14 | 244 |
| Example 44 | 1-241 | 4.31 | 121.62 | 222 |
| Example 45 | 1-242 | 4.39 | 121.97 | 211 |
| Example 46 | 1-247 | 4.32 | 121.67 | 210 |
| Example 47 | 1-261 | 4.27 | 123.68 | 238 |
| Example 48 | 1-266 | 4.29 | 123.33 | 238 |
| Example 49 | 1-267 | 4.25 | 123.55 | 236 |
| Example 50 | 1-275 | 4.19 | 124.99 | 237 |
| Example 51 | 1-281 | 4.40 | 120.76 | 200 |
| Example 52 | 1-292 | 4.42 | 120.11 | 203 |
| Example 53 | 1-298 | 4.40 | 120.62 | 190 |
| Example 54 | 1-308 | 4.40 | 120.69 | 197 |
| Example 55 | 1-318 | 4.47 | 120.63 | 194 |
| Example 56 | 1-332 | 4.42 | 121.11 | 203 |
| Example 57 | 1-341 | 4.41 | 120.33 | 200 |
| Example 58 | 1-349 | 4.47 | 120.29 | 197 |
| Example 59 | 1-359 | 4.45 | 120.63 | 194 |
| Example 60 | 1-374 | 4.43 | 120.66 | 198 |
| Comparative | NPB | 4.51 | 110.59 | 150 |
| Example 1 | | | | |
| Comparative | M1 | 4.66 | 116.32 | 165 |
| Example 2 | | | | |
| Comparative | M2 | 4.57 | 117.51 | 171 |
| Example 3 | | | | |
| Comparative | M3 | 4.51 | 118.92 | 182 |
| Example 4 | | | | |
| Comparative | M4 | 4.68 | 116.49 | 167 |
| Example 5 | | | | |
| Comparative | M5 | 4.64 | 116.58 | 163 |
| Example 6 | | | | |
| Comparative | M6 | 4.59 | 117.42 | 173 |
| Example 7 | | | | |

As described in Table 8, the organic light emitting devices of Examples 1 to 60 in which a hole transfer layer is formed using the heterocyclic compound of Chemical Formula 1 according to the present disclosure have properties of long lifetime, low voltage and high efficiency.

Specifically, the organic light emitting devices of Examples 1 to 60 have a structure in which benzo[kl]xanthene is substituted with two types of substituents, that is, an amine group and an aryl group, which delocalizes a HOMO (highest occupied molecular orbital) energy level and thereby increases a hole transfer ability, and stabilizes the HOMO energy. Accordingly, when using the heterocyclic compound of Chemical Formula 1 as a material of a hole transfer layer in an organic light emitting device, proper energy level and band gap are formed increasing excitons in a light emitting region. Increasing excitons in a light emitting region means having an effect of reducing a driving voltage of a device and an effect of increasing efficiency. It is identified that the organic light emitting devices of Comparative Examples 1 to 7 not using the compound according to the present application when forming the hole transfer layer have reduced light emission efficiency and lifetime compared to the organic light emitting devices of Examples 1 to 60.

Particularly, it is identified that Examples 6 and 29 using the compounds substituted with deuterium are effective in further improving driving voltage, light emission efficiency and lifetime. Specifically, Compound 1-145 of Example 27 and Compound 1-149 of Example 29 have the same structure, but is different in the substitution of deuterium, and it is seen that Example 29 substituted with deuterium has more superior driving voltage, light emission efficiency and lifetime compared to Example 27.

### Experimental Example 2

### (1) Manufacture of Organic Light Emitting Device

### Comparative Example 8

A transparent ITO electrode thin film obtained from glass for an organic light emitting device (manufactured by Samsung-Corning Co., Ltd.) was ultrasonic cleaned using trichloroethylene, acetone, ethanol and distilled water consecutively for 5 minutes each, stored in isopropanol, and used. Next, the ITO substrate was installed in a substrate holder of a vacuum deposition apparatus, and the following 4,4′,4″-tris (N,N-(2-naphthyl)-phenylamino) triphenylamine (2-TNATA) was introduced to a cell in the vacuum deposition apparatus.

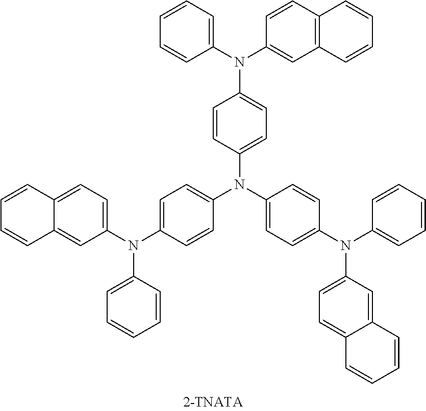 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate. To another cell in the vacuum deposition apparatus, the following N,N′-bis (α-naphthyl)-N,N′-diphenyl-4,4′-diamine (NPB) was introduced, and evaporated by applying a current to the cell to deposit a hole transfer layer having a thickness of 300 Å on the hole injection layer.

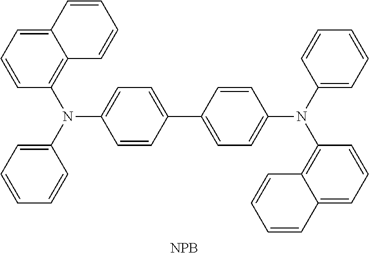 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, M7 was deposited to 50 Å as an electron blocking layer, and a blue light emitting material having a structure as below was deposited thereon as a light emitting layer. Specifically, in one side cell in the vacuum deposition apparatus, H1, a blue light emitting host material, was vacuum deposited to a thickness of 200 Å, and Dl, a blue light emitting dopant material, was vacuum deposited thereon by 5% with respect to the host material.

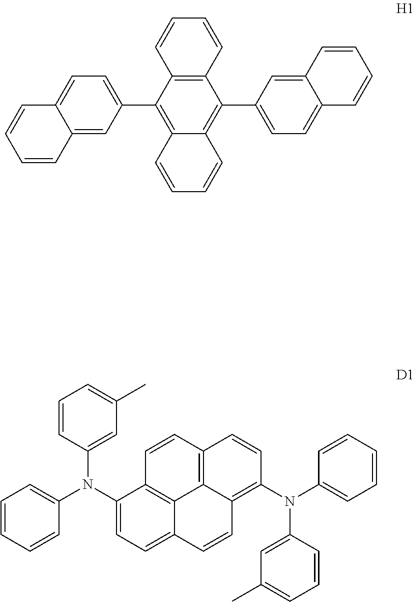

Subsequently, a compound of the following Structural Formula E1 was deposited to a thickness of 300 Å as an electron transfer layer.

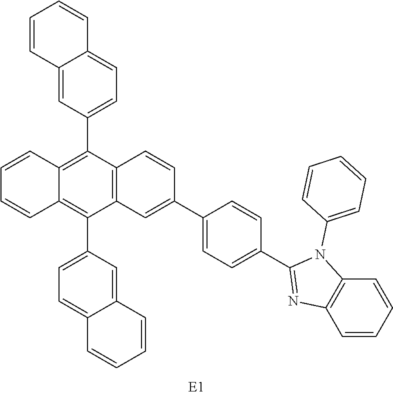 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

As an electron injection layer, lithium fluoride (LiF) was deposited to a thickness of 10 Å, and an A1 cathode was employed to a thickness of 1,000 Å, and as a result, an organic light emitting device was manufactured. Meanwhile, all the organic compounds required to manufacture the organic light emitting device were vacuum sublimation purified under 10⁻⁸ torr to 10⁻⁶ torr by each material to be used in the organic light emitting device manufacture.

### Comparative Examples 9 and 10 and Examples 61 to 120

Organic light emitting devices were manufactured in the same manner as in the method for manufacturing an organic light emitting device of Comparative Example 8 except that the electron blocking layer was formed using compounds of the following Table 9 instead of M7.

Herein, electron blocking layer Compounds M7 to M9 of Comparative Examples 8 to 10 are as follows.

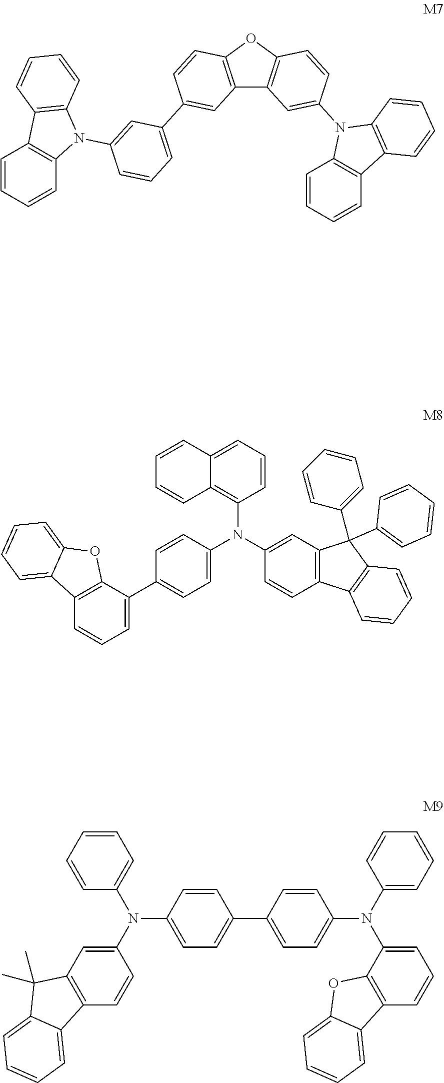 [SMILES: c1cc(-c2ccc3oc4ccc(-n5c6ccccc6c6ccccc65)cc4c3c2)cc(-n2c3ccccc3c3ccccc32)c1 ; c1ccc(C2(c3ccccc3)c3ccccc3-c3ccc(N(c4ccc(-c5cccc6c5oc5ccccc56)cc4)c4cccc5ccccc45)cc32)cc1 ; CC1(C)c2ccccc2-c2ccc(N(c3ccccc3)c3ccc(-c4ccc(N(c5ccccc5)c5cccc6c5oc5ccccc56)cc4)cc3)cc21]

### (2) Driving Voltage and Light Emission Efficiency of Organic Light Emitting Device

For each of the organic light emitting devices of Examples 61 to 120 and Comparative Examples 8 to 10 manufactured as above, electroluminescent (EL) properties were measured using M7000 manufactured by McScience Inc., and with the measurement results, a lifetime T₉₅ (unit: h, hour), time taken to become 95% with respect to initial luminance, was measured when standard luminance was 6,000 cd/m² through a lifetime measurement system (M6000) manufactured by McScience Inc.

Properties of the organic light emitting devices of the present disclosure shown as the measurement results are as shown in the following Table 9.

TABLE 9

| | | Driving | Light Emission | Lifetime |
|---|---|---|---|---|
| | Compound | Voltage (V) | Efficiency (cd/A) | (T₉₅) |
| Example 61 | 1-1 | 4.16 | 6.87 | 53 |
| Example 62 | 1-3 | 4.19 | 6.81 | 57 |
| Example 63 | 1-5 | 4.21 | 6.86 | 57 |
| Example 64 | 1-8 | 4.28 | 6.85 | 59 |
| Example 65 | 1-13 | 4.23 | 6.81 | 56 |
| Example 66 | 1-18 | 4.08 | 6.92 | 62 |
| Example 67 | 1-21 | 4.38 | 6.71 | 51 |
| Example 68 | 1-23 | 4.35 | 6.67 | 50 |
| Example 69 | 1-34 | 4.37 | 6.61 | 51 |
| Example 70 | 1-41 | 4.48 | 6.52 | 49 |
| Example 71 | 1-47 | 4.49 | 6.50 | 48 |
| Example 72 | 1-60 | 4.58 | 6.51 | 47 |
| Example 73 | 1-61 | 4.83 | 6.31 | 45 |
| Example 74 | 1-72 | 4.87 | 6.27 | 44 |
| Example 75 | 1-80 | 4.81 | 6.28 | 46 |
| Example 76 | 1-81 | 4.69 | 6.43 | 45 |
| Example 77 | 1-87 | 4.60 | 6.40 | 46 |
| Example 78 | 1-93 | 4.65 | 6.40 | 45 |
| Example 79 | 1-101 | 4.93 | 6.13 | 44 |
| Example 80 | 1-111 | 4.92 | 6.11 | 45 |
| Example 81 | 1-120 | 4.94 | 6.21 | 44 |
| Example 82 | 1-121 | 5.02 | 6.07 | 45 |
| Example 83 | 1-127 | 4.99 | 6.09 | 43 |
| Example 84 | 1-129 | 4.98 | 6.08 | 43 |
| Example 85 | 1-140 | 5.01 | 6.11 | 44 |
| Example 86 | 1-141 | 4.26 | 6.81 | 55 |
| Example 87 | 1-145 | 4.24 | 6.76 | 58 |
| Example 88 | 1-148 | 4.21 | 6.76 | 57 |
| Example 89 | 1-149 | 4.13 | 6.90 | 61 |
| Example 90 | 1-157 | 4.27 | 6.78 | 55 |
| Example 91 | 1-160 | 4.16 | 6.70 | 54 |
| Example 92 | 1-161 | 4.39 | 6.67 | 51 |
| Example 93 | 1-169 | 4.35 | 6.66 | 51 |
| Example 94 | 1-180 | 4.31 | 6.61 | 50 |
| Example 95 | 1-181 | 4.63 | 6.41 | 49 |
| Example 96 | 1-186 | 4.68 | 6.44 | 48 |
| Example 97 | 1-192 | 4.72 | 6.40 | 48 |
| Example 98 | 1-201 | 4.88 | 6.16 | 44 |
| Example 99 | 1-207 | 4.85 | 6.18 | 44 |
| Example 100 | 1-217 | 4.83 | 6.17 | 45 |
| Example 101 | 1-221 | 4.73 | 6.39 | 45 |
| Example 102 | 1-225 | 4.79 | 6.38 | 46 |
| Example 103 | 1-234 | 4.78 | 6.34 | 47 |
| Example 104 | 1-241 | 4.99 | 6.07 | 43 |
| Example 105 | 1-242 | 4.97 | 6.11 | 44 |
| Example 106 | 1-247 | 4.92 | 6.09 | 45 |
| Example 107 | 1-261 | 5.04 | 6.09 | 45 |
| Example 108 | 1-266 | 5.09 | 6.07 | 44 |
| Example 109 | 1-267 | 5.10 | 6.07 | 43 |
| Example 110 | 1-275 | 5.11 | 6.04 | 43 |
| Example 111 | 1-281 | 5.15 | 6.00 | 42 |
| Example 112 | 1-292 | 5.16 | 6.02 | 41 |
| Example 113 | 1-298 | 5.12 | 6.03 | 43 |
| Example 114 | 1-308 | 5.27 | 6.00 | 42 |
| Example 115 | 1-318 | 5.26 | 6.03 | 42 |
| Example 116 | 1-332 | 5.30 | 6.04 | 41 |
| Example 117 | 1-341 | 5.19 | 5.97 | 40 |
| Example 118 | 1-349 | 5.31 | 5.95 | 42 |
| Example 119 | 1-359 | 5.16 | 6.02 | 41 |
| Example 120 | 1-374 | 5.22 | 5.99 | 43 |
| Comparative | M7 | 6.49 | 5.87 | 39 |
| Example 8 | | | | |
| Comparative | M8 | 6.36 | 5.59 | 35 |
| Example 9 | | | | |
| Comparative | M9 | 6.41 | 5.75 | 38 |
| Example 10 | | | | |

From Experimental Example 2, it was identified that the organic light emitting devices of Examples 61 to 120 using the compound according to the present application when forming the electron blocking layer had an excellent electron blocking ability by delocalizing a HOMO (highest occupied molecular orbital) energy level and stabilizing the HOMO energy, and had excellent light emission efficiency and lifetime compared to the organic light emitting devices of Comparative Examples 8 to 10 not using the compound according to the present application when forming the electron blocking layer.

1. A heterocyclic compound of the following Chemical Formula 1: 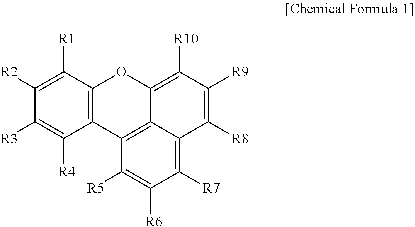
wherein, in Chemical Formula 1,
R1 to R10 are the same as or different from each other, and each independently hydrogen; deuterium; a halogen group; a cyano group; —Si(R11)(R12)(R13); —N(R14)(R15); a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group;
R11 to R15 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group;
at least one of R1 to R10 is -(L)l-N(R24)(R25);
at least one of the rest of R1 to R10 is a substituted or unsubstituted C6 to C60 aryl group;
L is a direct bond; a substituted or unsubstituted C6 to C60 arylene group; or a substituted or unsubstituted C2 to C60 heteroarylene group;
1 is an integer of 1 to 3, and when 1 is 2 or greater, Ls are the same as or different from each other; and
R24 and R25 are the same as or different from each other, and each independently a substituted or unsubstituted C6 to C60 aryl group; or a substituted or unsubstituted C2 to C60 heteroaryl group.

2. The heterocyclic compound of claim 1, wherein Chemical Formula 1 is represented by any one of the following Chemical Formulae 1-1 to 1-4: 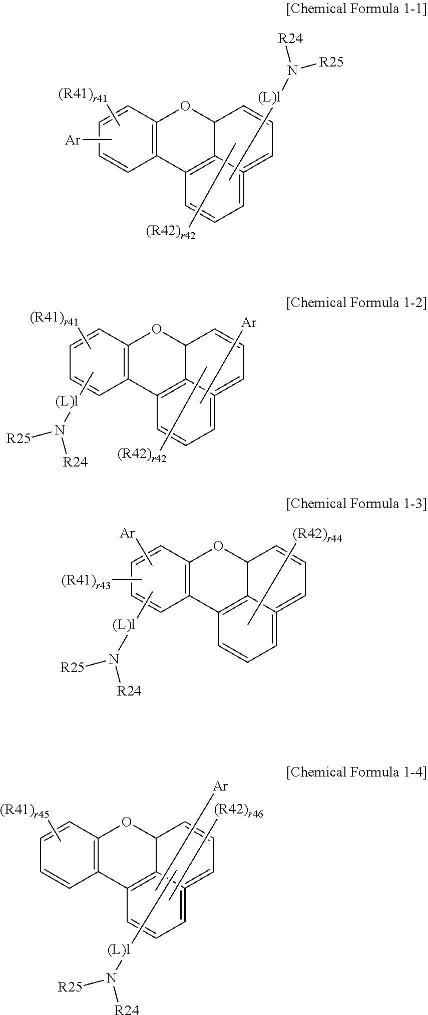
in Chemical Formulae 1-1 to 1-4,
L, 1, R24 and R25 have the same definitions as in Chemical Formula 1;
Ar is a substituted or unsubstituted C6 to C60 aryl group;
R41 and R42 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group;
r41 is an integer of 0 to 3, r42 is an integer of 0 to 5, r43 is an integer of 0 to 2, r44 is an integer of 0 to 6, and r45 and r46 are each an integer of 0 to 4; and
when r41, r42 and r44 to r46 are 2 or greater or r43 is 2, substituents in the parentheses are the same as or different from each other.

3. The heterocyclic compound of claim 1, wherein -(L)l-N(R24)(R25) is represented by any one of the following Chemical Formulae N-1 to N-3: 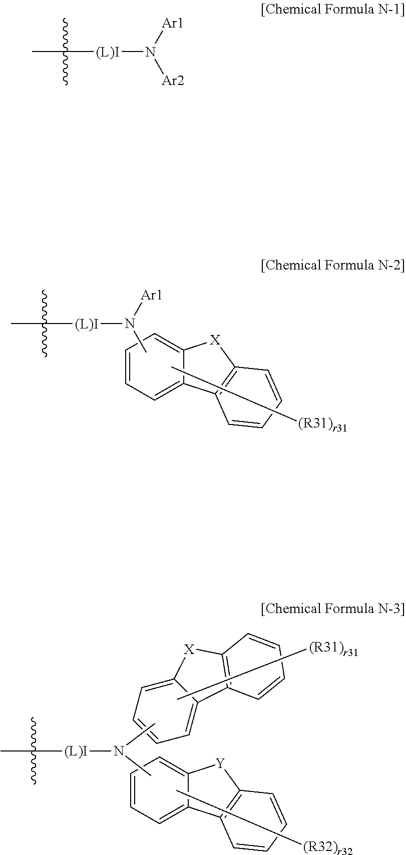
in Chemical Formulae N-1 to N-3,
L and 1 have the same definitions as in Chemical Formula 1;
X and Y are each independently 0; or S;
Ar1 and Ar2 are the same as or different from each other, and each independently a substituted or unsubstituted C6 to C60 aryl group;
R31 and R32 are each independently hydrogen; deuterium; or a substituted or unsubstituted C6 to C30 aryl group; and
r31 and r32 are each an integer of 0 to 7, and when r31 and r32 are each 2 or greater, substituents in the parentheses are the same as or different from each other.

4. The heterocyclic compound of claim 1, wherein Chemical Formula 1 has a deuterium content of either 0%, or 10% to 100%.

5. The heterocyclic compound of claim 1, wherein Chemical Formula 1 is represented by any one of the following compounds:

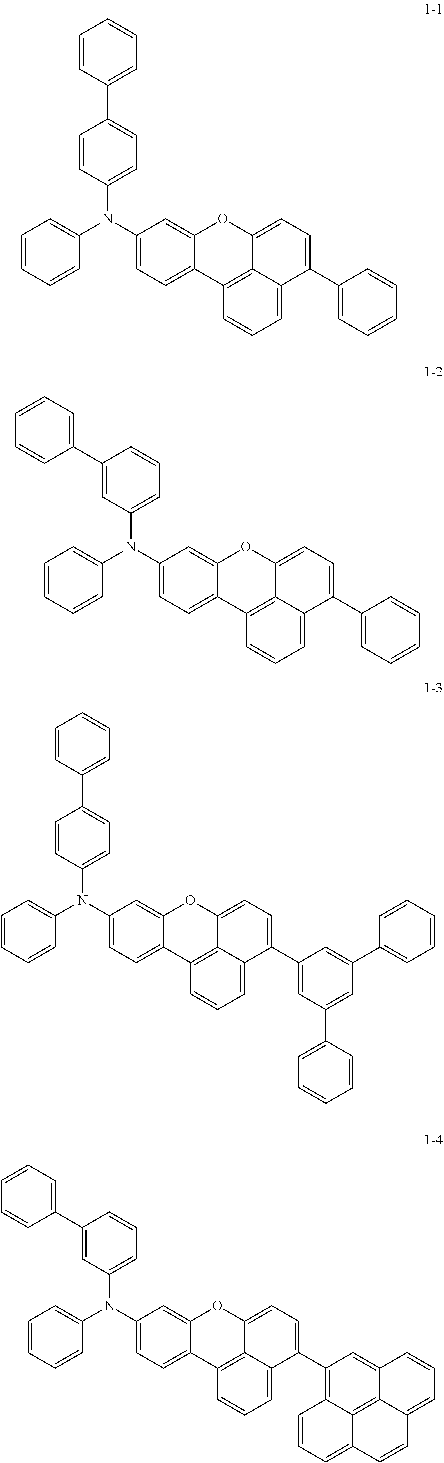 [SMILES: c1ccc(-c2ccc(N(c3ccccc3)c3ccc4c(c3)Oc3ccc(-c5ccccc5)c5cccc-4c35)cc2)cc1 ; c1ccc(-c2cccc(N(c3ccccc3)c3ccc4c(c3)Oc3ccc(-c5ccccc5)c5cccc-4c35)c2)cc1 ; c1ccc(-c2ccc(N(c3ccccc3)c3ccc4c(c3)Oc3ccc(-c5cc(-c6ccccc6)cc(-c6ccccc6)c5)c5cccc-4c35)cc2)cc1 ; c1ccc(-c2cccc(N(c3ccccc3)c3ccc4c(c3)Oc3ccc(-c5cc6cccc7ccc8cccc5c8c76)c5cccc-4c35)c2)cc1]

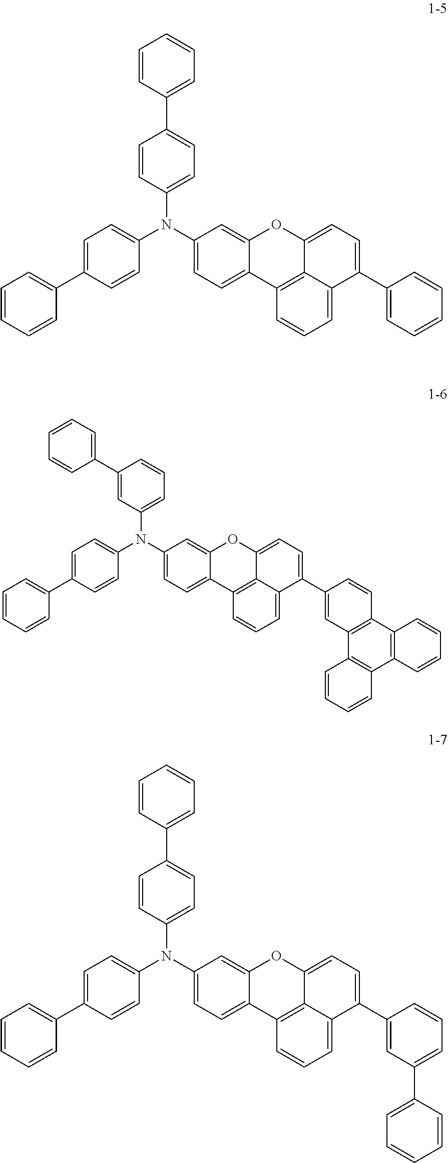 [SMILES: c1ccc(-c2ccc(N(c3ccc(-c4ccccc4)cc3)c3ccc4c(c3)Oc3ccc(-c5ccccc5)c5cccc-4c35)cc2)cc1 ; c1ccc(-c2ccc(N(c3cccc(-c4ccccc4)c3)c3ccc4c(c3)Oc3ccc(-c5ccc6c7ccccc7c7ccccc7c6c5)c5cccc-4c35)cc2)cc1 ; c1ccc(-c2ccc(N(c3ccc(-c4ccccc4)cc3)c3ccc4c(c3)Oc3ccc(-c5cccc(-c6ccccc6)c5)c5cccc-4c35)cc2)cc1]

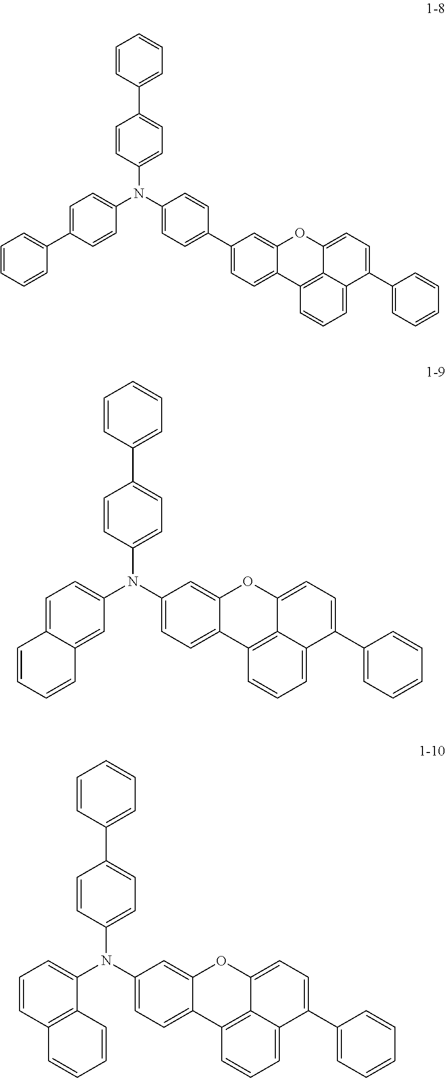 [SMILES: c1ccc(-c2ccc(N(c3ccc(-c4ccccc4)cc3)c3ccc(-c4ccc5c(c4)Oc4ccc(-c6ccccc6)c6cccc-5c46)cc3)cc2)cc1 ; c1ccc(-c2ccc(N(c3ccc4c(c3)Oc3ccc(-c5ccccc5)c5cccc-4c35)c3ccc4ccccc4c3)cc2)cc1 ; c1ccc(-c2ccc(N(c3ccc4c(c3)Oc3ccc(-c5ccccc5)c5cccc-4c35)c3cccc4ccccc34)cc2)cc1]

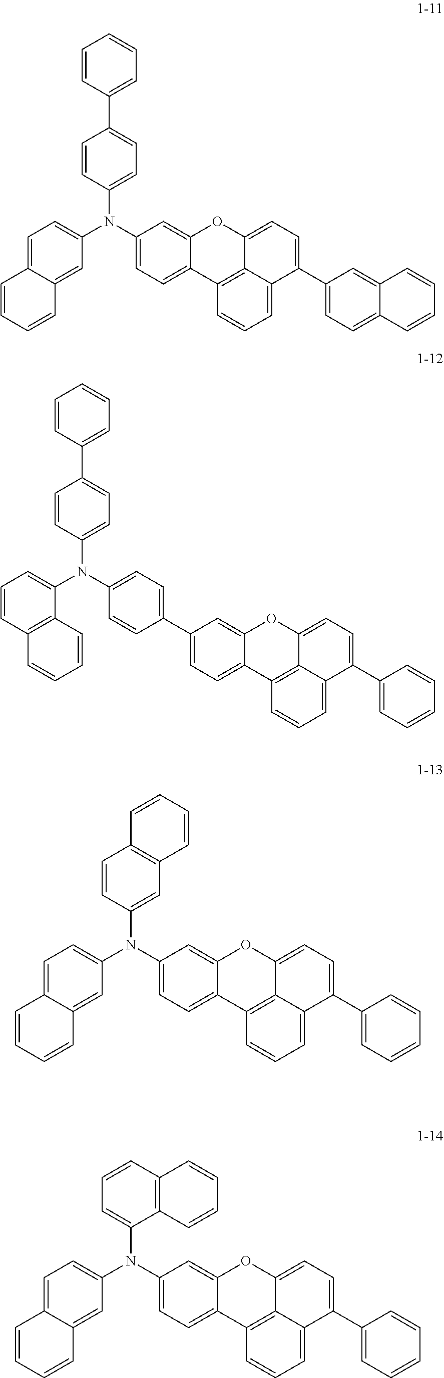 [SMILES: c1ccc(-c2ccc(N(c3ccc4c(c3)Oc3ccc(-c5ccc6ccccc6c5)c5cccc-4c35)c3ccc4ccccc4c3)cc2)cc1 ; c1ccc(-c2ccc(N(c3ccc(-c4ccc5c(c4)Oc4ccc(-c6ccccc6)c6cccc-5c46)cc3)c3cccc4ccccc34)cc2)cc1 ; c1ccc(-c2ccc3c4c(cccc24)-c2ccc(N(c4ccc5ccccc5c4)c4ccc5ccccc5c4)cc2O3)cc1 ; c1ccc(-c2ccc3c4c(cccc24)-c2ccc(N(c4ccc5ccccc5c4)c4cccc5ccccc45)cc2O3)cc1]

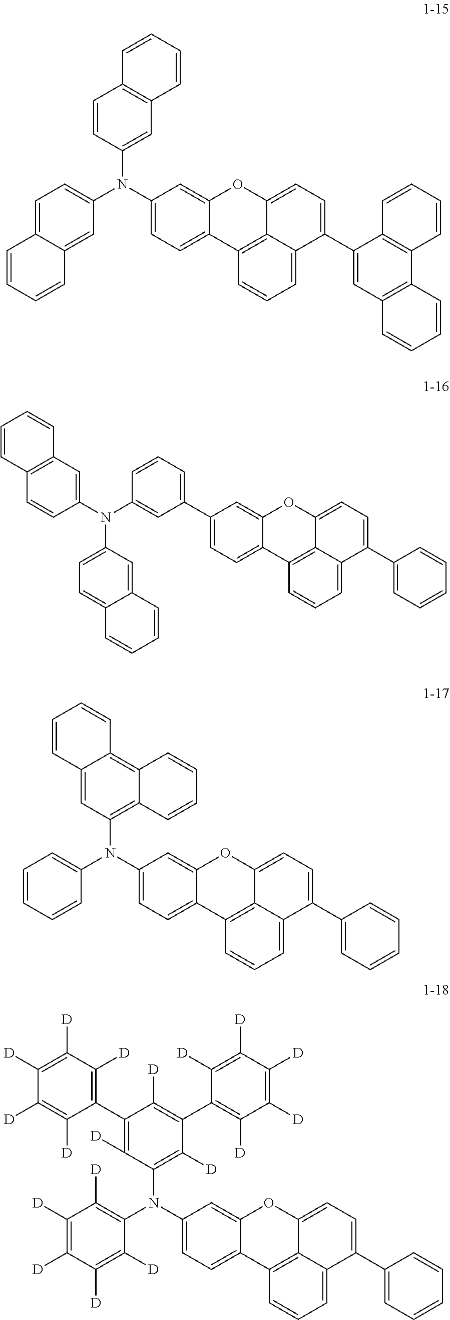 [SMILES: c1ccc2cc(N(c3ccc4c(c3)Oc3ccc(-c5cc6ccccc6c6ccccc56)c5cccc-4c35)c3ccc4ccccc4c3)ccc2c1 ; c1ccc(-c2ccc3c4c(cccc24)-c2ccc(-c4cccc(N(c5ccc6ccccc6c5)c5ccc6ccccc6c5)c4)cc2O3)cc1 ; c1ccc(-c2ccc3c4c(cccc24)-c2ccc(N(c4ccccc4)c4cc5ccccc5c5ccccc45)cc2O3)cc1 ; [2H]c1c([2H])c([2H])c(-c2c([2H])c(-c3c([2H])c([2H])c([2H])c([2H])c3[2H])c([2H])c(N(c3ccc4c(c3)Oc3ccc(-c5ccccc5)c5cccc-4c35)c3c([2H])c([2H])c([2H])c([2H])c3[2H])c2[2H])c([2H])c1[2H]]

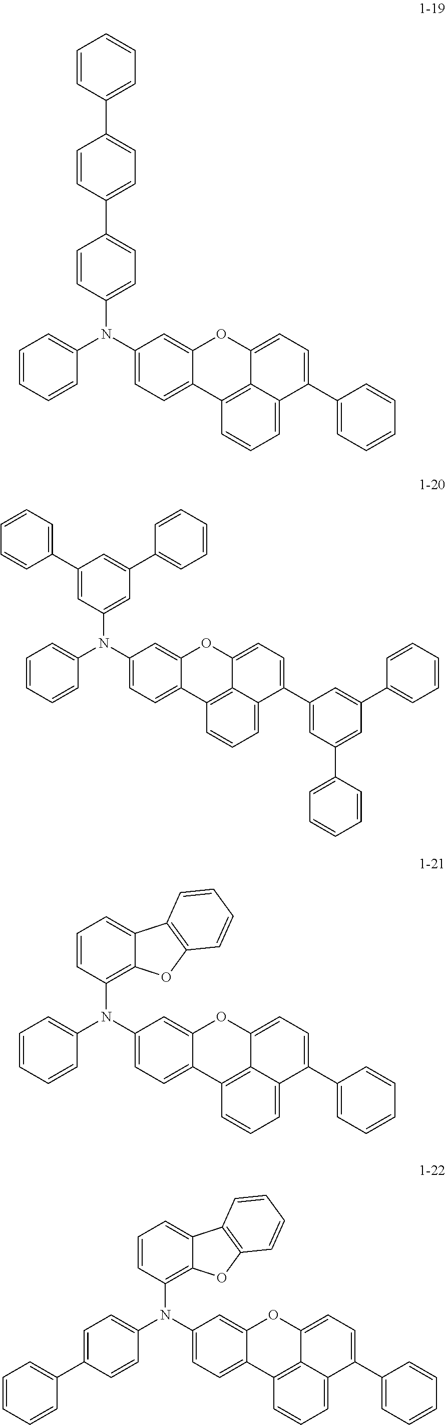 [SMILES: c1ccc(-c2ccc(-c3ccc(N(c4ccccc4)c4ccc5c(c4)Oc4ccc(-c6ccccc6)c6cccc-5c46)cc3)cc2)cc1 ; c1ccc(-c2cc(-c3ccccc3)cc(-c3ccc4c5c(cccc35)-c3ccc(N(c5ccccc5)c5cc(-c6ccccc6)cc(-c6ccccc6)c5)cc3O4)c2)cc1 ; c1ccc(-c2ccc3c4c(cccc24)-c2ccc(N(c4ccccc4)c4cccc5c4oc4ccccc45)cc2O3)cc1 ; c1ccc(-c2ccc(N(c3ccc4c(c3)Oc3ccc(-c5ccccc5)c5cccc-4c35)c3cccc4c3oc3ccccc34)cc2)cc1]

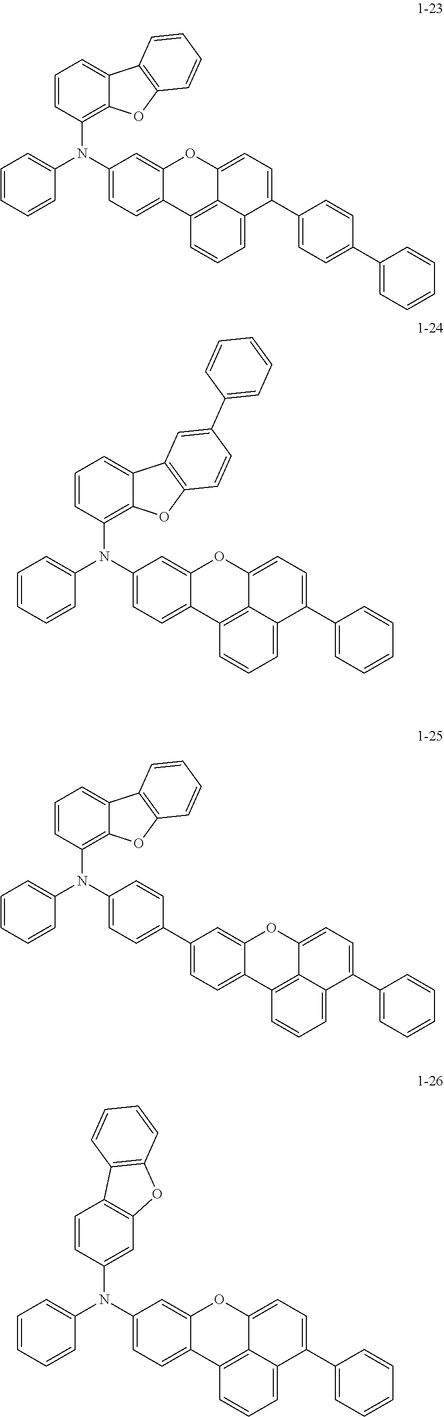 [SMILES: c1ccc(-c2ccc(-c3ccc4c5c(cccc35)-c3ccc(N(c5ccccc5)c5cccc6c5oc5ccccc56)cc3O4)cc2)cc1 ; c1ccc(-c2ccc3oc4c(N(c5ccccc5)c5ccc6c(c5)Oc5ccc(-c7ccccc7)c7cccc-6c57)cccc4c3c2)cc1 ; c1ccc(-c2ccc3c4c(cccc24)-c2ccc(-c4ccc(N(c5ccccc5)c5cccc6c5oc5ccccc56)cc4)cc2O3)cc1 ; c1ccc(-c2ccc3c4c(cccc24)-c2ccc(N(c4ccccc4)c4ccc5c(c4)oc4ccccc45)cc2O3)cc1]

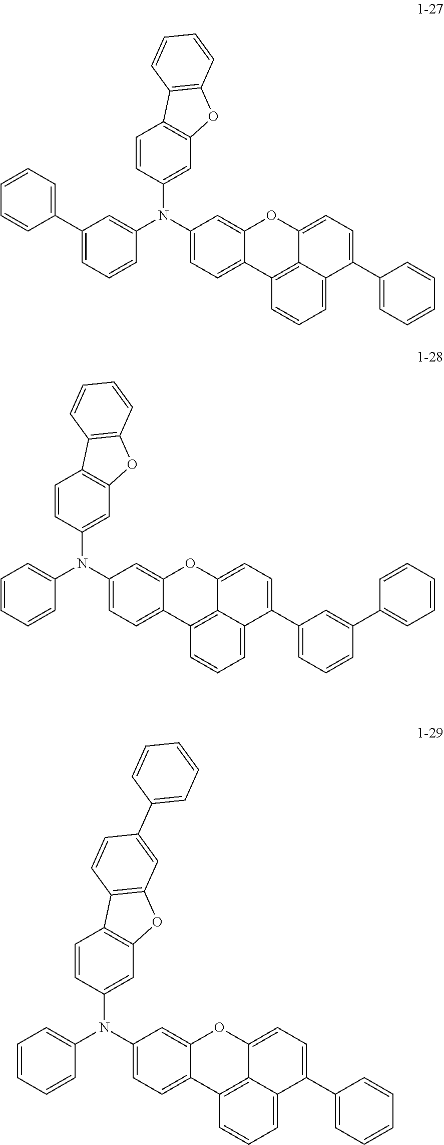 [SMILES: c1ccc(-c2cccc(N(c3ccc4c(c3)Oc3ccc(-c5ccccc5)c5cccc-4c35)c3ccc4c(c3)oc3ccccc34)c2)cc1 ; c1ccc(-c2cccc(-c3ccc4c5c(cccc35)-c3ccc(N(c5ccccc5)c5ccc6c(c5)oc5ccccc56)cc3O4)c2)cc1 ; c1ccc(-c2ccc3c(c2)oc2cc(N(c4ccccc4)c4ccc5c(c4)Oc4ccc(-c6ccccc6)c6cccc-5c46)ccc23)cc1]

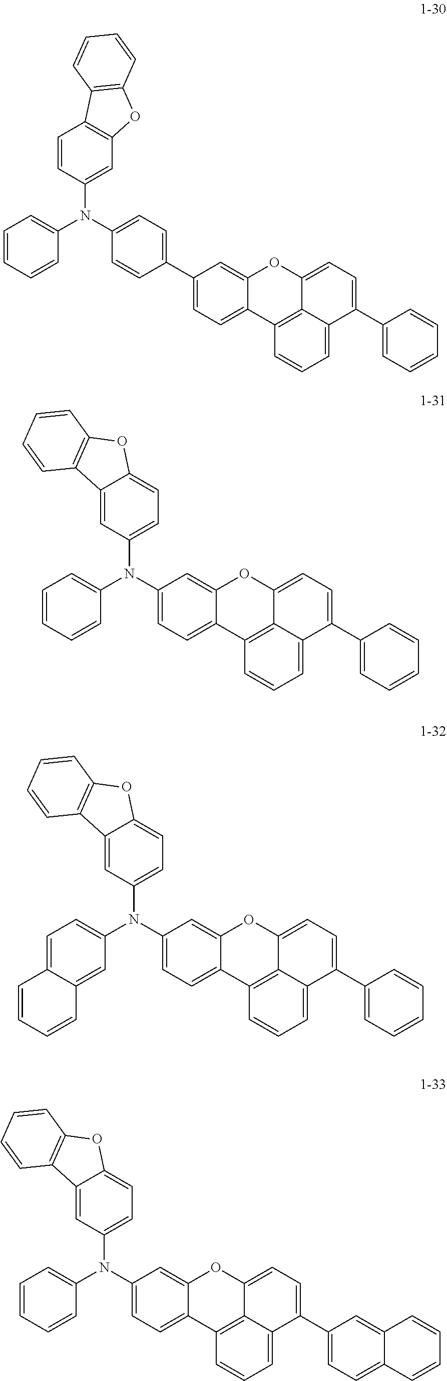 [SMILES: c1ccc(-c2ccc3c4c(cccc24)-c2ccc(-c4ccc(N(c5ccccc5)c5ccc6c(c5)oc5ccccc56)cc4)cc2O3)cc1 ; c1ccc(-c2ccc3c4c(cccc24)-c2ccc(N(c4ccccc4)c4ccc5oc6ccccc6c5c4)cc2O3)cc1 ; c1ccc(-c2ccc3c4c(cccc24)-c2ccc(N(c4ccc5ccccc5c4)c4ccc5oc6ccccc6c5c4)cc2O3)cc1 ; c1ccc(N(c2ccc3c(c2)Oc2ccc(-c4ccc5ccccc5c4)c4cccc-3c24)c2ccc3oc4ccccc4c3c2)cc1]

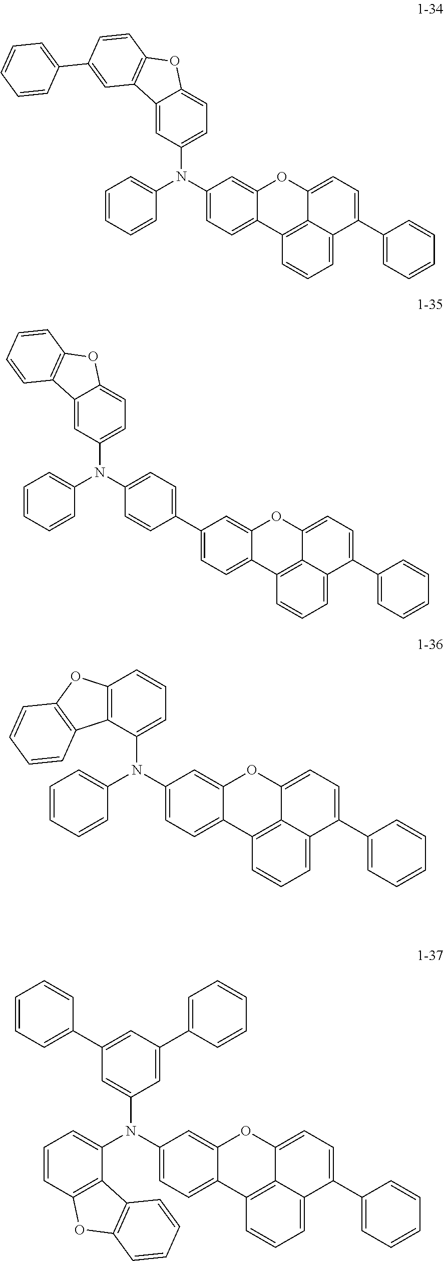 [SMILES: c1ccc(-c2ccc3oc4ccc(N(c5ccccc5)c5ccc6c(c5)Oc5ccc(-c7ccccc7)c7cccc-6c57)cc4c3c2)cc1 ; c1ccc(-c2ccc3c4c(cccc24)-c2ccc(-c4ccc(N(c5ccccc5)c5ccc6oc7ccccc7c6c5)cc4)cc2O3)cc1 ; c1ccc(-c2ccc3c4c(cccc24)-c2ccc(N(c4ccccc4)c4cccc5oc6ccccc6c45)cc2O3)cc1 ; c1ccc(-c2cc(-c3ccccc3)cc(N(c3ccc4c(c3)Oc3ccc(-c5ccccc5)c5cccc-4c35)c3cccc4oc5ccccc5c34)c2)cc1]

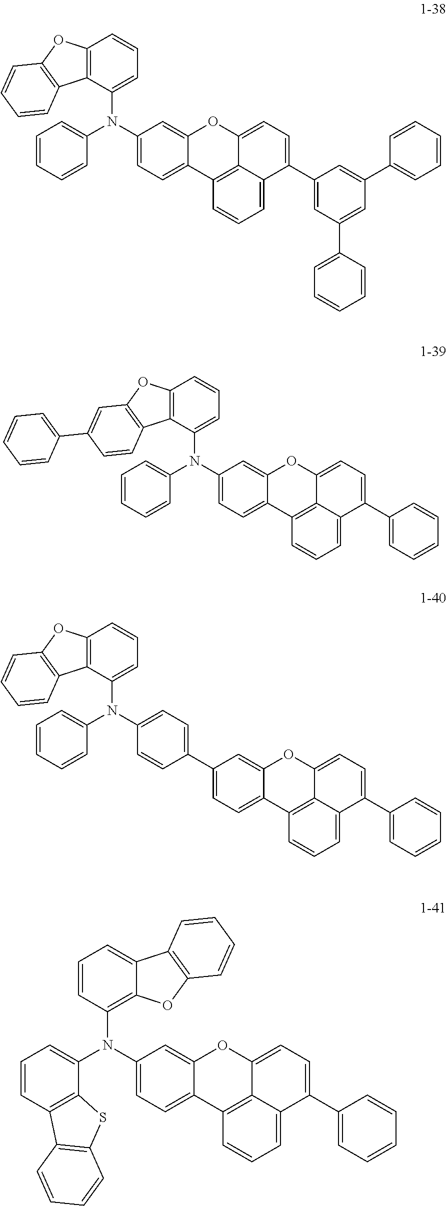 [SMILES: c1ccc(-c2cc(-c3ccccc3)cc(-c3ccc4c5c(cccc35)-c3ccc(N(c5ccccc5)c5cccc6oc7ccccc7c56)cc3O4)c2)cc1 ; c1ccc(-c2ccc3c(c2)oc2cccc(N(c4ccccc4)c4ccc5c(c4)Oc4ccc(-c6ccccc6)c6cccc-5c46)c23)cc1 ; c1ccc(-c2ccc3c4c(cccc24)-c2ccc(-c4ccc(N(c5ccccc5)c5cccc6oc7ccccc7c56)cc4)cc2O3)cc1 ; c1ccc(-c2ccc3c4c(cccc24)-c2ccc(N(c4cccc5c4oc4ccccc45)c4cccc5c4sc4ccccc45)cc2O3)cc1]

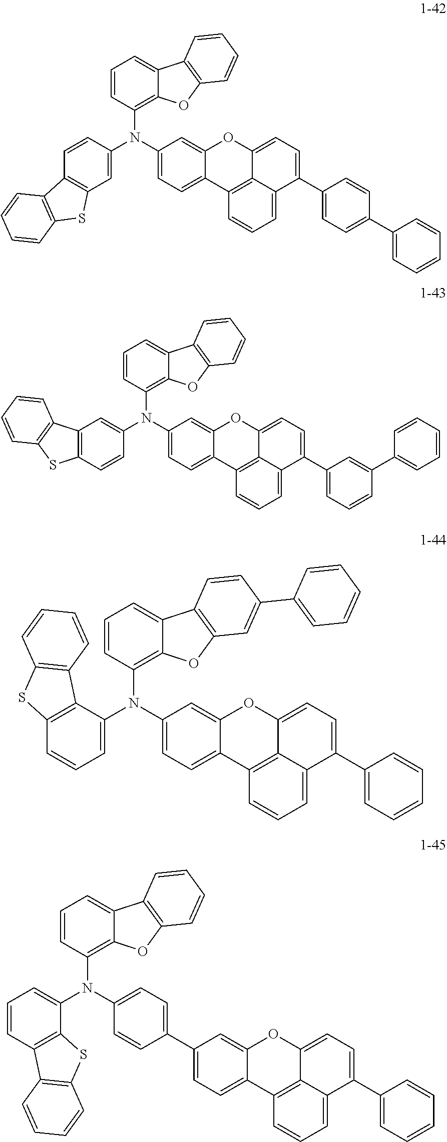 [SMILES: c1ccc(-c2ccc(-c3ccc4c5c(cccc35)-c3ccc(N(c5ccc6c(c5)sc5ccccc56)c5cccc6c5oc5ccccc56)cc3O4)cc2)cc1 ; c1ccc(-c2cccc(-c3ccc4c5c(cccc35)-c3ccc(N(c5ccc6sc7ccccc7c6c5)c5cccc6c5oc5ccccc56)cc3O4)c2)cc1 ; c1ccc(-c2ccc3c(c2)oc2c(N(c4ccc5c(c4)Oc4ccc(-c6ccccc6)c6cccc-5c46)c4cccc5sc6ccccc6c45)cccc23)cc1 ; c1ccc(-c2ccc3c4c(cccc24)-c2ccc(-c4ccc(N(c5cccc6c5oc5ccccc56)c5cccc6c5sc5ccccc56)cc4)cc2O3)cc1]

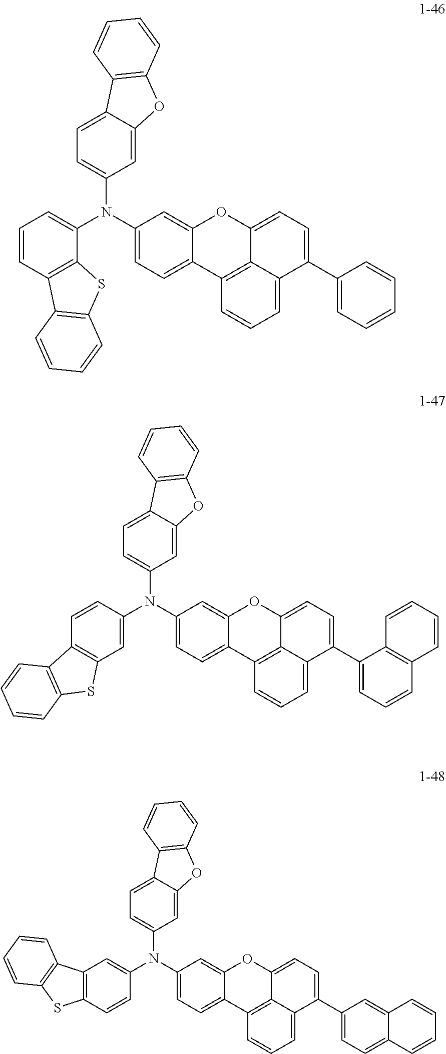 [SMILES: c1ccc(-c2ccc3c4c(cccc24)-c2ccc(N(c4ccc5c(c4)oc4ccccc45)c4cccc5c4sc4ccccc45)cc2O3)cc1 ; c1ccc2c(-c3ccc4c5c(cccc35)-c3ccc(N(c5ccc6c(c5)oc5ccccc56)c5ccc6c(c5)sc5ccccc56)cc3O4)cccc2c1 ; c1ccc2cc(-c3ccc4c5c(cccc35)-c3ccc(N(c5ccc6c(c5)oc5ccccc56)c5ccc6sc7ccccc7c6c5)cc3O4)ccc2c1]

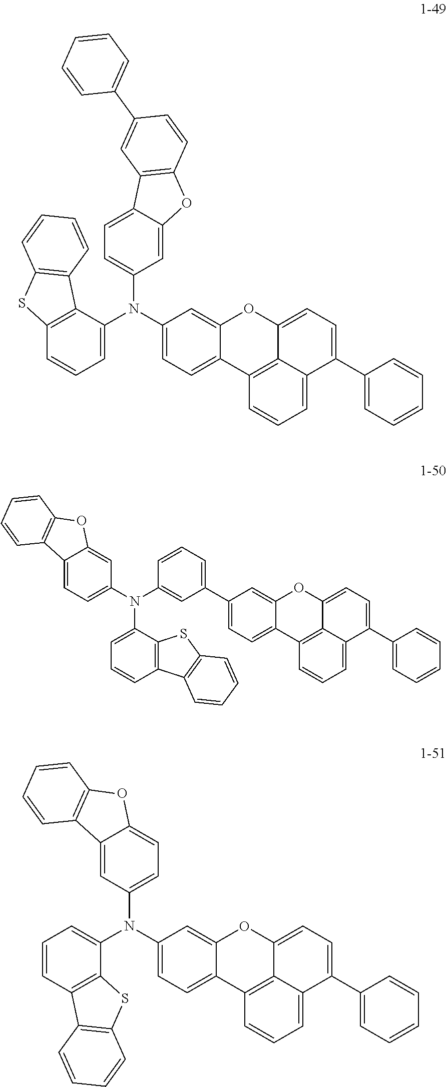 [SMILES: c1ccc(-c2ccc3oc4cc(N(c5ccc6c(c5)Oc5ccc(-c7ccccc7)c7cccc-6c57)c5cccc6sc7ccccc7c56)ccc4c3c2)cc1 ; c1ccc(-c2ccc3c4c(cccc24)-c2ccc(-c4cccc(N(c5ccc6c(c5)oc5ccccc56)c5cccc6c5sc5ccccc56)c4)cc2O3)cc1 ; c1ccc(-c2ccc3c4c(cccc24)-c2ccc(N(c4ccc5oc6ccccc6c5c4)c4cccc5c4sc4ccccc45)cc2O3)cc1]

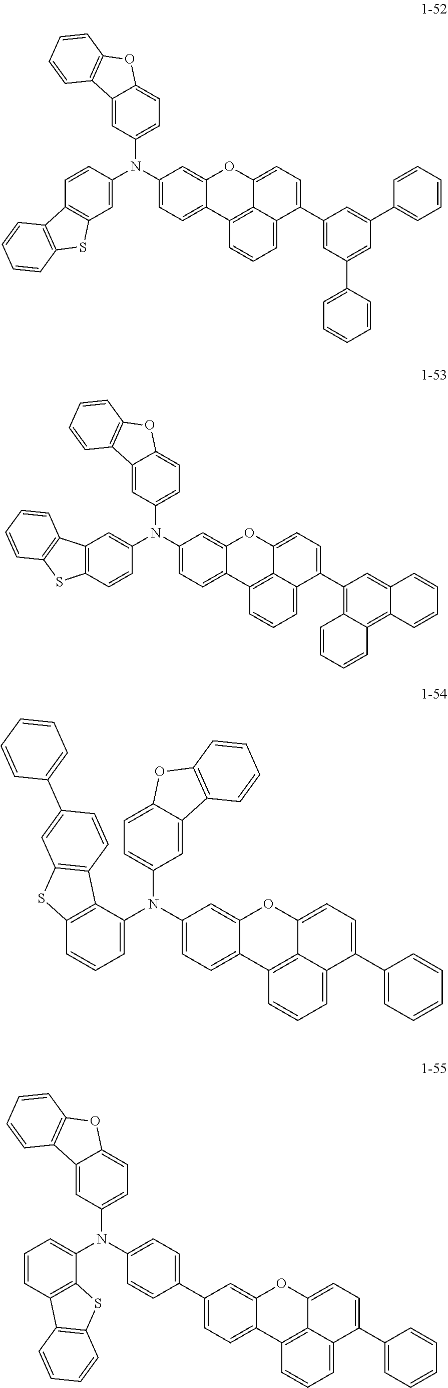 [SMILES: c1ccc(-c2cc(-c3ccccc3)cc(-c3ccc4c5c(cccc35)-c3ccc(N(c5ccc6c(c5)sc5ccccc56)c5ccc6oc7ccccc7c6c5)cc3O4)c2)cc1 ; c1ccc2c(c1)cc(-c1ccc3c4c(cccc14)-c1ccc(N(c4ccc5oc6ccccc6c5c4)c4ccc5sc6ccccc6c5c4)cc1O3)c1ccccc12 ; c1ccc(-c2ccc3c(c2)sc2cccc(N(c4ccc5c(c4)Oc4ccc(-c6ccccc6)c6cccc-5c46)c4ccc5oc6ccccc6c5c4)c23)cc1 ; c1ccc(-c2ccc3c4c(cccc24)-c2ccc(-c4ccc(N(c5ccc6oc7ccccc7c6c5)c5cccc6c5sc5ccccc56)cc4)cc2O3)cc1]

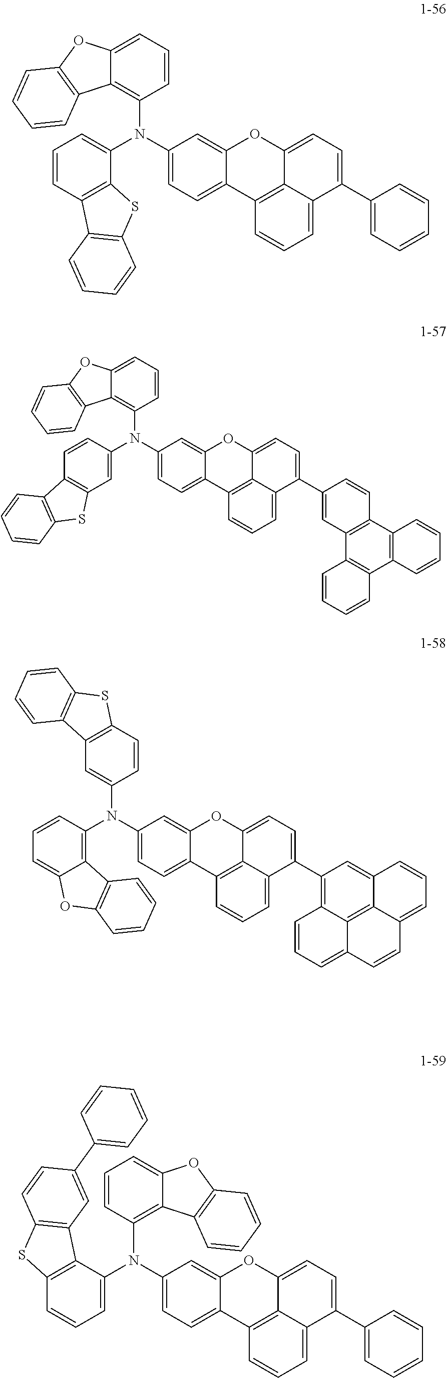 [SMILES: c1ccc(-c2ccc3c4c(cccc24)-c2ccc(N(c4cccc5c4sc4ccccc45)c4cccc5oc6ccccc6c45)cc2O3)cc1 ; c1ccc2c(c1)oc1cccc(N(c3ccc4c(c3)Oc3ccc(-c5ccc6c7ccccc7c7ccccc7c6c5)c5cccc-4c35)c3ccc4c(c3)sc3ccccc34)c12 ; c1cc2ccc3cccc4c(-c5ccc6c7c(cccc57)-c5ccc(N(c7ccc8sc9ccccc9c8c7)c7cccc8oc9ccccc9c78)cc5O6)cc(c1)c2c34 ; c1ccc(-c2ccc3sc4cccc(N(c5ccc6c(c5)Oc5ccc(-c7ccccc7)c7cccc-6c57)c5cccc6oc7ccccc7c56)c4c3c2)cc1]

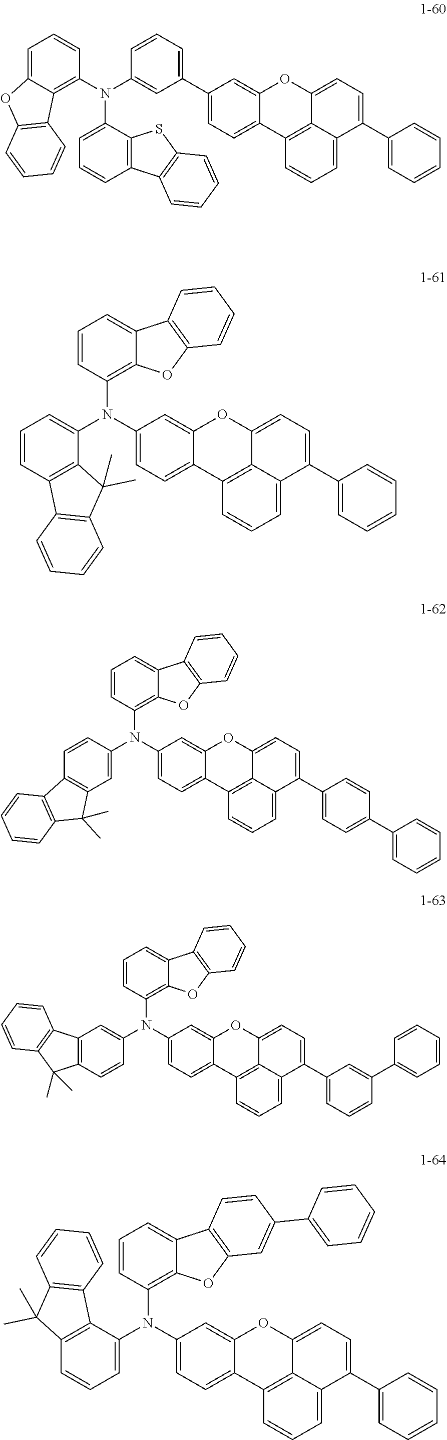 [SMILES: c1ccc(-c2ccc3c4c(cccc24)-c2ccc(-c4cccc(N(c5cccc6c5sc5ccccc56)c5cccc6oc7ccccc7c56)c4)cc2O3)cc1 ; CC1(C)c2ccccc2-c2cccc(N(c3ccc4c(c3)Oc3ccc(-c5ccccc5)c5cccc-4c35)c3cccc4c3oc3ccccc34)c21 ; CC1(C)c2ccccc2-c2ccc(N(c3ccc4c(c3)Oc3ccc(-c5ccc(-c6ccccc6)cc5)c5cccc-4c35)c3cccc4c3oc3ccccc34)cc21 ; CC1(C)c2ccccc2-c2cc(N(c3ccc4c(c3)Oc3ccc(-c5cccc(-c6ccccc6)c5)c5cccc-4c35)c3cccc4c3oc3ccccc34)ccc21 ; CC1(C)c2ccccc2-c2c(N(c3ccc4c(c3)Oc3ccc(-c5ccccc5)c5cccc-4c35)c3cccc4c3oc3cc(-c5ccccc5)ccc34)cccc21]

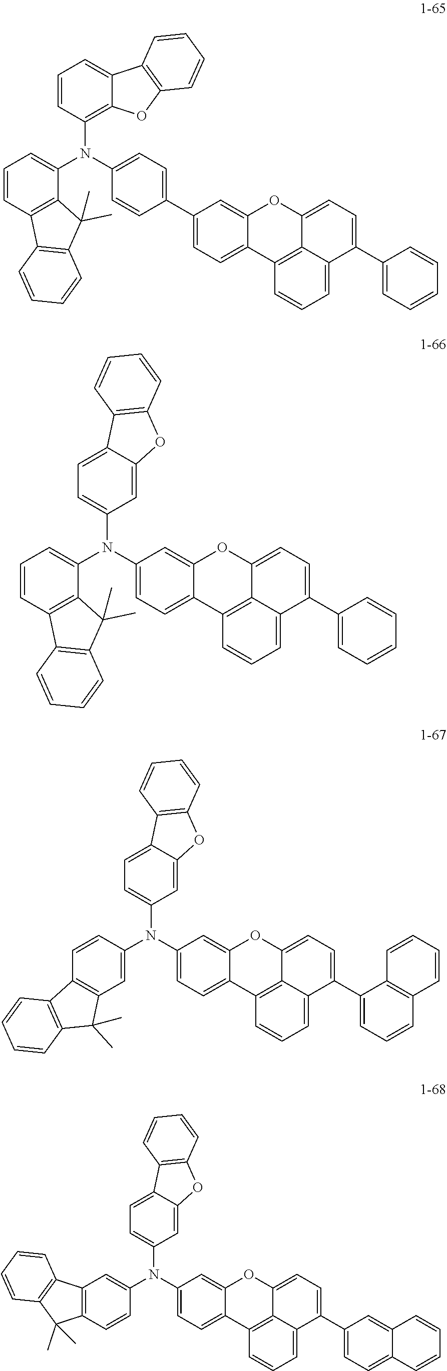 [SMILES: CC1(C)c2ccccc2-c2cccc(N(c3ccc(-c4ccc5c(c4)Oc4ccc(-c6ccccc6)c6cccc-5c46)cc3)c3cccc4c3oc3ccccc34)c21 ; CC1(C)c2ccccc2-c2cccc(N(c3ccc4c(c3)Oc3ccc(-c5ccccc5)c5cccc-4c35)c3ccc4c(c3)oc3ccccc34)c21 ; CC1(C)c2ccccc2-c2ccc(N(c3ccc4c(c3)Oc3ccc(-c5cccc6ccccc56)c5cccc-4c35)c3ccc4c(c3)oc3ccccc34)cc21 ; CC1(C)c2ccccc2-c2cc(N(c3ccc4c(c3)Oc3ccc(-c5ccc6ccccc6c5)c5cccc-4c35)c3ccc4c(c3)oc3ccccc34)ccc21]

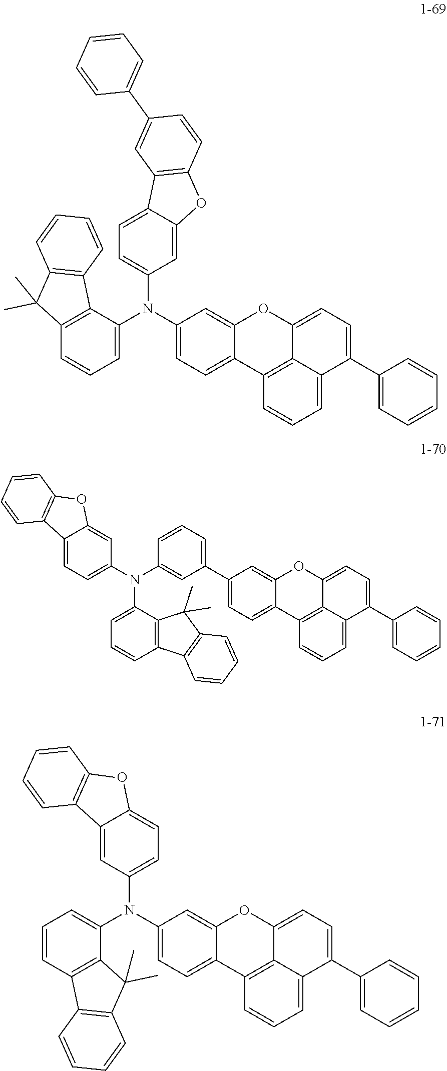 [SMILES: CC1(C)c2ccccc2-c2c(N(c3ccc4c(c3)Oc3ccc(-c5ccccc5)c5cccc-4c35)c3ccc4c(c3)oc3ccc(-c5ccccc5)cc34)cccc21 ; CC1(C)c2ccccc2-c2cccc(N(c3cccc(-c4ccc5c(c4)Oc4ccc(-c6ccccc6)c6cccc-5c46)c3)c3ccc4c(c3)oc3ccccc34)c21 ; CC1(C)c2ccccc2-c2cccc(N(c3ccc4c(c3)Oc3ccc(-c5ccccc5)c5cccc-4c35)c3ccc4oc5ccccc5c4c3)c21]

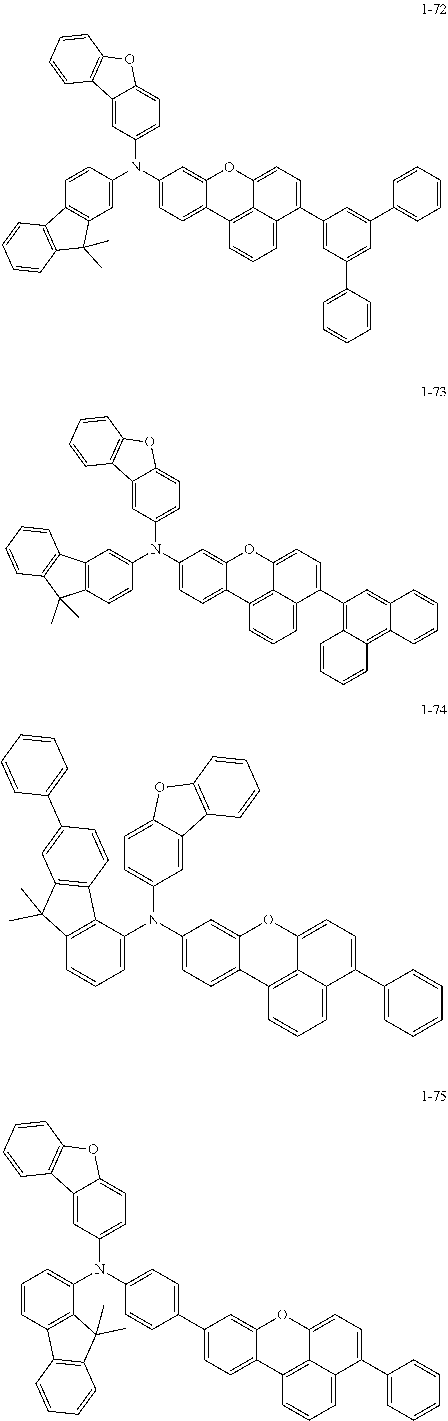 [SMILES: CC1(C)c2ccccc2-c2ccc(N(c3ccc4c(c3)Oc3ccc(-c5cc(-c6ccccc6)cc(-c6ccccc6)c5)c5cccc-4c35)c3ccc4oc5ccccc5c4c3)cc21 ; CC1(C)c2ccccc2-c2cc(N(c3ccc4c(c3)Oc3ccc(-c5cc6ccccc6c6ccccc56)c5cccc-4c35)c3ccc4oc5ccccc5c4c3)ccc21 ; CC1(C)c2cc(-c3ccccc3)ccc2-c2c(N(c3ccc4c(c3)Oc3ccc(-c5ccccc5)c5cccc-4c35)c3ccc4oc5ccccc5c4c3)cccc21 ; CC1(C)c2ccccc2-c2cccc(N(c3ccc(-c4ccc5c(c4)Oc4ccc(-c6ccccc6)c6cccc-5c46)cc3)c3ccc4oc5ccccc5c4c3)c21]

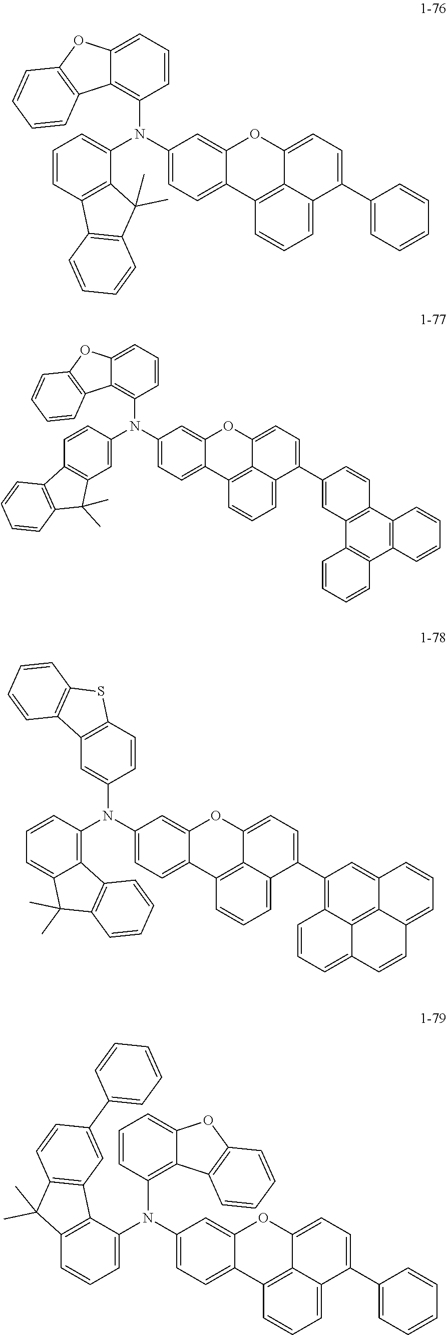 [SMILES: CC1(C)c2ccccc2-c2cccc(N(c3ccc4c(c3)Oc3ccc(-c5ccccc5)c5cccc-4c35)c3cccc4oc5ccccc5c34)c21 ; CC1(C)c2ccccc2-c2ccc(N(c3ccc4c(c3)Oc3ccc(-c5ccc6c7ccccc7c7ccccc7c6c5)c5cccc-4c35)c3cccc4oc5ccccc5c34)cc21 ; CC1(C)c2ccccc2-c2c(N(c3ccc4c(c3)Oc3ccc(-c5cc6cccc7ccc8cccc5c8c76)c5cccc-4c35)c3ccc4sc5ccccc5c4c3)cccc21 ; CC1(C)c2ccc(-c3ccccc3)cc2-c2c(N(c3ccc4c(c3)Oc3ccc(-c5ccccc5)c5cccc-4c35)c3cccc4oc5ccccc5c34)cccc21]

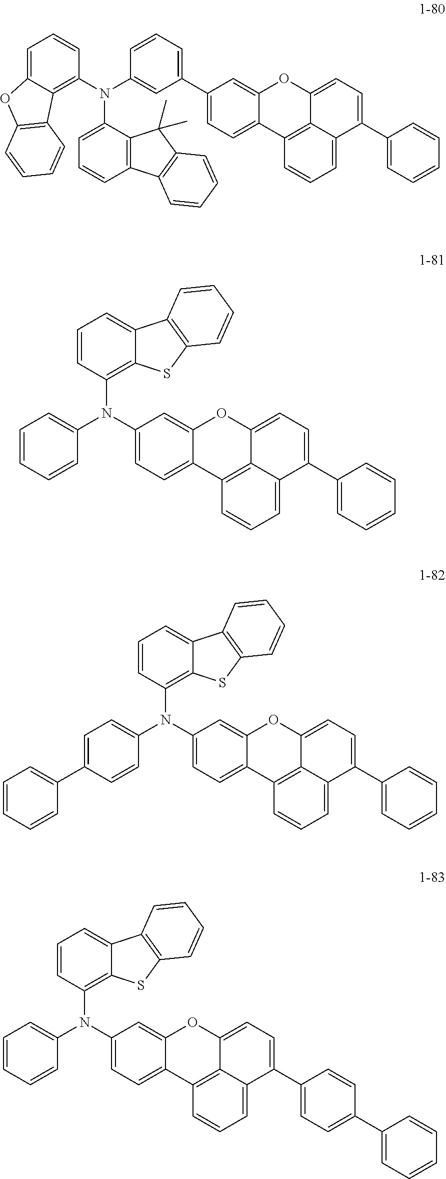 [SMILES: CC1(C)c2ccccc2-c2cccc(N(c3cccc(-c4ccc5c(c4)Oc4ccc(-c6ccccc6)c6cccc-5c46)c3)c3cccc4oc5ccccc5c34)c21 ; c1ccc(-c2ccc3c4c(cccc24)-c2ccc(N(c4ccccc4)c4cccc5c4sc4ccccc45)cc2O3)cc1 ; c1ccc(-c2ccc(N(c3ccc4c(c3)Oc3ccc(-c5ccccc5)c5cccc-4c35)c3cccc4c3sc3ccccc34)cc2)cc1 ; c1ccc(-c2ccc(-c3ccc4c5c(cccc35)-c3ccc(N(c5ccccc5)c5cccc6c5sc5ccccc56)cc3O4)cc2)cc1]

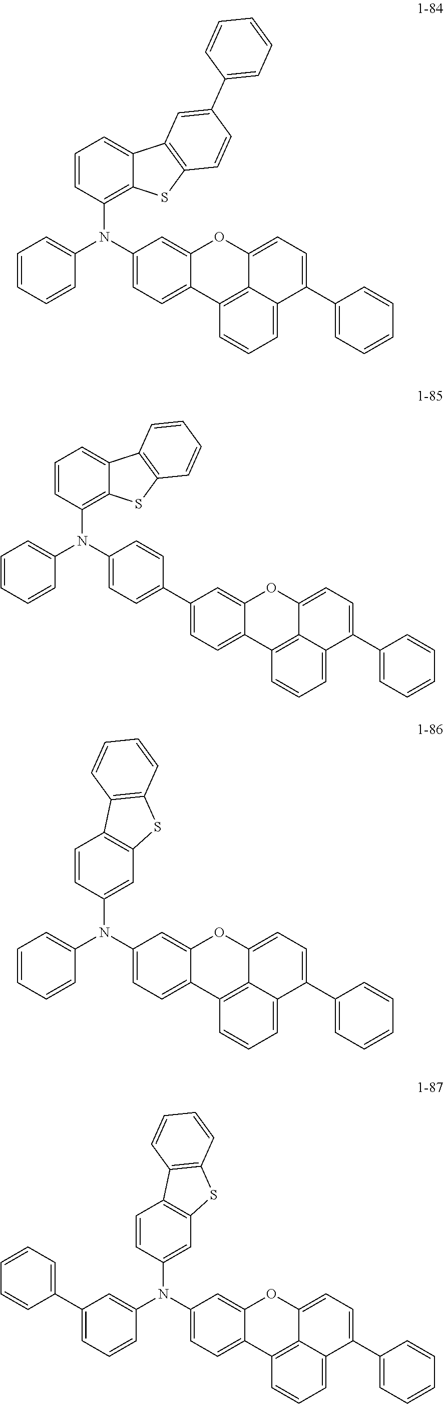 [SMILES: c1ccc(-c2ccc3sc4c(N(c5ccccc5)c5ccc6c(c5)Oc5ccc(-c7ccccc7)c7cccc-6c57)cccc4c3c2)cc1 ; c1ccc(-c2ccc3c4c(cccc24)-c2ccc(-c4ccc(N(c5ccccc5)c5cccc6c5sc5ccccc56)cc4)cc2O3)cc1 ; c1ccc(-c2ccc3c4c(cccc24)-c2ccc(N(c4ccccc4)c4ccc5c(c4)sc4ccccc45)cc2O3)cc1 ; c1ccc(-c2cccc(N(c3ccc4c(c3)Oc3ccc(-c5ccccc5)c5cccc-4c35)c3ccc4c(c3)sc3ccccc34)c2)cc1]

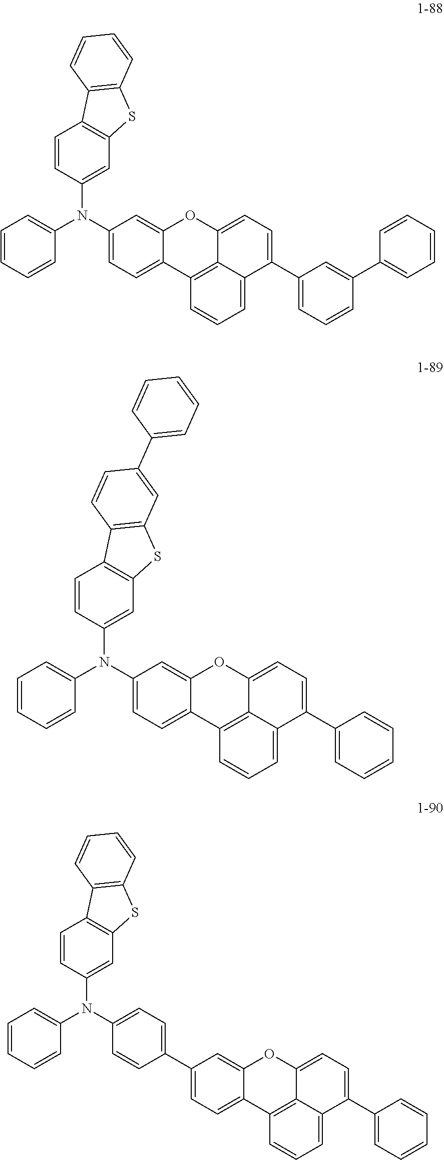 [SMILES: c1ccc(-c2cccc(-c3ccc4c5c(cccc35)-c3ccc(N(c5ccccc5)c5ccc6c(c5)sc5ccccc56)cc3O4)c2)cc1 ; c1ccc(-c2ccc3c(c2)sc2cc(N(c4ccccc4)c4ccc5c(c4)Oc4ccc(-c6ccccc6)c6cccc-5c46)ccc23)cc1 ; c1ccc(-c2ccc3c4c(cccc24)-c2ccc(-c4ccc(N(c5ccccc5)c5ccc6c(c5)sc5ccccc56)cc4)cc2O3)cc1]

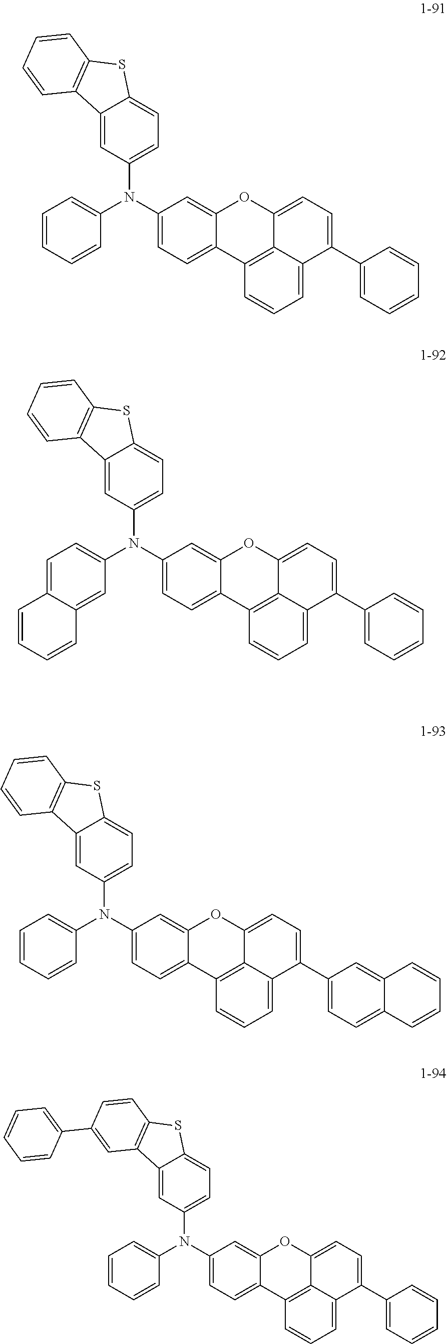 [SMILES: c1ccc(-c2ccc3c4c(cccc24)-c2ccc(N(c4ccccc4)c4ccc5sc6ccccc6c5c4)cc2O3)cc1 ; c1ccc(-c2ccc3c4c(cccc24)-c2ccc(N(c4ccc5ccccc5c4)c4ccc5sc6ccccc6c5c4)cc2O3)cc1 ; c1ccc(N(c2ccc3c(c2)Oc2ccc(-c4ccc5ccccc5c4)c4cccc-3c24)c2ccc3sc4ccccc4c3c2)cc1 ; c1ccc(-c2ccc3sc4ccc(N(c5ccccc5)c5ccc6c(c5)Oc5ccc(-c7ccccc7)c7cccc-6c57)cc4c3c2)cc1]

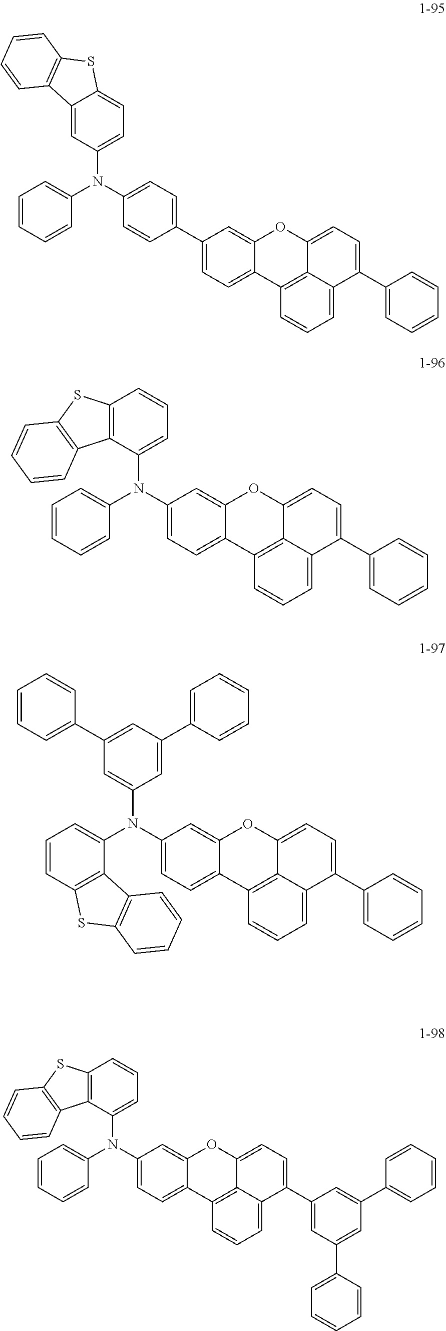 [SMILES: c1ccc(-c2ccc3c4c(cccc24)-c2ccc(-c4ccc(N(c5ccccc5)c5ccc6sc7ccccc7c6c5)cc4)cc2O3)cc1 ; c1ccc(-c2ccc3c4c(cccc24)-c2ccc(N(c4ccccc4)c4cccc5sc6ccccc6c45)cc2O3)cc1 ; c1ccc(-c2cc(-c3ccccc3)cc(N(c3ccc4c(c3)Oc3ccc(-c5ccccc5)c5cccc-4c35)c3cccc4sc5ccccc5c34)c2)cc1 ; c1ccc(-c2cc(-c3ccccc3)cc(-c3ccc4c5c(cccc35)-c3ccc(N(c5ccccc5)c5cccc6sc7ccccc7c56)cc3O4)c2)cc1]

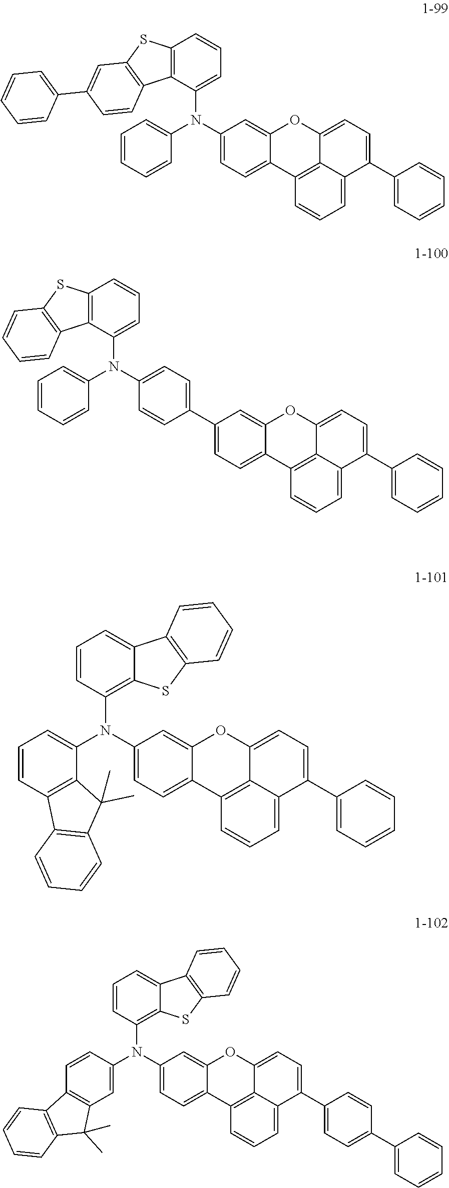 [SMILES: c1ccc(-c2ccc3c(c2)sc2cccc(N(c4ccccc4)c4ccc5c(c4)Oc4ccc(-c6ccccc6)c6cccc-5c46)c23)cc1 ; c1ccc(-c2ccc3c4c(cccc24)-c2ccc(-c4ccc(N(c5ccccc5)c5cccc6sc7ccccc7c56)cc4)cc2O3)cc1 ; CC1(C)c2ccccc2-c2cccc(N(c3ccc4c(c3)Oc3ccc(-c5ccccc5)c5cccc-4c35)c3cccc4c3sc3ccccc34)c21 ; CC1(C)c2ccccc2-c2ccc(N(c3ccc4c(c3)Oc3ccc(-c5ccc(-c6ccccc6)cc5)c5cccc-4c35)c3cccc4c3sc3ccccc34)cc21]

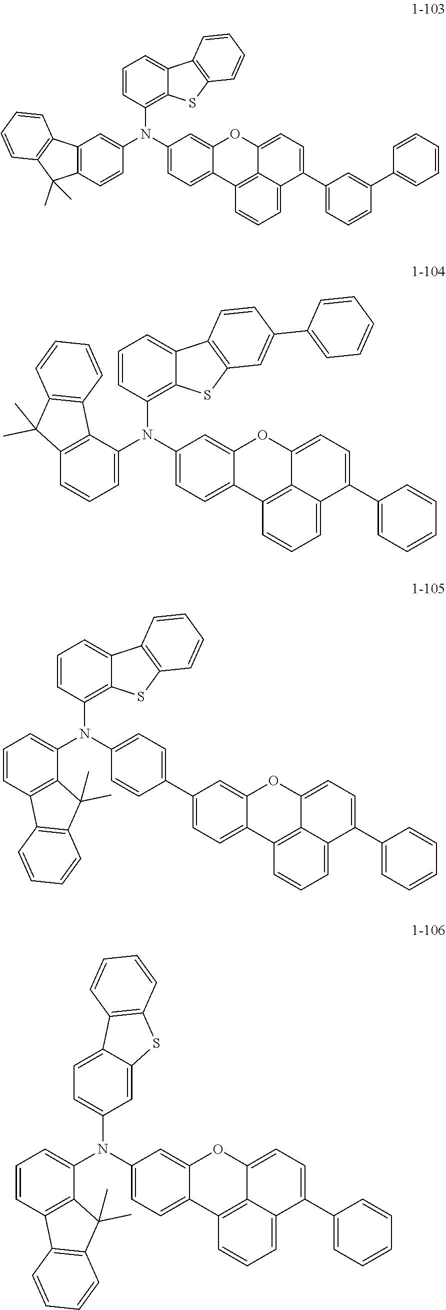 [SMILES: CC1(C)c2ccccc2-c2cc(N(c3ccc4c(c3)Oc3ccc(-c5cccc(-c6ccccc6)c5)c5cccc-4c35)c3cccc4c3sc3ccccc34)ccc21 ; CC1(C)c2ccccc2-c2c(N(c3ccc4c(c3)Oc3ccc(-c5ccccc5)c5cccc-4c35)c3cccc4c3sc3cc(-c5ccccc5)ccc34)cccc21 ; CC1(C)c2ccccc2-c2cccc(N(c3ccc(-c4ccc5c(c4)Oc4ccc(-c6ccccc6)c6cccc-5c46)cc3)c3cccc4c3sc3ccccc34)c21 ; CC1(C)c2ccccc2-c2cccc(N(c3ccc4c(c3)Oc3ccc(-c5ccccc5)c5cccc-4c35)c3ccc4c(c3)sc3ccccc34)c21]

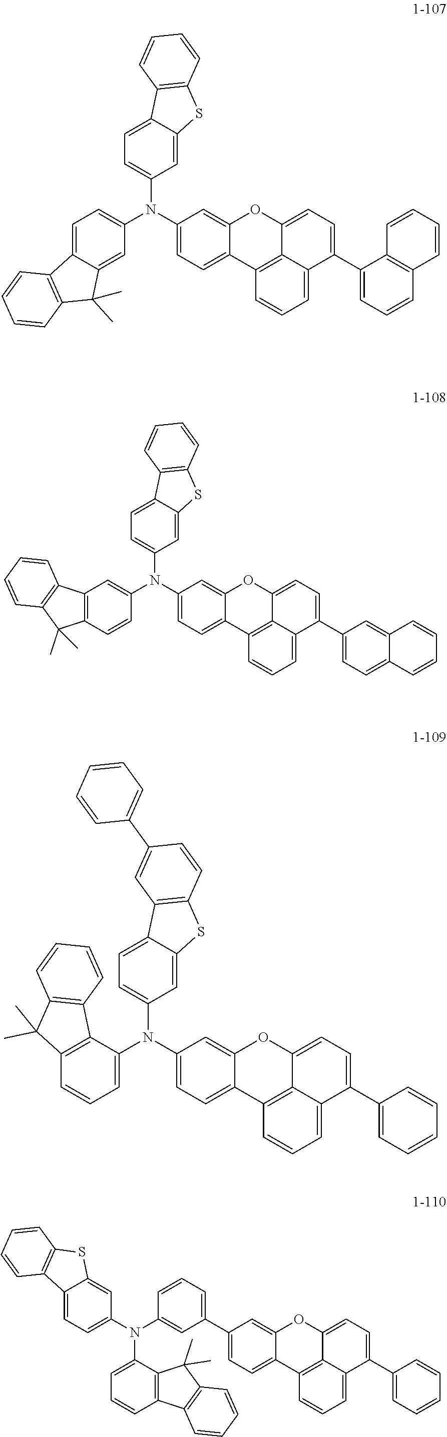 [SMILES: CC1(C)c2ccccc2-c2ccc(N(c3ccc4c(c3)Oc3ccc(-c5cccc6ccccc56)c5cccc-4c35)c3ccc4c(c3)sc3ccccc34)cc21 ; CC1(C)c2ccccc2-c2cc(N(c3ccc4c(c3)Oc3ccc(-c5ccc6ccccc6c5)c5cccc-4c35)c3ccc4c(c3)sc3ccccc34)ccc21 ; CC1(C)c2ccccc2-c2c(N(c3ccc4c(c3)Oc3ccc(-c5ccccc5)c5cccc-4c35)c3ccc4c(c3)sc3ccc(-c5ccccc5)cc34)cccc21 ; CC1(C)c2ccccc2-c2cccc(N(c3cccc(-c4ccc5c(c4)Oc4ccc(-c6ccccc6)c6cccc-5c46)c3)c3ccc4c(c3)sc3ccccc34)c21]

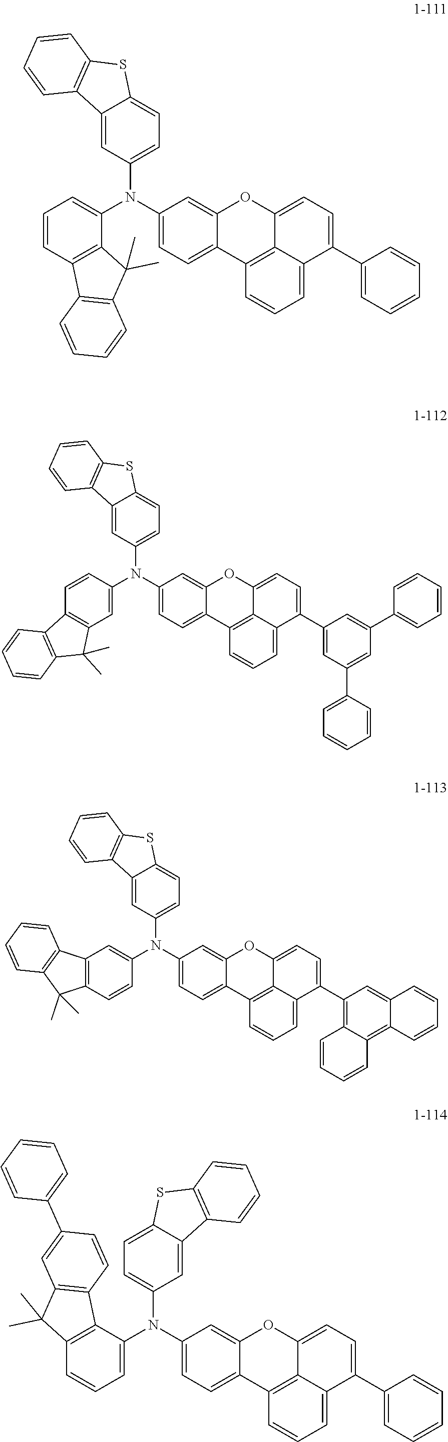 [SMILES: CC1(C)c2ccccc2-c2cccc(N(c3ccc4c(c3)Oc3ccc(-c5ccccc5)c5cccc-4c35)c3ccc4sc5ccccc5c4c3)c21 ; CC1(C)c2ccccc2-c2ccc(N(c3ccc4c(c3)Oc3ccc(-c5cc(-c6ccccc6)cc(-c6ccccc6)c5)c5cccc-4c35)c3ccc4sc5ccccc5c4c3)cc21 ; CC1(C)c2ccccc2-c2cc(N(c3ccc4c(c3)Oc3ccc(-c5cc6ccccc6c6ccccc56)c5cccc-4c35)c3ccc4sc5ccccc5c4c3)ccc21 ; CC1(C)c2cc(-c3ccccc3)ccc2-c2c(N(c3ccc4c(c3)Oc3ccc(-c5ccccc5)c5cccc-4c35)c3ccc4sc5ccccc5c4c3)cccc21]

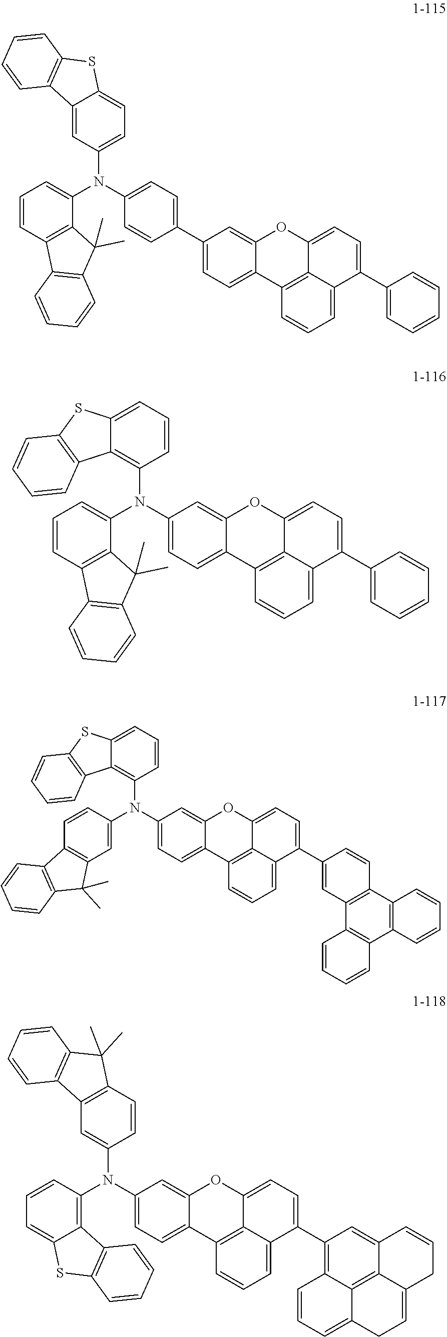 [SMILES: CC1(C)c2ccccc2-c2cccc(N(c3ccc(-c4ccc5c(c4)Oc4ccc(-c6ccccc6)c6cccc-5c46)cc3)c3ccc4sc5ccccc5c4c3)c21 ; CC1(C)c2ccccc2-c2cccc(N(c3ccc4c(c3)Oc3ccc(-c5ccccc5)c5cccc-4c35)c3cccc4sc5ccccc5c34)c21 ; CC1(C)c2ccccc2-c2ccc(N(c3ccc4c(c3)Oc3ccc(-c5ccc6c7ccccc7c7ccccc7c6c5)c5cccc-4c35)c3cccc4sc5ccccc5c34)cc21 ; CC1(C)c2ccccc2-c2cc(N(c3ccc4c(c3)Oc3ccc(-c5cc6c7c8c(cccc58)CC=C7CC=C6)c5cccc-4c35)c3cccc4sc5ccccc5c34)ccc21]

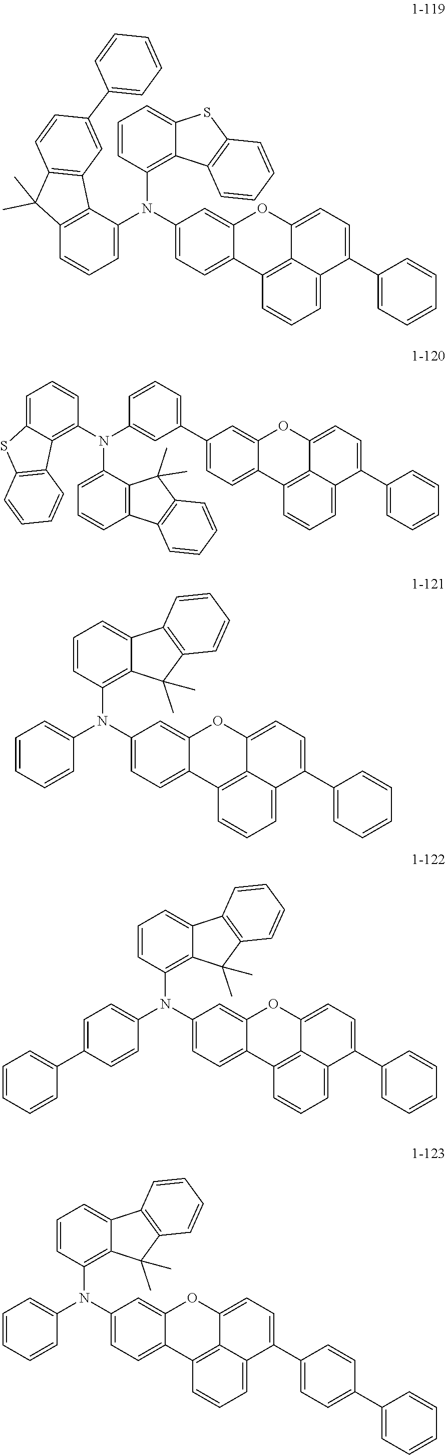

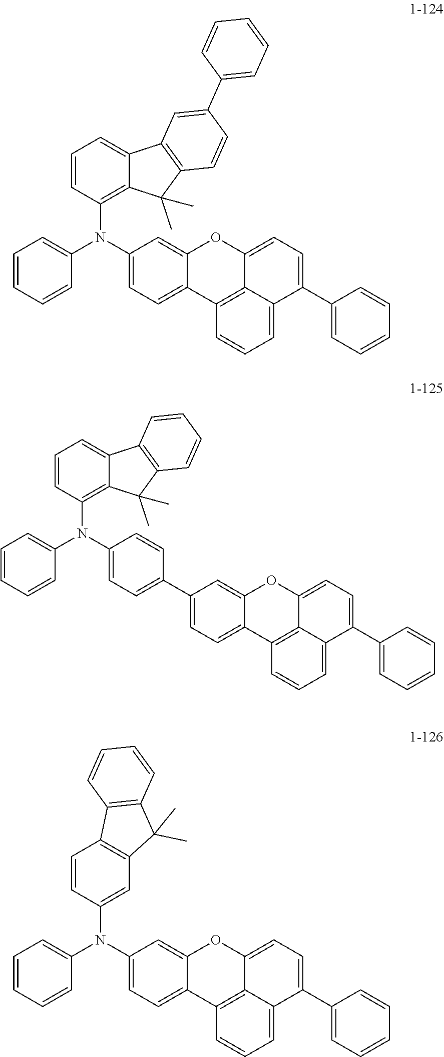 [SMILES: CC1(C)c2ccc(-c3ccccc3)cc2-c2cccc(N(c3ccccc3)c3ccc4c(c3)Oc3ccc(-c5ccccc5)c5cccc-4c35)c21 ; CC1(C)c2ccccc2-c2cccc(N(c3ccccc3)c3ccc(-c4ccc5c(c4)Oc4ccc(-c6ccccc6)c6cccc-5c46)cc3)c21 ; CC1(C)c2ccccc2-c2ccc(N(c3ccccc3)c3ccc4c(c3)Oc3ccc(-c5ccccc5)c5cccc-4c35)cc21]

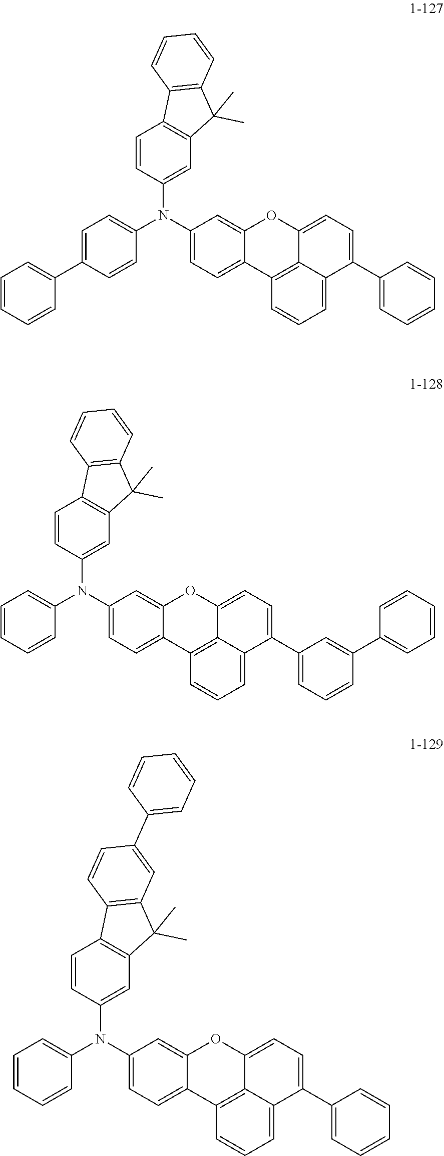 [SMILES: CC1(C)c2ccccc2-c2ccc(N(c3ccc(-c4ccccc4)cc3)c3ccc4c(c3)Oc3ccc(-c5ccccc5)c5cccc-4c35)cc21 ; CC1(C)c2ccccc2-c2ccc(N(c3ccccc3)c3ccc4c(c3)Oc3ccc(-c5cccc(-c6ccccc6)c5)c5cccc-4c35)cc21 ; CC1(C)c2cc(-c3ccccc3)ccc2-c2ccc(N(c3ccccc3)c3ccc4c(c3)Oc3ccc(-c5ccccc5)c5cccc-4c35)cc21]

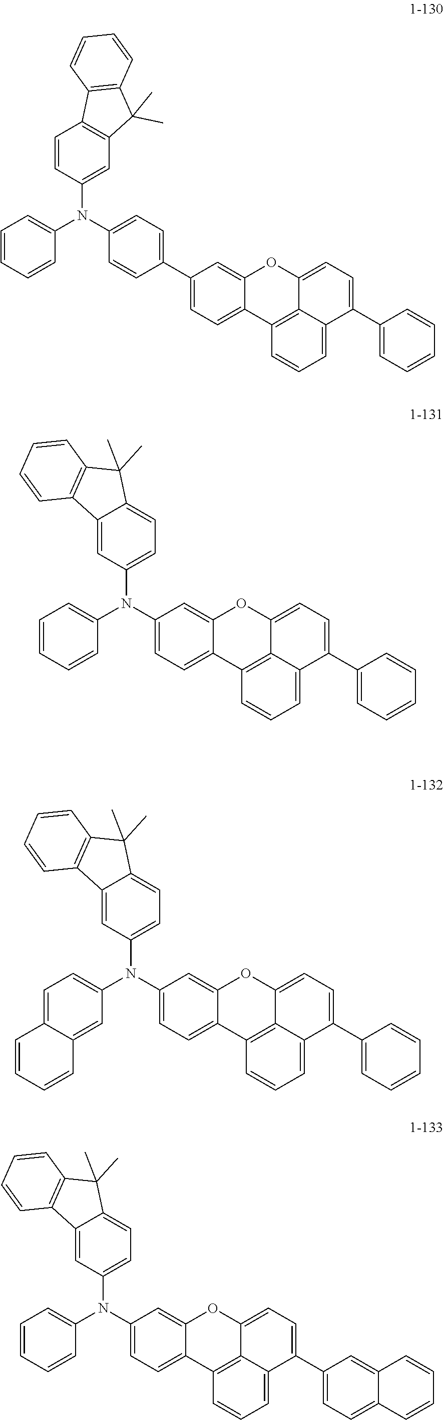 [SMILES: CC1(C)c2ccccc2-c2ccc(N(c3ccccc3)c3ccc(-c4ccc5c(c4)Oc4ccc(-c6ccccc6)c6cccc-5c46)cc3)cc21 ; CC1(C)c2ccccc2-c2cc(N(c3ccccc3)c3ccc4c(c3)Oc3ccc(-c5ccccc5)c5cccc-4c35)ccc21 ; CC1(C)c2ccccc2-c2cc(N(c3ccc4c(c3)Oc3ccc(-c5ccccc5)c5cccc-4c35)c3ccc4ccccc4c3)ccc21 ; CC1(C)c2ccccc2-c2cc(N(c3ccccc3)c3ccc4c(c3)Oc3ccc(-c5ccc6ccccc6c5)c5cccc-4c35)ccc21]

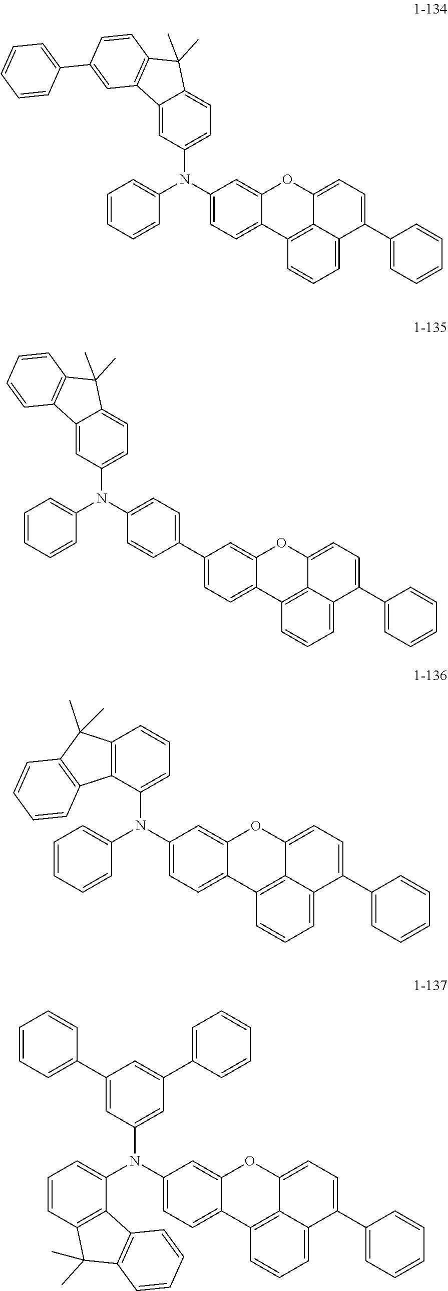 [SMILES: CC1(C)c2ccc(-c3ccccc3)cc2-c2cc(N(c3ccccc3)c3ccc4c(c3)Oc3ccc(-c5ccccc5)c5cccc-4c35)ccc21 ; CC1(C)c2ccccc2-c2cc(N(c3ccccc3)c3ccc(-c4ccc5c(c4)Oc4ccc(-c6ccccc6)c6cccc-5c46)cc3)ccc21 ; CC1(C)c2ccccc2-c2c(N(c3ccccc3)c3ccc4c(c3)Oc3ccc(-c5ccccc5)c5cccc-4c35)cccc21 ; CC1(C)c2ccccc2-c2c(N(c3cc(-c4ccccc4)cc(-c4ccccc4)c3)c3ccc4c(c3)Oc3ccc(-c5ccccc5)c5cccc-4c35)cccc21]

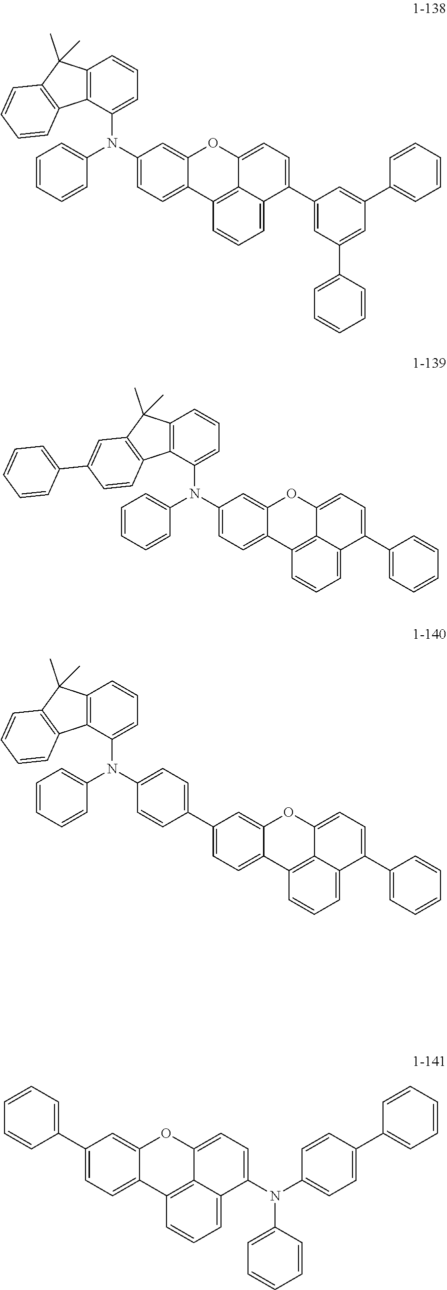 [SMILES: CC1(C)c2ccccc2-c2c(N(c3ccccc3)c3ccc4c(c3)Oc3ccc(-c5cc(-c6ccccc6)cc(-c6ccccc6)c5)c5cccc-4c35)cccc21 ; CC1(C)c2cc(-c3ccccc3)ccc2-c2c(N(c3ccccc3)c3ccc4c(c3)Oc3ccc(-c5ccccc5)c5cccc-4c35)cccc21 ; CC1(C)c2ccccc2-c2c(N(c3ccccc3)c3ccc(-c4ccc5c(c4)Oc4ccc(-c6ccccc6)c6cccc-5c46)cc3)cccc21 ; c1ccc(-c2ccc(N(c3ccccc3)c3ccc4c5c(cccc35)-c3ccc(-c5ccccc5)cc3O4)cc2)cc1]

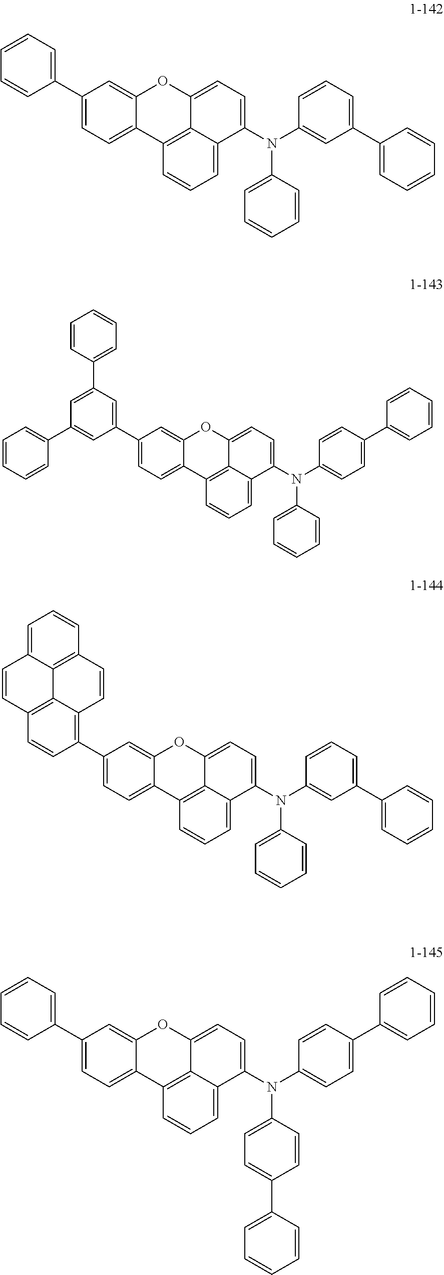 [SMILES: c1ccc(-c2cccc(N(c3ccccc3)c3ccc4c5c(cccc35)-c3ccc(-c5ccccc5)cc3O4)c2)cc1 ; c1ccc(-c2ccc(N(c3ccccc3)c3ccc4c5c(cccc35)-c3ccc(-c5cc(-c6ccccc6)cc(-c6ccccc6)c5)cc3O4)cc2)cc1 ; c1ccc(-c2cccc(N(c3ccccc3)c3ccc4c5c(cccc35)-c3ccc(-c5ccc6ccc7cccc8ccc5c6c78)cc3O4)c2)cc1 ; c1ccc(-c2ccc(N(c3ccc(-c4ccccc4)cc3)c3ccc4c5c(cccc35)-c3ccc(-c5ccccc5)cc3O4)cc2)cc1]

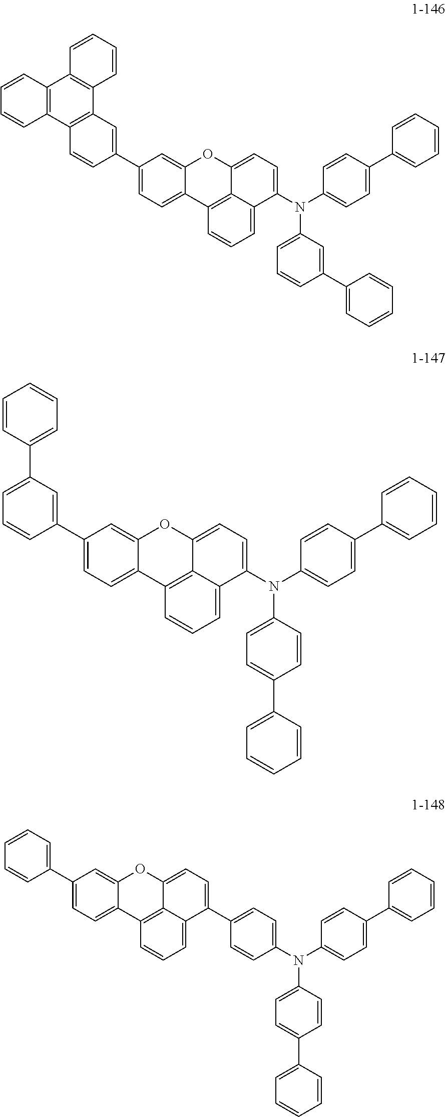 [SMILES: c1ccc(-c2ccc(N(c3cccc(-c4ccccc4)c3)c3ccc4c5c(cccc35)-c3ccc(-c5ccc6c7ccccc7c7ccccc7c6c5)cc3O4)cc2)cc1 ; c1ccc(-c2ccc(N(c3ccc(-c4ccccc4)cc3)c3ccc4c5c(cccc35)-c3ccc(-c5cccc(-c6ccccc6)c5)cc3O4)cc2)cc1 ; c1ccc(-c2ccc(N(c3ccc(-c4ccccc4)cc3)c3ccc(-c4ccc5c6c(cccc46)-c4ccc(-c6ccccc6)cc4O5)cc3)cc2)cc1]

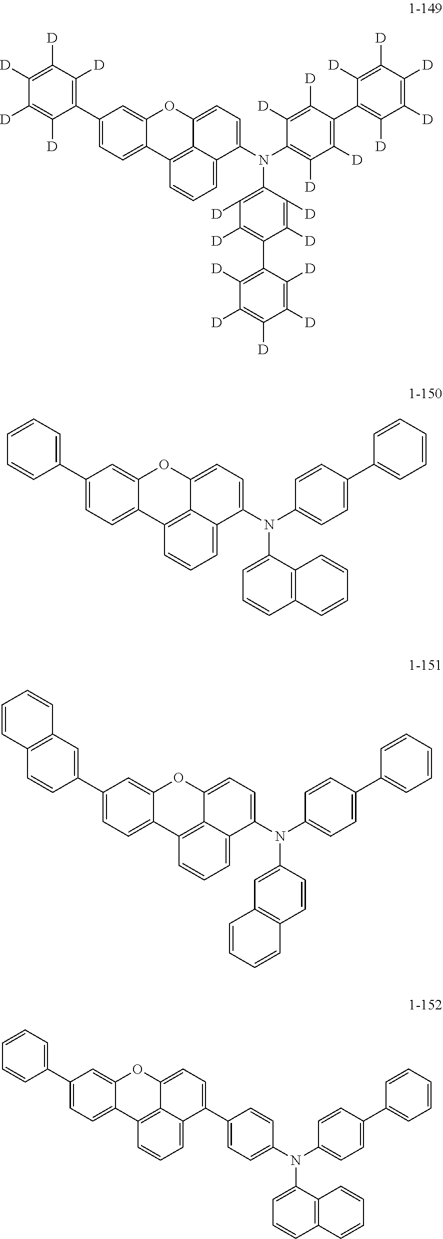 [SMILES: [2H]c1c([2H])c([2H])c(-c2ccc3c(c2)Oc2ccc(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])c4cccc-3c24)c([2H])c1[2H] ; c1ccc(-c2ccc(N(c3cccc4ccccc34)c3ccc4c5c(cccc35)-c3ccc(-c5ccccc5)cc3O4)cc2)cc1 ; c1ccc(-c2ccc(N(c3ccc4ccccc4c3)c3ccc4c5c(cccc35)-c3ccc(-c5ccc6ccccc6c5)cc3O4)cc2)cc1 ; c1ccc(-c2ccc(N(c3ccc(-c4ccc5c6c(cccc46)-c4ccc(-c6ccccc6)cc4O5)cc3)c3cccc4ccccc34)cc2)cc1]

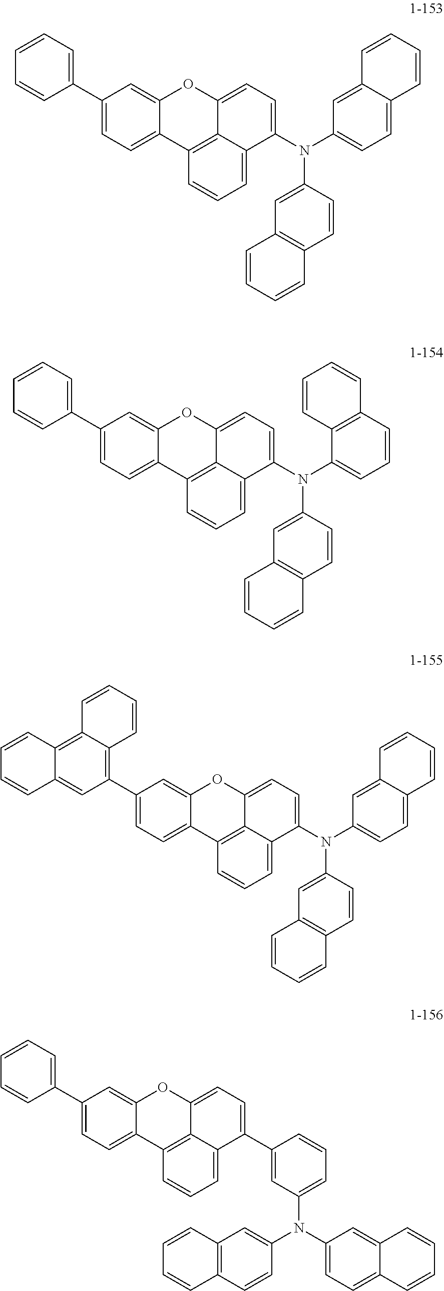 [SMILES: c1ccc(-c2ccc3c(c2)Oc2ccc(N(c4ccc5ccccc5c4)c4ccc5ccccc5c4)c4cccc-3c24)cc1 ; c1ccc(-c2ccc3c(c2)Oc2ccc(N(c4ccc5ccccc5c4)c4cccc5ccccc45)c4cccc-3c24)cc1 ; c1ccc2cc(N(c3ccc4ccccc4c3)c3ccc4c5c(cccc35)-c3ccc(-c5cc6ccccc6c6ccccc56)cc3O4)ccc2c1 ; c1ccc(-c2ccc3c(c2)Oc2ccc(-c4cccc(N(c5ccc6ccccc6c5)c5ccc6ccccc6c5)c4)c4cccc-3c24)cc1]

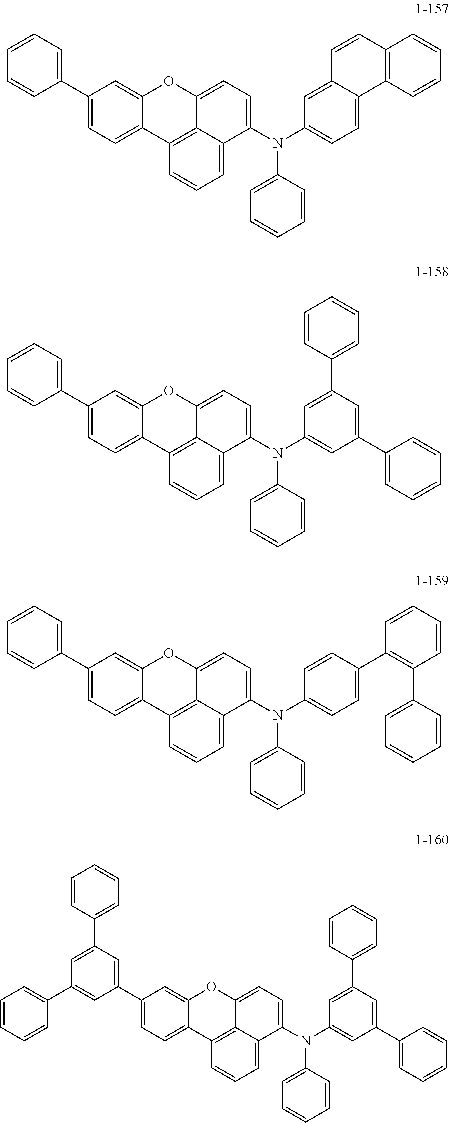 [SMILES: c1ccc(-c2ccc3c(c2)Oc2ccc(N(c4ccccc4)c4ccc5c(ccc6ccccc65)c4)c4cccc-3c24)cc1 ; c1ccc(-c2cc(-c3ccccc3)cc(N(c3ccccc3)c3ccc4c5c(cccc35)-c3ccc(-c5ccccc5)cc3O4)c2)cc1 ; c1ccc(-c2ccc3c(c2)Oc2ccc(N(c4ccccc4)c4ccc(-c5ccccc5-c5ccccc5)cc4)c4cccc-3c24)cc1 ; c1ccc(-c2cc(-c3ccccc3)cc(-c3ccc4c(c3)Oc3ccc(N(c5ccccc5)c5cc(-c6ccccc6)cc(-c6ccccc6)c5)c5cccc-4c35)c2)cc1]

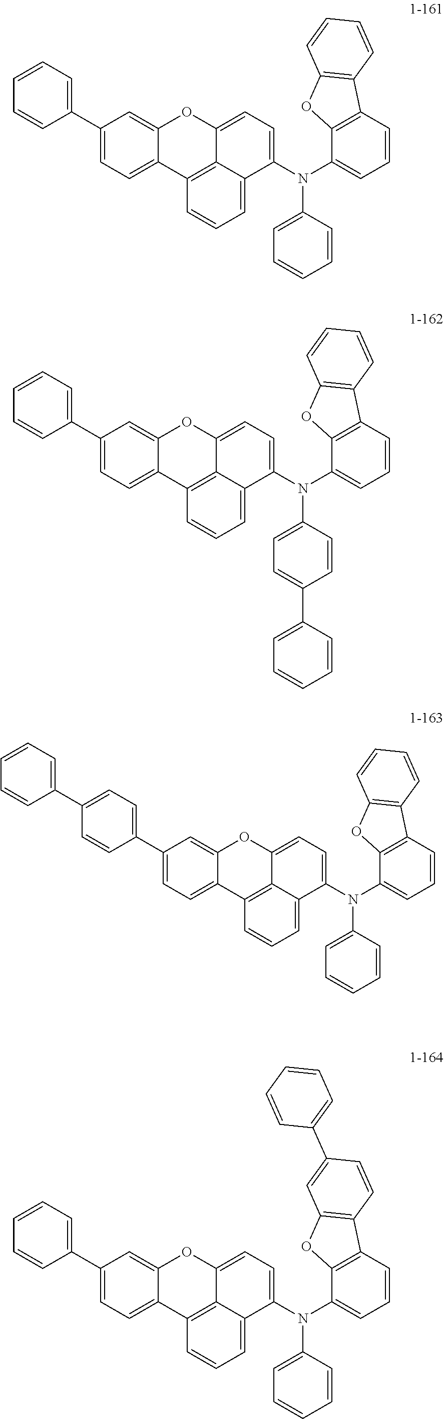 [SMILES: c1ccc(-c2ccc3c(c2)Oc2ccc(N(c4ccccc4)c4cccc5c4oc4ccccc45)c4cccc-3c24)cc1 ; c1ccc(-c2ccc(N(c3ccc4c5c(cccc35)-c3ccc(-c5ccccc5)cc3O4)c3cccc4c3oc3ccccc34)cc2)cc1 ; c1ccc(-c2ccc(-c3ccc4c(c3)Oc3ccc(N(c5ccccc5)c5cccc6c5oc5ccccc56)c5cccc-4c35)cc2)cc1 ; c1ccc(-c2ccc3c(c2)Oc2ccc(N(c4ccccc4)c4cccc5c4oc4cc(-c6ccccc6)ccc45)c4cccc-3c24)cc1]

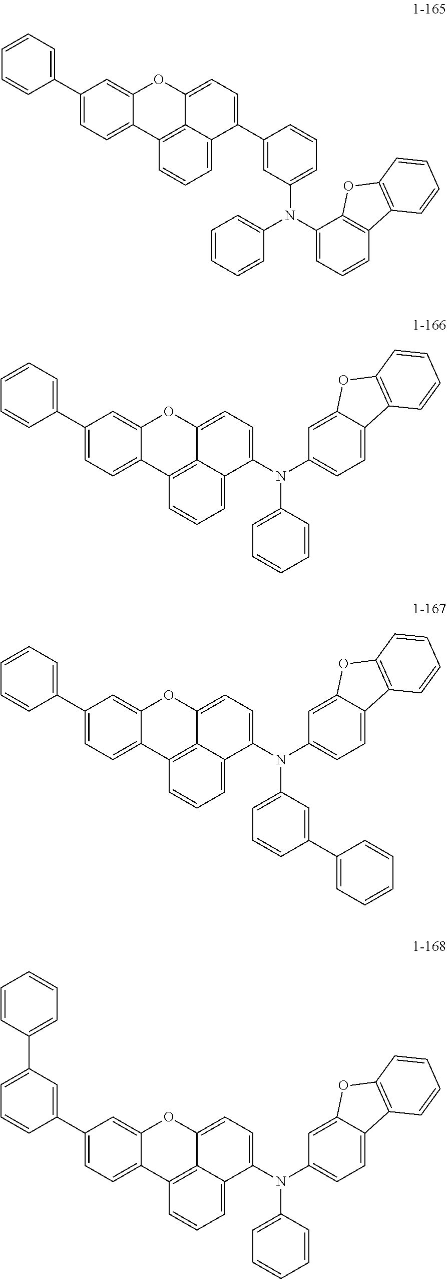 [SMILES: c1ccc(-c2ccc3c(c2)Oc2ccc(-c4cccc(N(c5ccccc5)c5cccc6c5oc5ccccc56)c4)c4cccc-3c24)cc1 ; c1ccc(-c2ccc3c(c2)Oc2ccc(N(c4ccccc4)c4ccc5c(c4)oc4ccccc45)c4cccc-3c24)cc1 ; c1ccc(-c2cccc(N(c3ccc4c(c3)oc3ccccc34)c3ccc4c5c(cccc35)-c3ccc(-c5ccccc5)cc3O4)c2)cc1 ; c1ccc(-c2cccc(-c3ccc4c(c3)Oc3ccc(N(c5ccccc5)c5ccc6c(c5)oc5ccccc56)c5cccc-4c35)c2)cc1]

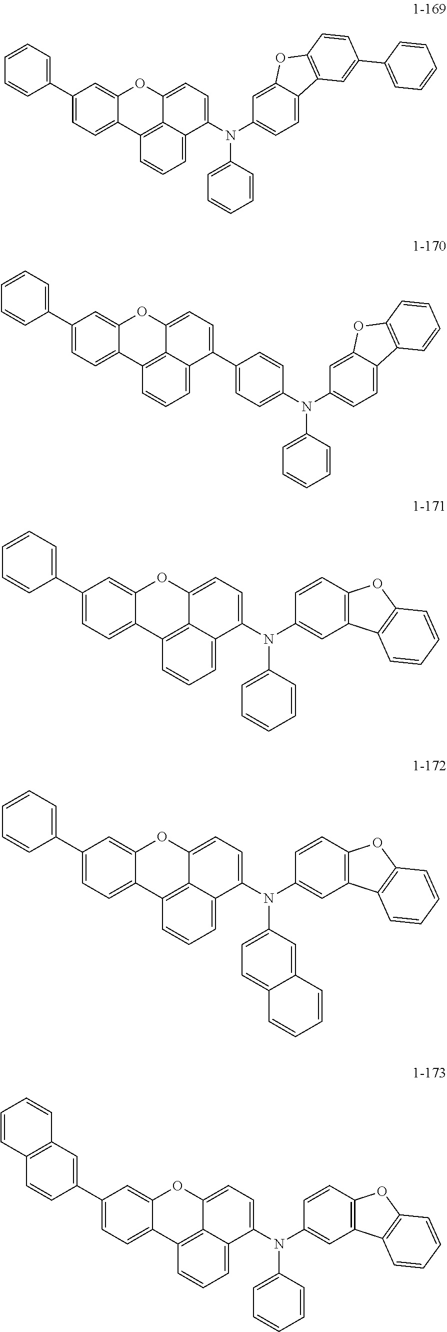 [SMILES: c1ccc(-c2ccc3c(c2)Oc2ccc(N(c4ccccc4)c4ccc5c(c4)oc4ccc(-c6ccccc6)cc45)c4cccc-3c24)cc1 ; c1ccc(-c2ccc3c(c2)Oc2ccc(-c4ccc(N(c5ccccc5)c5ccc6c(c5)oc5ccccc56)cc4)c4cccc-3c24)cc1 ; c1ccc(-c2ccc3c(c2)Oc2ccc(N(c4ccccc4)c4ccc5oc6ccccc6c5c4)c4cccc-3c24)cc1 ; c1ccc(-c2ccc3c(c2)Oc2ccc(N(c4ccc5ccccc5c4)c4ccc5oc6ccccc6c5c4)c4cccc-3c24)cc1 ; c1ccc(N(c2ccc3oc4ccccc4c3c2)c2ccc3c4c(cccc24)-c2ccc(-c4ccc5ccccc5c4)cc2O3)cc1]

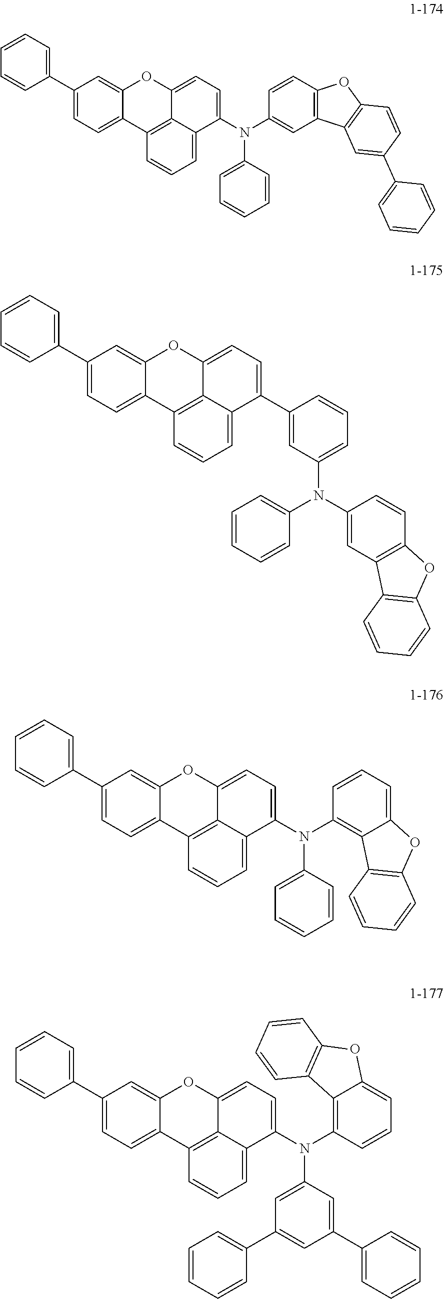 [SMILES: c1ccc(-c2ccc3c(c2)Oc2ccc(N(c4ccccc4)c4ccc5oc6ccc(-c7ccccc7)cc6c5c4)c4cccc-3c24)cc1 ; c1ccc(-c2ccc3c(c2)Oc2ccc(-c4cccc(N(c5ccccc5)c5ccc6oc7ccccc7c6c5)c4)c4cccc-3c24)cc1 ; c1ccc(-c2ccc3c(c2)Oc2ccc(N(c4ccccc4)c4cccc5oc6ccccc6c45)c4cccc-3c24)cc1 ; c1ccc(-c2cc(-c3ccccc3)cc(N(c3ccc4c5c(cccc35)-c3ccc(-c5ccccc5)cc3O4)c3cccc4oc5ccccc5c34)c2)cc1]

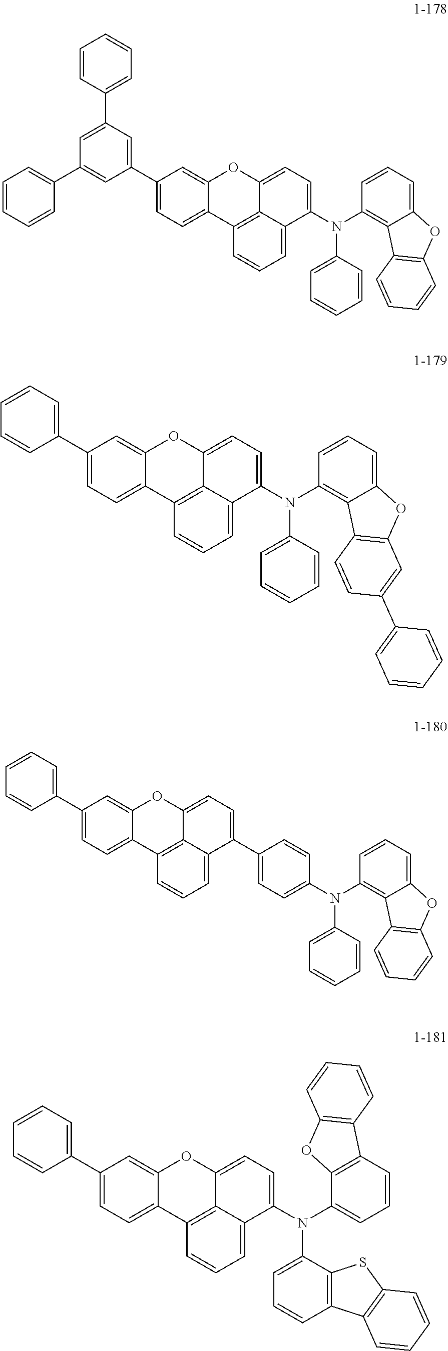 [SMILES: c1ccc(-c2cc(-c3ccccc3)cc(-c3ccc4c(c3)Oc3ccc(N(c5ccccc5)c5cccc6oc7ccccc7c56)c5cccc-4c35)c2)cc1 ; c1ccc(-c2ccc3c(c2)Oc2ccc(N(c4ccccc4)c4cccc5oc6cc(-c7ccccc7)ccc6c45)c4cccc-3c24)cc1 ; c1ccc(-c2ccc3c(c2)Oc2ccc(-c4ccc(N(c5ccccc5)c5cccc6oc7ccccc7c56)cc4)c4cccc-3c24)cc1 ; c1ccc(-c2ccc3c(c2)Oc2ccc(N(c4cccc5c4oc4ccccc45)c4cccc5c4sc4ccccc45)c4cccc-3c24)cc1]

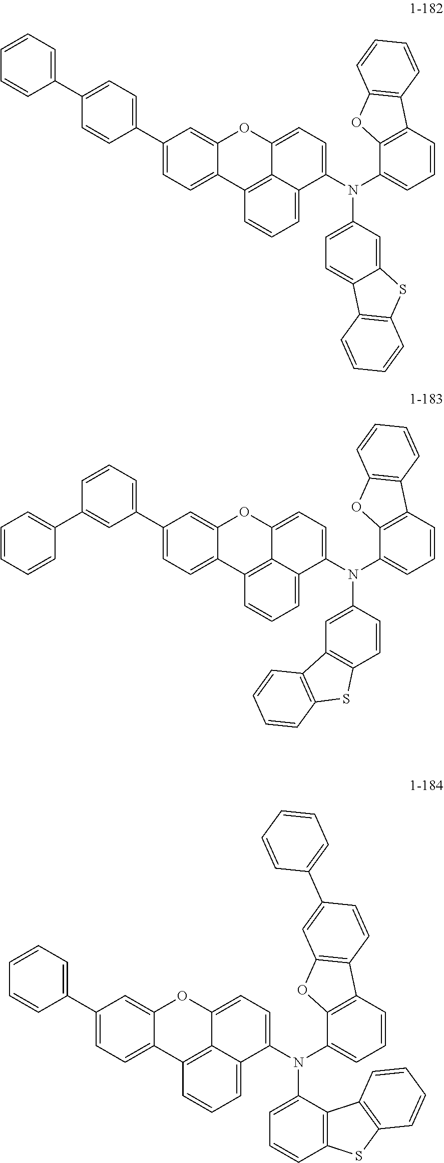 [SMILES: c1ccc(-c2ccc(-c3ccc4c(c3)Oc3ccc(N(c5ccc6c(c5)sc5ccccc56)c5cccc6c5oc5ccccc56)c5cccc-4c35)cc2)cc1 ; c1ccc(-c2cccc(-c3ccc4c(c3)Oc3ccc(N(c5ccc6sc7ccccc7c6c5)c5cccc6c5oc5ccccc56)c5cccc-4c35)c2)cc1 ; c1ccc(-c2ccc3c(c2)Oc2ccc(N(c4cccc5c4oc4cc(-c6ccccc6)ccc45)c4cccc5sc6ccccc6c45)c4cccc-3c24)cc1]

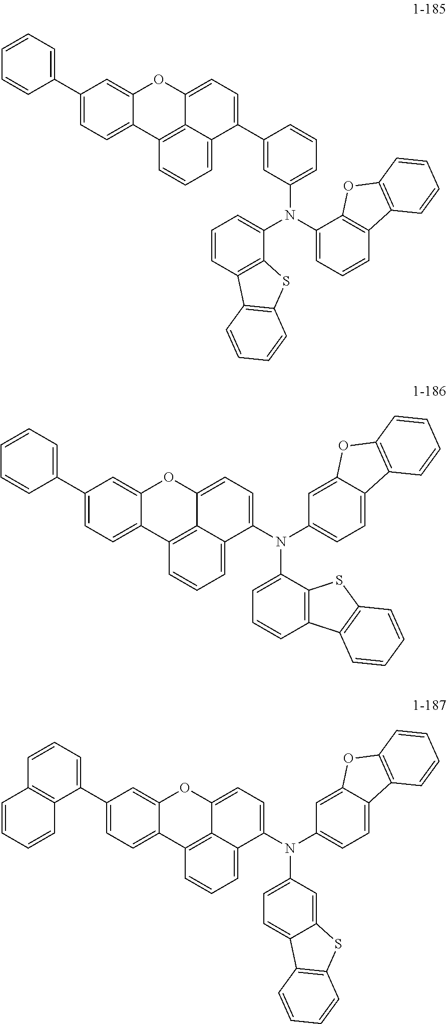 [SMILES: c1ccc(-c2ccc3c(c2)Oc2ccc(-c4cccc(N(c5cccc6c5oc5ccccc56)c5cccc6c5sc5ccccc56)c4)c4cccc-3c24)cc1 ; c1ccc(-c2ccc3c(c2)Oc2ccc(N(c4ccc5c(c4)oc4ccccc45)c4cccc5c4sc4ccccc45)c4cccc-3c24)cc1 ; c1ccc2c(-c3ccc4c(c3)Oc3ccc(N(c5ccc6c(c5)oc5ccccc56)c5ccc6c(c5)sc5ccccc56)c5cccc-4c35)cccc2c1]

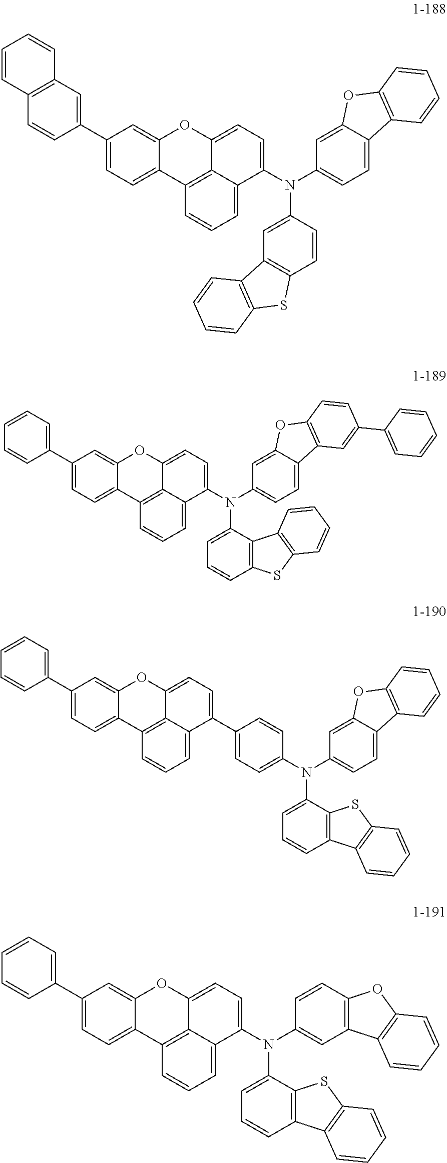 [SMILES: c1ccc2cc(-c3ccc4c(c3)Oc3ccc(N(c5ccc6c(c5)oc5ccccc56)c5ccc6sc7ccccc7c6c5)c5cccc-4c35)ccc2c1 ; c1ccc(-c2ccc3c(c2)Oc2ccc(N(c4ccc5c(c4)oc4ccc(-c6ccccc6)cc45)c4cccc5sc6ccccc6c45)c4cccc-3c24)cc1 ; c1ccc(-c2ccc3c(c2)Oc2ccc(-c4ccc(N(c5ccc6c(c5)oc5ccccc56)c5cccc6c5sc5ccccc56)cc4)c4cccc-3c24)cc1 ; c1ccc(-c2ccc3c(c2)Oc2ccc(N(c4ccc5oc6ccccc6c5c4)c4cccc5c4sc4ccccc45)c4cccc-3c24)cc1]

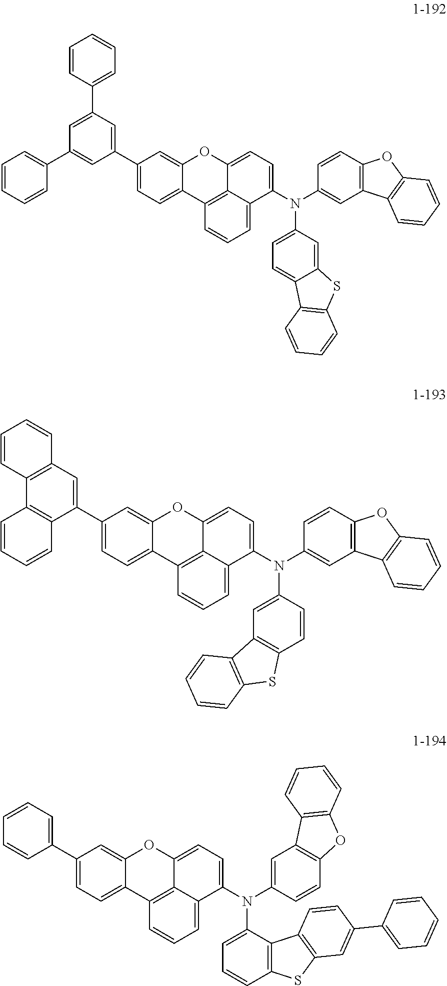 [SMILES: c1ccc(-c2cc(-c3ccccc3)cc(-c3ccc4c(c3)Oc3ccc(N(c5ccc6c(c5)sc5ccccc56)c5ccc6oc7ccccc7c6c5)c5cccc-4c35)c2)cc1 ; c1ccc2c(c1)cc(-c1ccc3c(c1)Oc1ccc(N(c4ccc5oc6ccccc6c5c4)c4ccc5sc6ccccc6c5c4)c4cccc-3c14)c1ccccc12 ; c1ccc(-c2ccc3c(c2)Oc2ccc(N(c4ccc5oc6ccccc6c5c4)c4cccc5sc6cc(-c7ccccc7)ccc6c45)c4cccc-3c24)cc1]

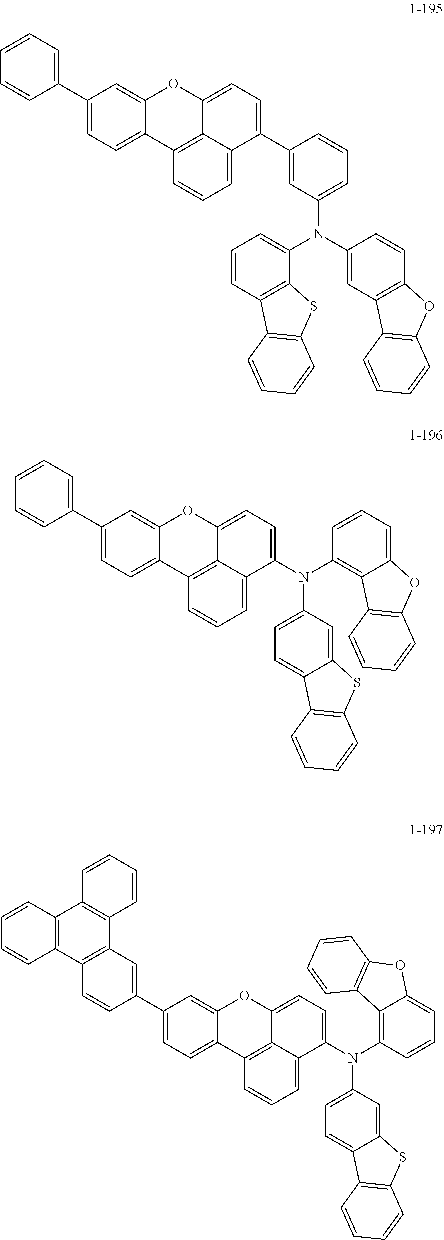 [SMILES: c1ccc(-c2ccc3c(c2)Oc2ccc(-c4cccc(N(c5ccc6oc7ccccc7c6c5)c5cccc6c5sc5ccccc56)c4)c4cccc-3c24)cc1 ; c1ccc(-c2ccc3c(c2)Oc2ccc(N(c4ccc5c(c4)sc4ccccc45)c4cccc5oc6ccccc6c45)c4cccc-3c24)cc1 ; c1ccc2c(c1)oc1cccc(N(c3ccc4c(c3)sc3ccccc34)c3ccc4c5c(cccc35)-c3ccc(-c5ccc6c7ccccc7c7ccccc7c6c5)cc3O4)c12]

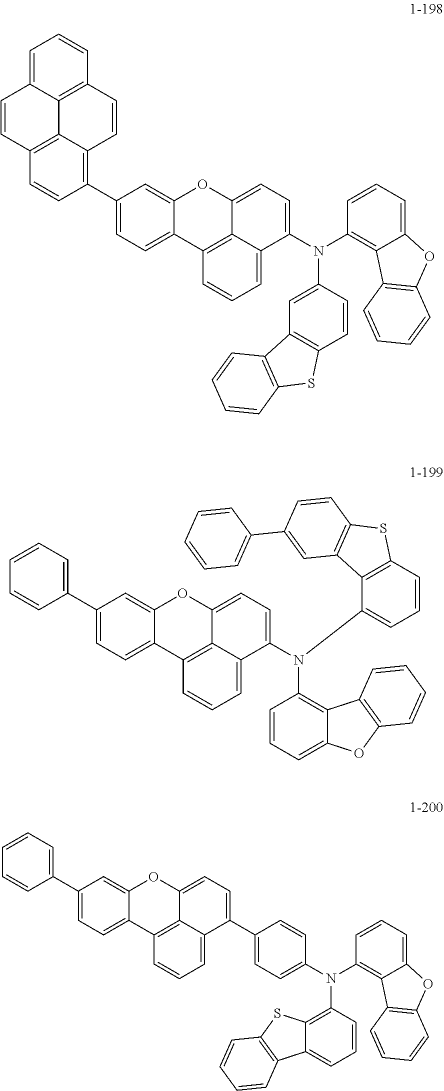 [SMILES: c1cc2ccc3ccc(-c4ccc5c(c4)Oc4ccc(N(c6ccc7sc8ccccc8c7c6)c6cccc7oc8ccccc8c67)c6cccc-5c46)c4ccc(c1)c2c34 ; c1ccc(-c2ccc3c(c2)Oc2ccc(N(c4cccc5oc6ccccc6c45)c4cccc5sc6ccc(-c7ccccc7)cc6c45)c4cccc-3c24)cc1 ; c1ccc(-c2ccc3c(c2)Oc2ccc(-c4ccc(N(c5cccc6c5sc5ccccc56)c5cccc6oc7ccccc7c56)cc4)c4cccc-3c24)cc1]

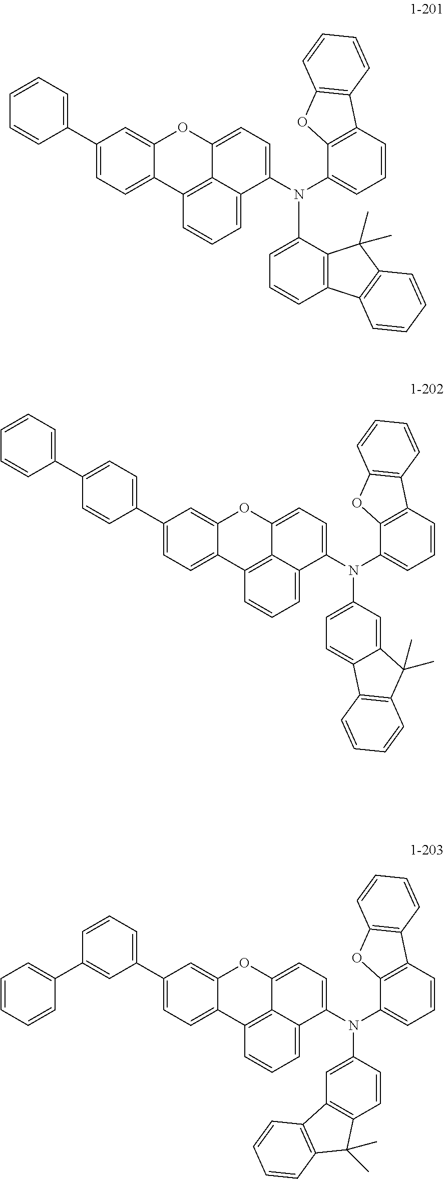 [SMILES: CC1(C)c2ccccc2-c2cccc(N(c3ccc4c5c(cccc35)-c3ccc(-c5ccccc5)cc3O4)c3cccc4c3oc3ccccc34)c21 ; CC1(C)c2ccccc2-c2ccc(N(c3ccc4c5c(cccc35)-c3ccc(-c5ccc(-c6ccccc6)cc5)cc3O4)c3cccc4c3oc3ccccc34)cc21 ; CC1(C)c2ccccc2-c2cc(N(c3ccc4c5c(cccc35)-c3ccc(-c5cccc(-c6ccccc6)c5)cc3O4)c3cccc4c3oc3ccccc34)ccc21]

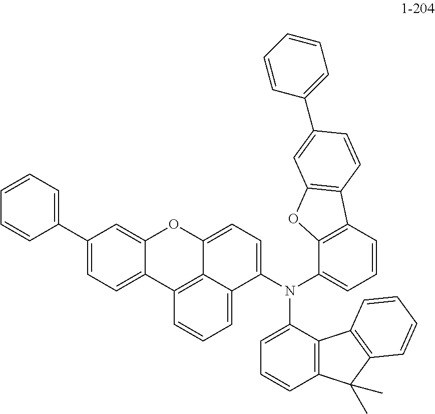 [SMILES: CC1(C)c2ccccc2-c2c(N(c3ccc4c5c(cccc35)-c3ccc(-c5ccccc5)cc3O4)c3cccc4c3oc3cc(-c5ccccc5)ccc34)cccc21]

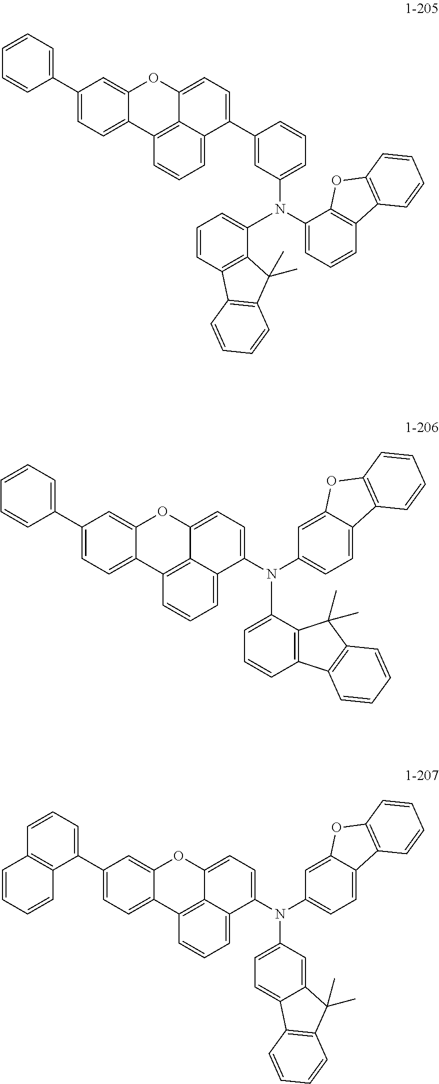 [SMILES: CC1(C)c2ccccc2-c2cccc(N(c3cccc(-c4ccc5c6c(cccc46)-c4ccc(-c6ccccc6)cc4O5)c3)c3cccc4c3oc3ccccc34)c21 ; CC1(C)c2ccccc2-c2cccc(N(c3ccc4c(c3)oc3ccccc34)c3ccc4c5c(cccc35)-c3ccc(-c5ccccc5)cc3O4)c21 ; CC1(C)c2ccccc2-c2ccc(N(c3ccc4c(c3)oc3ccccc34)c3ccc4c5c(cccc35)-c3ccc(-c5cccc6ccccc56)cc3O4)cc21]

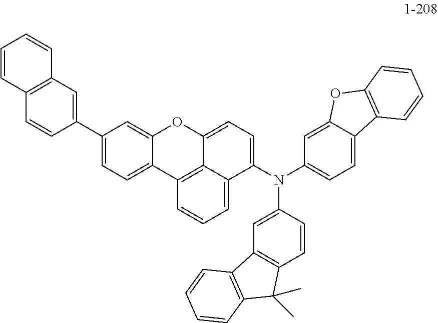 [SMILES: CC1(C)c2ccccc2-c2cc(N(c3ccc4c(c3)oc3ccccc34)c3ccc4c5c(cccc35)-c3ccc(-c5ccc6ccccc6c5)cc3O4)ccc21]

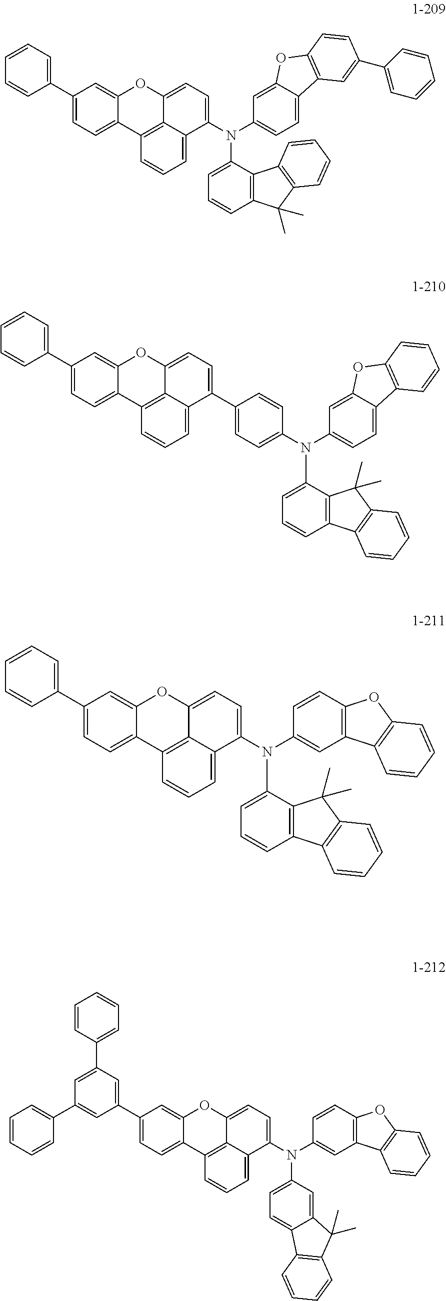 [SMILES: CC1(C)c2ccccc2-c2c(N(c3ccc4c(c3)oc3ccc(-c5ccccc5)cc34)c3ccc4c5c(cccc35)-c3ccc(-c5ccccc5)cc3O4)cccc21 ; CC1(C)c2ccccc2-c2cccc(N(c3ccc(-c4ccc5c6c(cccc46)-c4ccc(-c6ccccc6)cc4O5)cc3)c3ccc4c(c3)oc3ccccc34)c21 ; CC1(C)c2ccccc2-c2cccc(N(c3ccc4oc5ccccc5c4c3)c3ccc4c5c(cccc35)-c3ccc(-c5ccccc5)cc3O4)c21 ; CC1(C)c2ccccc2-c2ccc(N(c3ccc4oc5ccccc5c4c3)c3ccc4c5c(cccc35)-c3ccc(-c5cc(-c6ccccc6)cc(-c6ccccc6)c5)cc3O4)cc21]

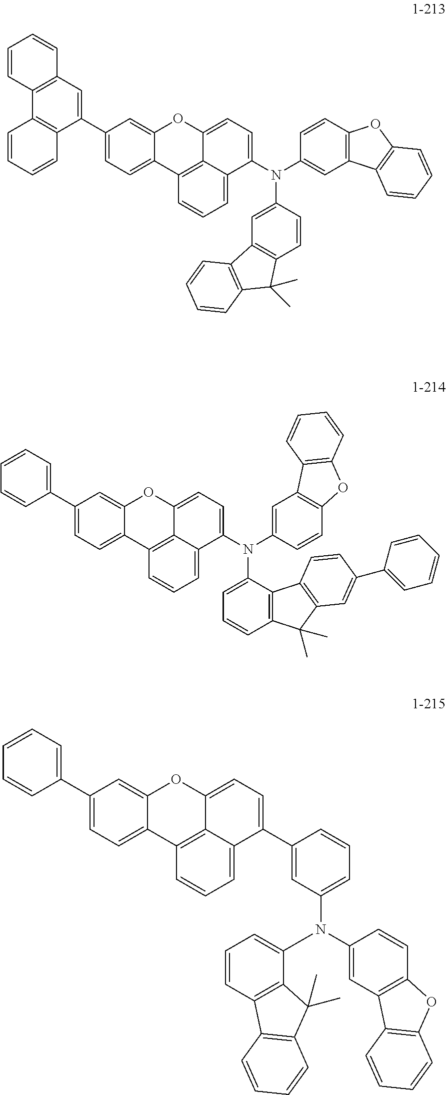 [SMILES: CC1(C)c2ccccc2-c2cc(N(c3ccc4oc5ccccc5c4c3)c3ccc4c5c(cccc35)-c3ccc(-c5cc6ccccc6c6ccccc56)cc3O4)ccc21 ; CC1(C)c2cc(-c3ccccc3)ccc2-c2c(N(c3ccc4oc5ccccc5c4c3)c3ccc4c5c(cccc35)-c3ccc(-c5ccccc5)cc3O4)cccc21 ; CC1(C)c2ccccc2-c2cccc(N(c3cccc(-c4ccc5c6c(cccc46)-c4ccc(-c6ccccc6)cc4O5)c3)c3ccc4oc5ccccc5c4c3)c21]

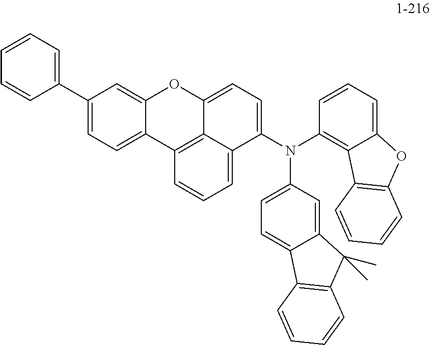 [SMILES: CC1(C)c2ccccc2-c2ccc(N(c3ccc4c5c(cccc35)-c3ccc(-c5ccccc5)cc3O4)c3cccc4oc5ccccc5c34)cc21]

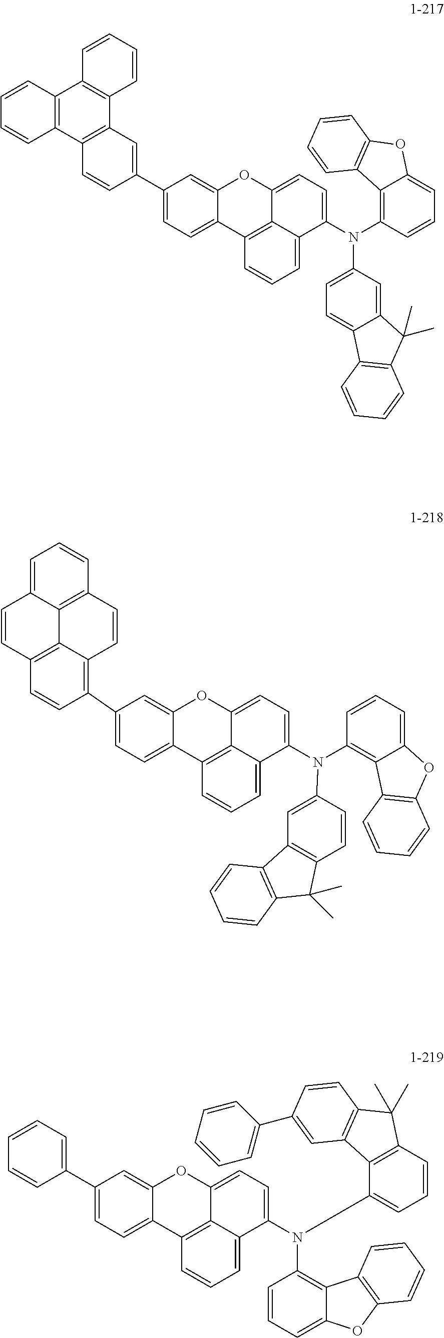 [SMILES: CC1(C)c2ccccc2-c2ccc(N(c3ccc4c5c(cccc35)-c3ccc(-c5ccc6c7ccccc7c7ccccc7c6c5)cc3O4)c3cccc4oc5ccccc5c34)cc21 ; CC1(C)c2ccccc2-c2cc(N(c3ccc4c5c(cccc35)-c3ccc(-c5ccc6ccc7cccc8ccc5c6c78)cc3O4)c3cccc4oc5ccccc5c34)ccc21 ; CC1(C)c2ccc(-c3ccccc3)cc2-c2c(N(c3ccc4c5c(cccc35)-c3ccc(-c5ccccc5)cc3O4)c3cccc4oc5ccccc5c34)cccc21]

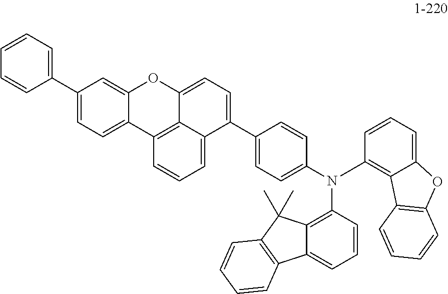 [SMILES: CC1(C)c2ccccc2-c2cccc(N(c3ccc(-c4ccc5c6c(cccc46)-c4ccc(-c6ccccc6)cc4O5)cc3)c3cccc4oc5ccccc5c34)c21]

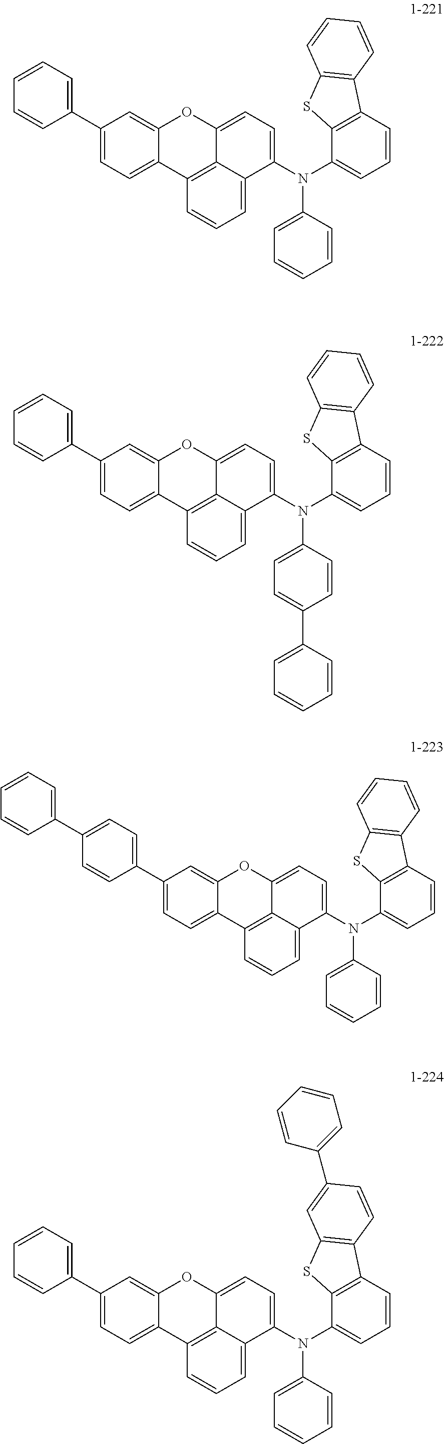 [SMILES: c1ccc(-c2ccc3c(c2)Oc2ccc(N(c4ccccc4)c4cccc5c4sc4ccccc45)c4cccc-3c24)cc1 ; c1ccc(-c2ccc(N(c3ccc4c5c(cccc35)-c3ccc(-c5ccccc5)cc3O4)c3cccc4c3sc3ccccc34)cc2)cc1 ; c1ccc(-c2ccc(-c3ccc4c(c3)Oc3ccc(N(c5ccccc5)c5cccc6c5sc5ccccc56)c5cccc-4c35)cc2)cc1 ; c1ccc(-c2ccc3c(c2)Oc2ccc(N(c4ccccc4)c4cccc5c4sc4cc(-c6ccccc6)ccc45)c4cccc-3c24)cc1]

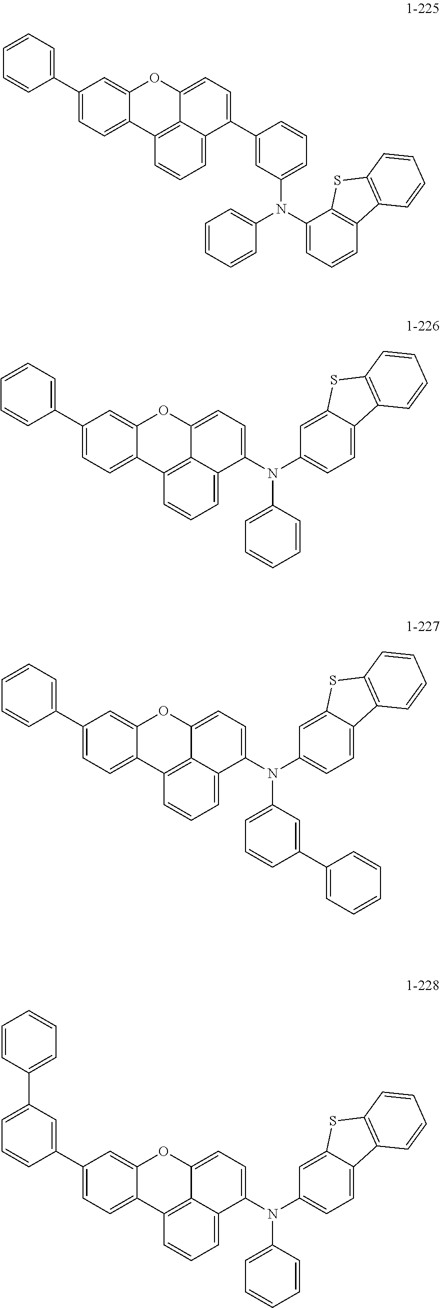 [SMILES: c1ccc(-c2ccc3c(c2)Oc2ccc(-c4cccc(N(c5ccccc5)c5cccc6c5sc5ccccc56)c4)c4cccc-3c24)cc1 ; c1ccc(-c2ccc3c(c2)Oc2ccc(N(c4ccccc4)c4ccc5c(c4)sc4ccccc45)c4cccc-3c24)cc1 ; c1ccc(-c2cccc(N(c3ccc4c(c3)sc3ccccc34)c3ccc4c5c(cccc35)-c3ccc(-c5ccccc5)cc3O4)c2)cc1 ; c1ccc(-c2cccc(-c3ccc4c(c3)Oc3ccc(N(c5ccccc5)c5ccc6c(c5)sc5ccccc56)c5cccc-4c35)c2)cc1]

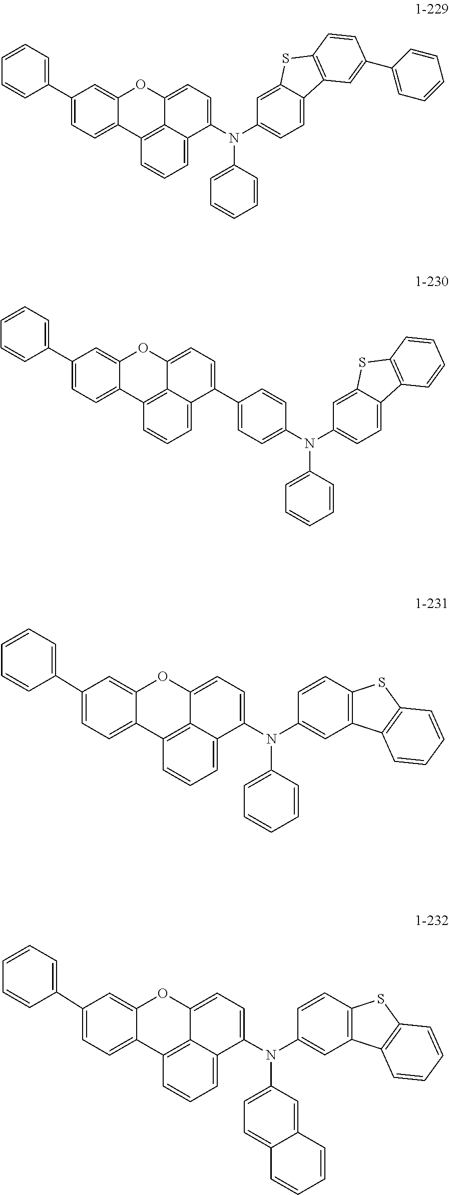 [SMILES: c1ccc(-c2ccc3c(c2)Oc2ccc(N(c4ccccc4)c4ccc5c(c4)sc4ccc(-c6ccccc6)cc45)c4cccc-3c24)cc1 ; c1ccc(-c2ccc3c(c2)Oc2ccc(-c4ccc(N(c5ccccc5)c5ccc6c(c5)sc5ccccc56)cc4)c4cccc-3c24)cc1 ; c1ccc(-c2ccc3c(c2)Oc2ccc(N(c4ccccc4)c4ccc5sc6ccccc6c5c4)c4cccc-3c24)cc1 ; c1ccc(-c2ccc3c(c2)Oc2ccc(N(c4ccc5ccccc5c4)c4ccc5sc6ccccc6c5c4)c4cccc-3c24)cc1]

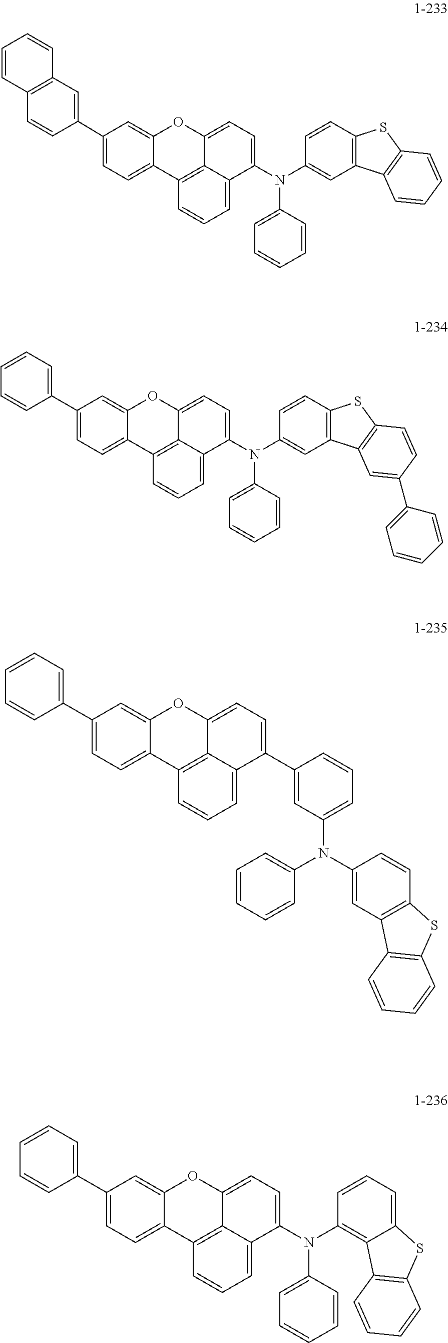 [SMILES: c1ccc(N(c2ccc3sc4ccccc4c3c2)c2ccc3c4c(cccc24)-c2ccc(-c4ccc5ccccc5c4)cc2O3)cc1 ; c1ccc(-c2ccc3c(c2)Oc2ccc(N(c4ccccc4)c4ccc5sc6ccc(-c7ccccc7)cc6c5c4)c4cccc-3c24)cc1 ; c1ccc(-c2ccc3c(c2)Oc2ccc(-c4cccc(N(c5ccccc5)c5ccc6sc7ccccc7c6c5)c4)c4cccc-3c24)cc1 ; c1ccc(-c2ccc3c(c2)Oc2ccc(N(c4ccccc4)c4cccc5sc6ccccc6c45)c4cccc-3c24)cc1]

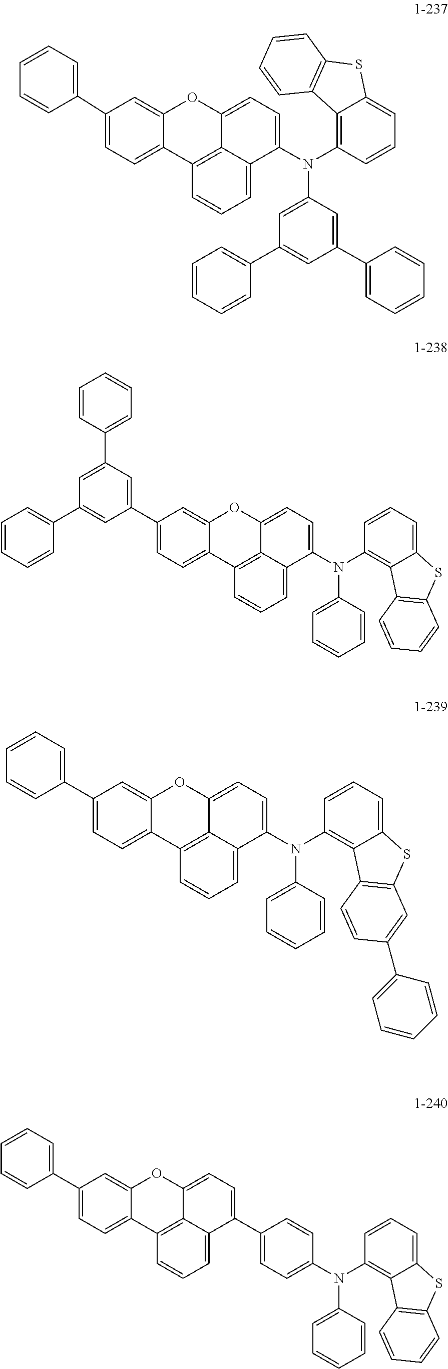 [SMILES: c1ccc(-c2cc(-c3ccccc3)cc(N(c3ccc4c5c(cccc35)-c3ccc(-c5ccccc5)cc3O4)c3cccc4sc5ccccc5c34)c2)cc1 ; c1ccc(-c2cc(-c3ccccc3)cc(-c3ccc4c(c3)Oc3ccc(N(c5ccccc5)c5cccc6sc7ccccc7c56)c5cccc-4c35)c2)cc1 ; c1ccc(-c2ccc3c(c2)Oc2ccc(N(c4ccccc4)c4cccc5sc6cc(-c7ccccc7)ccc6c45)c4cccc-3c24)cc1 ; c1ccc(-c2ccc3c(c2)Oc2ccc(-c4ccc(N(c5ccccc5)c5cccc6sc7ccccc7c56)cc4)c4cccc-3c24)cc1]

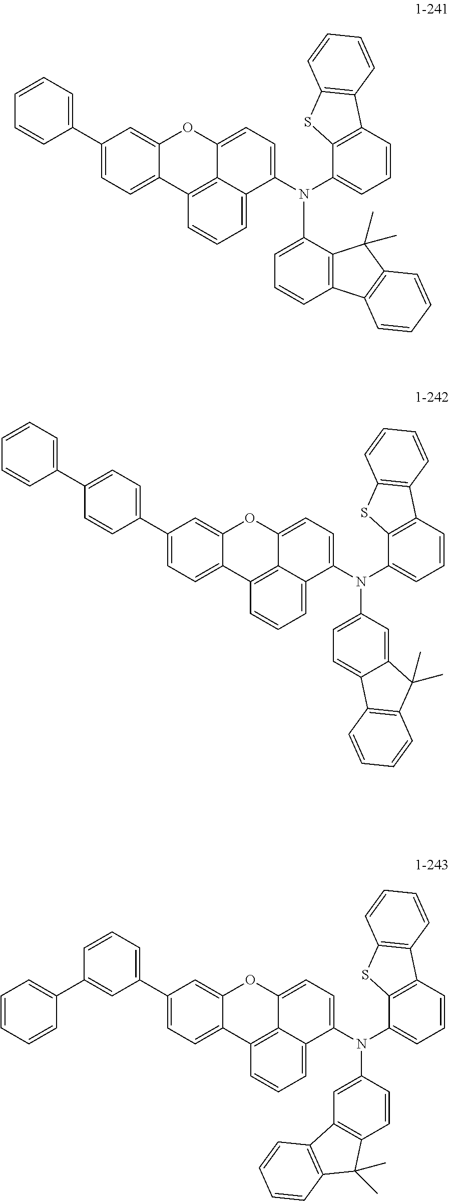 [SMILES: CC1(C)c2ccccc2-c2cccc(N(c3ccc4c5c(cccc35)-c3ccc(-c5ccccc5)cc3O4)c3cccc4c3sc3ccccc34)c21 ; CC1(C)c2ccccc2-c2ccc(N(c3ccc4c5c(cccc35)-c3ccc(-c5ccc(-c6ccccc6)cc5)cc3O4)c3cccc4c3sc3ccccc34)cc21 ; CC1(C)c2ccccc2-c2cc(N(c3ccc4c5c(cccc35)-c3ccc(-c5cccc(-c6ccccc6)c5)cc3O4)c3cccc4c3sc3ccccc34)ccc21]

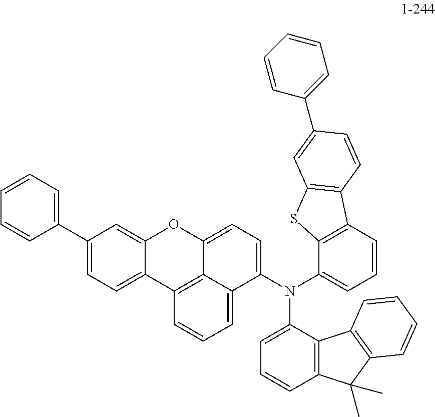 [SMILES: CC1(C)c2ccccc2-c2c(N(c3ccc4c5c(cccc35)-c3ccc(-c5ccccc5)cc3O4)c3cccc4c3sc3cc(-c5ccccc5)ccc34)cccc21]

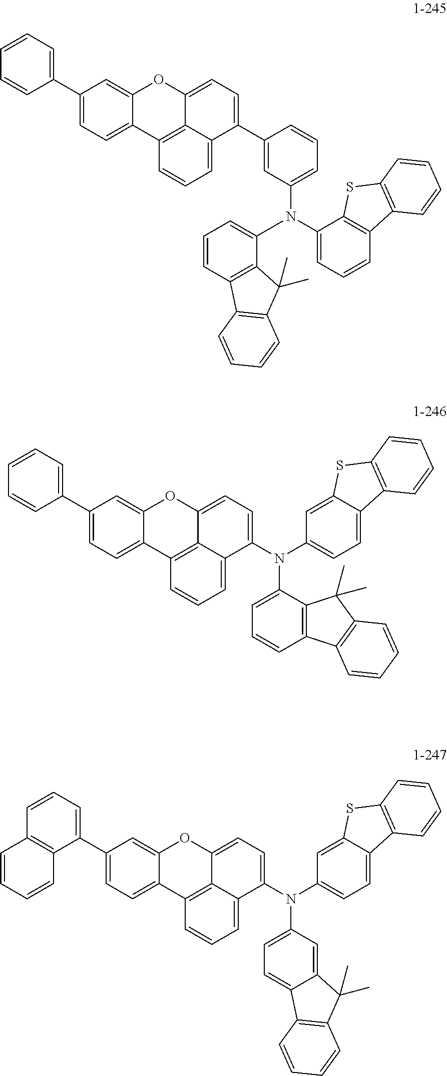 [SMILES: CC1(C)c2ccccc2-c2cccc(N(c3cccc(-c4ccc5c6c(cccc46)-c4ccc(-c6ccccc6)cc4O5)c3)c3cccc4c3sc3ccccc34)c21 ; CC1(C)c2ccccc2-c2cccc(N(c3ccc4c(c3)sc3ccccc34)c3ccc4c5c(cccc35)-c3ccc(-c5ccccc5)cc3O4)c21 ; CC1(C)c2ccccc2-c2ccc(N(c3ccc4c(c3)sc3ccccc34)c3ccc4c5c(cccc35)-c3ccc(-c5cccc6ccccc56)cc3O4)cc21]

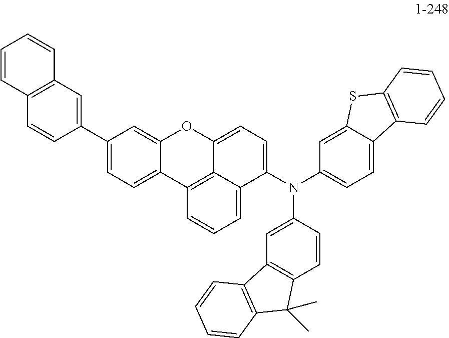 [SMILES: CC1(C)c2ccccc2-c2cc(N(c3ccc4c(c3)sc3ccccc34)c3ccc4c5c(cccc35)-c3ccc(-c5ccc6ccccc6c5)cc3O4)ccc21]

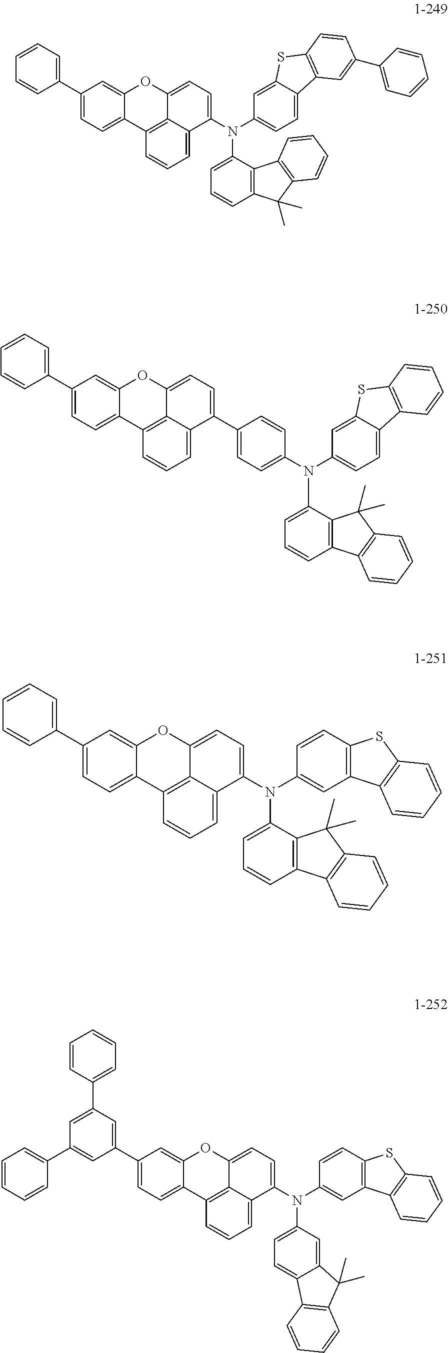 [SMILES: CC1(C)c2ccccc2-c2c(N(c3ccc4c(c3)sc3ccc(-c5ccccc5)cc34)c3ccc4c5c(cccc35)-c3ccc(-c5ccccc5)cc3O4)cccc21 ; CC1(C)c2ccccc2-c2cccc(N(c3ccc(-c4ccc5c6c(cccc46)-c4ccc(-c6ccccc6)cc4O5)cc3)c3ccc4c(c3)sc3ccccc34)c21 ; CC1(C)c2ccccc2-c2cccc(N(c3ccc4sc5ccccc5c4c3)c3ccc4c5c(cccc35)-c3ccc(-c5ccccc5)cc3O4)c21 ; CC1(C)c2ccccc2-c2ccc(N(c3ccc4sc5ccccc5c4c3)c3ccc4c5c(cccc35)-c3ccc(-c5cc(-c6ccccc6)cc(-c6ccccc6)c5)cc3O4)cc21]

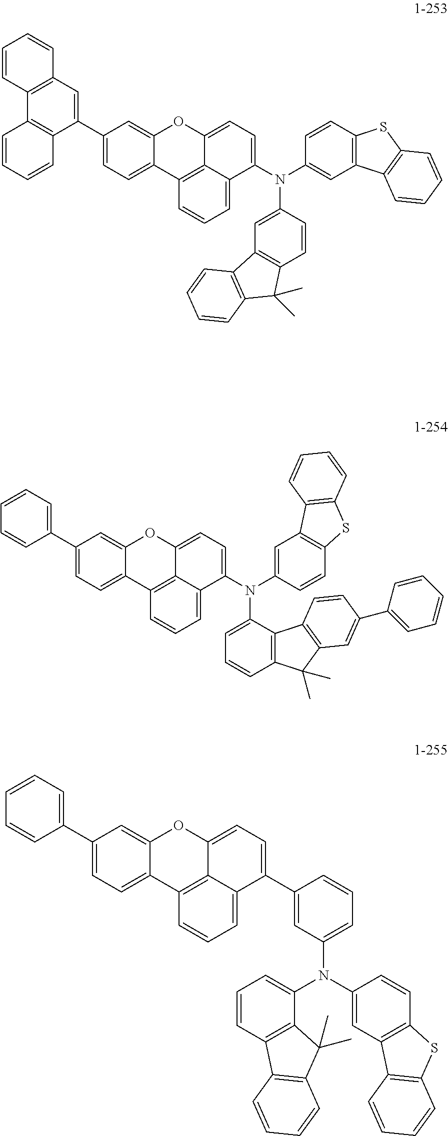 [SMILES: CC1(C)c2ccccc2-c2cc(N(c3ccc4sc5ccccc5c4c3)c3ccc4c5c(cccc35)-c3ccc(-c5cc6ccccc6c6ccccc56)cc3O4)ccc21 ; CC1(C)c2cc(-c3ccccc3)ccc2-c2c(N(c3ccc4sc5ccccc5c4c3)c3ccc4c5c(cccc35)-c3ccc(-c5ccccc5)cc3O4)cccc21 ; CC1(C)c2ccccc2-c2cccc(N(c3cccc(-c4ccc5c6c(cccc46)-c4ccc(-c6ccccc6)cc4O5)c3)c3ccc4sc5ccccc5c4c3)c21]

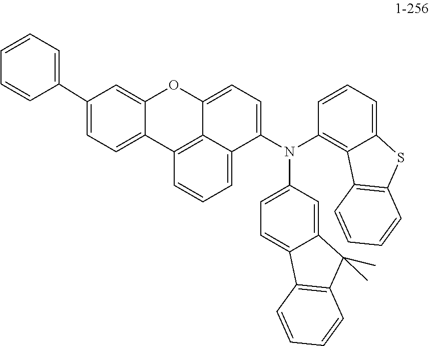 [SMILES: CC1(C)c2ccccc2-c2ccc(N(c3ccc4c5c(cccc35)-c3ccc(-c5ccccc5)cc3O4)c3cccc4sc5ccccc5c34)cc21]

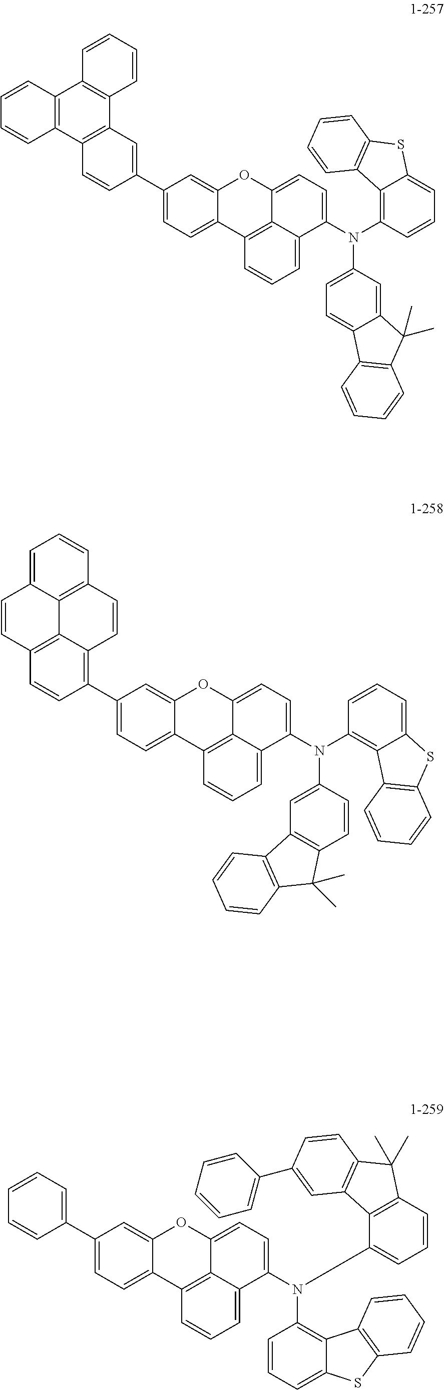 [SMILES: CC1(C)c2ccccc2-c2ccc(N(c3ccc4c5c(cccc35)-c3ccc(-c5ccc6c7ccccc7c7ccccc7c6c5)cc3O4)c3cccc4sc5ccccc5c34)cc21 ; CC1(C)c2ccccc2-c2cc(N(c3ccc4c5c(cccc35)-c3ccc(-c5ccc6ccc7cccc8ccc5c6c78)cc3O4)c3cccc4sc5ccccc5c34)ccc21 ; CC1(C)c2ccc(-c3ccccc3)cc2-c2c(N(c3ccc4c5c(cccc35)-c3ccc(-c5ccccc5)cc3O4)c3cccc4sc5ccccc5c34)cccc21]

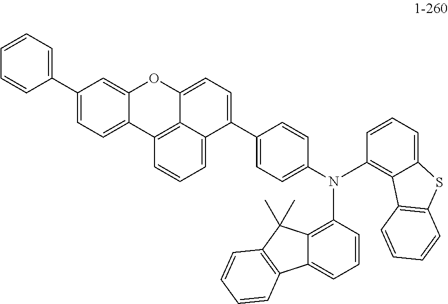 [SMILES: CC1(C)c2ccccc2-c2cccc(N(c3ccc(-c4ccc5c6c(cccc46)-c4ccc(-c6ccccc6)cc4O5)cc3)c3cccc4sc5ccccc5c34)c21]

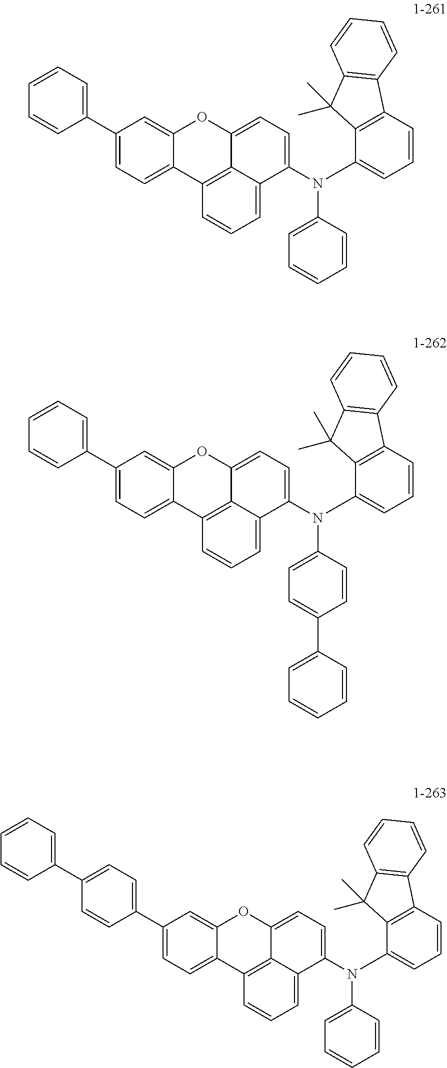 [SMILES: CC1(C)c2ccccc2-c2cccc(N(c3ccccc3)c3ccc4c5c(cccc35)-c3ccc(-c5ccccc5)cc3O4)c21 ; CC1(C)c2ccccc2-c2cccc(N(c3ccc(-c4ccccc4)cc3)c3ccc4c5c(cccc35)-c3ccc(-c5ccccc5)cc3O4)c21 ; CC1(C)c2ccccc2-c2cccc(N(c3ccccc3)c3ccc4c5c(cccc35)-c3ccc(-c5ccc(-c6ccccc6)cc5)cc3O4)c21]

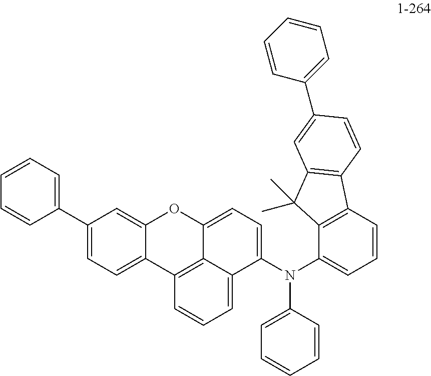 [SMILES: CC1(C)c2cc(-c3ccccc3)ccc2-c2cccc(N(c3ccccc3)c3ccc4c5c(cccc35)-c3ccc(-c5ccccc5)cc3O4)c21]

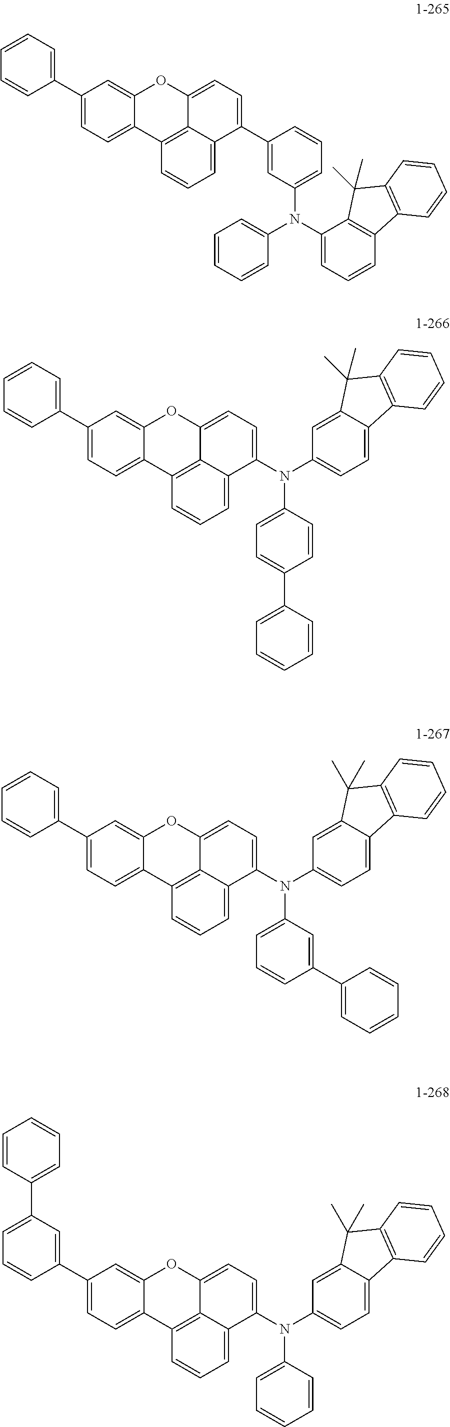 [SMILES: CC1(C)c2ccccc2-c2cccc(N(c3ccccc3)c3cccc(-c4ccc5c6c(cccc46)-c4ccc(-c6ccccc6)cc4O5)c3)c21 ; CC1(C)c2ccccc2-c2ccc(N(c3ccc(-c4ccccc4)cc3)c3ccc4c5c(cccc35)-c3ccc(-c5ccccc5)cc3O4)cc21 ; CC1(C)c2ccccc2-c2ccc(N(c3cccc(-c4ccccc4)c3)c3ccc4c5c(cccc35)-c3ccc(-c5ccccc5)cc3O4)cc21 ; CC1(C)c2ccccc2-c2ccc(N(c3ccccc3)c3ccc4c5c(cccc35)-c3ccc(-c5cccc(-c6ccccc6)c5)cc3O4)cc21]

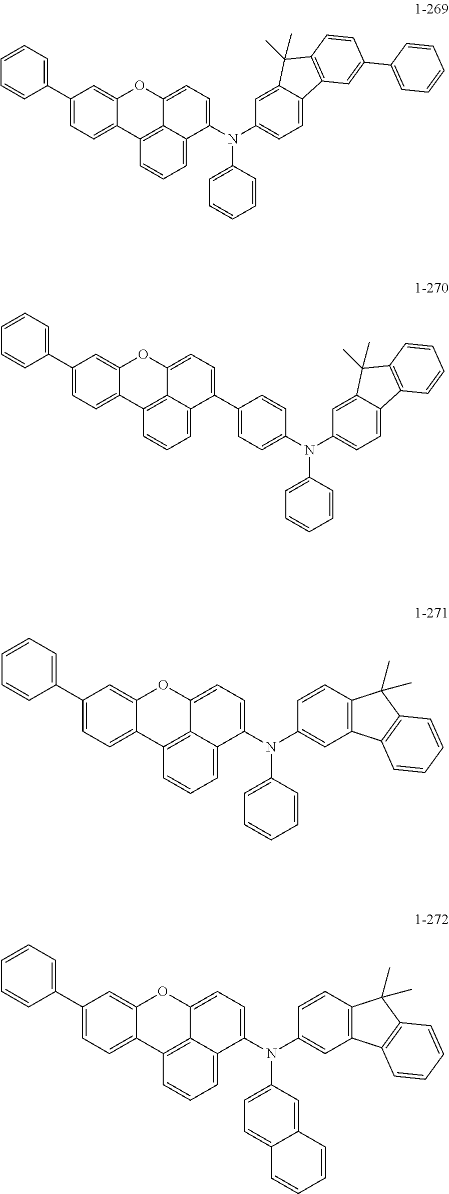 [SMILES: CC1(C)c2ccc(-c3ccccc3)cc2-c2ccc(N(c3ccccc3)c3ccc4c5c(cccc35)-c3ccc(-c5ccccc5)cc3O4)cc21 ; CC1(C)c2ccccc2-c2ccc(N(c3ccccc3)c3ccc(-c4ccc5c6c(cccc46)-c4ccc(-c6ccccc6)cc4O5)cc3)cc21 ; CC1(C)c2ccccc2-c2cc(N(c3ccccc3)c3ccc4c5c(cccc35)-c3ccc(-c5ccccc5)cc3O4)ccc21 ; CC1(C)c2ccccc2-c2cc(N(c3ccc4ccccc4c3)c3ccc4c5c(cccc35)-c3ccc(-c5ccccc5)cc3O4)ccc21]

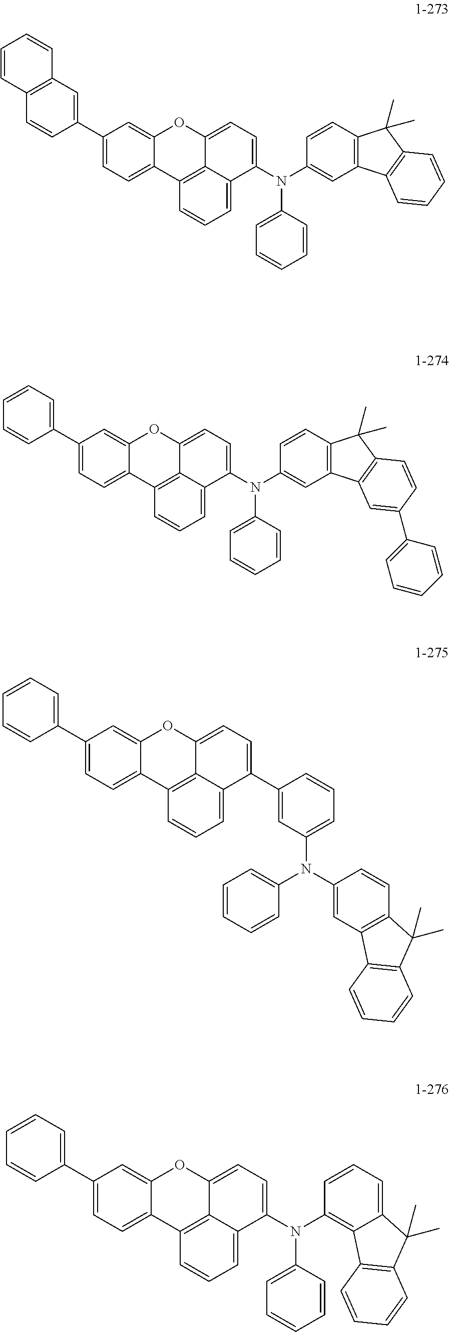 [SMILES: CC1(C)c2ccccc2-c2cc(N(c3ccccc3)c3ccc4c5c(cccc35)-c3ccc(-c5ccc6ccccc6c5)cc3O4)ccc21 ; CC1(C)c2ccc(-c3ccccc3)cc2-c2cc(N(c3ccccc3)c3ccc4c5c(cccc35)-c3ccc(-c5ccccc5)cc3O4)ccc21 ; CC1(C)c2ccccc2-c2cc(N(c3ccccc3)c3cccc(-c4ccc5c6c(cccc46)-c4ccc(-c6ccccc6)cc4O5)c3)ccc21 ; CC1(C)c2ccccc2-c2c(N(c3ccccc3)c3ccc4c5c(cccc35)-c3ccc(-c5ccccc5)cc3O4)cccc21]

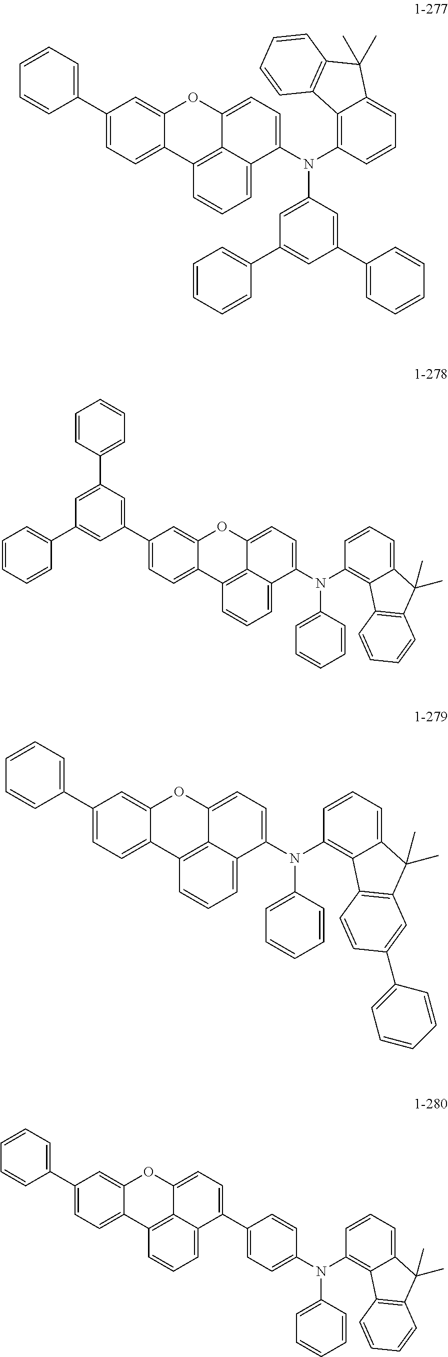 [SMILES: CC1(C)c2ccccc2-c2c(N(c3cc(-c4ccccc4)cc(-c4ccccc4)c3)c3ccc4c5c(cccc35)-c3ccc(-c5ccccc5)cc3O4)cccc21 ; CC1(C)c2ccccc2-c2c(N(c3ccccc3)c3ccc4c5c(cccc35)-c3ccc(-c5cc(-c6ccccc6)cc(-c6ccccc6)c5)cc3O4)cccc21 ; CC1(C)c2cc(-c3ccccc3)ccc2-c2c(N(c3ccccc3)c3ccc4c5c(cccc35)-c3ccc(-c5ccccc5)cc3O4)cccc21 ; CC1(C)c2ccccc2-c2c(N(c3ccccc3)c3ccc(-c4ccc5c6c(cccc46)-c4ccc(-c6ccccc6)cc4O5)cc3)cccc21]

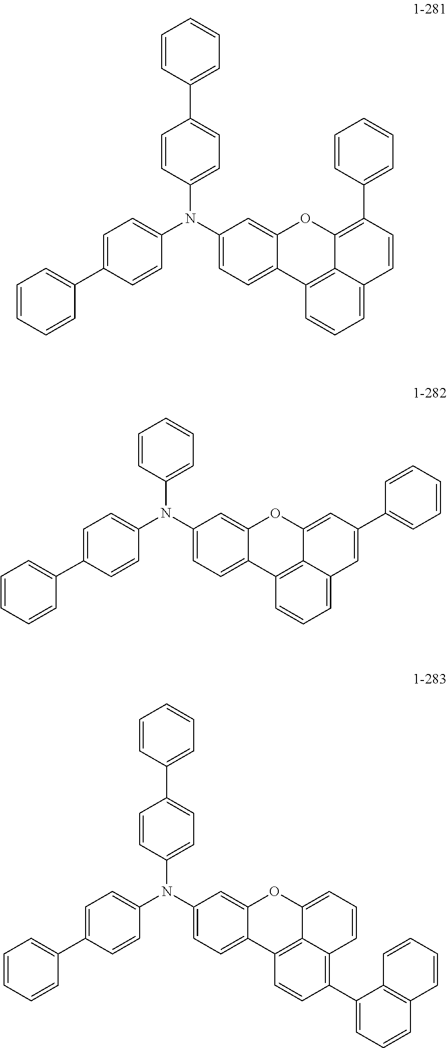 [SMILES: c1ccc(-c2ccc(N(c3ccc(-c4ccccc4)cc3)c3ccc4c(c3)Oc3c(-c5ccccc5)ccc5cccc-4c35)cc2)cc1 ; c1ccc(-c2ccc(N(c3ccccc3)c3ccc4c(c3)Oc3cc(-c5ccccc5)cc5cccc-4c35)cc2)cc1 ; c1ccc(-c2ccc(N(c3ccc(-c4ccccc4)cc3)c3ccc4c(c3)Oc3cccc5c(-c6cccc7ccccc67)ccc-4c35)cc2)cc1]

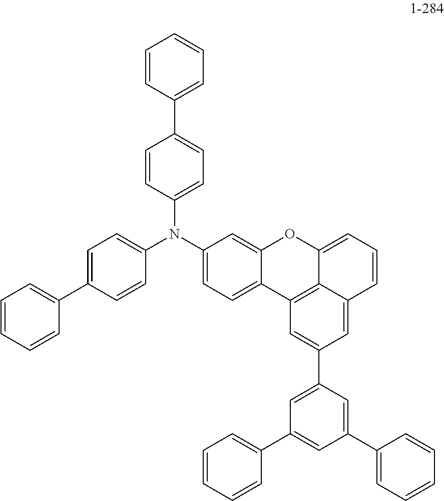 [SMILES: c1ccc(-c2ccc(N(c3ccc(-c4ccccc4)cc3)c3ccc4c(c3)Oc3cccc5cc(-c6cc(-c7ccccc7)cc(-c7ccccc7)c6)cc-4c35)cc2)cc1]

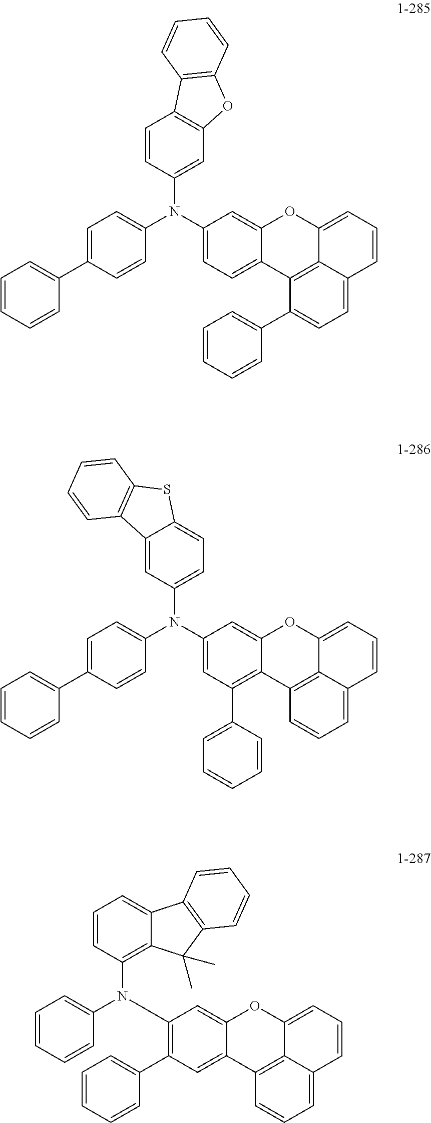 [SMILES: c1ccc(-c2ccc(N(c3ccc4c(c3)Oc3cccc5ccc(-c6ccccc6)c-4c35)c3ccc4c(c3)oc3ccccc34)cc2)cc1 ; c1ccc(-c2ccc(N(c3cc4c(c(-c5ccccc5)c3)-c3cccc5cccc(c35)O4)c3ccc4sc5ccccc5c4c3)cc2)cc1 ; CC1(C)c2ccccc2-c2cccc(N(c3ccccc3)c3cc4c(cc3-c3ccccc3)-c3cccc5cccc(c35)O4)c21]

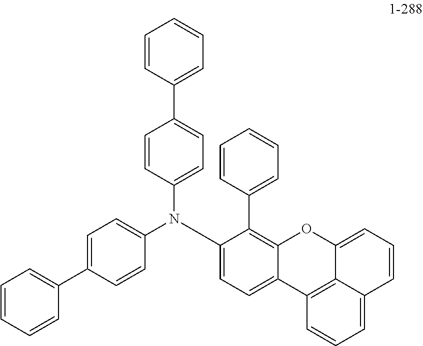 [SMILES: c1ccc(-c2ccc(N(c3ccc(-c4ccccc4)cc3)c3ccc4c(c3-c3ccccc3)Oc3cccc5cccc-4c35)cc2)cc1]

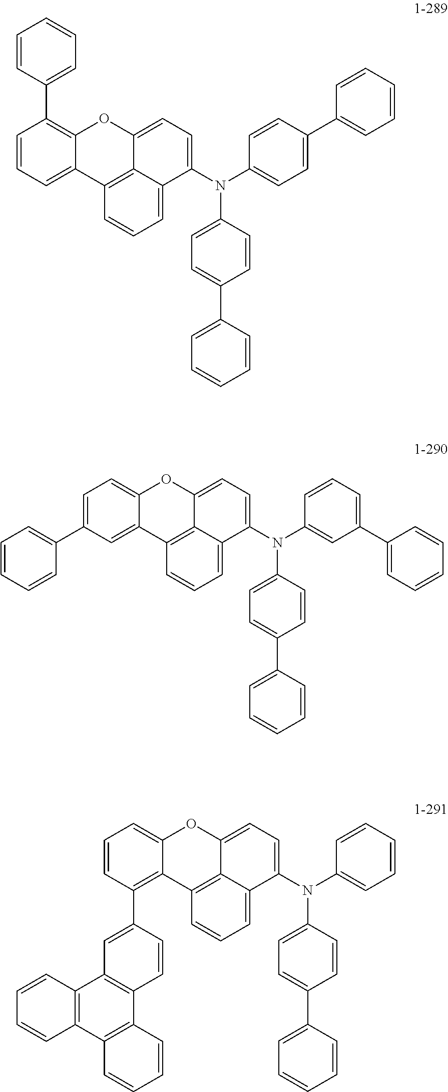 [SMILES: c1ccc(-c2ccc(N(c3ccc(-c4ccccc4)cc3)c3ccc4c5c(cccc35)-c3cccc(-c5ccccc5)c3O4)cc2)cc1 ; c1ccc(-c2ccc(N(c3cccc(-c4ccccc4)c3)c3ccc4c5c(cccc35)-c3cc(-c5ccccc5)ccc3O4)cc2)cc1 ; c1ccc(-c2ccc(N(c3ccccc3)c3ccc4c5c(cccc35)-c3c(cccc3-c3ccc5c6ccccc6c6ccccc6c5c3)O4)cc2)cc1]

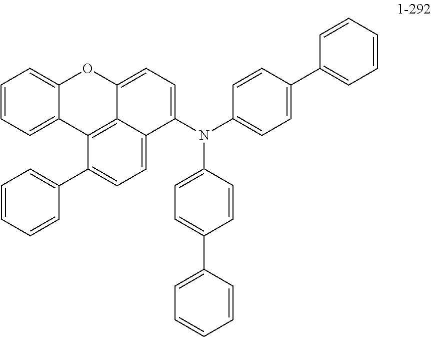 [SMILES: c1ccc(-c2ccc(N(c3ccc(-c4ccccc4)cc3)c3ccc4c5c(c(-c6ccccc6)ccc35)-c3ccccc3O4)cc2)cc1]

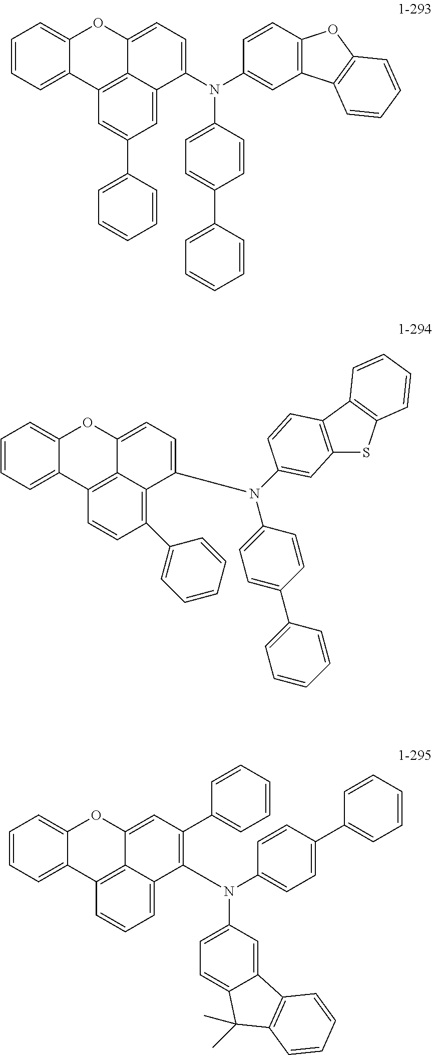 [SMILES: c1ccc(-c2ccc(N(c3ccc4oc5ccccc5c4c3)c3ccc4c5c(cc(-c6ccccc6)cc35)-c3ccccc3O4)cc2)cc1 ; c1ccc(-c2ccc(N(c3ccc4c(c3)sc3ccccc34)c3ccc4c5c(ccc(-c6ccccc6)c35)-c3ccccc3O4)cc2)cc1 ; CC1(C)c2ccccc2-c2cc(N(c3ccc(-c4ccccc4)cc3)c3c(-c4ccccc4)cc4c5c(cccc35)-c3ccccc3O4)ccc21]

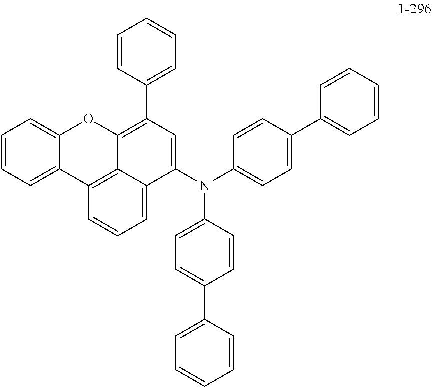 [SMILES: c1ccc(-c2ccc(N(c3ccc(-c4ccccc4)cc3)c3cc(-c4ccccc4)c4c5c(cccc35)-c3ccccc3O4)cc2)cc1]

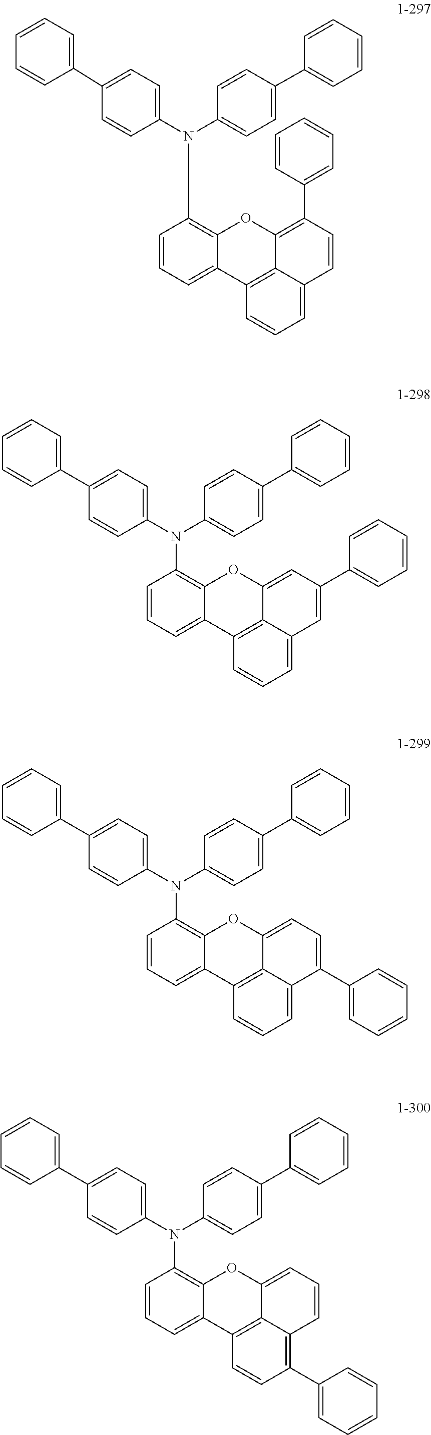 [SMILES: c1ccc(-c2ccc(N(c3ccc(-c4ccccc4)cc3)c3cccc4c3Oc3c(-c5ccccc5)ccc5cccc-4c35)cc2)cc1 ; c1ccc(-c2ccc(N(c3ccc(-c4ccccc4)cc3)c3cccc4c3Oc3cc(-c5ccccc5)cc5cccc-4c35)cc2)cc1 ; c1ccc(-c2ccc(N(c3ccc(-c4ccccc4)cc3)c3cccc4c3Oc3ccc(-c5ccccc5)c5cccc-4c35)cc2)cc1 ; c1ccc(-c2ccc(N(c3ccc(-c4ccccc4)cc3)c3cccc4c3Oc3cccc5c(-c6ccccc6)ccc-4c35)cc2)cc1]

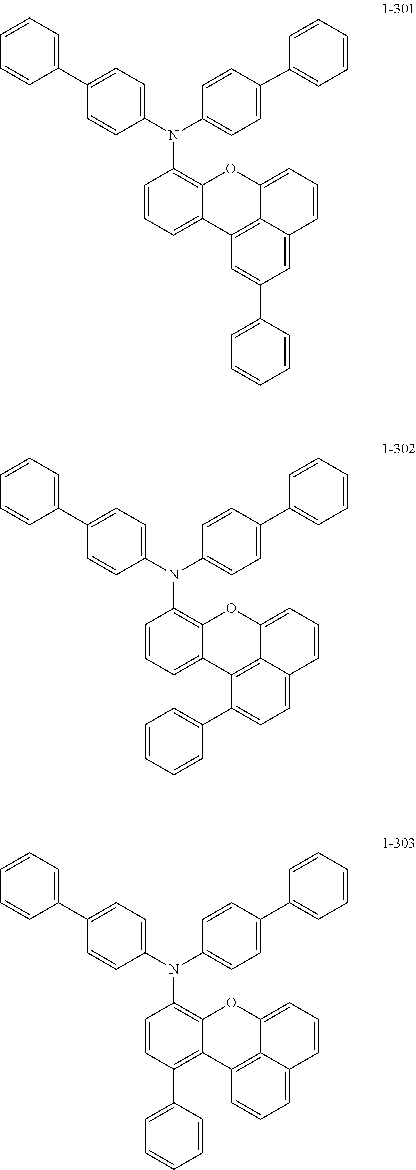 [SMILES: c1ccc(-c2ccc(N(c3ccc(-c4ccccc4)cc3)c3cccc4c3Oc3cccc5cc(-c6ccccc6)cc-4c35)cc2)cc1 ; c1ccc(-c2ccc(N(c3ccc(-c4ccccc4)cc3)c3cccc4c3Oc3cccc5ccc(-c6ccccc6)c-4c35)cc2)cc1 ; c1ccc(-c2ccc(N(c3ccc(-c4ccccc4)cc3)c3ccc(-c4ccccc4)c4c3Oc3cccc5cccc-4c35)cc2)cc1]

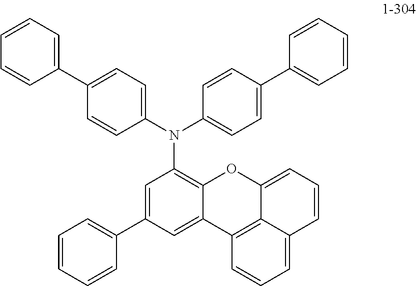 [SMILES: c1ccc(-c2ccc(N(c3ccc(-c4ccccc4)cc3)c3cc(-c4ccccc4)cc4c3Oc3cccc5cccc-4c35)cc2)cc1]

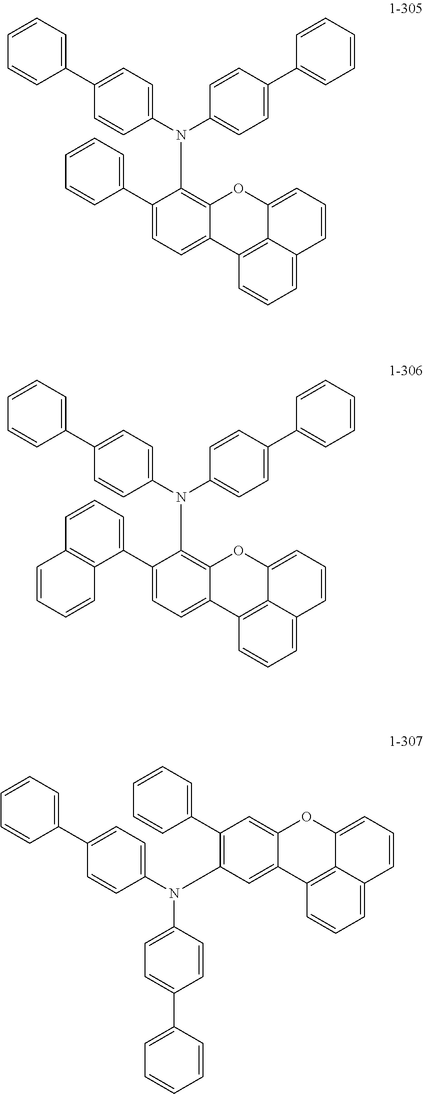 [SMILES: c1ccc(-c2ccc(N(c3ccc(-c4ccccc4)cc3)c3c(-c4ccccc4)ccc4c3Oc3cccc5cccc-4c35)cc2)cc1 ; c1ccc(-c2ccc(N(c3ccc(-c4ccccc4)cc3)c3c(-c4cccc5ccccc45)ccc4c3Oc3cccc5cccc-4c35)cc2)cc1 ; c1ccc(-c2ccc(N(c3ccc(-c4ccccc4)cc3)c3cc4c(cc3-c3ccccc3)Oc3cccc5cccc-4c35)cc2)cc1]

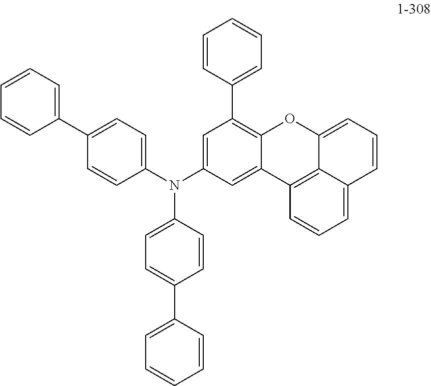 [SMILES: c1ccc(-c2ccc(N(c3ccc(-c4ccccc4)cc3)c3cc(-c4ccccc4)c4c(c3)-c3cccc5cccc(c35)O4)cc2)cc1]

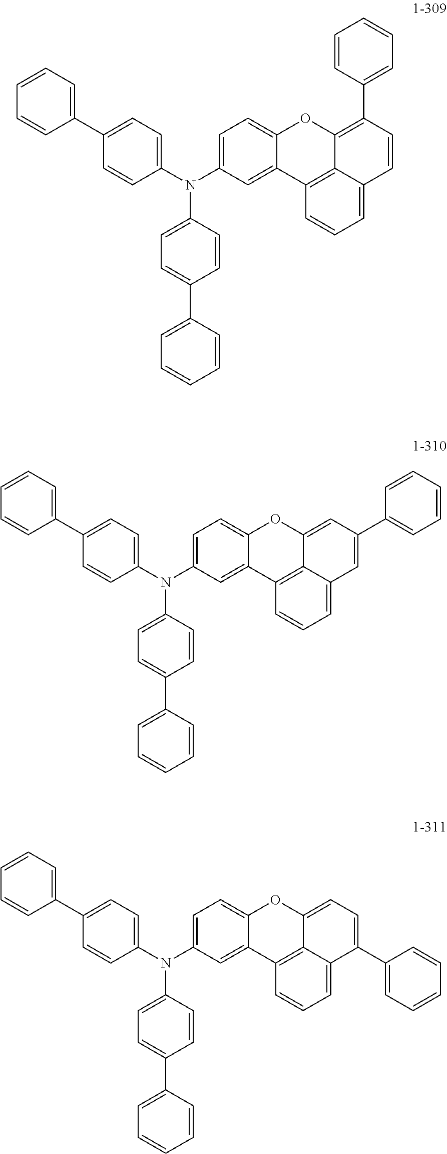 [SMILES: c1ccc(-c2ccc(N(c3ccc(-c4ccccc4)cc3)c3ccc4c(c3)-c3cccc5ccc(-c6ccccc6)c(c35)O4)cc2)cc1 ; c1ccc(-c2ccc(N(c3ccc(-c4ccccc4)cc3)c3ccc4c(c3)-c3cccc5cc(-c6ccccc6)cc(c35)O4)cc2)cc1 ; c1ccc(-c2ccc(N(c3ccc(-c4ccccc4)cc3)c3ccc4c(c3)-c3cccc5c(-c6ccccc6)ccc(c35)O4)cc2)cc1]

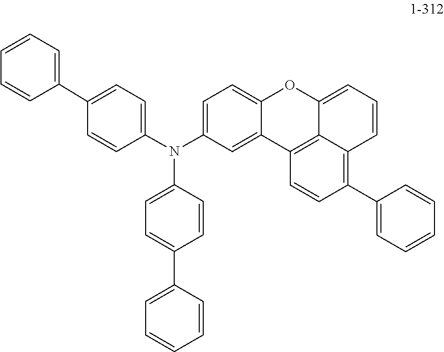 [SMILES: c1ccc(-c2ccc(N(c3ccc(-c4ccccc4)cc3)c3ccc4c(c3)-c3ccc(-c5ccccc5)c5cccc(c35)O4)cc2)cc1]

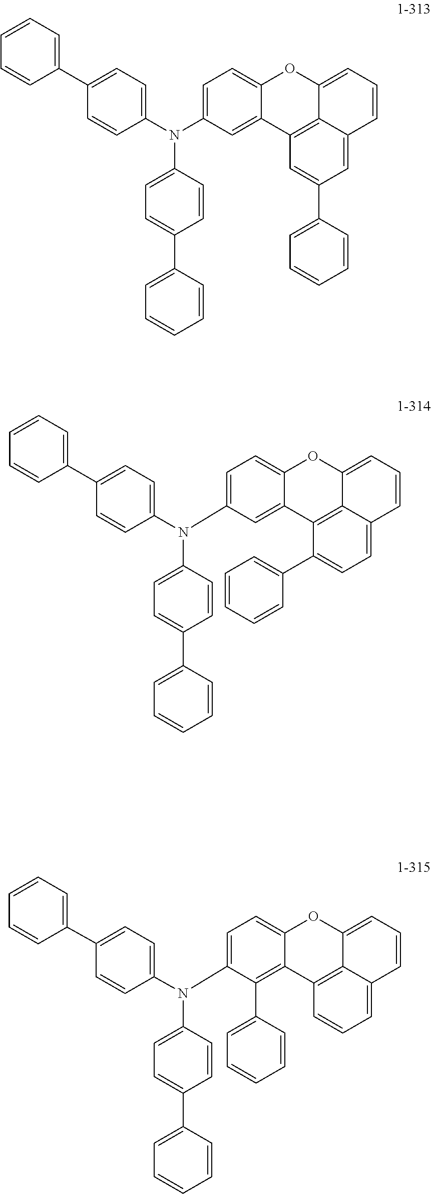 [SMILES: c1ccc(-c2ccc(N(c3ccc(-c4ccccc4)cc3)c3ccc4c(c3)-c3cc(-c5ccccc5)cc5cccc(c35)O4)cc2)cc1 ; c1ccc(-c2ccc(N(c3ccc(-c4ccccc4)cc3)c3ccc4c(c3)-c3c(-c5ccccc5)ccc5cccc(c35)O4)cc2)cc1 ; c1ccc(-c2ccc(N(c3ccc(-c4ccccc4)cc3)c3ccc4c(c3-c3ccccc3)-c3cccc5cccc(c35)O4)cc2)cc1]

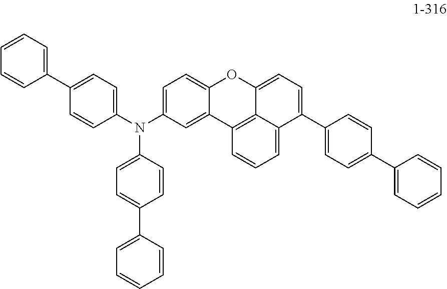 [SMILES: c1ccc(-c2ccc(-c3ccc4c5c(cccc35)-c3cc(N(c5ccc(-c6ccccc6)cc5)c5ccc(-c6ccccc6)cc5)ccc3O4)cc2)cc1]

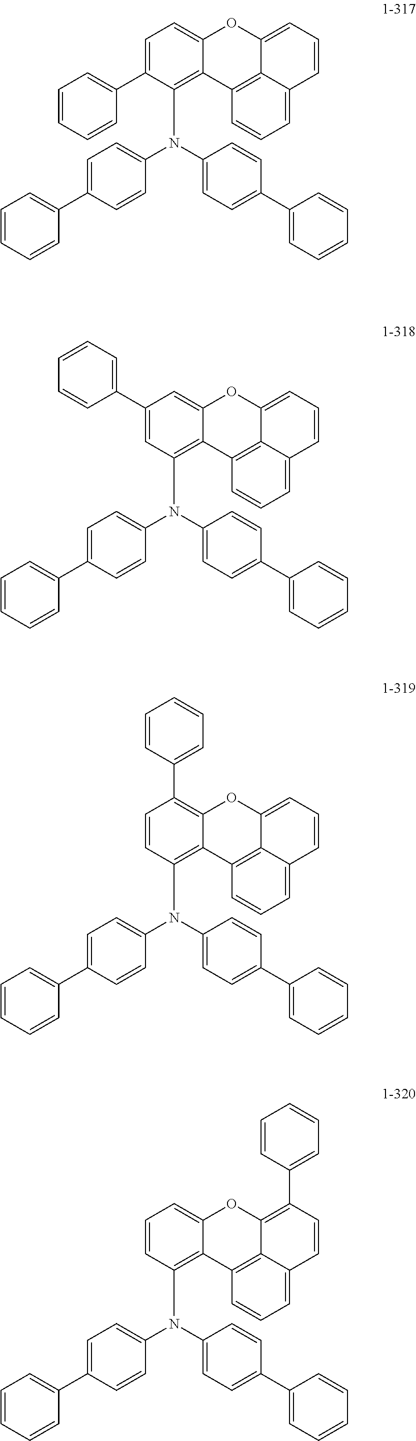 [SMILES: c1ccc(-c2ccc(N(c3ccc(-c4ccccc4)cc3)c3c(-c4ccccc4)ccc4c3-c3cccc5cccc(c35)O4)cc2)cc1 ; c1ccc(-c2ccc(N(c3ccc(-c4ccccc4)cc3)c3cc(-c4ccccc4)cc4c3-c3cccc5cccc(c35)O4)cc2)cc1 ; c1ccc(-c2ccc(N(c3ccc(-c4ccccc4)cc3)c3ccc(-c4ccccc4)c4c3-c3cccc5cccc(c35)O4)cc2)cc1 ; c1ccc(-c2ccc(N(c3ccc(-c4ccccc4)cc3)c3cccc4c3-c3cccc5ccc(-c6ccccc6)c(c35)O4)cc2)cc1]

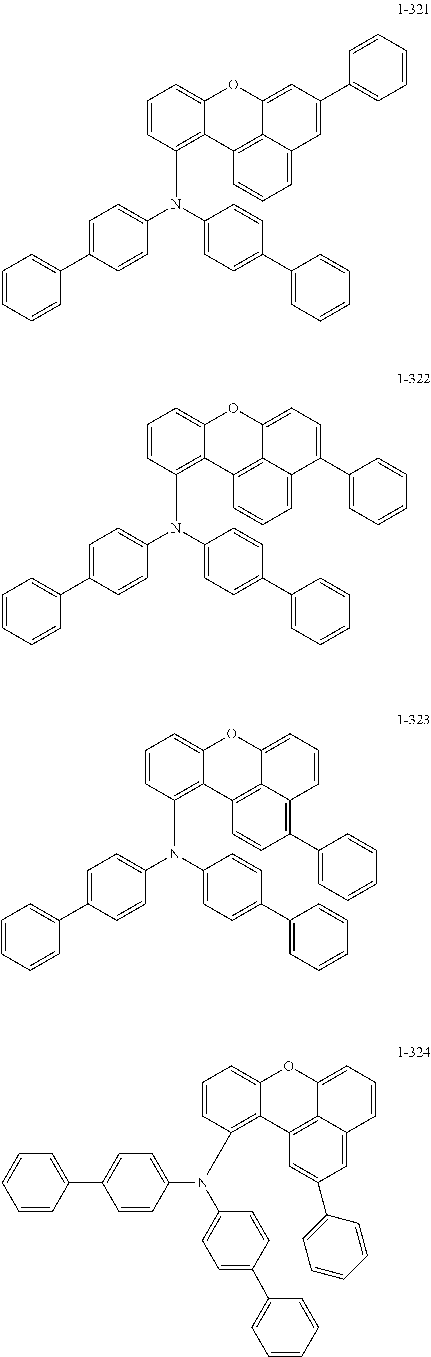 [SMILES: c1ccc(-c2ccc(N(c3ccc(-c4ccccc4)cc3)c3cccc4c3-c3cccc5cc(-c6ccccc6)cc(c35)O4)cc2)cc1 ; c1ccc(-c2ccc(N(c3ccc(-c4ccccc4)cc3)c3cccc4c3-c3cccc5c(-c6ccccc6)ccc(c35)O4)cc2)cc1 ; c1ccc(-c2ccc(N(c3ccc(-c4ccccc4)cc3)c3cccc4c3-c3ccc(-c5ccccc5)c5cccc(c35)O4)cc2)cc1 ; c1ccc(-c2ccc(N(c3ccc(-c4ccccc4)cc3)c3cccc4c3-c3cc(-c5ccccc5)cc5cccc(c35)O4)cc2)cc1]

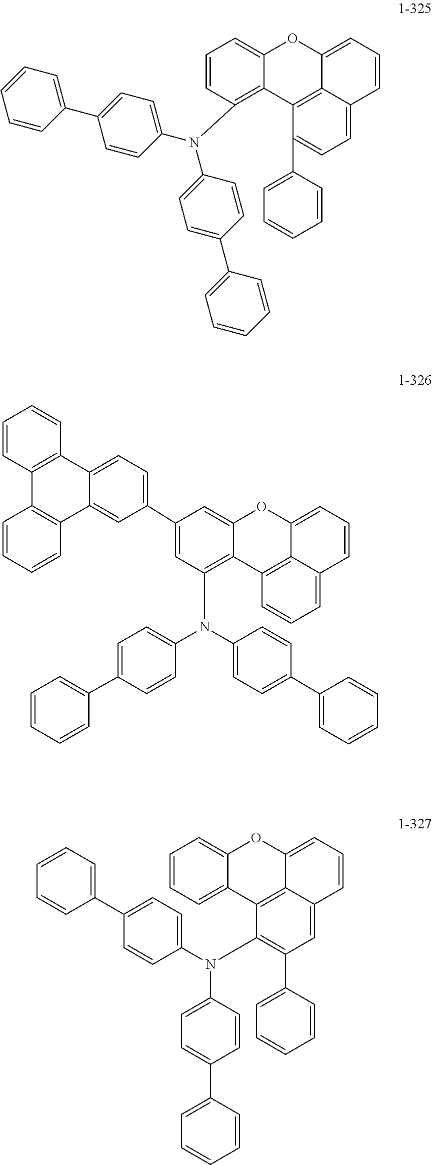 [SMILES: c1ccc(-c2ccc(N(c3ccc(-c4ccccc4)cc3)c3cccc4c3-c3c(-c5ccccc5)ccc5cccc(c35)O4)cc2)cc1 ; c1ccc(-c2ccc(N(c3ccc(-c4ccccc4)cc3)c3cc(-c4ccc5c6ccccc6c6ccccc6c5c4)cc4c3-c3cccc5cccc(c35)O4)cc2)cc1 ; c1ccc(-c2ccc(N(c3ccc(-c4ccccc4)cc3)c3c(-c4ccccc4)cc4cccc5c4c3-c3ccccc3O5)cc2)cc1]

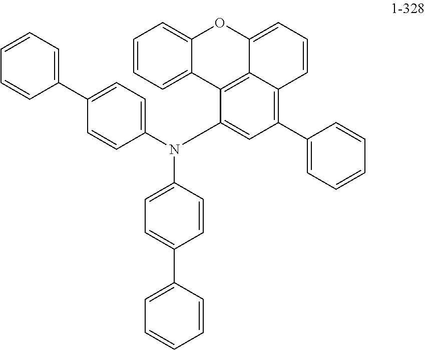 [SMILES: c1ccc(-c2ccc(N(c3ccc(-c4ccccc4)cc3)c3cc(-c4ccccc4)c4cccc5c4c3-c3ccccc3O5)cc2)cc1]

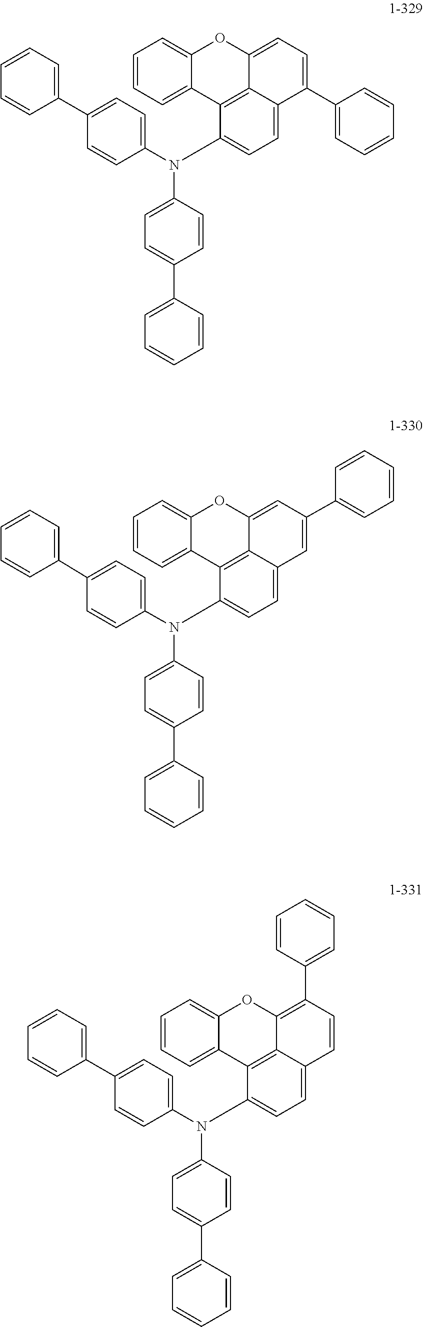 [SMILES: c1ccc(-c2ccc(N(c3ccc(-c4ccccc4)cc3)c3ccc4c(-c5ccccc5)ccc5c4c3-c3ccccc3O5)cc2)cc1 ; c1ccc(-c2ccc(N(c3ccc(-c4ccccc4)cc3)c3ccc4cc(-c5ccccc5)cc5c4c3-c3ccccc3O5)cc2)cc1 ; c1ccc(-c2ccc(N(c3ccc(-c4ccccc4)cc3)c3ccc4ccc(-c5ccccc5)c5c4c3-c3ccccc3O5)cc2)cc1]

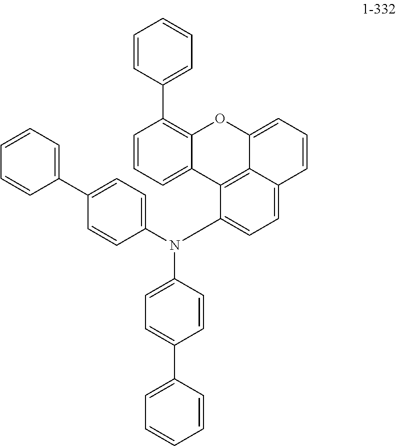 [SMILES: c1ccc(-c2ccc(N(c3ccc(-c4ccccc4)cc3)c3ccc4cccc5c4c3-c3cccc(-c4ccccc4)c3O5)cc2)cc1]

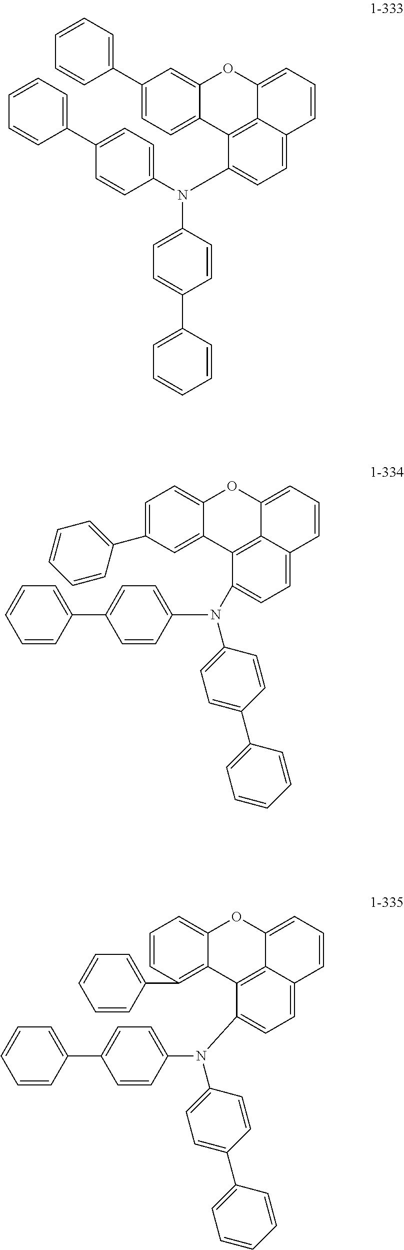 [SMILES: c1ccc(-c2ccc(N(c3ccc(-c4ccccc4)cc3)c3ccc4cccc5c4c3-c3ccc(-c4ccccc4)cc3O5)cc2)cc1 ; c1ccc(-c2ccc(N(c3ccc(-c4ccccc4)cc3)c3ccc4cccc5c4c3-c3cc(-c4ccccc4)ccc3O5)cc2)cc1 ; c1ccc(-c2ccc(N(c3ccc(-c4ccccc4)cc3)c3ccc4cccc5c4c3-c3c(cccc3-c3ccccc3)O5)cc2)cc1]

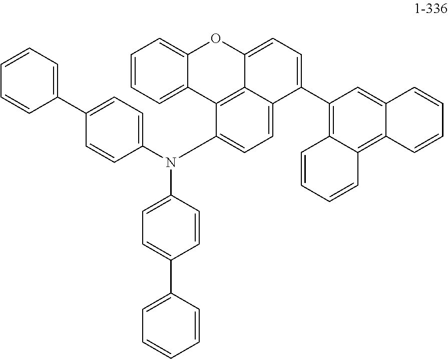 [SMILES: c1ccc(-c2ccc(N(c3ccc(-c4ccccc4)cc3)c3ccc4c(-c5cc6ccccc6c6ccccc56)ccc5c4c3-c3ccccc3O5)cc2)cc1]

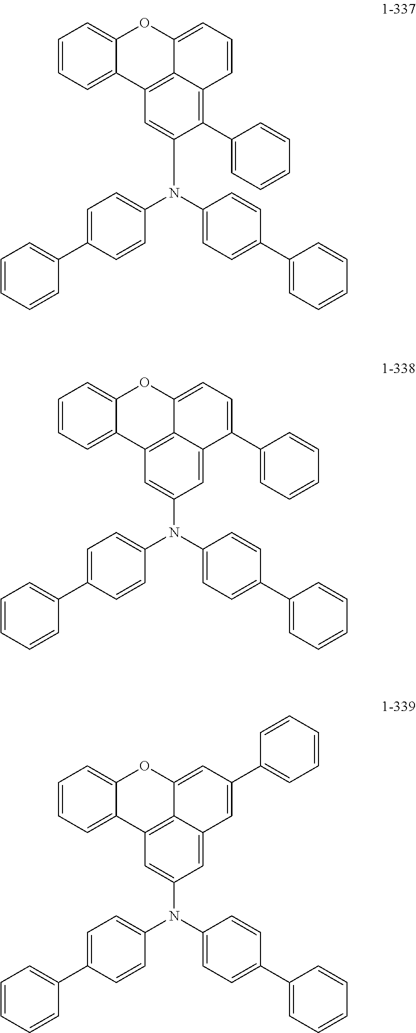 [SMILES: c1ccc(-c2ccc(N(c3ccc(-c4ccccc4)cc3)c3cc4c5c(cccc5c3-c3ccccc3)Oc3ccccc3-4)cc2)cc1 ; c1ccc(-c2ccc(N(c3ccc(-c4ccccc4)cc3)c3cc4c5c(ccc(-c6ccccc6)c5c3)Oc3ccccc3-4)cc2)cc1 ; c1ccc(-c2ccc(N(c3ccc(-c4ccccc4)cc3)c3cc4c5c(cc(-c6ccccc6)cc5c3)Oc3ccccc3-4)cc2)cc1]

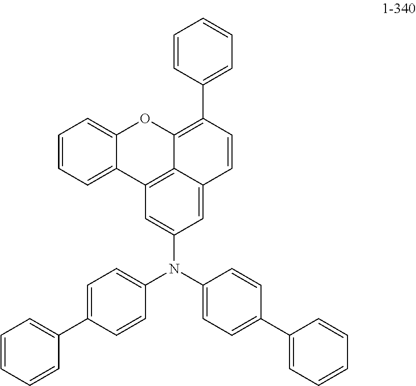 [SMILES: c1ccc(-c2ccc(N(c3ccc(-c4ccccc4)cc3)c3cc4c5c(c(-c6ccccc6)ccc5c3)Oc3ccccc3-4)cc2)cc1]

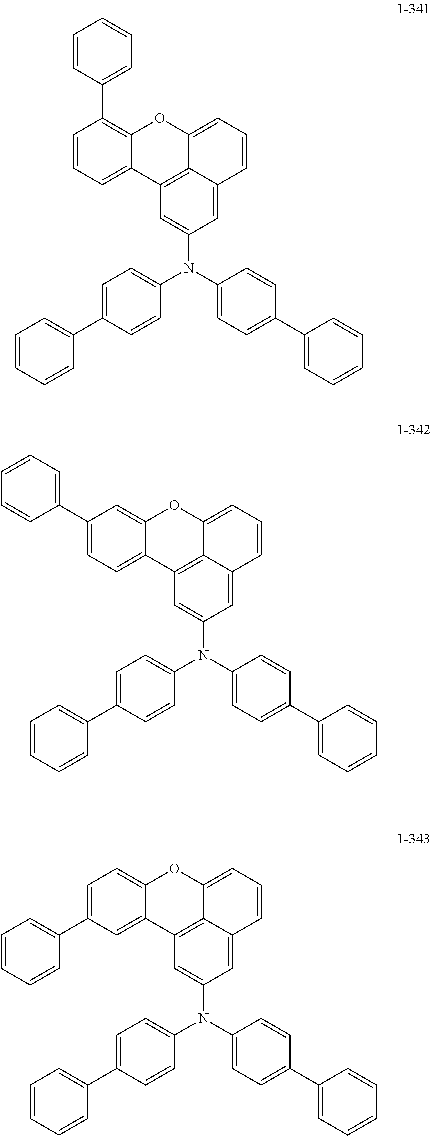 [SMILES: c1ccc(-c2ccc(N(c3ccc(-c4ccccc4)cc3)c3cc4c5c(cccc5c3)Oc3c(-c5ccccc5)cccc3-4)cc2)cc1 ; c1ccc(-c2ccc(N(c3ccc(-c4ccccc4)cc3)c3cc4c5c(cccc5c3)Oc3cc(-c5ccccc5)ccc3-4)cc2)cc1 ; c1ccc(-c2ccc(N(c3ccc(-c4ccccc4)cc3)c3cc4c5c(cccc5c3)Oc3ccc(-c5ccccc5)cc3-4)cc2)cc1]

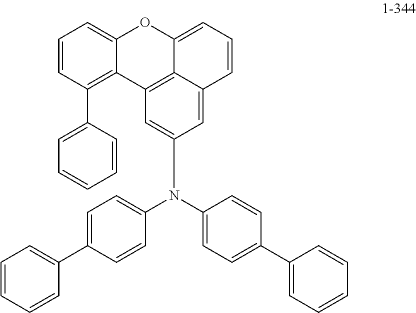 [SMILES: c1ccc(-c2ccc(N(c3ccc(-c4ccccc4)cc3)c3cc4c5c(cccc5c3)Oc3cccc(-c5ccccc5)c3-4)cc2)cc1]

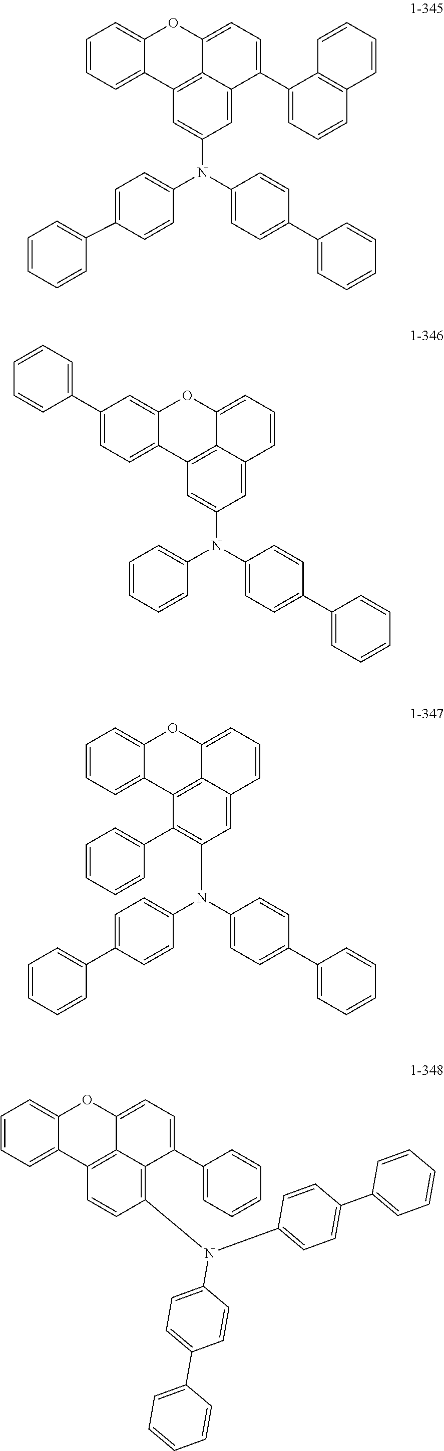 [SMILES: c1ccc(-c2ccc(N(c3ccc(-c4ccccc4)cc3)c3cc4c5c(ccc(-c6cccc7ccccc67)c5c3)Oc3ccccc3-4)cc2)cc1 ; c1ccc(-c2ccc(N(c3ccccc3)c3cc4c5c(cccc5c3)Oc3cc(-c5ccccc5)ccc3-4)cc2)cc1 ; c1ccc(-c2ccc(N(c3ccc(-c4ccccc4)cc3)c3cc4cccc5c4c(c3-c3ccccc3)-c3ccccc3O5)cc2)cc1 ; c1ccc(-c2ccc(N(c3ccc(-c4ccccc4)cc3)c3ccc4c5c(ccc(-c6ccccc6)c35)Oc3ccccc3-4)cc2)cc1]

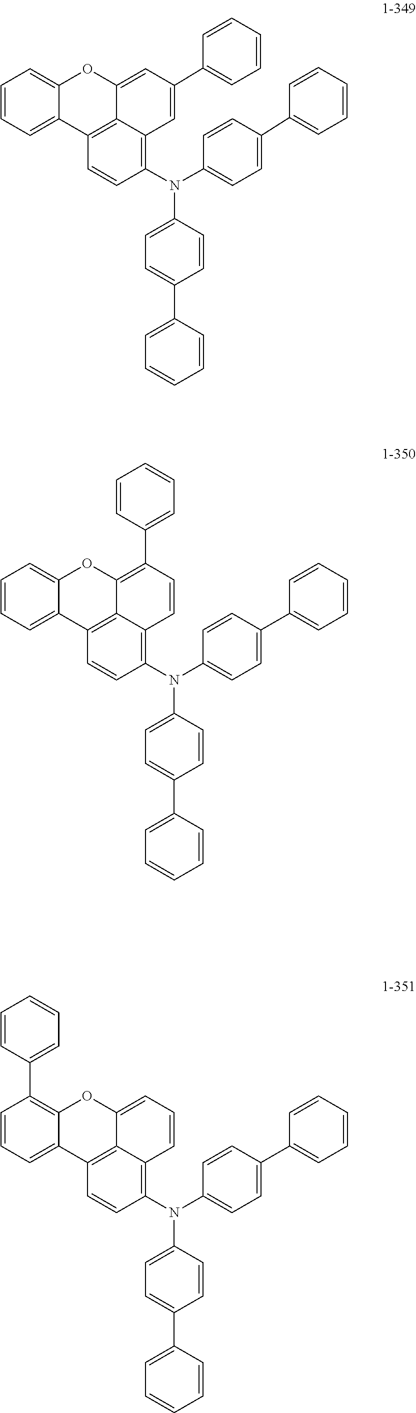 [SMILES: c1ccc(-c2ccc(N(c3ccc(-c4ccccc4)cc3)c3ccc4c5c(cc(-c6ccccc6)cc35)Oc3ccccc3-4)cc2)cc1 ; c1ccc(-c2ccc(N(c3ccc(-c4ccccc4)cc3)c3ccc4c5c(c(-c6ccccc6)ccc35)Oc3ccccc3-4)cc2)cc1 ; c1ccc(-c2ccc(N(c3ccc(-c4ccccc4)cc3)c3ccc4c5c(cccc35)Oc3c(-c5ccccc5)cccc3-4)cc2)cc1]

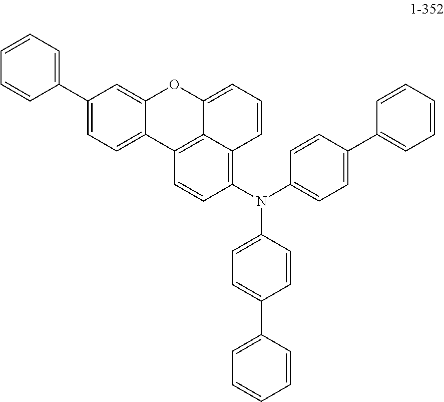 [SMILES: c1ccc(-c2ccc(N(c3ccc(-c4ccccc4)cc3)c3ccc4c5c(cccc35)Oc3cc(-c5ccccc5)ccc3-4)cc2)cc1]

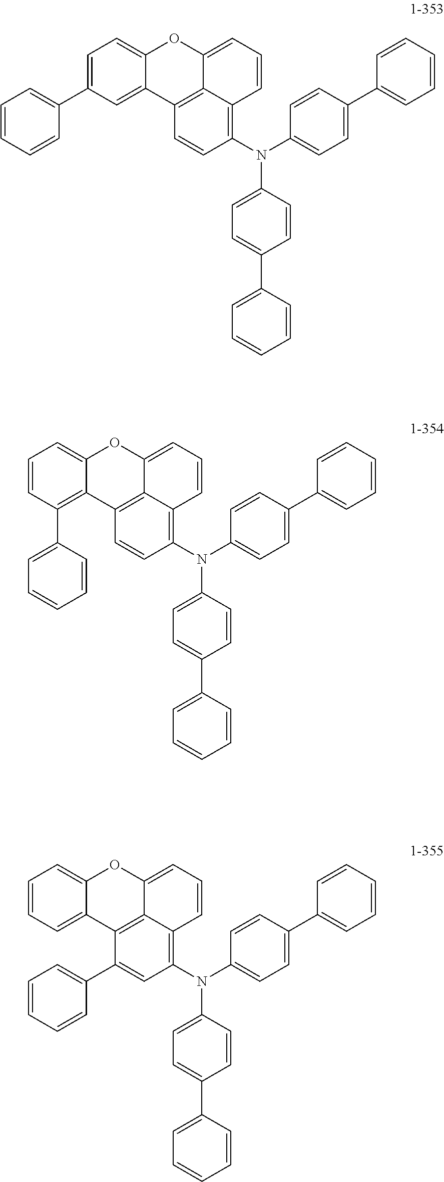 [SMILES: c1ccc(-c2ccc(N(c3ccc(-c4ccccc4)cc3)c3ccc4c5c(cccc35)Oc3ccc(-c5ccccc5)cc3-4)cc2)cc1 ; c1ccc(-c2ccc(N(c3ccc(-c4ccccc4)cc3)c3ccc4c5c(cccc35)Oc3cccc(-c5ccccc5)c3-4)cc2)cc1 ; c1ccc(-c2ccc(N(c3ccc(-c4ccccc4)cc3)c3cc(-c4ccccc4)c4c5c(cccc35)Oc3ccccc3-4)cc2)cc1]

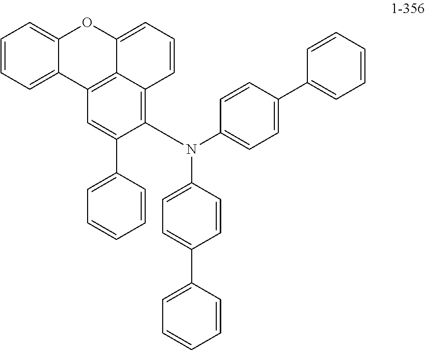 [SMILES: c1ccc(-c2ccc(N(c3ccc(-c4ccccc4)cc3)c3c(-c4ccccc4)cc4c5c(cccc35)Oc3ccccc3-4)cc2)cc1]

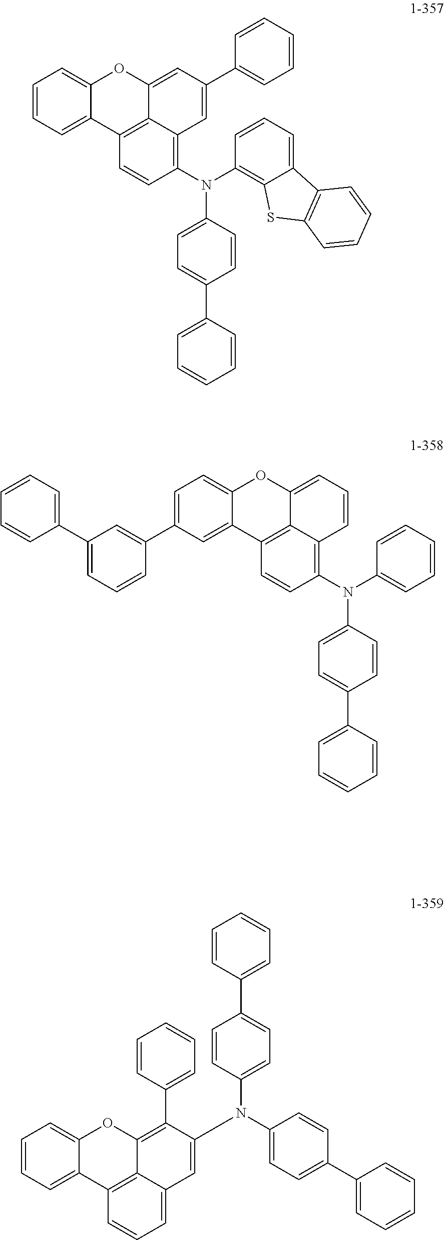 [SMILES: c1ccc(-c2ccc(N(c3ccc4c5c(cc(-c6ccccc6)cc35)Oc3ccccc3-4)c3cccc4c3sc3ccccc34)cc2)cc1 ; c1ccc(-c2ccc(N(c3ccccc3)c3ccc4c5c(cccc35)Oc3ccc(-c5cccc(-c6ccccc6)c5)cc3-4)cc2)cc1 ; c1ccc(-c2ccc(N(c3ccc(-c4ccccc4)cc3)c3cc4cccc5c4c(c3-c3ccccc3)Oc3ccccc3-5)cc2)cc1]

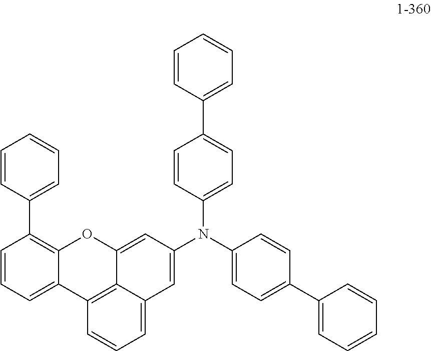 [SMILES: c1ccc(-c2ccc(N(c3ccc(-c4ccccc4)cc3)c3cc4c5c(cccc5c3)-c3cccc(-c5ccccc5)c3O4)cc2)cc1]

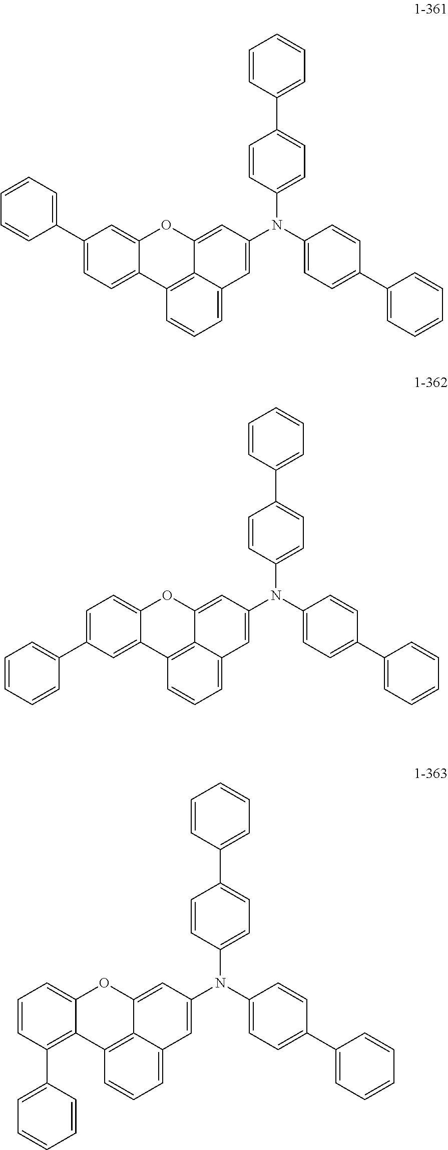 [SMILES: c1ccc(-c2ccc(N(c3ccc(-c4ccccc4)cc3)c3cc4c5c(cccc5c3)-c3ccc(-c5ccccc5)cc3O4)cc2)cc1 ; c1ccc(-c2ccc(N(c3ccc(-c4ccccc4)cc3)c3cc4c5c(cccc5c3)-c3cc(-c5ccccc5)ccc3O4)cc2)cc1 ; c1ccc(-c2ccc(N(c3ccc(-c4ccccc4)cc3)c3cc4c5c(cccc5c3)-c3c(cccc3-c3ccccc3)O4)cc2)cc1]

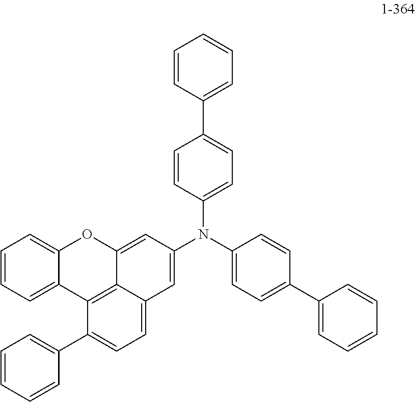 [SMILES: c1ccc(-c2ccc(N(c3ccc(-c4ccccc4)cc3)c3cc4c5c(c(-c6ccccc6)ccc5c3)-c3ccccc3O4)cc2)cc1]

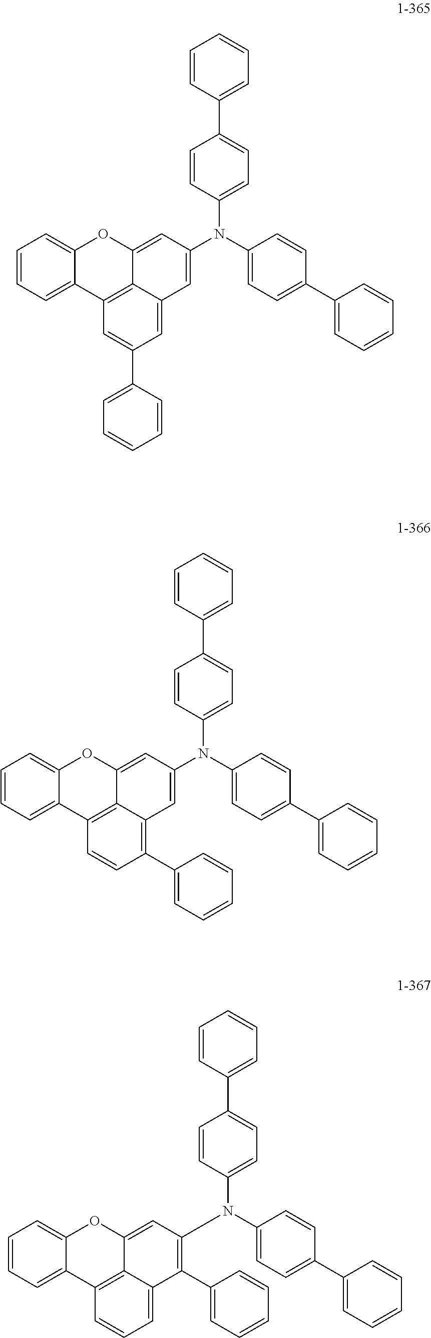 [SMILES: c1ccc(-c2ccc(N(c3ccc(-c4ccccc4)cc3)c3cc4c5c(cc(-c6ccccc6)cc5c3)-c3ccccc3O4)cc2)cc1 ; c1ccc(-c2ccc(N(c3ccc(-c4ccccc4)cc3)c3cc4c5c(ccc(-c6ccccc6)c5c3)-c3ccccc3O4)cc2)cc1 ; c1ccc(-c2ccc(N(c3ccc(-c4ccccc4)cc3)c3cc4c5c(cccc5c3-c3ccccc3)-c3ccccc3O4)cc2)cc1]

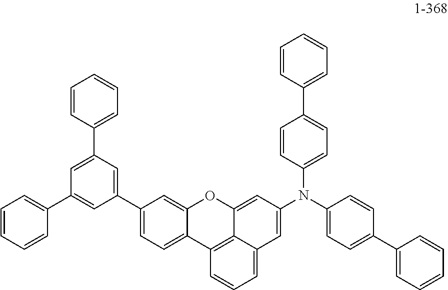 [SMILES: c1ccc(-c2ccc(N(c3ccc(-c4ccccc4)cc3)c3cc4c5c(cccc5c3)-c3ccc(-c5cc(-c6ccccc6)cc(-c6ccccc6)c5)cc3O4)cc2)cc1]

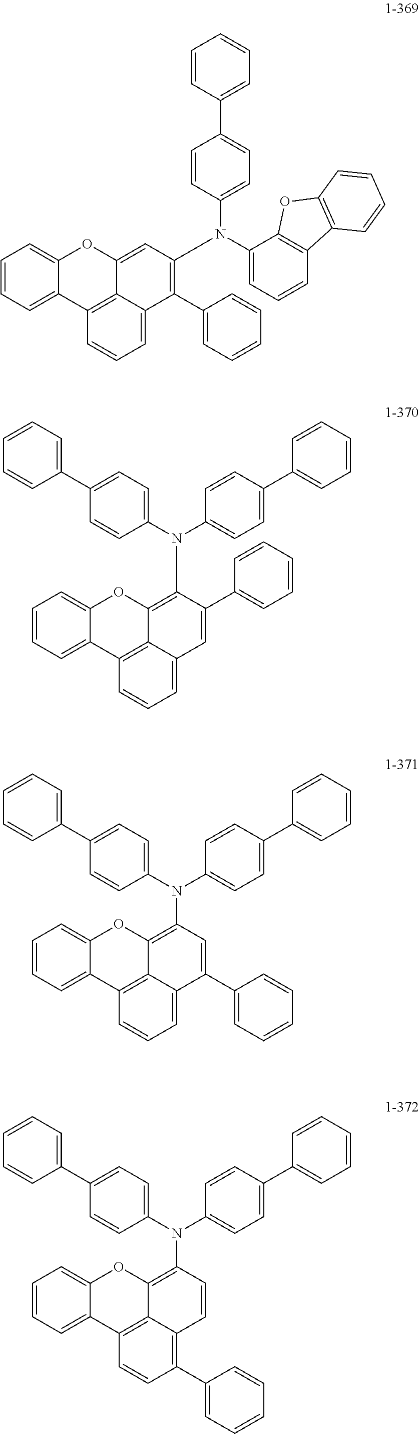 [SMILES: c1ccc(-c2ccc(N(c3cc4c5c(cccc5c3-c3ccccc3)-c3ccccc3O4)c3cccc4c3oc3ccccc34)cc2)cc1 ; c1ccc(-c2ccc(N(c3ccc(-c4ccccc4)cc3)c3c(-c4ccccc4)cc4cccc5c4c3Oc3ccccc3-5)cc2)cc1 ; c1ccc(-c2ccc(N(c3ccc(-c4ccccc4)cc3)c3cc(-c4ccccc4)c4cccc5c4c3Oc3ccccc3-5)cc2)cc1 ; c1ccc(-c2ccc(N(c3ccc(-c4ccccc4)cc3)c3ccc4c(-c5ccccc5)ccc5c4c3Oc3ccccc3-5)cc2)cc1]

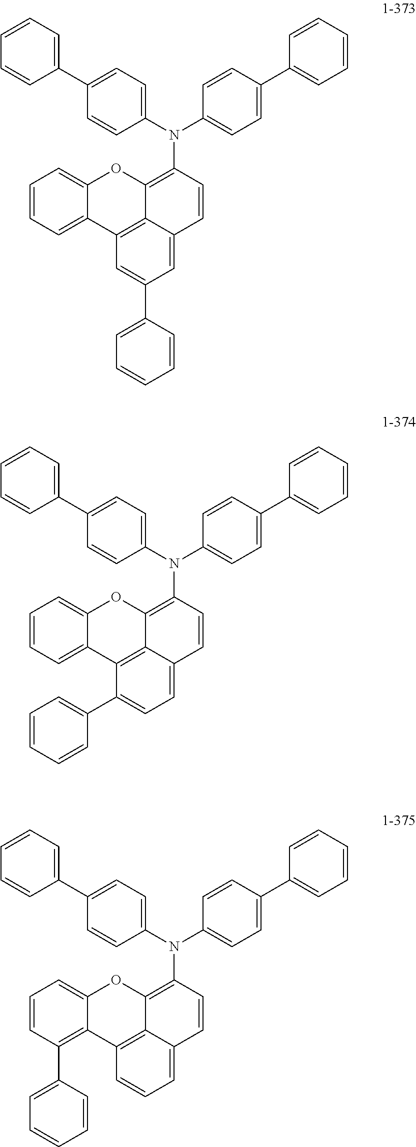 [SMILES: c1ccc(-c2ccc(N(c3ccc(-c4ccccc4)cc3)c3ccc4cc(-c5ccccc5)cc5c4c3Oc3ccccc3-5)cc2)cc1 ; c1ccc(-c2ccc(N(c3ccc(-c4ccccc4)cc3)c3ccc4ccc(-c5ccccc5)c5c4c3Oc3ccccc3-5)cc2)cc1 ; c1ccc(-c2ccc(N(c3ccc(-c4ccccc4)cc3)c3ccc4cccc5c4c3Oc3cccc(-c4ccccc4)c3-5)cc2)cc1]

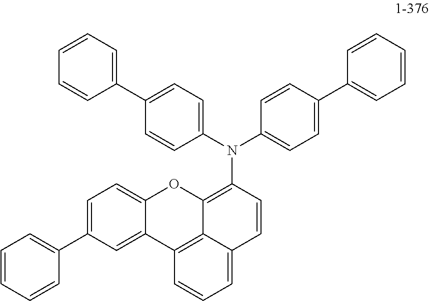 [SMILES: c1ccc(-c2ccc(N(c3ccc(-c4ccccc4)cc3)c3ccc4cccc5c4c3Oc3ccc(-c4ccccc4)cc3-5)cc2)cc1]

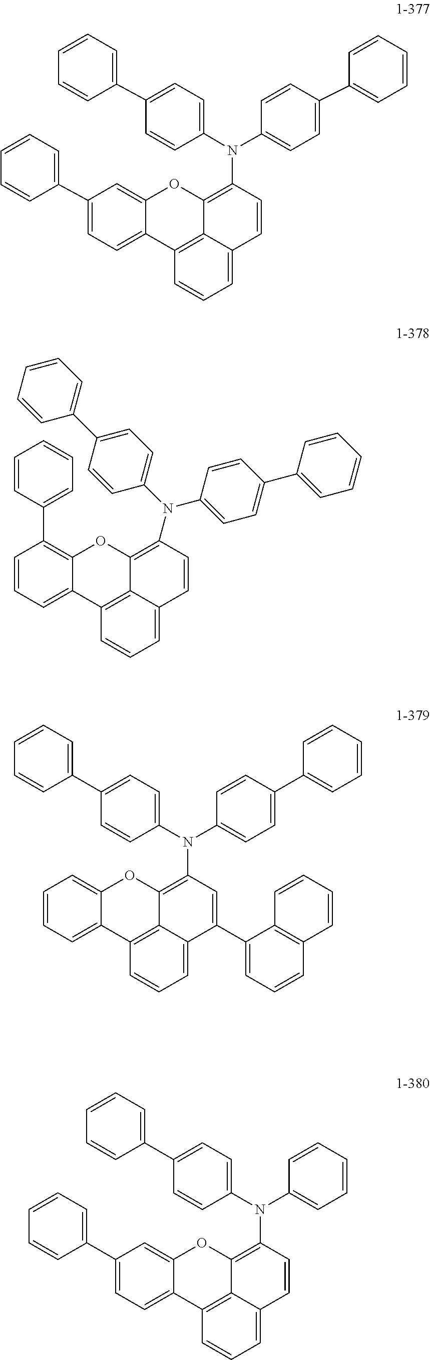 [SMILES: c1ccc(-c2ccc(N(c3ccc(-c4ccccc4)cc3)c3ccc4cccc5c4c3Oc3cc(-c4ccccc4)ccc3-5)cc2)cc1 ; c1ccc(-c2ccc(N(c3ccc(-c4ccccc4)cc3)c3ccc4cccc5c4c3Oc3c(-c4ccccc4)cccc3-5)cc2)cc1 ; c1ccc(-c2ccc(N(c3ccc(-c4ccccc4)cc3)c3cc(-c4cccc5ccccc45)c4cccc5c4c3Oc3ccccc3-5)cc2)cc1 ; c1ccc(-c2ccc(N(c3ccccc3)c3ccc4cccc5c4c3Oc3cc(-c4ccccc4)ccc3-5)cc2)cc1]

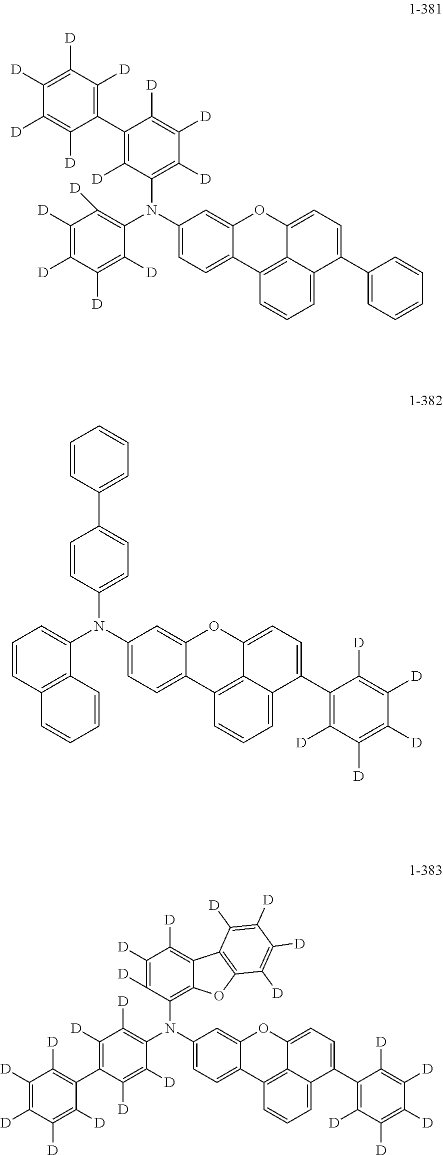 [SMILES: [2H]c1c([2H])c([2H])c(-c2c([2H])c([2H])c([2H])c(N(c3ccc4c(c3)Oc3ccc(-c5ccccc5)c5cccc-4c35)c3c([2H])c([2H])c([2H])c([2H])c3[2H])c2[2H])c([2H])c1[2H] ; [2H]c1c([2H])c([2H])c(-c2ccc3c4c(cccc24)-c2ccc(N(c4ccc(-c5ccccc5)cc4)c4cccc5ccccc45)cc2O3)c([2H])c1[2H] ; [2H]c1c([2H])c([2H])c(-c2c([2H])c([2H])c(N(c3ccc4c(c3)Oc3ccc(-c5c([2H])c([2H])c([2H])c([2H])c5[2H])c5cccc-4c35)c3c([2H])c([2H])c([2H])c4c3oc3c([2H])c([2H])c([2H])c([2H])c34)c([2H])c2[2H])c([2H])c1[2H]]

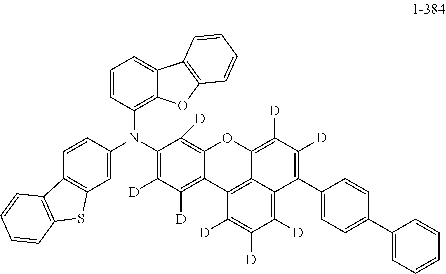 [SMILES: [2H]c1c([2H])c(N(c2ccc3c(c2)sc2ccccc23)c2cccc3c2oc2ccccc23)c([2H])c2c1-c1c([2H])c([2H])c([2H])c3c(-c4ccc(-c5ccccc5)cc4)c([2H])c([2H])c(c13)O2]

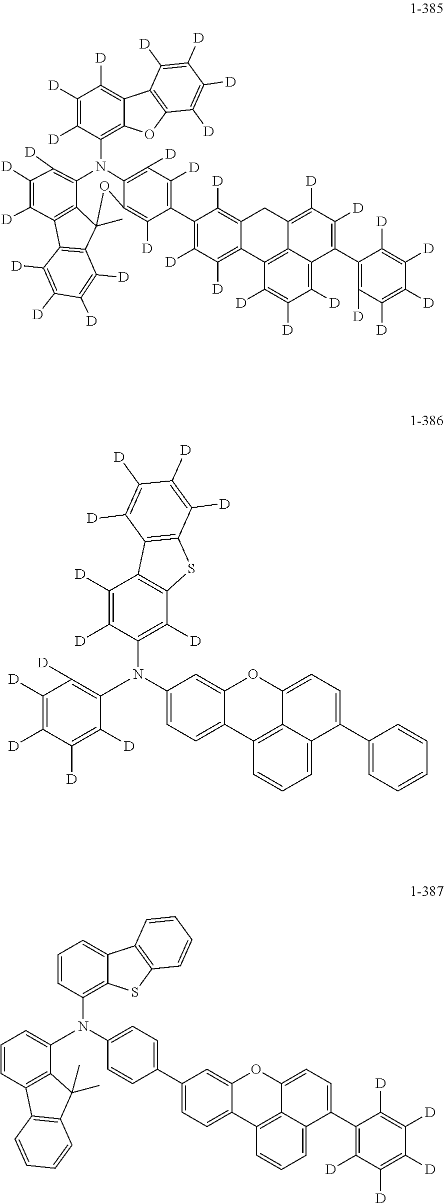

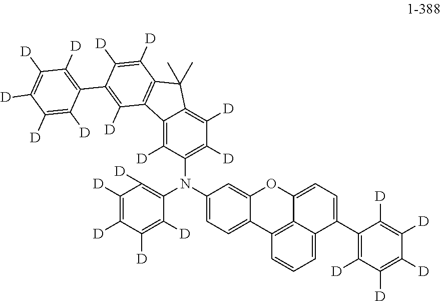 [SMILES: [2H]c1c([2H])c([2H])c(-c2c([2H])c([2H])c3c(c2[2H])-c2c([2H])c(N(c4ccc5c(c4)Oc4ccc(-c6c([2H])c([2H])c([2H])c([2H])c6[2H])c6cccc-5c46)c4c([2H])c([2H])c([2H])c([2H])c4[2H])c([2H])c([2H])c2C3(C)C)c([2H])c1[2H]]

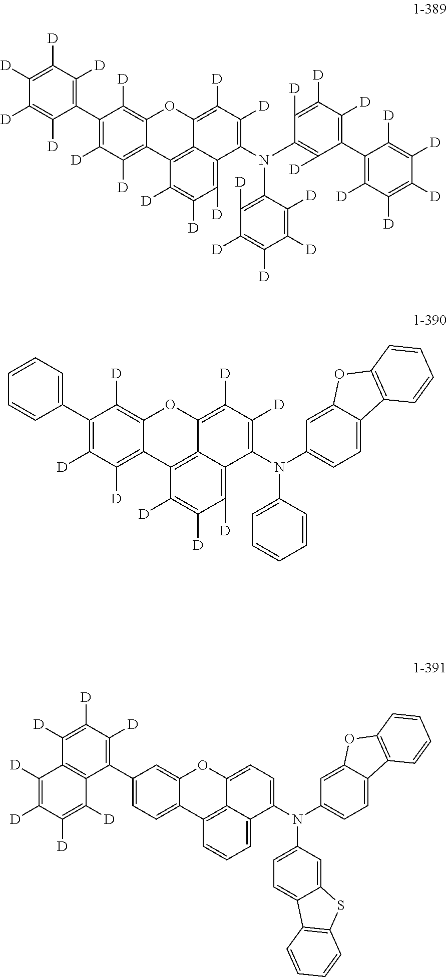 [SMILES: [2H]c1c([2H])c([2H])c(-c2c([2H])c([2H])c3c(c2[2H])Oc2c([2H])c([2H])c(N(c4c([2H])c([2H])c([2H])c([2H])c4[2H])c4c([2H])c([2H])c([2H])c(-c5c([2H])c([2H])c([2H])c([2H])c5[2H])c4[2H])c4c([2H])c([2H])c([2H])c-3c24)c([2H])c1[2H] ; [2H]c1c([2H])c2c(c([2H])c1-c1ccccc1)Oc1c([2H])c([2H])c(N(c3ccccc3)c3ccc4c(c3)oc3ccccc34)c3c([2H])c([2H])c([2H])c-2c13 ; [2H]c1c([2H])c([2H])c2c(-c3ccc4c(c3)Oc3ccc(N(c5ccc6c(c5)oc5ccccc56)c5ccc6c(c5)sc5ccccc56)c5cccc-4c35)c([2H])c([2H])c([2H])c2c1[2H]]

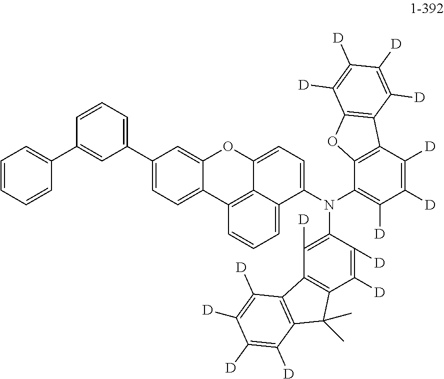 [SMILES: [2H]c1c([2H])c([2H])c2c(c1[2H])-c1c([2H])c(N(c3ccc4c5c(cccc35)-c3ccc(-c5cccc(-c6ccccc6)c5)cc3O4)c3c([2H])c([2H])c([2H])c4c3oc3c([2H])c([2H])c([2H])c([2H])c34)c([2H])c([2H])c1C2(C)C]

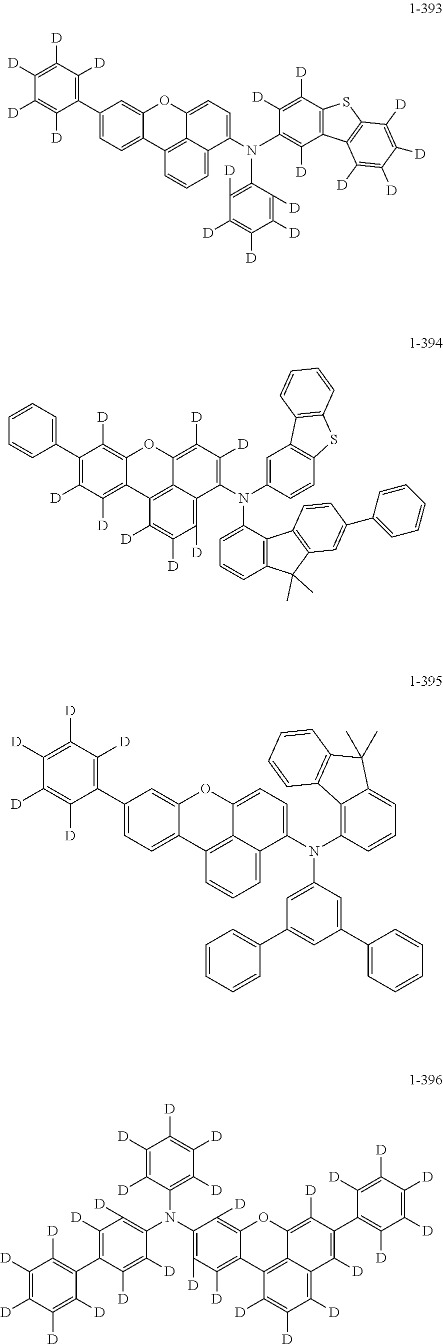

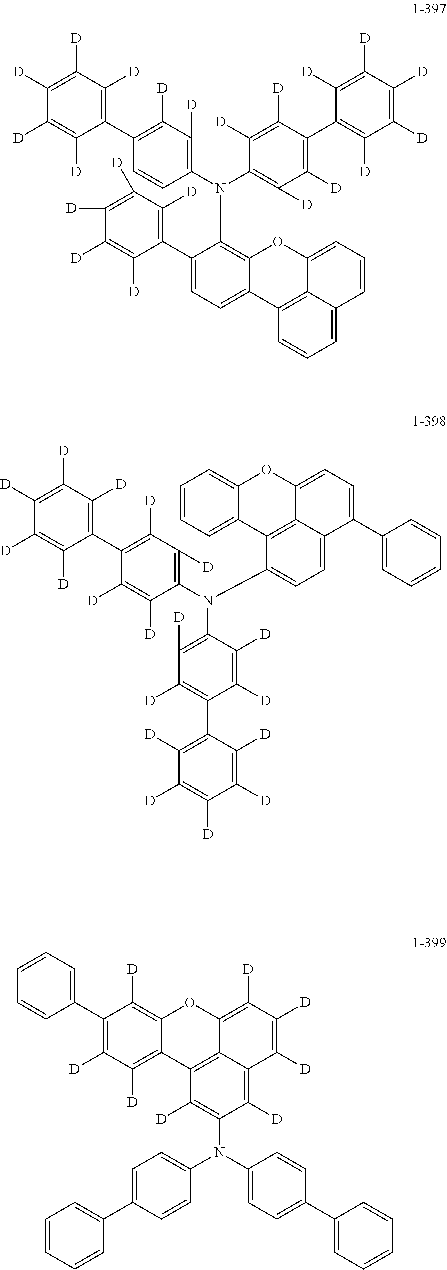

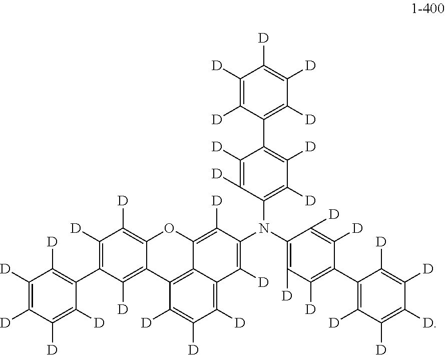

6. An organic light emitting device comprising:
a first electrode;
a second electrode; and
one or more organic material layers provided between the first electrode and the second electrode,
wherein one or more layers of the one or more organic material layers include the heterocyclic compound of claim 1.

7. The organic light emitting device of claim 6, wherein the organic material layer includes a hole transfer layer, and the hole transfer layer includes the heterocyclic compound.

8. The organic light emitting device of claim 6, wherein the organic material layer includes an electron blocking layer, and the electron blocking layer includes the heterocyclic compound.

9. The organic light emitting device of claim 6, further comprising one, two or more layers selected from the group consisting of a light emitting layer, a hole injection layer, a hole transfer layer, an electron injection layer, an electron transfer layer, an electron blocking layer and a hole blocking layer.
